(19) United States
(12) Patent Application Publication
(10) Pub. No.: US 2021/0002318 A1
(43) Pub. Date: Jan. 7, 2021

(54) HIGH-PURITY STEVIOL GLYCOSIDES

(71) Applicant: PureCircle USA Inc., Chicago, IL (US)

(72) Inventors: Avetik Markosyan, Yerevan (AM); Siew Yin Chow, Klang (MY); Khairul Nizam Bin Nawi, Nilai (MY); Kristina Chkhan, Moscow (RU); Mohamad Afzaal Bin Hasim, Kuala Lumpur (MY); Saravanan A/L Ramandach, Seremban (MY)

(73) Assignee: PureCircle USA Inc., Chicago, IL (US)

(21) Appl. No.: 16/981,684

(22) PCT Filed: Mar. 15, 2019
(86) PCT No.: PCT/US2019/022456

§ 371 Date: Sep. 16, 2020

Related U.S. Application Data
(60) Provisional application No. 62/644,065, filed on Mar. 16, 2018.
(60) Provisional application No. 62/644,407, filed on Mar. 17, 2018.
(60) Provisional application No. 62/682,461, filed on Jun. 8, 2018.

(30) Foreign Application Priority Data
Apr. 10, 2018 (US) PCT/US2018/026920

(51) Int. Cl. C07H 15/24; C07H 1/06; A23L 2/60; A23L 27/30

(52) U.S. Cl. CPC C07H 15/24; C07H 1/06; A23V 2002/00; A23L 27/36; A23L 2/60

(57) ABSTRACT

Methods of preparing highly purified steviol glycosides, particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 are described. The methods include utilizing enzyme preparations and recombinant microorganisms for converting various staring compositions to target steviol glycosides. The highly purified steviol glycosides are useful as non-caloric sweetener, flavor enhancer, sweetness enhancer, and foaming suppressor in edible and chewable compositions such as any beverages, confectioneries, bakery products, cookies, and chewing gums.

### TECHNICAL FIELD

The present invention relates to a process for preparing compositions comprising steviol glycosides, including highly purified steviol glycoside compositions.

### BACKGROUND OF THE INVENTION

High intensity sweeteners possess a sweetness level that is many times greater than the sweetness level of sucrose. They are essentially non-caloric and are commonly used in diet and reduced-calorie products, including foods and beverages. High intensity sweeteners do not elicit a glycemic response, making them suitable for use in products targeted to diabetics and others interested in controlling for their intake of carbohydrates.

Steviol glycosides are a class of compounds found in the leaves of Stevia rebaudiana Bertoni, a perennial shrub of the Asteraceae (Compositae) family native to certain regions of South America. They are characterized structurally by a single base, steviol, differing by the presence of carbohydrate residues at positions C13 and C19. They accumulate in Stevia leaves, composing approximately 10%-20% of the total dry weight. On a dry weight basis, the four major glycosides found in the leaves of Stevia typically include stevioside (9.1%), rebaudioside A (3.8%), rebaudioside C (0.6-1.0%) and dulcoside A (0.3%). Other known steviol glycosides include rebaudioside B, C, D, E, F and M, steviolbioside and rubusoside.

Although methods are known for preparing steviol glycosides from Stevia rebaudiana, many of these methods are unsuitable for use commercially.

Accordingly, there remains a need for simple, efficient, and economical methods for preparing compositions comprising steviol glycosides, including highly purified steviol glycoside compositions.

### SUMMARY OF THE INVENTION

As used herein, the abbreviation term “reb” refers to “rebaudioside”. Both terms have the same meaning and may be used interchangeably.

As used herein, “biocatalysis” or “biocatalytic” refers to the use of natural or genetically engineered biocatalysts, such as enzymes, or cells comprising one or more enzyme, capable of single or multiple step chemical transformations on organic compounds. Biocatalysis processes include fermentation, biosynthesis, bioconversion and biotransformation processes. Both isolated enzymes, and whole-cell biocatalysis methods are known in the art. Biocatalyst protein enzymes can be naturally occurring or recombinant proteins.

As used herein, the term “steviol glycoside(s)” refers to a glycoside of steviol, including, but not limited to, naturally occurring steviol glycosides, e.g. steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s, synthetic steviol glycosides, e.g. enzymatically glucosylated steviol glycosides and combinations thereof.

As used herein, the term “SvG7” refers to any naturally occurring steviol glycosides or any synthetic steviol glycosides, including enzymatically glucosylated steviol glycosides and combinations thereof, specifically a molecule comprising steviol having seven glucose residues attached covalently including, but not limited to reb 1a, reb 1b, reb 1c, reb 1d, reb 1e, reb 1f, reb 1g, reb 1h, reb 1i, reb 1j, reb 1k, reb 1l, reb 1m, reb 1n, reb 1o, reb 1p, reb 1q, reb 1r, reb 1s, reb 1t, reb 2a, reb 2b, reb 2c, reb 2d, reb 2e, reb 2f reb 2g, reb 2h, reb 2i, reb 2j, reb 2k, reb 2, reb 2m, reb 2n, reb 2o, reb 2p, reb 2q, reb 2r, and/or reb 2s. SvG7 can refer to a single steviol glycoside having seven glucose residues attached covalently or a mixture of steviol glycosides having seven glucose residues attached covalently.

The present invention provides a process for preparing a composition comprising a target steviol glycoside by contacting a starting composition comprising an organic substrate with a microbial cell and/or enzyme preparation, thereby producing a composition comprising a target steviol glycoside.

The starting composition can be any organic compound comprising at least one carbon atom. In one embodiment, the starting composition is selected from the group consisting of steviol glycosides, polyols or sugar alcohols, various carbohydrates.

The target steviol glycoside can be any steviol glycoside. In one embodiment, the target steviol glycoside is steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s, SvG7 or a synthetic steviol glycoside.

In one embodiment, the target steviol glycoside is rebaudioside 1a.

In one embodiment, the target steviol glycoside is rebaudioside 1b.

In one embodiment, the target steviol glycoside is rebaudioside 1c.

In one embodiment, the target steviol glycoside is rebaudioside 1d.

In one embodiment, the target steviol glycoside is rebaudioside 1e.

In one embodiment, the target steviol glycoside is rebaudioside 1f.

In one embodiment, the target steviol glycoside is rebaudioside 1g.

In one embodiment, the target steviol glycoside is rebaudioside 1h.

In one embodiment, the target steviol glycoside is rebaudioside 1i.

In one embodiment, the target steviol glycoside is rebaudioside 1j.

In one embodiment, the target steviol glycoside is rebaudioside 1k.

In one embodiment, the target steviol glycoside is rebaudioside 1l.

In one embodiment, the target steviol glycoside is rebaudioside 1m.

In one embodiment, the target steviol glycoside is rebaudioside 1n.

In one embodiment, the target steviol glycoside is rebaudioside 1o.

In one embodiment, the target steviol glycoside is rebaudioside 1p.

In one embodiment, the target steviol glycoside is rebaudioside 1q.

In one embodiment, the target steviol glycoside is rebaudioside 1r.

In one embodiment, the target steviol glycoside is rebaudioside 1s.

In one embodiment, the target steviol glycoside is rebaudioside 1t.

In one embodiment, the target steviol glycoside is rebaudioside 2a.

In one embodiment, the target steviol glycoside is rebaudioside 2b.

In one embodiment, the target steviol glycoside is rebaudioside 2c.

In one embodiment, the target steviol glycoside is rebaudioside 2d.

In one embodiment, the target steviol glycoside is rebaudioside 2e.

In one embodiment, the target steviol glycoside is rebaudioside 2f.

In one embodiment, the target steviol glycoside is rebaudioside 2g.

In one embodiment, the target steviol glycoside is rebaudioside 2h.

In one embodiment, the target steviol glycoside is rebaudioside 2i.

In one embodiment, the target steviol glycoside is rebaudioside 2j.

In one embodiment, the target steviol glycoside is rebaudioside 2k.

In one embodiment, the target steviol glycoside is rebaudioside 2l.

In one embodiment, the target steviol glycoside is rebaudioside 2m.

In one embodiment, the target steviol glycoside is rebaudioside 2n.

In one embodiment, the target steviol glycoside is rebaudioside 2o.

In one embodiment, the target steviol glycoside is rebaudioside 2p.

In one embodiment, the target steviol glycoside is rebaudioside 2q.

In one embodiment, the target steviol glycoside is rebaudioside 2r.

In one embodiment, the target steviol glycoside is rebaudioside 2s.

In one embodiment, the target steviol glycoside is rebaudioside M4.

In one embodiment, the target steviol glycoside is SvG7.

In some preferred embodiments enzyme preparation comprising one or more enzymes, or a microbial cell comprising one or more enzymes, capable of converting the starting composition to target steviol glycosides are used. The enzyme can be located on the surface and/or inside the cell. The enzyme preparation can be provided in the form of a whole cell suspension, a crude lysate or as purified enzyme(s). The enzyme preparation can be in free form or immobilized to a solid support made from inorganic or organic materials.

In some embodiments, a microbial cell comprises the necessary enzymes and genes encoding thereof for converting the starting composition to target steviol glycosides. Accordingly, the present invention also provides a process for preparing a composition comprising a target steviol glycoside by contacting a starting composition comprising an organic substrate with a microbial cell comprising at least one enzyme capable of converting the starting composition to target steviol glycosides, thereby producing a medium comprising at least one target steviol glycoside.

The enzymes necessary for converting the starting composition to target steviol glycosides include the steviol biosynthesis enzymes, NDP-glucosyltransferases (NGTs), ADP-glucosyltransferases (AGTs), CDP-glucosyltransferases (CGTs), GDP-glucosyltransferases (GGTs), TDP-glucosyltransferases (TDPs), UDP-glucosyltransferases (UGTs) and/or NDP-recycling enzyme, ADP-recycling enzyme, CDP-recycling enzyme, GDP-recycling enzyme, TDP-recycling enzyme, and/or UDP-recycling enzyme.

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

As used hereinafter, the term “SuSy_AT”, unless specified otherwise, refers to sucrose synthase having amino-acid sequence “SEQ ID 1” as described in Example 1, or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 1 polypeptide as well as isolated nucleic acid molecules that code for those polypeptides.

As used hereinafter, the term “UGTSl2”, unless specified otherwise, refers to UDP-glucosyltransferase having amino-acid sequence “SEQ ID 2” as described in Example 1 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 2 polypeptide as well as isolated nucleic acid molecules that code for those polypeptides.

As used hereinafter, the term “UGT76G1”, unless specified otherwise, refers to UDP-glucosyltransferase having amino-acid sequence “SEQ ID 3” as described in Example 1 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 3 polypeptide as well as isolated nucleic acid molecules that code for those polypeptides.

In one embodiment, steviol biosynthesis enzymes and UDP-glucosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases are synthesized.

In one embodiment, the UDP-glucosyltransferase is selected from group including UGT74G1, UGT85C2, UGT76G1, UGT91D2, UGTS2, EUGT11 and UGTs having substantial (>85%, >86%, >87%, >88%, >89%, >90%, >91%, >92%, >93%, >94%, >95%, >96%, >97%, >98%, >99%) amino-acid sequence identity to these polypeptides as well as isolated nucleic acid molecules that code for these UGTs.

In one embodiment, steviol biosynthesis enzymes, UGTs, and UDP-glucose recycling system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with UGT85C2.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with UGT74G1.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTSl2, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→4 glucopyranoside glycosidic linkage(s) at the newly formed glycosidic bond(s). In a particular embodiment, the UDP-glucosyltransferase is UGTSl2, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In another particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→6 glucopyranoside glycosidic linkage(s) at the newly formed glycosidic bond(s). In a particular embodiment, the UDP-glucosyltransferase is UGTSl2, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTSl2, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→4 glucopyranoside glycosidic linkage(s) at the newly formed glycosidic bond(s). In a particular embodiment, the UDP-glucosyltransferase is UGTS12, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In another particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→6 glucopyranoside glycosidic linkage(s) at the newly formed glycosidic bond(s). In a particular embodiment, the UDP-glucosyltransferase is UGTS12, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolmonoside to form steviolbioside. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside to form steviolbioside D. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with UGT74G1.

In one embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside A to form steviolbioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolbioside to form rebaudioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolbioside to form stevioside. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolbioside D to form rebaudioside B. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolbioside D to form rebaudioside G. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to rubusoside to form stevioside. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form rebaudioside G. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside A. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form stevioside B. In a particular embodiment, the UDP-glucosyltransferase is UGT76G1 or a UGT having >85% amino-acid sequence identity with UGT76G1.

In one embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolbioside A to form stevioside C. In a particular embodiment, the UDP-glucosyltransferase is UGT76G1 or a UGT having >85% amino-acid sequence identity with UGT76G1.

In one embodiment, the UDP-glucosyltransferase is any UDP-glucosyltransferase capable of adding at least one glucose unit to steviolbioside B to form stevioside B. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolbioside B to form stevioside C. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside B to form rebaudioside A. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to stevioside to form rebaudioside A. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside to form rebaudioside E. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside to form rebaudioside E2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside G to form rebaudioside A. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside G to form rebaudioside E4. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside G to form rebaudioside E6 In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside A to form rebaudioside E. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside A to form rebaudioside E4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside A to form rebaudioside E3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside B to form rebaudioside E2. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside B to form rebaudioside E6 In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside B to form rebaudioside E3. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to steviolbioside C to form rebaudioside E3. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to rebaudioside A to form rebaudioside D. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside A to form rebaudioside I. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E to form rebaudioside D. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E to form rebaudioside AM In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E2 to form rebaudioside I. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E2 to form rebaudioside AM In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E4 to form rebaudioside D. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E4 to form rebaudioside D7. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E6 to form rebaudioside I. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E6 to form rebaudioside D7. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E3 to form rebaudioside AM In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E3 to form rebaudioside D7. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside D to form rebaudioside M In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside I to form rebaudioside M In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside AM to form rebaudioside M In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside AM to form rebaudioside M4. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside D7 to form rebaudioside M In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside a. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1b. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1c. In a particular embodiment, the UDP-glucosyltransferase is UGTSl2 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1d. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1e. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1f. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1g. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1h. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1j. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1k. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1l. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1m. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1n. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside Jo. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1p. In a particular embodiment, the UDP-glucosyltransferase is UGTSl2 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1q. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside Jr. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1s. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1t. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2a. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2b. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2c. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2d. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2e. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2f. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2g. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2h. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2i. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2j. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2k. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2m. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2n. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2o. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2p. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2q. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2r. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2s. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 1q. In a particular embodiment, the UDP-glucosyltransferase is UGTSl2 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1. Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose.

In one embodiment, the recycling catalyst is sucrose synthase SuSy_At or a sucrose synthase having >85% amino-acid sequence identity with SuSy_At.

In one embodiment, the recycling substrate for UDP-glucose recycling catalyst is sucrose.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase, acid invertase, fructofuranosidase. In some embodiments, glucose and sugar(s) other than glucose, including but not limited to fructose, xylose, rhannose, arabinose, deoxyglucose, galactose are transferred to the recipient target steviol glycosides. In one embodiment, the recipient steviol glycoside is rebaudioside 1a, rebaudioside 1b, rebaudioside 1c, rebaudioside 1d rebaudioside 1e, rebaudioside 1f, rebaudioside 1g, rebaudioside 1h, rebaudioside 1i, rebaudioside 17, rebaudioside 1k, rebaudioside 1, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, and/or rebaudioside 2s. In another embodiment, the recipient steviol glycoside is rebaudioside M4. In another embodiment, the recipient steviol glycoside is SvG7.

Optionally, the method of the present invention further comprises separating the target steviol glycoside from the medium to provide a highly purified target steviol glycoside composition. The target steviol glycoside can be separated by at least one suitable method, such as, for example, crystallization, separation by membranes, centrifugation, extraction, chromatographic separation or a combination of such methods.

In one embodiment, the target steviol glycoside can be produced within the microorganism. In another embodiment, the target steviol glycoside can be secreted out in the medium. In one another embodiment, the released steviol glycoside can be continuously removed from the medium. In yet another embodiment, the target steviol glycoside is separated after the completion of the conversion reaction.

In one embodiment, separation produces a composition comprising greater than about 80% by weight of the target steviol glycoside on an anhydrous basis, i.e., a highly purified steviol glycoside composition. In another embodiment, separation produces a composition comprising greater than about 90% by weight of the target steviol glycoside. In particular embodiments, the composition comprises greater than about 95% by weight of the target steviol glycoside. In other embodiments, the composition comprises greater than about 99% by weight of the target steviol glycoside.

The target steviol glycoside can be in any polymorphic or amorphous form, including hydrates, solvates, anhydrous or combinations thereof.

Purified target steviol glycosides can be used in consumable products as a sweetener, flavor modifier, flavor with modifying properties and/or foaming suppressor. Suitable consumer products include, but are not limited to, food, beverages, pharmaceutical compositions, tobacco products, nutraceutical compositions, oral hygiene compositions, and cosmetic compositions.

### BRIEF DESCRIPTION OF THE DRAWINGS

FIG. 1a through FIG. 11 show the chemical structure of some SvG7 steviol glycosides rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s and rebaudioside 1t respectively.

FIG. 2a through FIG. 2s show the chemical structure of some SvG7 steviol glycosides rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r and rebaudioside 2s respectively.

FIG. 3 shows the chemical structure of rebaudioside M4.

FIG. 4a through FIG. 4s show the pathways of producing rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s and rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, and rebaudioside 2s, rebaudioside M4 and various steviol glycosides from steviol and the various intermediary steviol glycosides.

FIG. 5a through FIG. 5t show the biocatalytic production of rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m and rebaudioside 1n, rebaudioside 1o, rebaudioside 1p, rebaudioside 1q, rebaudioside 1r, rebaudioside 1s and rebaudioside 1t respectively, from rebaudioside A using the enzymes UGTS12 and UGT76G1 and concomitant recycling of UDP to UDP-glucose via sucrose synthase SuSy_At.

FIG. 6a through FIG. 6t show the biocatalytic production of rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and rebaudioside 1q, respectively, from stevioside using the enzymes UGTSl2 and UGT76G1 and concomitant recycling of UDP to UDP-glucose via sucrose synthase SuSy_At.

FIG. 7 shows the biocatalytic production of rebaudioside M4 from stevioside using the enzymes UGTSl2 and UGT76G1 and concomitant recycling of UDP to UDP-glucose via sucrose synthase SuSy_At.

FIG. 8a through FIG. 8t show the biocatalytic production of rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and rebaudioside 1q, respectively, from rebaudioside AM using the enzymes UGTSl2 and UGT76G1 and concomitant recycling of UDP to UDP-glucose via sucrose synthase SuSy_At.

FIG. 9 shows the biocatalytic production of rebaudioside M4 from rebaudioside AM using the enzymes UGTSl2 and UGT76G1 and concomitant recycling of UDP to UDP-glucose via sucrose synthase SuSy_At.

FIG. 10a through FIG. 1t show the biocatalytic production of rebaudioside 2a rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and rebaudioside 1q, respectively, from rebaudioside M4 using the enzymes UGTS12 and UGT76G1 and concomitant recycling of UDP to UDP-glucose via sucrose synthase SuSy_At.

FIG. 11a shows the HPLC chromatogram of stevioside. The peak with retention time of 20.958 minutes corresponds to stevioside. The peak with retention time 20.725 minutes corresponds to rebaudioside A. The peak at 32.925 minutes corresponds to rebaudioside B. The peak at 33.930 minutes corresponds to steviolbioside.

FIG. 11b shows the HPLC chromatogram of the product of the biocatalytic production of SvG7 molecules from stevioside. The peak at 5.089 minutes corresponds to rebaudioside 2m. The peak at 6.459 minutes corresponds to rebaudioside 2a. The peak at 9.825 minutes corresponds to rebaudioside AM. The peak at 13.845 minutes corresponds to rebaudioside M. The peak at 32.974 minutes corresponds to rebaudioside B. The peak at 33.979 minutes corresponds to steviolbioside.

FIG. 11c shows the HPLC chromatogram of rebaudioside 2a after purification by HPLC. The peak with retention time of 6.261 minutes correspond to rebaudioside 2a.

FIG. 11d shows the HPLC chromatogram of rebaudioside 2m after purification by HPLC. The peak with retention time of 5.197 minutes correspond to rebaudioside 2m.

FIG. 12a shows the 1H NMR spectrum of rebaudioside 2a (500 MHz, pyridine-d5).

FIG. 12b shows the HSQC spectrum of rebaudioside 2a (500 MHz, pyridine-d5).

FIG. 12c shows the H,H COSY spectrum of rebaudioside 2a (500 MHz, pyridine-d5).

FIG. 12d shows the HMBC spectrum of rebaudioside 2a (500 MHz, pyridine-d5).

FIG. 12e shows the HSQC-TOCSY spectrum of rebaudioside 2a (500 MHz, pyridine-d5).

FIG. 12f shows the 1D-NOESY spectrum of rebaudioside 2a (500 MHz, pyridine-d5).

FIG. 12g shows the LC chromatogram of rebaudioside 2a.

FIG. 12h shows the mass spectrum of rebaudioside 2a.

FIG. 13a shows the 1H NMR spectrum of rebaudioside 2m (500 MHz, pyridine-d5).

FIG. 13b shows the HSQC spectrum of rebaudioside 2m (500 MHz, pyridine-d5).

FIG. 13c shows the H,H COSY spectrum of rebaudioside 2m (500 MHz, pyridine-d5).

FIG. 13d shows the HMBC spectrum of rebaudioside 2m (500 MHz, pyridine-d5).

FIG. 13e shows the HSQC-TOCSY spectrum of rebaudioside 2m (500 MHz, pyridine-d5).

FIG. 13f shows the 1D-NOESY spectrum of rebaudioside 2m (500 MHz, pyridine-d5).

FIG. 13g shows the LC chromatogram of rebaudioside 2m.

FIG. 13h shows the mass spectrum of rebaudioside 2m.

### DETAILED DESCRIPTION

The present invention provides a process for preparing a composition comprising a target steviol glycoside by contacting a starting composition comprising an organic substrate with a microbial cell and/or enzyme preparation, thereby producing a composition comprising a target steviol glycoside.

One object of the invention is to provide an efficient biocatalytic method for preparing target steviol glycosides, particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s, and/or SvG7 or a synthetic steviol glycoside from various starting compositions.

### Starting Composition

As used herein, “starting composition” refers to any composition (generally an aqueous solution) containing one or more organic compound comprising at least one carbon atom.

In one embodiment, the starting composition is selected from the group consisting of steviol, steviol glycosides, polyols and various carbohydrates.

The starting composition steviol glycoside is selected from the group consisting of steviol, steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, and/or rebaudioside M4 or other glycoside of steviol occurring in Stevia rebaudiana plant, synthetic steviol glycosides, e.g. enzymatically glucosylated steviol glycosides and combinations thereof.

In one embodiment, the starting composition is steviol.

In another embodiment, the starting composition steviol glycoside is steviolmonoside.

In yet another embodiment, the starting composition steviol glycoside is steviolmonoside A.

In another embodiment, the starting composition steviol glycoside is steviolbioside.

In another embodiment, the starting composition steviol glycoside is steviolbioside D.

In another embodiment, the starting composition steviol glycoside is rubusoside.

In another embodiment, the starting composition steviol glycoside is rubusoside.

In another embodiment, the starting composition steviol glycoside is steviolbioside A.

In another embodiment, the starting composition steviol glycoside is steviolbioside B.

In another embodiment, the starting composition steviol glycoside is rebaudioside B.

In another embodiment, the starting composition steviol glycoside is stevioside.

In another embodiment, the starting composition steviol glycoside is rebaudioside G.

In another embodiment, the starting composition steviol glycoside is stevioside A.

In another embodiment, the starting composition steviol glycoside is stevioside B.

In another embodiment, the starting composition steviol glycoside is stevioside C.

In another embodiment, the starting composition steviol glycoside is rebaudioside A.

In another embodiment, the starting composition steviol glycoside is rebaudioside E.

In another embodiment, the starting composition steviol glycoside is rebaudioside E2.

In another embodiment, the starting composition steviol glycoside is rebaudioside E4.

In another embodiment, the starting composition steviol glycoside is rebaudioside E6.

In another embodiment, the starting composition steviol glycoside is rebaudioside E3.

In another embodiment, the starting composition steviol glycoside is rebaudioside D.

In another embodiment, the starting composition steviol glycoside is rebaudioside I.

In another embodiment, the starting composition steviol glycoside is rebaudioside AM In another embodiment, the starting composition steviol glycoside is rebaudioside D7.

In another embodiment, the starting composition steviol glycoside is rebaudioside M In another embodiment, the starting composition steviol glycoside is rebaudioside M4.

The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced.

The term “carbohydrate” refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

The starting composition may be synthetic or purified (partially or entirely), commercially available or prepared.

In one embodiment, the starting composition is glycerol.

In another embodiment, the starting composition is glucose.

In another embodiment, the starting composition is rhamnose.

In still another embodiment, the starting composition is sucrose.

In yet another embodiment, the starting composition is starch.

In another embodiment, the starting composition is maltodextrin.

In yet another embodiment, the starting composition is cellulose.

In still another embodiment, the starting composition is amylose.

The organic compound(s) of starting composition serve as a substrate(s) for the production of the target steviol glycoside(s), as described herein.

### Target Steviol Glycoside

The target steviol glycoside of the present method can be any steviol glycoside that can be prepared by the process disclosed herein. In one embodiment, the target steviol glycoside is selected from the group consisting of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s, SvG7 or other glycoside of steviol occurring in Stevia rebaudiana plant, synthetic steviol glycosides, e.g. enzymatically glucosylated steviol glycosides and combinations thereof.

In one embodiment, the target steviol glycoside is steviolmonoside.

In another embodiment, the target steviol glycoside is steviolmonoside A.

In another embodiment, the target steviol glycoside is steviolbioside.

In another embodiment, the target steviol glycoside is steviolbioside D.

In another embodiment, the target steviol glycoside is rubusoside.

In another embodiment, the target steviol glycoside is steviolbioside A.

In another embodiment, the target steviol glycoside is steviolbioside B.

In another embodiment, the target steviol glycoside is rebaudioside B.

In another embodiment, the target steviol glycoside is stevioside.

In another embodiment, the target steviol glycoside is rebaudioside G.

In another embodiment, the target steviol glycoside is stevioside A.

In another embodiment, the target steviol glycoside is stevioside B.

In another embodiment, the target steviol glycoside is stevioside C.

In another embodiment, the target steviol glycoside is rebaudioside A.

In another embodiment, the target steviol glycoside is rebaudioside E.

In another embodiment, the target steviol glycoside is rebaudioside E2.

In another embodiment, the target steviol glycoside is rebaudioside E4.

In another embodiment, the target steviol glycoside is rebaudioside E6.

In another embodiment, the target steviol glycoside is rebaudioside E3.

In another embodiment, the target steviol glycoside is rebaudioside D.

In another embodiment, the target steviol glycoside is rebaudioside I.

In another embodiment, the target steviol glycoside is rebaudioside AM.

In another embodiment, the target steviol glycoside is rebaudioside D7.

In another embodiment, the target steviol glycoside is rebaudioside M.

In another embodiment, the target steviol glycoside is rebaudioside M4.

In another embodiment, the target steviol glycoside is rebaudioside 1a.

In another embodiment, the target steviol glycoside is rebaudioside 1b.

In another embodiment, the target steviol glycoside is rebaudioside 1c.

In another embodiment, the target steviol glycoside is rebaudioside 1d.

In another embodiment, the target steviol glycoside is rebaudioside 1e.

In another embodiment, the target steviol glycoside is rebaudioside 1f.

In another embodiment, the target steviol glycoside is rebaudioside 1g.

In another embodiment, the target steviol glycoside is rebaudioside 1h.

In another embodiment, the target steviol glycoside is rebaudioside 1.

In another embodiment, the target steviol glycoside is rebaudioside 1j.

In another embodiment, the target steviol glycoside is rebaudioside 1k.

In another embodiment, the target steviol glycoside is rebaudioside 1l.

In another embodiment, the target steviol glycoside is rebaudioside 1m.

In another embodiment, the target steviol glycoside is rebaudioside 1n.

In another embodiment, the target steviol glycoside is rebaudioside Jo.

In another embodiment, the target steviol glycoside is rebaudioside 1p.

In another embodiment, the target steviol glycoside is rebaudioside 1q.

In another embodiment, the target steviol glycoside is rebaudioside Jr.

In another embodiment, the target steviol glycoside is rebaudioside 1s.

In another embodiment, the target steviol glycoside is rebaudioside 1t.

In another embodiment, the target steviol glycoside is rebaudioside 2a.

In another embodiment, the target steviol glycoside is rebaudioside 2b.

In another embodiment, the target steviol glycoside is rebaudioside 2c.

In another embodiment, the target steviol glycoside is rebaudioside 2d.

In another embodiment, the target steviol glycoside is rebaudioside 2e.

In another embodiment, the target steviol glycoside is rebaudioside 2f.

In another embodiment, the target steviol glycoside is rebaudioside 2g.

In another embodiment, the target steviol glycoside is rebaudioside 2h.

In another embodiment, the target steviol glycoside is rebaudioside 2i.

In another embodiment, the target steviol glycoside is rebaudioside 2j.

In another embodiment, the target steviol glycoside is rebaudioside 2k.

In another embodiment, the target steviol glycoside is rebaudioside 21.

In another embodiment, the target steviol glycoside is rebaudioside 2m.

In another embodiment, the target steviol glycoside is rebaudioside 2n.

In another embodiment, the target steviol glycoside is rebaudioside 2o.

In another embodiment, the target steviol glycoside is rebaudioside 2p.

In another embodiment, the target steviol glycoside is rebaudioside 2q.

In another embodiment, the target steviol glycoside is rebaudioside 2r.

In another embodiment, the target steviol glycoside is rebaudioside 2s.

In another embodiment, the target steviol glycoside is SvG7.

The target steviol glycoside can be in any polymorphic or amorphous form, including hydrates, solvates, anhydrous or combinations thereof.

In one embodiment, the present invention is a biocatalytic process for the production of steviolmonoside.

In one embodiment, the present invention is a biocatalytic process for the production of steviolmonoside A.

In one embodiment, the present invention is a biocatalytic process for the production of steviolbioside.

In one embodiment, the present invention is a biocatalytic process for the production of steviolbioside D.

In one embodiment, the present invention is a biocatalytic process for the production of rubusoside.

In one embodiment, the present invention is a biocatalytic process for the production of steviolbioside A.

In one embodiment, the present invention is a biocatalytic process for the production of steviolbioside B.

In one embodiment, the present invention is a biocatalytic process for the production of rebaudioside B.

In one embodiment, the present invention is a biocatalytic process for the production of stevioside.

In one embodiment, the present invention is a biocatalytic process for the production of rebaudioside G.

In one embodiment, the present invention is a biocatalytic process for the production of stevioside A.

In one embodiment, the present invention is a biocatalytic process for the production of stevioside B.

In one embodiment, the present invention is a biocatalytic process for the production of stevioside C.

In one embodiment, the present invention is a biocatalytic process for the production of rebaudioside A.

In one embodiment, the present invention is a biocatalytic process for the production of rebaudioside E.

In one embodiment, the present invention is a biocatalytic process for the production of rebaudioside E2.

In one embodiment, the present invention is a biocatalytic process for the production of rebaudioside E4.

In one embodiment, the present invention is a biocatalytic process for the production of rebaudioside E6 In one embodiment, the present invention is a biocatalytic process for the production of rebaudioside E3.

In one embodiment, the present invention is a biocatalytic process for the production of rebaudioside D.

In one embodiment, the present invention is a biocatalytic process for the production of rebaudioside 1 In one embodiment, the present invention is a biocatalytic process for the production of rebaudioside AM In one embodiment, the present invention is a biocatalytic process for the production of rebaudioside D7.

In one embodiment, the present invention is a biocatalytic process for the production of rebaudioside E3.

In one embodiment, the present invention is a biocatalytic process for the production of rebaudioside M In one embodiment, the present invention is a biocatalytic process for the production of rebaudioside M4.

In one embodiment, the present invention is a biocatalytic process for the production of rebaudioside 1a.

In one embodiment, the present invention is a biocatalytic process for the production of rebaudioside 1b.

In one embodiment, the present invention is a biocatalytic process for the production of rebaudioside 1c.

In one embodiment, the present invention is a biocatalytic process for the production of rebaudioside 1d.

In one embodiment, the present invention is a biocatalytic process for the production of rebaudioside 1e.

In one embodiment, the present invention is a biocatalytic process for the production of rebaudioside 1f.

In one embodiment, the present invention is a biocatalytic process for the production of rebaudioside 1g.

In one embodiment, the present invention is a biocatalytic process for the production of rebaudioside 1h.

In one embodiment, the present invention is a biocatalytic process for the production of rebaudioside 1i.

In one embodiment, the present invention is a biocatalytic process for the production of rebaudioside 1j.

In one embodiment, the present invention is a biocatalytic process for the production of rebaudioside 1k.

In one embodiment, the present invention is a biocatalytic process for the production of rebaudioside 1l.

In one embodiment, the present invention is a biocatalytic process for the production of rebaudioside 1m.

In one embodiment, the present invention is a biocatalytic process for the production of rebaudioside 1n.

In one embodiment, the present invention is a biocatalytic process for the production of rebaudioside 1o.

In one embodiment, the present invention is a biocatalytic process for the production of rebaudioside 1p.

In one embodiment, the present invention is a biocatalytic process for the production of rebaudioside 1q.

In one embodiment, the present invention is a biocatalytic process for the production of rebaudioside Jr.

In one embodiment, the present invention is a biocatalytic process for the production of rebaudioside 1s.

In one embodiment, the present invention is a biocatalytic process for the production of rebaudioside 1t.

In one embodiment, the present invention is a biocatalytic process for the production of rebaudioside 2a.

In one embodiment, the present invention is a biocatalytic process for the production of rebaudioside 2b.

In one embodiment, the present invention is a biocatalytic process for the production of rebaudioside 2c.

In one embodiment, the present invention is a biocatalytic process for the production of rebaudioside 2d.

In one embodiment, the present invention is a biocatalytic process for the production of rebaudioside 2e.

In one embodiment, the present invention is a biocatalytic process for the production of rebaudioside 2f.

In one embodiment, the present invention is a biocatalytic process for the production of rebaudioside 2g.

In one embodiment, the present invention is a biocatalytic process for the production of rebaudioside 2h.

In one embodiment, the present invention is a biocatalytic process for the production of rebaudioside 2i.

In one embodiment, the present invention is a biocatalytic process for the production of rebaudioside 2j.

In one embodiment, the present invention is a biocatalytic process for the production of rebaudioside 2k.

In one embodiment, the present invention is a biocatalytic process for the production of rebaudioside 2l.

In one embodiment, the present invention is a biocatalytic process for the production of rebaudioside 2m.

In one embodiment, the present invention is a biocatalytic process for the production of rebaudioside 2n.

In one embodiment, the present invention is a biocatalytic process for the production of rebaudioside 2o.

In one embodiment, the present invention is a biocatalytic process for the production of rebaudioside 2p.

In one embodiment, the present invention is a biocatalytic process for the production of rebaudioside 2q.

In one embodiment, the present invention is a biocatalytic process for the production of rebaudioside 2r.

In one embodiment, the present invention is a biocatalytic process for the production of rebaudioside 2s.

In one embodiment, the present invention is a biocatalytic process for the production of SvG7.

In a particular embodiment, the present invention provides for the biocatalytic process for the production of rebaudioside 1a from a starting composition comprising rebaudioside A and UDP-glucose.

In a particular embodiment, the present invention provides for the biocatalytic process for the production of rebaudioside 1b from a starting composition comprising rebaudioside A and UDP-glucose.

In a particular embodiment, the present invention provides for the biocatalytic process for the production of rebaudioside 1c from a starting composition comprising rebaudioside A and UDP-glucose.

In a particular embodiment, the present invention provides for the biocatalytic process for the production of rebaudioside 1d from a starting composition comprising rebaudioside A and UDP-glucose.

In a particular embodiment, the present invention provides for the biocatalytic process for the production of rebaudioside 1e from a starting composition comprising rebaudioside A and UDP-glucose.

In a particular embodiment, the present invention provides for the biocatalytic process for the production of rebaudioside 1f from a starting composition comprising rebaudioside A and UDP-glucose.

In a particular embodiment, the present invention provides for the biocatalytic process for the production of rebaudioside 1g from a starting composition comprising rebaudioside A and UDP-glucose.

In a particular embodiment, the present invention provides for the biocatalytic process for the production of rebaudioside 1h from a starting composition comprising rebaudioside A and UDP-glucose.

In a particular embodiment, the present invention provides for the biocatalytic process for the production of rebaudioside 1i from a starting composition comprising rebaudioside A and UDP-glucose.

In a particular embodiment, the present invention provides for the biocatalytic process for the production of rebaudioside 1j from a starting composition comprising rebaudioside A and UDP-glucose.

In a particular embodiment, the present invention provides for the biocatalytic process for the production of rebaudioside 1k from a starting composition comprising rebaudioside A and UDP-glucose.

In a particular embodiment, the present invention provides for the biocatalytic process for the production of rebaudioside 1l from a starting composition comprising rebaudioside A and UDP-glucose.

In a particular embodiment, the present invention provides for the biocatalytic process for the production of rebaudioside 1m from a starting composition comprising rebaudioside A and UDP-glucose.

In a particular embodiment, the present invention provides for the biocatalytic process for the production of rebaudioside 1n from a starting composition comprising rebaudioside A and UDP-glucose.

In a particular embodiment, the present invention provides for the biocatalytic process for the production of rebaudioside 1o from a starting composition comprising rebaudioside A and UDP-glucose.

In a particular embodiment, the present invention provides for the biocatalytic process for the production of rebaudioside 1p from a starting composition comprising rebaudioside A and UDP-glucose.

In a particular embodiment, the present invention provides for the biocatalytic process for the production of rebaudioside 1q from a starting composition comprising rebaudioside A and UDP-glucose.

In a particular embodiment, the present invention provides for the biocatalytic process for the production of rebaudioside 1r from a starting composition comprising rebaudioside A and UDP-glucose.

In a particular embodiment, the present invention provides for the biocatalytic process for the production of rebaudioside 1s from a starting composition comprising rebaudioside A and UDP-glucose.

In a particular embodiment, the present invention provides for the biocatalytic process for the production of rebaudioside 1t from a starting composition comprising rebaudioside A and UDP-glucose.

In a particular embodiment, the present invention provides for the biocatalytic process for the production of rebaudioside 2a from a starting composition comprising stevioside and UDP-glucose.

In a particular embodiment, the present invention provides for the biocatalytic process for the production of rebaudioside 2b from a starting composition comprising stevioside and UDP-glucose.

In a particular embodiment, the present invention provides for the biocatalytic process for the production of rebaudioside 2c from a starting composition comprising stevioside and UDP-glucose.

In a particular embodiment, the present invention provides for the biocatalytic process for the production of rebaudioside 2d from a starting composition comprising stevioside and UDP-glucose.

In a particular embodiment, the present invention provides for the biocatalytic process for the production of rebaudioside 2e from a starting composition comprising stevioside and UDP-glucose.

In a particular embodiment, the present invention provides for the biocatalytic process for the production of rebaudioside 2f from a starting composition comprising stevioside and UDP-glucose.

In a particular embodiment, the present invention provides for the biocatalytic process for the production of rebaudioside 2g from a starting composition comprising stevioside and UDP-glucose.

In a particular embodiment, the present invention provides for the biocatalytic process for the production of rebaudioside 2h from a starting composition comprising stevioside and UDP-glucose.

In a particular embodiment, the present invention provides for the biocatalytic process for the production of rebaudioside 2i from a starting composition comprising stevioside and UDP-glucose.

In a particular embodiment, the present invention provides for the biocatalytic process for the production of rebaudioside 2j from a starting composition comprising stevioside and UDP-glucose.

In a particular embodiment, the present invention provides for the biocatalytic process for the production of rebaudioside 2k from a starting composition comprising stevioside and UDP-glucose.

In a particular embodiment, the present invention provides for the biocatalytic process for the production of rebaudioside 2 from a starting composition comprising stevioside and UDP-glucose.

In a particular embodiment, the present invention provides for the biocatalytic process for the production of rebaudioside 2m from a starting composition comprising stevioside and UDP-glucose.

In a particular embodiment, the present invention provides for the biocatalytic process for the production of rebaudioside 2n from a starting composition comprising stevioside and UDP-glucose.

In a particular embodiment, the present invention provides for the biocatalytic process for the production of rebaudioside 2o from a starting composition comprising stevioside and UDP-glucose.

In a particular embodiment, the present invention provides for the biocatalytic process for the production of rebaudioside 2p from a starting composition comprising stevioside and UDP-glucose.

In a particular embodiment, the present invention provides for the biocatalytic process for the production of rebaudioside 2q from a starting composition comprising stevioside and UDP-glucose.

In a particular embodiment, the present invention provides for the biocatalytic process for the production of rebaudioside 2r from a starting composition comprising stevioside and UDP-glucose.

In a particular embodiment, the present invention provides for the biocatalytic process for the production of rebaudioside 2s from a starting composition comprising stevioside and UDP-glucose.

In a particular embodiment, the present invention provides for the biocatalytic process for the production of rebaudioside 1q from a starting composition comprising stevioside and UDP-glucose.

In a particular embodiment, the present invention provides for the biocatalytic process for the production of rebaudioside M4 from a starting composition comprising stevioside and UDP-glucose.

In a particular embodiment, the present invention provides for the biocatalytic process for the production of rebaudioside 2a from a starting composition comprising rebaudioside AM and UDP-glucose.

In a particular embodiment, the present invention provides for the biocatalytic process for the production of rebaudioside 2b from a starting composition comprising rebaudioside rebaudioside AM and UDP-glucose.

In a particular embodiment, the present invention provides for the biocatalytic process for the production of rebaudioside 2c from a starting composition comprising rebaudioside AM and UDP-glucose.

In a particular embodiment, the present invention provides for the biocatalytic process for the production of rebaudioside 2d from a starting composition comprising rebaudioside AM and UDP-glucose.

In a particular embodiment, the present invention provides for the biocatalytic process for the production of rebaudioside 2e from a starting composition comprising rebaudioside AM and UDP-glucose.

In a particular embodiment, the present invention provides for the biocatalytic process for the production of rebaudioside 2f from a starting composition comprising rebaudioside AM and UDP-glucose.

In a particular embodiment, the present invention provides for the biocatalytic process for the production of rebaudioside 2g from a starting composition comprising rebaudioside AM and UDP-glucose.

In a particular embodiment, the present invention provides for the biocatalytic process for the production of rebaudioside 2h from a starting composition comprising rebaudioside AM and UDP-glucose.

In a particular embodiment, the present invention provides for the biocatalytic process for the production of rebaudioside 2i from a starting composition comprising rebaudioside AM and UDP-glucose.

In a particular embodiment, the present invention provides for the biocatalytic process for the production of rebaudioside 2j from a starting composition comprising rebaudioside AM and UDP-glucose.

In a particular embodiment, the present invention provides for the biocatalytic process for the production of rebaudioside 2k from a starting composition comprising rebaudioside AM and UDP-glucose.

In a particular embodiment, the present invention provides for the biocatalytic process for the production of rebaudioside 2 from a starting composition comprising rebaudioside AM and UDP-glucose.

In a particular embodiment, the present invention provides for the biocatalytic process for the production of rebaudioside 2m from a starting composition comprising rebaudioside AM and UDP-glucose.

In a particular embodiment, the present invention provides for the biocatalytic process for the production of rebaudioside 2n from a starting composition comprising rebaudioside AM and UDP-glucose.

In a particular embodiment, the present invention provides for the biocatalytic process for the production of rebaudioside 2o from a starting composition comprising rebaudioside AM and UDP-glucose.

In a particular embodiment, the present invention provides for the biocatalytic process for the production of rebaudioside 2p from a starting composition comprising rebaudioside AM and UDP-glucose.

In a particular embodiment, the present invention provides for the biocatalytic process for the production of rebaudioside 2q from a starting composition comprising rebaudioside AM and UDP-glucose.

In a particular embodiment, the present invention provides for the biocatalytic process for the production of rebaudioside 2r from a starting composition comprising rebaudioside AM and UDP-glucose.

In a particular embodiment, the present invention provides for the biocatalytic process for the production of rebaudioside 2s from a starting composition comprising rebaudioside AM and UDP-glucose.

In a particular embodiment, the present invention provides for the biocatalytic process for the production of rebaudioside 1q from a starting composition comprising rebaudioside AM and UDP-glucose.

In a particular embodiment, the present invention provides for the biocatalytic process for the production of rebaudioside M4 from a starting composition comprising rebaudioside AM and UDP-glucose.

In a particular embodiment, the present invention provides for the biocatalytic process for the production of rebaudioside 2a from a starting composition comprising rebaudioside M4 and UDP-glucose.

In a particular embodiment, the present invention provides for the biocatalytic process for the production of rebaudioside 2b from a starting composition comprising rebaudioside M4 and UDP-glucose.

In a particular embodiment, the present invention provides for the biocatalytic process for the production of rebaudioside 2c from a starting composition comprising rebaudioside M4 and UDP-glucose.

In a particular embodiment, the present invention provides for the biocatalytic process for the production of rebaudioside 2d from a starting composition comprising rebaudioside M4 and UDP-glucose.

In a particular embodiment, the present invention provides for the biocatalytic process for the production of rebaudioside 2e from a starting composition comprising rebaudioside M4 and UDP-glucose.

In a particular embodiment, the present invention provides for the biocatalytic process for the production of rebaudioside 2f from a starting composition comprising rebaudioside M4 and UDP-glucose.

In a particular embodiment, the present invention provides for the biocatalytic process for the production of rebaudioside 2g from a starting composition comprising rebaudioside M4 and UDP-glucose.

In a particular embodiment, the present invention provides for the biocatalytic process for the production of rebaudioside 2h from a starting composition comprising rebaudioside M4 and UDP-glucose.

In a particular embodiment, the present invention provides for the biocatalytic process for the production of rebaudioside 2i from a starting composition comprising rebaudioside M4 and UDP-glucose.

In a particular embodiment, the present invention provides for the biocatalytic process for the production of rebaudioside 2j from a starting composition comprising rebaudioside M4 and UDP-glucose.

In a particular embodiment, the present invention provides for the biocatalytic process for the production of rebaudioside 2k from a starting composition comprising rebaudioside M4 and UDP-glucose.

In a particular embodiment, the present invention provides for the biocatalytic process for the production of rebaudioside 2 from a starting composition comprising rebaudioside M4 and UDP-glucose.

In a particular embodiment, the present invention provides for the biocatalytic process for the production of rebaudioside 2m from a starting composition comprising rebaudioside M4 and UDP-glucose.

In a particular embodiment, the present invention provides for the biocatalytic process for the production of rebaudioside 2n from a starting composition comprising rebaudioside M4 and UDP-glucose.

In a particular embodiment, the present invention provides for the biocatalytic process for the production of rebaudioside 2o from a starting composition comprising rebaudioside M4 and UDP-glucose.

In a particular embodiment, the present invention provides for the biocatalytic process for the production of rebaudioside 2p from a starting composition comprising rebaudioside M4 and UDP-glucose.

In a particular embodiment, the present invention provides for the biocatalytic process for the production of rebaudioside 2q from a starting composition comprising rebaudioside M4 and UDP-glucose.

In a particular embodiment, the present invention provides for the biocatalytic process for the production of rebaudioside 2r from a starting composition comprising rebaudioside M4 and UDP-glucose.

In a particular embodiment, the present invention provides for the biocatalytic process for the production of rebaudioside 2s from a starting composition comprising rebaudioside M4 and UDP-glucose.

In a particular embodiment, the present invention provides for the biocatalytic process for the production of rebaudioside 1q from a starting composition comprising rebaudioside M4 and UDP-glucose.

In a particular embodiment, the present invention provides for the biocatalytic process for the production of rebaudioside 1a from a starting composition comprising rebaudioside M and UDP-glucose.

In a particular embodiment, the present invention provides for the biocatalytic process for the production of rebaudioside 1b from a starting composition comprising rebaudioside M and UDP-glucose.

In a particular embodiment, the present invention provides for the biocatalytic process for the production of rebaudioside 1c from a starting composition comprising rebaudioside M and UDP-glucose.

In a particular embodiment, the present invention provides for the biocatalytic process for the production of rebaudioside 1d from a starting composition comprising rebaudioside M and UDP-glucose.

In a particular embodiment, the present invention provides for the biocatalytic process for the production of rebaudioside 1e from a starting composition comprising rebaudioside M and UDP-glucose.

In a particular embodiment, the present invention provides for the biocatalytic process for the production of rebaudioside 1f from a starting composition comprising rebaudioside M and UDP-glucose.

In a particular embodiment, the present invention provides for the biocatalytic process for the production of rebaudioside 1g from a starting composition comprising rebaudioside M and UDP-glucose.

In a particular embodiment, the present invention provides for the biocatalytic process for the production of rebaudioside 1h from a starting composition comprising rebaudioside M and UDP-glucose.

In a particular embodiment, the present invention provides for the biocatalytic process for the production of rebaudioside 1i from a starting composition comprising rebaudioside M and UDP-glucose.

In a particular embodiment, the present invention provides for the biocatalytic process for the production of rebaudioside 1j from a starting composition comprising rebaudioside M and UDP-glucose.

In a particular embodiment, the present invention provides for the biocatalytic process for the production of rebaudioside 1k from a starting composition comprising rebaudioside M and UDP-glucose.

In a particular embodiment, the present invention provides for the biocatalytic process for the production of rebaudioside 1l from a starting composition comprising rebaudioside M and UDP-glucose.

In a particular embodiment, the present invention provides for the biocatalytic process for the production of rebaudioside 1m from a starting composition comprising rebaudioside M and UDP-glucose.

In a particular embodiment, the present invention provides for the biocatalytic process for the production of rebaudioside 1n from a starting composition comprising rebaudioside M and UDP-glucose.

In a particular embodiment, the present invention provides for the biocatalytic process for the production of rebaudioside 1o from a starting composition comprising rebaudioside M and UDP-glucose.

In a particular embodiment, the present invention provides for the biocatalytic process for the production of rebaudioside 1p from a starting composition comprising rebaudioside M and UDP-glucose.

In a particular embodiment, the present invention provides for the biocatalytic process for the production of rebaudioside 1q from a starting composition comprising rebaudioside M and UDP-glucose.

In a particular embodiment, the present invention provides for the biocatalytic process for the production of rebaudioside 1r from a starting composition comprising rebaudioside M and UDP-glucose.

In a particular embodiment, the present invention provides for the biocatalytic process for the production of rebaudioside 1s from a starting composition comprising rebaudioside M and UDP-glucose.

In a particular embodiment, the present invention provides for the biocatalytic process for the production of rebaudioside 1t from a starting composition comprising rebaudioside M and UDP-glucose.

In a particular embodiment, the present invention provides for the biocatalytic process for the production of SvG7 from a starting composition comprising stevioside and UDP-glucose.

In a particular embodiment, the present invention provides for the biocatalytic process for the production of SvG7 from a starting composition comprising rebaudioside A and UDP-glucose.

In a particular embodiment, the present invention provides for the biocatalytic process for the production of SvG7 from a starting composition comprising stevioside, rebaudioside A and UDP-glucose.

In a particular embodiment, the present invention provides for the biocatalytic process for the production of SvG7 from a starting composition comprising rebaudioside AM and UDP-glucose.

In a particular embodiment, the present invention provides for the biocatalytic process for the production of SvG7 from a starting composition comprising rebaudioside M and UDP-glucose.

In a particular embodiment, the present invention provides for the biocatalytic process for the production of SvG7 from a starting composition comprising rebaudioside M4 and UDP-glucose.

Optionally, the method of the present invention further comprises separating the target steviol glycoside from the medium to provide a highly purified target steviol glycoside composition. The target steviol glycoside can be separated by any suitable method, such as, for example, crystallization, separation by membranes, centrifugation, extraction, chromatographic separation or a combination of such methods.

In particular embodiments, the process described herein results in a highly purified target steviol glycoside composition. The term “highly purified”, as used herein, refers to a composition having greater than about 80% by weight of the target steviol glycoside on an anhydrous (dried) basis. In one embodiment, the highly purified target steviol glycoside composition contains greater than about 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside M4, the process described herein provides a composition having greater than about 90% rebaudioside M4 content by weight on a dried basis. In another particular embodiment, when the target steviol glycoside is rebaudioside M4, the process described herein provides a composition comprising greater than about 95% content by weight on a dried basis.

In one embodiment, when the target steviol glycoside is rebaudioside a, the process described herein provides a composition having greater than about 90% rebaudioside 1a content by weight on a dried basis. In another particular embodiment, when the target steviol glycoside is rebaudioside a, the process described herein provides a composition comprising greater than about 95% content by weight on a dried basis.

In one embodiment, when the target steviol glycoside is rebaudioside 1b, the process described herein provides a composition having greater than about 90% rebaudioside 1b content by weight on a dried basis. In another particular embodiment, when the target steviol glycoside is rebaudioside 1b, the process described herein provides a composition comprising greater than about 95% content by weight on a dried basis.

In one embodiment, when the target steviol glycoside is rebaudioside 1c, the process described herein provides a composition having greater than about 90% rebaudioside 1c content by weight on a dried basis. In another particular embodiment, when the target steviol glycoside is rebaudioside 1c, the process described herein provides a composition comprising greater than about 95% content by weight on a dried basis.

In one embodiment, when the target steviol glycoside is rebaudioside 1d, the process described herein provides a composition having greater than about 90% rebaudioside 1d content by weight on a dried basis. In another particular embodiment, when the target steviol glycoside is rebaudioside 1d, the process described herein provides a composition comprising greater than about 95% content by weight on a dried basis.

In one embodiment, when the target steviol glycoside is rebaudioside 1e, the process described herein provides a composition having greater than about 90% rebaudioside 1e content by weight on a dried basis. In another particular embodiment, when the target steviol glycoside is rebaudioside 1e, the process described herein provides a composition comprising greater than about 95% content by weight on a dried basis.

In one embodiment, when the target steviol glycoside is rebaudioside 1f, the process described herein provides a composition having greater than about 90% rebaudioside 1f content by weight on a dried basis. In another particular embodiment, when the target steviol glycoside is rebaudioside 1f, the process described herein provides a composition comprising greater than about 95% content by weight on a dried basis.

In one embodiment, when the target steviol glycoside is rebaudioside 1g, the process described herein provides a composition having greater than about 90% rebaudioside 1g content by weight on a dried basis. In another particular embodiment, when the target steviol glycoside is rebaudioside 1g, the process described herein provides a composition comprising greater than about 95% content by weight on a dried basis.

In one embodiment, when the target steviol glycoside is rebaudioside 1h, the process described herein provides a composition having greater than about 90% rebaudioside 1h content by weight on a dried basis. In another particular embodiment, when the target steviol glycoside is rebaudioside 1h, the process described herein provides a composition comprising greater than about 95% content by weight on a dried basis.

In one embodiment, when the target steviol glycoside is rebaudioside 1i, the process described herein provides a composition having greater than about 90% rebaudioside 1i content by weight on a dried basis. In another particular embodiment, when the target steviol glycoside is rebaudioside 1i, the process described herein provides a composition comprising greater than about 95% content by weight on a dried basis.

In one embodiment, when the target steviol glycoside is rebaudioside 1j, the process described herein provides a composition having greater than about 90% rebaudioside 1j content by weight on a dried basis. In another particular embodiment, when the target steviol glycoside is rebaudioside 1j, the process described herein provides a composition comprising greater than about 95% content by weight on a dried basis.

In one embodiment, when the target steviol glycoside is rebaudioside 1k, the process described herein provides a composition having greater than about 90% rebaudioside 1k content by weight on a dried basis. In another particular embodiment, when the target steviol glycoside is rebaudioside 1k, the process described herein provides a composition comprising greater than about 95% content by weight on a dried basis.

In one embodiment, when the target steviol glycoside is rebaudioside 1l, the process described herein provides a composition having greater than about 90% rebaudioside 1l content by weight on a dried basis. In another particular embodiment, when the target steviol glycoside is rebaudioside 1l, the process described herein provides a composition comprising greater than about 95% content by weight on a dried basis.

In one embodiment, when the target steviol glycoside is rebaudioside 1m, the process described herein provides a composition having greater than about 90% rebaudioside 1m content by weight on a dried basis. In another particular embodiment, when the target steviol glycoside is rebaudioside 1m, the process described herein provides a composition comprising greater than about 95% content by weight on a dried basis.

In one embodiment, when the target steviol glycoside is rebaudioside 1n, the process described herein provides a composition having greater than about 90% rebaudioside 1n content by weight on a dried basis. In another particular embodiment, when the target steviol glycoside is rebaudioside 1n, the process described herein provides a composition comprising greater than about 95% content by weight on a dried basis.

In one embodiment, when the target steviol glycoside is rebaudioside 1o, the process described herein provides a composition having greater than about 90% rebaudioside 1o content by weight on a dried basis. In another particular embodiment, when the target steviol glycoside is rebaudioside 1o, the process described herein provides a composition comprising greater than about 95% content by weight on a dried basis.

In one embodiment, when the target steviol glycoside is rebaudioside 1p, the process described herein provides a composition having greater than about 90% rebaudioside 1p content by weight on a dried basis. In another particular embodiment, when the target steviol glycoside is rebaudioside 1p, the process described herein provides a composition comprising greater than about 95% content by weight on a dried basis. In one embodiment, when the target steviol glycoside is rebaudioside 1q, the process described herein provides a composition having greater than about 90% rebaudioside 1q content by weight on a dried basis. In another particular embodiment, when the target steviol glycoside is rebaudioside 1q, the process described herein provides a composition comprising greater than about 95% content by weight on a dried basis.

In one embodiment, when the target steviol glycoside is rebaudioside 1r, the process described herein provides a composition having greater than about 90% rebaudioside 1r content by weight on a dried basis. In another particular embodiment, when the target steviol glycoside is rebaudioside 1r, the process described herein provides a composition comprising greater than about 95% content by weight on a dried basis.

In one embodiment, when the target steviol glycoside is rebaudioside 1s, the process described herein provides a composition having greater than about 90% rebaudioside 1s content by weight on a dried basis. In another particular embodiment, when the target steviol glycoside is rebaudioside 1s, the process described herein provides a composition comprising greater than about 95% content by weight on a dried basis.

In one embodiment, when the target steviol glycoside is rebaudioside 1t, the process described herein provides a composition having greater than about 90% rebaudioside 1t content by weight on a dried basis. In another particular embodiment, when the target steviol glycoside is rebaudioside 1t, the process described herein provides a composition comprising greater than about 95% content by weight on a dried basis.

In one embodiment, when the target steviol glycoside is rebaudioside 2a, the process described herein provides a composition having greater than about 90% rebaudioside 2a content by weight on a dried basis. In another particular embodiment, when the target steviol glycoside is rebaudioside 2a, the process described herein provides a composition comprising greater than about 95% content by weight on a dried basis.

In one embodiment, when the target steviol glycoside is rebaudioside 2b, the process described herein provides a composition having greater than about 90% rebaudioside 2b content by weight on a dried basis. In another particular embodiment, when the target steviol glycoside is rebaudioside 2b, the process described herein provides a composition comprising greater than about 95% content by weight on a dried basis.

In one embodiment, when the target steviol glycoside is rebaudioside 2c, the process described herein provides a composition having greater than about 90% rebaudioside 2c content by weight on a dried basis. In another particular embodiment, when the target steviol glycoside is rebaudioside 2c, the process described herein provides a composition comprising greater than about 95% content by weight on a dried basis.

In one embodiment, when the target steviol glycoside is rebaudioside 2d, the process described herein provides a composition having greater than about 90% rebaudioside 2d content by weight on a dried basis. In another particular embodiment, when the target steviol glycoside is rebaudioside 2d, the process described herein provides a composition comprising greater than about 95% content by weight on a dried basis.

In one embodiment, when the target steviol glycoside is rebaudioside 2e, the process described herein provides a composition having greater than about 90% rebaudioside 2e content by weight on a dried basis. In another particular embodiment, when the target steviol glycoside is rebaudioside 2e, the process described herein provides a composition comprising greater than about 95% content by weight on a dried basis.

In one embodiment, when the target steviol glycoside is rebaudioside 2f the process described herein provides a composition having greater than about 90% rebaudioside 2f content by weight on a dried basis. In another particular embodiment, when the target steviol glycoside is rebaudioside 2f, the process described herein provides a composition comprising greater than about 95% content by weight on a dried basis.

In one embodiment, when the target steviol glycoside is rebaudioside 2g, the process described herein provides a composition having greater than about 90% rebaudioside 2g content by weight on a dried basis. In another particular embodiment, when the target steviol glycoside is rebaudioside 2g, the process described herein provides a composition comprising greater than about 95% content by weight on a dried basis.

In one embodiment, when the target steviol glycoside is rebaudioside 2h, the process described herein provides a composition having greater than about 90% rebaudioside 2h content by weight on a dried basis. In another particular embodiment, when the target steviol glycoside is rebaudioside 2h, the process described herein provides a composition comprising greater than about 95% content by weight on a dried basis.

In one embodiment, when the target steviol glycoside is rebaudioside 2i, the process described herein provides a composition having greater than about 90% rebaudioside 2i content by weight on a dried basis. In another particular embodiment, when the target steviol glycoside is rebaudioside 2i, the process described herein provides a composition comprising greater than about 95% content by weight on a dried basis.

In one embodiment, when the target steviol glycoside is rebaudioside 2j, the process described herein provides a composition having greater than about 90% rebaudioside 2j content by weight on a dried basis. In another particular embodiment, when the target steviol glycoside is rebaudioside 2j, the process described herein provides a composition comprising greater than about 95% content by weight on a dried basis.

In one embodiment, when the target steviol glycoside is rebaudioside 2k, the process described herein provides a composition having greater than about 90% rebaudioside 2k content by weight on a dried basis. In another particular embodiment, when the target steviol glycoside is rebaudioside 2k, the process described herein provides a composition comprising greater than about 95% content by weight on a dried basis.

In one embodiment, when the target steviol glycoside is rebaudioside 2l, the process described herein provides a composition having greater than about 90% rebaudioside 21 content by weight on a dried basis. In another particular embodiment, when the target steviol glycoside is rebaudioside 2, the process described herein provides a composition comprising greater than about 95% content by weight on a dried basis.

In one embodiment, when the target steviol glycoside is rebaudioside 2m, the process described herein provides a composition having greater than about 90% rebaudioside 2m content by weight on a dried basis. In another particular embodiment, when the target steviol glycoside is rebaudioside 2m, the process described herein provides a composition comprising greater than about 95% content by weight on a dried basis.

In one embodiment, when the target steviol glycoside is rebaudioside 2n, the process described herein provides a composition having greater than about 90% rebaudioside 2n content by weight on a dried basis. In another particular embodiment, when the target steviol glycoside is rebaudioside 2n, the process described herein provides a composition comprising greater than about 95% content by weight on a dried basis.

In one embodiment, when the target steviol glycoside is rebaudioside 2o, the process described herein provides a composition having greater than about 90% rebaudioside 2o content by weight on a dried basis. In another particular embodiment, when the target steviol glycoside is rebaudioside 2o, the process described herein provides a composition comprising greater than about 95% content by weight on a dried basis.

In one embodiment, when the target steviol glycoside is rebaudioside 2p, the process described herein provides a composition having greater than about 90% rebaudioside 2p content by weight on a dried basis. In another particular embodiment, when the target steviol glycoside is rebaudioside 2p, the process described herein provides a composition comprising greater than about 95% content by weight on a dried basis.

In one embodiment, when the target steviol glycoside is rebaudioside 2q, the process described herein provides a composition having greater than about 90% rebaudioside 2q content by weight on a dried basis. In another particular embodiment, when the target steviol glycoside is rebaudioside 2q, the process described herein provides a composition comprising greater than about 95% content by weight on a dried basis.

In one embodiment, when the target steviol glycoside is rebaudioside 2r, the process described herein provides a composition having greater than about 90% rebaudioside 2r content by weight on a dried basis. In another particular embodiment, when the target steviol glycoside is rebaudioside 2r, the process described herein provides a composition comprising greater than about 95% content by weight on a dried basis.

In one embodiment, when the target steviol glycoside is rebaudioside 2s, the process described herein provides a composition having greater than about 90% rebaudioside 2s content by weight on a dried basis. In another particular embodiment, when the target steviol glycoside is rebaudioside 2s, the process described herein provides a composition comprising greater than about 95% content by weight on a dried basis.

In one embodiment, when the target steviol glycoside is SvG7, the process described herein provides a composition having greater than about 90% SvG7 content by weight on a dried basis. In another particular embodiment, when the target steviol glycoside is SvG7, the process described herein provides a composition comprising greater than about 95% SvG7 content by weight on a dried basis.

### Microorganisms and Enzyme Preparations

In one embodiment of present invention, a microorganism (microbial cell) and/or enzyme preparation is contacted with a medium containing the starting composition to produce target steviol glycosides.

The enzyme can be provided in the form of a whole cell suspension, a crude lysate, a purified enzyme or a combination thereof. In one embodiment, the biocatalyst is a purified enzyme capable of converting the starting composition to the target steviol glycoside. In another embodiment, the biocatalyst is a crude lysate comprising at least one enzyme capable of converting the starting composition to the target steviol glycoside. In still another embodiment, the biocatalyst is a whole cell suspension comprising at least one enzyme capable of converting the starting composition to the target steviol glycoside.

In another embodiment, the biocatalyst is one or more microbial cells comprising enzyme(s) capable of converting the starting composition to the target steviol glycoside. The enzyme can be located on the surface of the cell, inside the cell or located both on the surface of the cell and inside the cell.

Suitable enzymes for converting the starting composition to target steviol glycosides include, but are not limited to, the steviol biosynthesis enzymes, NDP-glucosyltransferases (NGTs), ADP-glucosyltransferases (AGTs), CDP-glucosyltransferases (CGTs), GDP-glucosyltransferases (GGTs), TDP-glucosyltransferases (TDPs), UDP-glucosyltransferases (UGTs). Optionally it may include NDP-recycling enzyme(s), ADP-recycling enzyme(s), CDP-recycling enzyme(s), GDP-recycling enzyme(s), TDP-recycling enzyme(s), and/or UDP-recycling enzyme(s).

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

In one embodiment, steviol biosynthesis enzymes and UDP-glucosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases are synthesized.

In one embodiment, the UDP-glucosyltransferase is selected from group including UGT74G1, UGT85C2, UGT76G1, UGT91D2, UGTS2, EUGT11 and UGTs having substantial (>85%, >86%, >87%, >88%, >89%, >90%, >91%, >92%, >93%, >94%, >95%, >96%, >97%, >98%, >99%) amino-acid sequence identity to these polypeptides as well as isolated nucleic acid molecules that code for these UGTs.

In one embodiment, steviol biosynthesis enzymes, UGTs and UDP-glucose recycling system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with UGT85C2.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with UGT74G1.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTSl2, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→4 glucopyranoside glycosidic linkage(s) at the newly formed glycosidic bond(s). In a particular embodiment, the UDP-glucosyltransferase is UGTSl2, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In another particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1→6 glucopyranoside glycosidic linkage(s) at the newly formed glycosidic bond(s). In a particular embodiment, the UDP-glucosyltransferase is UGTSl2, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTSl2, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→4 glucopyranoside glycosidic linkage(s) at the newly formed glycosidic bond(s). In a particular embodiment, the UDP-glucosyltransferase is UGTSl2, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In another particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1→6 glucopyranoside glycosidic linkage(s) at the newly formed glycosidic bond(s). In a particular embodiment, the UDP-glucosyltransferase is UGTSl2, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolmonoside to form steviolbioside. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside to form steviolbioside D. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with UGT74G1.

In one embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside A to form steviolbioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolbioside to form rebaudioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolbioside to form stevioside. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolbioside D to form rebaudioside B. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolbioside D to form rebaudioside G. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to rubusoside to form stevioside. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form rebaudioside G. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside A. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form stevioside B. In a particular embodiment, the UDP-glucosyltransferase is UGT76G1 or a UGT having >85% amino-acid sequence identity with UGT76G1.

In one embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolbioside A to form stevioside C. In a particular embodiment, the UDP-glucosyltransferase is UGT76G1 or a UGT having >85% amino-acid sequence identity with UGT76G1.

In one embodiment, the UDP-glucosyltransferase is any UDP-glucosyltransferase capable of adding at least one glucose unit to steviolbioside B to form stevioside B. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolbioside B to form stevioside C. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside B to form rebaudioside A. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to stevioside to form rebaudioside A. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside to form rebaudioside E. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside to form rebaudioside E2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside G to form rebaudioside A. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside G to form rebaudioside E4. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside G to form rebaudioside E6 In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside A to form rebaudioside E. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside A to form rebaudioside E4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside A to form rebaudioside E3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside B to form rebaudioside E2. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside B to form rebaudioside E6. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside B to form rebaudioside E3. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to steviolbioside C to form rebaudioside E3. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to rebaudioside A to form rebaudioside D. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside A to form rebaudioside I. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E to form rebaudioside D. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E to form rebaudioside AM In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E2 to form rebaudioside I. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E2 to form rebaudioside AM In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E4 to form rebaudioside D. In a particular embodiment, the UDP-glucosyltransferase is UGTSl2 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E4 to form rebaudioside D7. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E6 to form rebaudioside I. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E6 to form rebaudioside D7. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E3 to form rebaudioside AM In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E3 to form rebaudioside D7. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside D to form rebaudioside M In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside I to form rebaudioside M In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside AM to form rebaudioside M In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside AM to form rebaudioside M4. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside D7 to form rebaudioside M In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside a. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1b. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1c. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1d. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1e. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1f. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1g. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1h. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1i. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1j. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1k. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1l. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1m. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1n. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M to form rebaudioside 1o. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1p. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1q. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1r. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1s. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M to form rebaudioside 1t. In a particular embodiment, the UDP-glucosyltransferase is UGTSl2 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2a. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2b. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2c. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2d. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2e. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2f. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2g. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2h. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2i. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2j. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2k. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2l. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2m. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2n. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside M4 to form rebaudioside 2o. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2p. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2q. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2r. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 2s. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside M4 to form rebaudioside 1q. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2. In a particular embodiment, the UDP-glucosyltransferase is UGT76G1 or a UGT having >85% amino-acid sequence identity with UGT76G1.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose.

In one embodiment, the recycling catalyst is sucrose synthase SuSy_At or a sucrose synthase having >85% amino-acid sequence identity with SuSy_At.

In one embodiment, the recycling substrate for UDP-glucose recycling catalyst is sucrose.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase, acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, and/or rebaudioside 2s. In another embodiment, the recipient steviol glycoside is rebaudioside M4. In another embodiment, the recipient steviol glycoside is SvG7.

In another embodiment, the UDP-glucosyltransferase capable of adding at least one glucose unit to starting composition steviol glycoside has >85% amino-acid sequence identity with UGTs selected from the following listing of GenInfo identifier numbers, preferably from the group presented in Table 1, and Table 2.

| | | | | | |
|---|---|---|---|---|---|
| 397567 | 30680413 | 115480946 | 147798902 | 218193594 | 225443294 |
| 454245 | 32816174 | 116310259 | 147811764 | 218193942 | 225444853 |
| 1359905 | 32816178 | 116310985 | 147827151 | 219885307 | 225449296 |
| 1685003 | 34393978 | 116788066 | 147836230 | 222615927 | 225449700 |
| 1685005 | 37993665 | 116788606 | 147839909 | 222619587 | 225454338 |
| 2191136 | 37993671 | 116789315 | 147846163 | 222623142 | 225454340 |
| 2501497 | 37993675 | 119394507 | 147855977 | 222625633 | 225454342 |
| 2911049 | 39104603 | 119640480 | 148905778 | 222625635 | 225454473 |
| 4218003 | 41469414 | 122209731 | 148905999 | 222636620 | 225454475 |
| 4314356 | 41469452 | 125526997 | 148906835 | 222636621 | 225458362 |
| 13492674 | 42566366 | 125534279 | 148907340 | 222636628 | 225461551 |
| 13492676 | 42570280 | 125534461 | 148908935 | 222636629 | 225461556 |
| 15217773 | 42572855 | 125540090 | 148909182 | 224053242 | 225461558 |
| 15217796 | 44890129 | 125541516 | 148909920 | 224053386 | 225469538 |
| 15223396 | 46806235 | 125545408 | 148910082 | 224055535 | 225469540 |
| 15223589 | 50284482 | 125547340 | 148910154 | 224056138 | 226316457 |
| 15227766 | 51090402 | 125547520 | 148910612 | 224056160 | 226492603 |
| 15230017 | 51090594 | 125554547 | 148910769 | 224067918 | 226494221 |
| 15231757 | 52839682 | 125557592 | 156138791 | 224072747 | 226495389 |
| 15234056 | 56550539 | 125557593 | 156138797 | 224080189 | 226495945 |
| 15234195 | 62734263 | 125557608 | 156138799 | 224091845 | 226502400 |
| 15234196 | 62857204 | 125559566 | 156138803 | 224094703 | 226507980 |
| 15238503 | 62857206 | 125563266 | 165972256 | 224100653 | 226531147 |
| 15239523 | 62857210 | 125571055 | 168016721 | 224100657 | 226532094 |
| 15239525 | 62857212 | 125579728 | 171674071 | 224101569 | 238477377 |
| 15239543 | 75265643 | 125588307 | 171906258 | 224103105 | 240254512 |
| 15239937 | 75285934 | 125589492 | 183013901 | 224103633 | 242032615 |
| 15240305 | 75288884 | 125599469 | 183013903 | 224103637 | 242032621 |
| 15240534 | 77550661 | 125601477 | 186478321 | 224109218 | 242038423 |
| 15982889 | 77556148 | 126635837 | 187373030 | 224114583 | 242043290 |
| 18086351 | 82791223 | 126635845 | 187373042 | 224116284 | 242044836 |
| 18418378 | 83778990 | 126635847 | 190692175 | 224120552 | 242051252 |
| 18418380 | 89953335 | 126635863 | 194701936 | 224121288 | 242056217 |
| 18418382 | 110741436 | 126635867 | 195620060 | 224121296 | 242056219 |
| 19743740 | 110743955 | 126635883 | 209954691 | 224121300 | 242056663 |
| 19911201 | 115438196 | 126635887 | 209954719 | 224130358 | 242059339 |
| 20149064 | 115438785 | 133874210 | 209954725 | 224140703 | 242059341 |
| 20260654 | 115441237 | 133874212 | 209954733 | 224143404 | 242060922 |
| 21435782 | 115454819 | 145358033 | 210063105 | 224143406 | 242067411 |
| 21553613 | 115456047 | 147772508 | 210063107 | 224144306 | 242067413 |
| 21593514 | 115457492 | 147776893 | 212275846 | 224285244 | 242076258 |
| 22759895 | 115459312 | 147776894 | 216296854 | 225431707 | 242076396 |
| 23955910 | 115464719 | 147776895 | 217074506 | 225435532 | 242084750 |
| 26452040 | 115471069 | 147786916 | 218185693 | 225436321 | 242091005 |
| 28393204 | 115471071 | 147798900 | 218187075 | 225440041 | 242095206 |
| 30679796 | 115474009 | 147798901 | 218189427 | 225441116 | 242345159 |
| 242345161 | 297724601 | 326492035 | 356523945 | 357140904 | 359486938 |
| 255536859 | 297725463 | 326493430 | 356523957 | 357165849 | 359487055 |
| 255538228 | 297728331 | 326500410 | 356523959 | 357165852 | 359488135 |
| 255541676 | 297738632 | 326506816 | 356523961 | 357168415 | 359488708 |
| 255547075 | 297745347 | 326507826 | 356523963 | 357437837 | 359493630 |
| 255552620 | 297745348 | 326508394 | 356524387 | 357442755 | 359493632 |
| 255552622 | 297795735 | 326509445 | 356524403 | 357442757 | 359493634 |
| 255555343 | 297796253 | 326511261 | 356527181 | 357445729 | 359493636 |
| 255555361 | 297796257 | 326511866 | 356533209 | 357445731 | 359493815 |
| 255555363 | 297796261 | 326512412 | 356533852 | 357445733 | 359495856 |
| 255555365 | 297797587 | 326517673 | 356534718 | 357446799 | 359495858 |
| 255555369 | 297798502 | 326518800 | 356535480 | 357446805 | 359495869 |
| 255555373 | 297799226 | 326521124 | 356542996 | 357452779 | 359495871 |
| 255555377 | 297805988 | 326525567 | 356543136 | 357452781 | 359497638 |
| 255556812 | 297807499 | 326525957 | 356543932 | 357452783 | 359807261 |
| 255556818 | 297809125 | 326526607 | 356549841 | 357452787 | 374256637 |
| 255563008 | 297809127 | 326527141 | 356549843 | 357452789 | 377655465 |
| 255564074 | 297811403 | 326530093 | 356554358 | 357452791 | 378405177 |
| 255564531 | 297820040 | 326534036 | 356554360 | 357452797 | 378829085 |
| 255572878 | 297821483 | 326534312 | 356558606 | 357452799 | 387135070 |
| 255577901 | 297825217 | 332071132 | 356560333 | 357470367 | 387135072 |
| 255583249 | 297832276 | 339715876 | 356560599 | 357472193 | 387135078 |
| 255583253 | 297832280 | 342306012 | 356560749 | 357472195 | 387135092 |
| 255583255 | 297832518 | 342306016 | 356566018 | 357474295 | 387135094 |
| 255585664 | 297832520 | 343457675 | 356566169 | 357474493 | 387135098 |
| 255585666 | 297840825 | 343457677 | 356566173 | 357474497 | 387135100 |
| 255634688 | 297840827 | 350534960 | 356567761 | 357474499 | 387135134 |
| 255644801 | 297847402 | 356498085 | 356574704 | 357490035 | 387135136 |
| 255645821 | 297849372 | 356499771 | 356576401 | 357493567 | 387135174 |
| 255647456 | 300078590 | 356499777 | 356577660 | 357497139 | 387135176 |
| 255648275 | 300669727 | 356499779 | 357114993 | 357497581 | 387135184 |
| 260279126 | 302142947 | 356501328 | 357115447 | 357497671 | 387135186 |
| 260279128 | 302142948 | 356502523 | 357115451 | 357500579 | 387135188 |
| 261343326 | 302142950 | 356503180 | 357115453 | 357504663 | 387135190 |
| 283132367 | 302142951 | 356503184 | 357116080 | 357504691 | 387135192 |
| 283362112 | 302765302 | 356503295 | 357116928 | 357504699 | 387135194 |
| 289188052 | 302796334 | 356504436 | 357117461 | 357504707 | 387135282 |
| 295841350 | 302811470 | 356504523 | 357117463 | 357505859 | 387135284 |
| 296088529 | 302821107 | 356504765 | 357117829 | 357510851 | 387135294 |
| 296090415 | 302821679 | 356511113 | 357117839 | 357516975 | 387135298 |
| 296090524 | 319759260 | 356515120 | 357125059 | 359477003 | 387135300 |
| 296090526 | 319759266 | 356517088 | 357126015 | 359477998 | 387135302 |
| 297599503 | 320148814 | 356520732 | 357134488 | 359478043 | 387135304 |
| 297601531 | 326489963 | 356522586 | 357135657 | 359478286 | 387135312 |
| 297611791 | 326490273 | 356522588 | 357138503 | 359484299 | 387135314 |
| 297722841 | 326491131 | 356522590 | 357139683 | 359486936 | 387135316 |
| 387135318 | 449440433 | 460376293 | 460413408 | 462423864 | 475546199 |
| 387135320 | 449445896 | 460378310 | 460416351 | 470101924 | 475556485 |
| 387135322 | 449446454 | 460380744 | 462394387 | 470102280 | 475559699 |
| 387135324 | 449447657 | 460381726 | 462394433 | 470102858 | 475578293 |
| 387135326 | 449449002 | 460382093 | 462394557 | 470104211 | 475591753 |
| 387135328 | 449449004 | 460382095 | 462395646 | 470104264 | 475593742 |
| 388493506 | 449449006 | 460382754 | 462395678 | 470104266 | 475612072 |
| 388495496 | 449451379 | 460384935 | 462396388 | 470106317 | 475622476 |
| 388498446 | 449451589 | 460384937 | 462396389 | 470106357 | 475622507 |
| 388499220 | 449451591 | 460385076 | 462396419 | 470115448 | 475623787 |
| 388502176 | 449451593 | 460385872 | 462396542 | 470130404 | 482550481 |
| 388517521 | 449453712 | 460386018 | 462397507 | 470131550 | 482550499 |
| 388519407 | 449453714 | 460389217 | 462399998 | 470136482 | 482550740 |
| 388521413 | 449453716 | 460394872 | 462400798 | 470136484 | 482550999 |
| 388827901 | 449453732 | 460396139 | 462401217 | 470136488 | 482552352 |
| 388827903 | 449457075 | 460397862 | 462402118 | 470136492 | 482554970 |
| 388827907 | 449467555 | 460397864 | 462402237 | 470137933 | 482555336 |
| 388827909 | 449468742 | 460398541 | 462402284 | 470137937 | 482555478 |
| 388827913 | 449495638 | 460403139 | 462402416 | 470140422 | 482556454 |
| 393887637 | 449495736 | 460403141 | 462404228 | 470140426 | 482557289 |
| 393887646 | 449499880 | 460403143 | 462406358 | 470140908 | 482558462 |
| 393887649 | 449502786 | 460403145 | 462408262 | 470141232 | 482558508 |
| 393990627 | 449503471 | 460405998 | 462409325 | 470142008 | 482558547 |
| 397746860 | 449503473 | 460407578 | 462409359 | 470142010 | 482561055 |
| 397789318 | 449515857 | 460407590 | 462409777 | 470142012 | 482561555 |
| 413924864 | 449518643 | 460409128 | 462411467 | 470143607 | 482562795 |
| 414590349 | 449519559 | 460409134 | 462414311 | 470143939 | 482562850 |
| 414590661 | 449522783 | 460409136 | 462414416 | 470145404 | 482565074 |
| 414591157 | 449524530 | 460409459 | 462414476 | 473923244 | 482566269 |
| 414879558 | 449524591 | 460409461 | 462415526 | 474114354 | 482566296 |
| 414879559 | 449528823 | 460409463 | 462415603 | 474143634 | 482566307 |
| 414879560 | 449528825 | 460409465 | 462415731 | 474202268 | 482568689 |
| 414888074 | 449534021 | 460409467 | 462416307 | 474299266 | 482570049 |
| 431812559 | 460365546 | 460410124 | 462416920 | 474363119 | 482570572 |
| 449432064 | 460366882 | 460410126 | 462416922 | 474366157 | 482575121 |
| 449432066 | 460369823 | 460410128 | 462416923 | 474429346 | |
| 449433069 | 460369829 | 460410130 | 462416924 | 475432777 | |
| 449436944 | 460369831 | 460410132 | 462417401 | 475473002 | |
| 449438665 | 460369833 | 460410134 | 462419769 | 475489790 | |
| 449438667 | 460370755 | 460410213 | 462420317 | 475511330 | |
| 449440431 | 460374714 | 460411200 | 462423366 | 475516200 | |

TABLE 1

| GI number | Accession | Origin |
|---|---|---|
| 190692175 | ACE87855.1 | Stevia rebaudiana |
| 41469452 | AAS07253.1 | Oryza sativa |
| 62857204 | BAD95881.1 | Ipomoea nil |
| 62857206 | BAD95882.1 | Ipomoea purperea |
| 56550539 | BAD77944.1 | Bellis perennis |
| 115454819 | NP_001051010.1 | Oryza sativa Japonica Group |
| 115459312 | NP_001053256.1 | Oryza sativa Japonica Group |
| 115471069 | NP_001059133.1 | Oryza sativa Japonica Group |
| 115471071 | NP_001059134.1 | Oryza sativa Japonica Group |
| 116310985 | CAH67920.1 | Oryza sativa Indica Group |
| 116788066 | ABK24743.1 | Picea sitchensis |
| 122209731 | Q2V6J9.1 | Fragaria x ananassa |
| 125534461 | EAY81009.1 | Oryza sativa Indica Group |
| 125559566 | EAZ05102.1 | Oryza sativa Indica Group |
| 125588307 | EAZ28971.1 | Oryza sativa Japonica Group |
| 148907340 | ABR16806.1 | Picea sitchensis |
| 148910082 | ABR18123.1 | Picea sitchensis |
| 148910612 | ABR18376.1 | Picea sitchensis |
| 15234195 | NP_194486.1 | Arabidopsis thaliana |
| 15239523 | NP_200210.1 | Arabidopsis thaliana |
| 15239937 | NP_196793.1 | Arabidopsis thaliana |
| 1685005 | AAB36653.1 | Nicotiana tabacum |
| 183013903 | ACC38471.1 | Medicago truncatula |
| 186478321 | NP_172511.3 | Arabidopsis thaliana |
| 187373030 | ACD03249.1 | Avena strigosa |
| 194701936 | ACF85052.1 | Zea mays |
| 19743740 | AAL92461.1 | Solanum lycopersicum |
| 212275846 | NP_001131009.1 | Zea mays |
| 222619587 | EEE55719.1 | Oryza sativa Japonica Group |
| 224055535 | XP_002298527.1 | Populus trichocarpa |
| 224101569 | XP_002334266.1 | Populus trichocarpa |
| 224120552 | XP_002318358.1 | Populus trichocarpa |
| 224121288 | XP_002330790.1 | Populus trichocarpa |
| 225444853 | XP_002281094 | Vitis vinifera |
| 225454342 | XP_002275850.1 | Vitis vinifera |
| 225454475 | XP_002280923.1 | Vitis vinifera |
| 225461556 | XP_002285222 | Vitis vinifera |
| 225469540 | XP_002270294.1 | Vitis vinifera |
| 226495389 | NP_001148083.1 | Zea mays |
| 226502400 | NP_001147674.1 | Zea mays |
| 238477377 | ACR43489.1 | Triticum aestivum |
| 240254512 | NP_565540.4 | Arabidopsis thaliana |
| 2501497 | Q43716.1 | Petunia x hybrida |
| 255555369 | XP_002518721.1 | Ricinus communis |
| 26452040 | BAC43110.1 | Arabidopsis thaliana |
| 296088529 | CBI37520.3 | Vitis vinifera |
| 297611791 | NP_001067852.2 | Oryza sativa Japonica Group |
| 297795735 | XP_002865752.1 | Arabidopsis lyrata subsp. lyrata |
| 297798502 | XP_002867135.1 | Arabidopsis lyrata subsp. lyrata |
| 297820040 | XP_002877903.1 | Arabidopsis lyrata subsp. lyrata |
| 297832276 | XP_002884020.1 | Arabidopsis lyrata subsp. lyrata |
| 302821107 | XP_002992218.1 | Selaginella moellendorffii |
| 30680413 | NP_179446.2 | Arabidopsis thaliana |
| 319759266 | ADV71369.1 | Pueraria montana var. lobata |
| 326507826 | BAJ86656.1 | Hordeum vulgare subsp. Vulgare |
| 343457675 | AEM37036.1 | Brassica rapa subsp. oleifera |
| 350534960 | NP_001234680.1 | Solanum lycopersicum |
| 356501328 | XP_003519477.1 | Glycine max |
| 356522586 | XP_003529927.1 | Glycine max |
| 356535480 | XP_003536273.1 | Glycine max |
| 357445733 | XP_003593144.1 | Medicago truncatula |
| 357452783 | XP_003596668.1 | Medicago truncatula |
| 357474493 | XP_003607531.1 | Medicago truncatula |
| 357500579 | XP_003620578.1 | Medicago truncatula |
| 357504691 | XP_003622634.1 | Medicago truncatula |
| 359477998 | XP_003632051.1 | Vitis vinifera |
| 359487055 | XP_002271587 | Vitis vinifera |
| 359495869 | XP_003635104.1 | Vitis vinifera |
| 387135134 | AFJ52948.1 | Linum usitatissimum |
| 387135176 | AFJ52969.1 | Linum usitatissimum |
| 387135192 | AFJ52977.1 | Linum usitatissimum |
| 387135282 | AFJ53022.1 | Linum usitatissimum |
| 387135302 | AFJ53032.1 | Linum usitatissimum |
| 387135312 | AFJ53037.1 | Linum usitatissimum |
| 388519407 | AFK47765.1 | Medicago truncatula |
| 393887646 | AFN26668.1 | Barbarea vulgaris subsp. arcuata |
| 414888074 | DAA64088.1 | Zea mays |
| 42572855 | NP_974524.1 | Arabidopsis thaliana |
| 449440433 | XP_004137989.1 | Cucumis sativus |
| 449446454 | XP_004140986.1 | Cucumis sativus |
| 449449004 | XP_004142255.1 | Cucumis sativus |
| 449451593 | XP_004143546.1 | Cucumis sativus |
| 449515857 | XP_004164964.1 | Cucumis sativus |
| 460382095 | XP_004236775.1 | Solanum lycopersicum |
| 460409128 | XP_004249992.1 | Solanum lycopersicum |
| 460409461 | XP_004250157.1 | Solanum lycopersicum |
| 460409465 | XP_004250159.1 | Solanum lycopersicum |
| 462396388 | EMJ02187.1 | Prunus persica |
| 462402118 | EMJ07675.1 | Prunus persica |
| 462409359 | EMJ14693.1 | Prunus persica |
| 462416923 | EMJ21660.1 | Prunus persica |
| 46806235 | BAD17459.1 | Oryza sativa Japonica Group |
| 470104266 | XP_004288529.1 | Fragaria vesca subsp. vesca |
| 470142008 | XP_004306714.1 | Fragaria vesca subsp. vesca |
| 475432777 | EMT01232.1 | Aegilops tauschb |
| 51090402 | BAD35324.1 | Oryza sativa Japonica Group |

TABLE 2

| GI number | Accession | Origin | Internal reference |
|---|---|---|---|
| 460409128 | XP.004249992.1 | Solanum lycopersicum | UGTSl |
| 460386018 | XP.004238697.1 | Solanum lycopersicum | — |
| 460409134 | XP.004249995.1 | Solanum lycopersicum | — |
| 460410132 | XP.004250485.1 | Solanum lycopersicum | UGTSl2 |
| 460410130 | XP.004250484.1 | Solanum lycopersicum | — |
| 460410128 | XP.004250483.1 | Solanum lycopersicum | — |
| 460378310 | XP.004234916.1 | Solanum lycopersicum | — |
| 209954733 | BAG80557.1 | Lycium barbarum | UGTLB |
| 209954725 | BAG80553.1 | Lycium barbarum | — |

One embodiment of the present invention is a microbial cell comprising an enzyme, i.e. an enzyme capable of converting the starting composition to the target steviol glycoside. Accordingly, some embodiments of the present method include contacting a microorganism with a medium containing the starting composition to provide a medium comprising at least one target steviol glycoside.

The microorganism can be any microorganism possessing the necessary enzyme(s) for converting the starting composition to target steviol glycoside(s). These enzymes are encoded within the microorganism's genome.

Suitable microorganisms include, but are not limited to, E. coli, Saccharomyces sp., Aspergillus sp., Pichia sp., Bacillus sp., Yarrowia sp. etc.

In one embodiment, the microorganism is free when contacted with the starting composition.

In another embodiment, the microorganism is immobilized when contacted with the starting composition. For example, the microorganism may be immobilized to a solid support made from inorganic or organic materials. Non-limiting examples of solid supports suitable to immobilize the microorganism include derivatized cellulose or glass, ceramics, metal oxides or membranes. The microorganism may be immobilized to the solid support, for example, by covalent attachment, adsorption, cross-linking, entrapment or encapsulation.

In still another embodiment, the enzyme capable of converting the starting composition to the target steviol glycoside is secreted out of the microorganism and into the reaction medium.

The target steviol glycoside is optionally purified. Purification of the target steviol glycoside from the reaction medium can be achieved by at least one suitable method to provide a highly purified target steviol glycoside composition. Suitable methods include crystallization, separation by membranes, centrifugation, extraction (liquid or solid phase), chromatographic separation, HPLC (preparative or analytical) or a combination of such methods.

### Uses

Highly purified target glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and/or SvG7 obtained according to this invention can be used “as-is” or in combination with other sweeteners, flavors, food ingredients and combinations thereof.

Non-limiting examples of flavors include, but are not limited to, lime, lemon, orange, fruit, banana, grape, pear, pineapple, mango, berry, bitter almond, cola, cinnamon, sugar, cotton candy, vanilla and combinations thereof.

Non-limiting examples of other food ingredients include, but are not limited to, acidulants, organic and amino acids, coloring agents, bulking agents, modified starches, gums, texturizers, preservatives, caffeine, antioxidants, emulsifiers, stabilizers, thickeners, gelling agents and combinations thereof.

Highly purified target glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and/or SvG7 obtained according to this invention can be prepared in various polymorphic forms, including but not limited to hydrates, solvates, anhydrous, amorphous forms and combinations thereof.

Highly purified target glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and/or SvG7 obtained according to this invention may be incorporated as a high intensity natural sweetener in foodstuffs, beverages, pharmaceutical compositions, cosmetics, chewing gums, table top products, cereals, dairy products, toothpastes and other oral cavity compositions, etc.

In some embodiments, the highly purified target glycoside(s) of present invention are present in foodstuffs, beverages, pharmaceutical compositions, cosmetics, chewing gums, table top products, cereals, dairy products, toothpastes and other oral cavity compositions, etc in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

In a particular embodiment, the sweetener is present in the beverage in an amount from about 0.0001% by weight to about 8% by weight, such as for example, from about 0.0001% by weight to about 0.0005% by weight, from about 0.0005% by weight to about 0.001% by weight, from about 0.001% by weight to about 0.005% by weight, from about 0.005% by weight to about 0.01% by weight, from about 0.01% by weight to about 0.05% by weight, from about 0.05% by weight to about 0.1% by weight, from about 0.1% by weight to about 0.5% by weight, from about 0.5% by weight to about 1% by weight, from about 1% by weight to about 2% by weight, from about 2% by weight to about 3% by weight, from about 3% by weight to about 4% by weight, from about 4% by weight to about 5% by weight, from about 5% by weight to about 6% by weight, from about 6% by weight to about 7% by weight, and from about 7% by weight to about 8% by weight.

Highly purified target glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s SvG7 and/or combinations thereof, obtained according to this invention, may be employed as a sweetening compound, or it may be used together with at least one naturally occurring high intensity sweeteners such as dulcoside A, dulcoside B, dulcoside C, dulcoside D, rebaudioside A2, rebaudioside A3, rebaudioside A4, rebaudioside B2, rebaudioside C, rebaudioside C2, rebaudioside C3, rebaudioside C4, rebaudioside C5, rebaudioside C6, rebaudioside D2, rebaudioside D3, rebaudioside D4, rebaudioside D5, rebaudioside D6, rebaudioside D8, rebaudioside E5, rebaudioside E7, rebaudioside F, rebaudioside F2, rebaudioside F3, rebaudioside H, rebaudioside H2, rebaudioside H3, rebaudioside H4, rebaudioside H5, rebaudioside H6, rebaudioside 12, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside V3, rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G, stevioside H, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, camosiflosides, cyclocarioside, pterocaryosides, polypodoside A, brazilin, hemandulcin, phillodulcin, glycyphyllin, phlorizin, trilobatin, dihydroflavonol, dihydroquercetin-3-acetate, neoastilibin, trans-cinnamaldehyde, monatin, monatin salts, other indole derivative sweeteners, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, mogroside V, siamenoside, siratose and combinations thereof.

In a particular embodiment, steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and/or SvG7 can be used in a sweetener composition comprising a compound selected from the group consisting of dulcoside A, dulcoside B, dulcoside C, dulcoside D, rebaudioside A2, rebaudioside A3, rebaudioside A4, rebaudioside B2, rebaudioside C, rebaudioside C2, rebaudioside C3, rebaudioside C4, rebaudioside C5, rebaudioside C6, rebaudioside D2, rebaudioside D3, rebaudioside D4, rebaudioside D5, rebaudioside D6, rebaudioside D8, rebaudioside E5, rebaudioside E7, rebaudioside F, rebaudioside F2, rebaudioside F3, rebaudioside H, rebaudioside H2, rebaudioside H3, rebaudioside H4, rebaudioside H5, rebaudioside H6, rebaudioside 12, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside V3, rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G, stevioside H, NSF-02, Mogroside V, siratose, Luo Han Guo, allulose, allose, D-tagatose, erythritol and combinations thereof.

Highly purified target glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and/or SvG7 may also be used in combination with synthetic high intensity sweeteners such as sucralose, potassium acesulfame, aspartame, alitame, saccharin, neohesperidin dihydrochalcone, cyclamate, neotame, dulcin, suosan advantame, salts thereof, and combinations thereof.

Moreover, highly purified target steviol glycoside(s) particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 can be used in combination with natural sweetener suppressors such as gymnemic acid, hodulcin, ziziphin, lactisole, and others.

Steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 may also be combined with various umami taste enhancers. Steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and/or SvG7 can be mixed with umami tasting and sweet amino acids such as glutamate, aspartic acid, glycine, alanine, threonine, proline, serine, glutamate, lysine, tryptophan and combinations thereof.

Highly purified target steviol glycoside(s) particularly, steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 can be used in combination with one or more additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof.

Highly purified target steviol glycoside(s) particularly, steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 may be combined with polyols or sugar alcohols. The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced which do not adversely affect the taste of the sweetener composition.

Highly purified target steviol glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 may be combined with reduced calorie sweeteners such as, for example, D-tagatose, L-sugars, L-sorbose, L-arabinose and combinations thereof.

Highly purified target steviol glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 may also be combined with various carbohydrates. The term “carbohydrate” generally refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, psicose, turanose, allose, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

Highly purified target steviol glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 obtained according to this invention can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols); polyols; prebiotics, probiotics; phytoestrogens; soy protein; sulfides/thiols; amino acids; proteins; vitamins; and minerals. Functional ingredients also may be classified based on their health benefits, such as cardiovascular, cholesterol-reducing, and anti-inflammatory. Exemplary functional ingredients are provided in WO2013/096420, the contents of which is hereby incorporated by reference.

Highly purified target steviol glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 obtained according to this invention may be applied as a high intensity sweetener to produce zero calorie, reduced calorie or diabetic beverages and food products with improved taste characteristics. It may also be used in drinks, foodstuffs, pharmaceuticals, and other products in which sugar cannot be used. In addition, highly purified target steviol glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 can be used as a sweetener not only for drinks, foodstuffs, and other products dedicated for human consumption, but also in animal feed and fodder with improved characteristics.

Highly purified target steviol glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 obtained according to this invention may be applied as a foaming suppressor to produce zero calorie, reduced calorie or diabetic beverages and food products.

Examples of consumable products in which highly purified target steviol glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and/or SvG7 may be used as a sweetening compound include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others. In principle it can have unlimited applications.

Examples of consumable products in which highly purified target steviol glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and/or SvG7 may be used as a flavor modifier or flavor with modifying properties include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others. In principle it can have unlimited applications.

During the manufacturing of products such as foodstuffs, drinks, pharmaceuticals, cosmetics, table top products, and chewing gum, the conventional methods such as mixing, kneading, dissolution, pickling, permeation, percolation, sprinkling, atomizing, infusing and other methods may be used.

Moreover, the highly purified target steviol glycoside(s) steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 obtained in this invention may be used in dry or liquid forms.

The highly purified target steviol glycoside can be added before or after heat treatment of food products. The amount of the highly purified target steviol glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 depends on the purpose of usage. As discussed above, it can be added alone or in combination with other compounds.

The present invention is also directed to sweetness enhancement in beverages using steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and/or SvG7 as a sweetness enhancer, wherein steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2, rebaudioside 2m, rebaudioside 2n, rebaudioside 2o, rebaudioside 2p, rebaudioside 2q, rebaudioside 2r, rebaudioside 2s and/or SvG7 is present in a concentration at or below their respective sweetness recognition thresholds.

As used herein, the term “sweetness enhancer” refers to a compound capable of enhancing or intensifying the perception of sweet taste in a composition, such as a beverage. The term “sweetness enhancer” is synonymous with the terms “sweet taste potentiator,” “sweetness potentiator,” “sweetness amplifier,” and “sweetness intensifier.”

The term “sweetness recognition threshold concentration,” as generally used herein, is the lowest known concentration of a sweet compound that is perceivable by the human sense of taste, typically around 1.0% sucrose equivalence (1.0% SE). Generally, the sweetness enhancers may enhance or potentiate the sweet taste of sweeteners without providing any noticeable sweet taste by themselves when present at or below the sweetness recognition threshold concentration of a given sweetness enhancer; however, the sweetness enhancers may themselves provide sweet taste at concentrations above their sweetness recognition threshold concentration. The sweetness recognition threshold concentration is specific for a particular enhancer and can vary based on the beverage matrix. The sweetness recognition threshold concentration can be easily determined by taste testing increasing concentrations of a given enhancer until greater than 1.0% sucrose equivalence in a given beverage matrix is detected. The concentration that provides about 1.0% sucrose equivalence is considered the sweetness recognition threshold.

In some embodiments, sweetener is present in the beverage in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

In a particular embodiment, the sweetener is present in the beverage in an amount from about 0.0001% by weight to about 10% by weight, such as for example, from about 0.0001% by weight to about 0.0005% by weight, from about 0.0005% by weight to about 0.001% by weight, from about 0.001% by weight to about 0.005% by weight, from about 0.005% by weight to about 0.01% by weight, from about 0.01% by weight to about 0.05% by weight, from about 0.05% by weight to about 0.1% by weight, from about 0.1% by weight to about 0.5% by weight, from about 0.5% by weight to about 1% by weight, from about 1% by weight to about 2% by weight, from about 2% by weight to about 3% by weight, from about 3% by weight to about 4% by weight, from about 4% by weight to about 5% by weight, from about 5% by weight to about 6% by weight, from about 6% by weight to about 7% by weight, from about 7% by weight to about 8% by weight, from about 8% by weight to about 9% by weight, or from about 9% by weight to about 10% by weight. In a particular embodiment, the sweetener is present in the beverage in an amount from about 0.5% by weight to about 10% by weight. In another particular embodiment, the sweetener is present in the beverage in an amount from about 2% by weight to about 8% by weight.

In one embodiment, the sweetener is a traditional caloric sweetener. Suitable sweeteners include, but are not limited to, sucrose, fructose, glucose, high fructose corn syrup and high fructose starch syrup.

In another embodiment, the sweetener is erythritol.

In still another embodiment, the sweetener is a rare sugar. Suitable rare sugars include, but are not limited to, D-allose, D-psicose, D-ribose, D-tagatose, L-glucose, L-fucose, L-arabinose, D-turanose, D-leucrose and combinations thereof.

It is contemplated that a sweetener can be used alone, or in combination with other sweeteners.

In one embodiment, the rare sugar is D-allose. In a more particular embodiment, D-allose is present in the beverage in an amount of about 0.5% to about 10% by weight, such as, for example, from about 2% to about 8%.

In another embodiment, the rare sugar is D-psicose. In a more particular embodiment, D-psicose is present in the beverage in an amount of about 0.5% to about 10% by weight, such as, for example, from about 2% to about 8%.

In still another embodiment, the rare sugar is D-ribose. In a more particular embodiment, D-ribose is present in the beverage in an amount of about 0.5% to about 10% by weight, such as, for example, from about 2% to about 8%.

In yet another embodiment, the rare sugar is D-tagatose. In a more particular embodiment, D-tagatose is present in the beverage in an amount of about 0.5% to about 10% by weight, such as, for example, from about 2% to about 8%.

In a further embodiment, the rare sugar is L-glucose. In a more particular embodiment, L-glucose is present in the beverage in an amount of about 0.5% to about 10% by weight, such as, for example, from about 2% to about 8%.

In one embodiment, the rare sugar is L-fucose. In a more particular embodiment, L-fucose is present in the beverage in an amount of about 0.5% to about 10% by weight, such as, for example, from about 2% to about 8%.

In another embodiment, the rare sugar is L-arabinose. In a more particular embodiment, L-arabinose is present in the beverage in an amount of about 0.5% to about 10% by weight, such as, for example, from about 2% to about 8%.

In yet another embodiment, the rare sugar is D-turanose. In a more particular embodiment, D-turanose is present in the beverage in an amount of about 0.5% to about 10% by weight, such as, for example, from about 2% to about 8%.

In yet another embodiment, the rare sugar is D-leucrose. In a more particular embodiment, D-leucrose is present in the beverage in an amount of about 0.5% to about 10% by weight, such as, for example, from about 2% to about 8%.

The addition of the sweetness enhancer at a concentration at or below its sweetness recognition threshold increases the detected sucrose equivalence of the beverage comprising the sweetener and the sweetness enhancer compared to a corresponding beverage in the absence of the sweetness enhancer. Moreover, sweetness can be increased by an amount more than the detectable sweetness of a solution containing the same concentration of the at least one sweetness enhancer in the absence of any sweetener.

Accordingly, the present invention also provides a method for enhancing the sweetness of a beverage comprising a sweetener comprising providing a beverage comprising a sweetener and adding a sweetness enhancer selected from steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 or a combination thereof, wherein steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 are present in a concentration at or below their sweetness recognition thresholds.

Addition of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7 in a concentration at or below the sweetness recognition threshold to a beverage containing a sweetener may increase the detected sucrose equivalence from about 1.0% to about 5.0%, such as, for example, about 1.0%, about 1.5%, about 2.0%, about 2.5%, about 3.0%, about 3.5%, about 4.0%, about 4.5% or about 5.0%.

The following examples illustrate preferred embodiments of the invention for the preparation of highly purified target steviol glycoside(s), particularly steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s and/or SvG7. It will be understood that the invention is not limited to the materials, proportions, conditions and procedures set forth in the examples, which are only illustrative.

### EXAMPLES

### Example 1

### Protein Sequences of Engineered Enzymes Used in the Biocatalytic Process

SEQ ID 1:
>SuSy_At, variant PM1-54-2-E05 (engineered sucrose
synthase; source of WT gene: Arabidopsis thaliana)
MANAERMITRVHSQRERLNETLVSERNEVLALLSRVEAKGKGILQQNQII
AEFEALPEQTRKKLEGGPFFDLLKSTQEAIVLPPWVALAVRPRPGVWEYL
RVNLHALVVEELQPAEFLHFKEELVDGVKNGNFTLELDFEPFNASIPRPT
LHKYIGNGVDFLNRHLSAKLFHDKESLLPLLDFLRLHSHQGKNLMLSEKI
QNLNTLQHTLRKAEEYLAELKSETLYEEFEAKFEEIGLERGWGDNAERVL
DMIRLLLDLLEAPDPSTLETFLGRVPMVFNVVILSPHGYFAQDNVLGYPD
TGGQVVYILDQVRALEIEMLQRIKQQGLNIKPRILILTRLLPDAVGTTCG
ERLERVYDSEYCDILRVPFRTEKGIVRKWISRFEVWPYLETYTEDAAVEL
SKELNGKPDLIIGNYSDGNLVASLLAHKLGVTQCTIAHALEKTKYPDSDI
YWKKLDDKYHFSCQFTADIFAMNHTDFIITSTFQEIAGSKETVGQYESHT
AFTLPGLYRVVHGIDVFDPKFNIVSPGADMSIYFPYTEEKRRLTKFHSEI
EELLYSDVENDEHLCVLKDKKKPILFTMARLDRVKNLSGLVEWYGKNTRL
RELVNLVVVGGDRRKESKDNEEKAEMKKMYDLIEEYKLNGQFRWISSQMD
RVRNGELYRYICDTKGAFVQPALYEAFGLTVVEAMTCGLPTFATCKGGPA
EIIVHGKSGFHIDPYHGDQAADLLADFFTKCKEDPSHWDEISKGGLQRIE
EKYTWQIYSQRLLTLTGVYGFWKHVSNLDRLEHRRYLEMFYALKYRPLAQ
AVPLAQDD
SEQ ID 2:
>UGTS12 variant 0234 (engineered
glucosyltransferase; source of WT gene: Solanum
lycopersicum)
MATNLRVLMFPWLAYGHISPFLNIAKQLADRGFLIYLCSTRINLESIIKK
IPEKYADSIHLIELQLPELPELPPHYHTTNGLPPHLNPTLHKALKMSKPN
FSRILQNLKPDLLIYDVLQPWAEHVANEQGIPAGKLLVSCAAVFSYFFSF
RKNPGVEFPFPAIHLPEVEKVKIREILAKEPEEGGRLDEGNKQMMLMCTS
RTIEAKYIDYCTELCNWKVVPVGPPFQDLITNDADNKELIDWLGTKPENS
TVFVSFGSEYFLSKEDMEEIAFALEASNVNFIWVVRFPKGEERNLEDALP
EGFLERIGERGRVLDKFAPQPRILNHPSTGGFISHCGWNSVMESIDFGVP
IIAMPIHNDQPINAKLMVELGVAVEIVRDDDGKIHRGEIAEALKSVVTGE
TGEILRAKVREISKNLKSIRDEEMDAVAEELIQLCRNSNKSK
SEQ ID 3:
>UGT76G1 variant 0042 (engineered
glucosyltransferase; source of WT gene: Stevia
rebaudiana)
MENKTETTVRRRRRIILFPVPFQGHINPILQLANVLYSKGFAITILHTNF
NKPKTSNYPHFTFRFILDNDPQDERISNLPTHGPLAGMRIPIINEHGADE
LRRELELLMLASEEDEEVSCLITDALWYFAQDVADSLNLRRLVLMTSSLF
NFHAHVSLPQFDELGYLDPDDKTRLEEQASGFPMLKVKDIKSAYSNWQIG
KEILGKMIKQTKASSGVIWNSFKELEESELETVIREIPAPSFLIPLPKHL
TASSSSLLDHDRTVFEWLDQQAPSSVLYVSFGSTSEVDEKDFLEIARGLV
DSGQSFLWVVRPGFVKGSTWVEPLPDGFLGERGKIVKWVPQQEVLAHPAI
GAFWTHSGWNSTLESVCEGVPMIFSSFGGDQPLNARYMSDVLRVGVYLEN
GWERGEVVNAIRRVMVDEEGEYIRQNARVLKQKADVSLMKGGSSYESLES
LVSYISSL

### Example 2

### Expression and Formulation of SuSy_At Variant of SEQ ID 1

The gene coding for the SuSy_At variant of SEQ ID 1 (EXAMPLE 1) was cloned into the expression vector pLETA17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/l) at 37° C. Expression of the genes was induced at logarithmic phase by IPTG (0.2 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with distilled water, resulting in an enzymatic active preparation.

For enzymatic active preparations of SuSy_At, activity in Units is defined as follows: 1 mU of SuSy_At turns over 1 nmol of sucrose into fructose in 1 minute. Reaction conditions for the assay are 30° C., 50 mM potassium phosphate buffer pH 7.0, 400 mM sucrose at to, 3 mM MgCl₂, and 15 mM uridine diphosphate (UDP).

### Example 3

Expression and formulation of UGTS12 variant of SEQ ID 2 The gene coding for the UGTS12 variant of SEQ ID 2 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/l) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGTSl2, activity in Units is defined as follows: 1 mU of UGTSl2 turns over 1 nmol of rebaudioside A (Reb A) into rebaudioside D (Reb D) in 1 minute. Reaction conditions for the assay are 30° C., 50 mM potassium phosphate buffer pH 7.0, 10 mM Reb A at to, 500 mM sucrose, 3 mM MgCl₂, 0.25 mM uridine diphosphate (UDP) and 3 U/mL of SuSy_At.

### Example 4

### Expression and Formulation of UGT76G1 Variant of SEQ ID 3

The gene coding for the UGT76G1 variant of SEQ ID 3 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/l) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGT76G1, activity in Units is defined as follows: 1 mU of UGT76G1 turns over 1 nmol of rebaudioside D (Reb D) into rebaudioside M (Reb M) in 1 minute. Reaction conditions for the assay are 30° C., 50 mM potassium phosphate buffer pH 7.0, 10 mM Reb D at to, 500 mM sucrose, 3 mM MgCl₂, 0.25 mM uridine diphosphate (UDP) and 3 U/mL of SuSy_At.

### Example 5

### Synthesis of SvG7 in a One-Pot Reaction, Adding UGTS12, SuSy_At and UGT76G1 at the Same Time.

Various SvG7 molecules were synthesized directly from stevioside (see FIG. 11a) in a one-pot reaction, utilizing the three enzymes (see EXAMPLES 1, 2, 3 and 4): UGTSl2 (variant of SEQ ID 2), SuSy_At (variant of SEQ ID 1) and UGT76G1 (variant of SEQ ID 3).

The final reaction solution contained 348 U/L UGTS2, 1341 U/L SuSy_At, 10 U/L UGT76G1, 47 mM stevioside, 0.32 mM uridine diphosphate (UDP), 0.99 M sucrose, 3.9 mM MgCl₂ and potassium phosphate buffer (pH 6.6). First, 206 mL of distilled water were mixed with 0.24 gMgCl₂.6H₂O, 102 g sucrose, 9.8 mL of 1.5 M potassium phosphate buffer (pH 6.6) and 15 g stevioside. The final volume of the reaction mixture was adjusted to 300 mL.

After dissolving the components, the temperature was adjusted to 45° C. and UGTS12, SuSy_At, UGT76G1 and 39 mg UDP were added. The reaction mixture was incubated at 45° C. shaker for 24 hrs. Additional 39 mg UDP was added at 12 hours, 24 hours, and 36 hours. The content of reb 2a, reb 2m and various SvG7 at the end of the reaction (48 hours) was analyzed by HPLC.

### Example 6

### HPLC Analysis

For analysis, biotransformation samples were inactivated by adjusting the reaction mixture to pH5.5 using 17% H₃PO₄ and then boiled for 10 minutes. Resulting samples were filtered, the filtrates were diluted 10 times and used as samples for HPLC analysis. HPLC assay was carried out on Agilent HP 1200 HPLC system, comprised of a pump, a column thermostat, an auto sampler, a UV detector capable of background correction and a data acquisition system. Analytes were separated using Agilent Poroshell 120 SB-C18, 4.6 mm×150 mm, 2.7 μm at 40° C. The mobile phase consisted of two premixes:
premix 1 containing 75% 10 mM phosphate buffer (pH2.6) and 25% acetonitrile, and
premix 2 containing 68% 10 mM phosphate buffer (pH2.6) and 32% acetonitrile.

Elution gradient started with premix 1, changed to premix 2 to 50% at 12.5 minute, changed to premix 2 to 100% at 13 minutes. Total run time was 45 minutes. The column temperature was maintained at 40° C. The injection volume was 5 μL. Rebaudioside species were detected by UV at 210 nm.

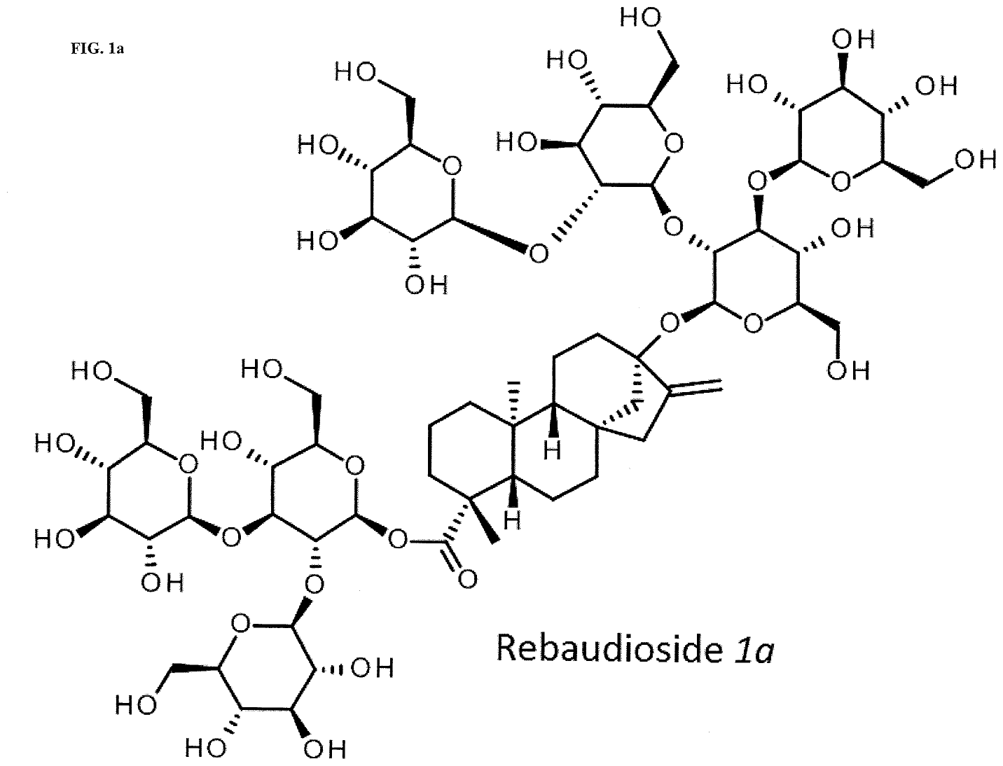

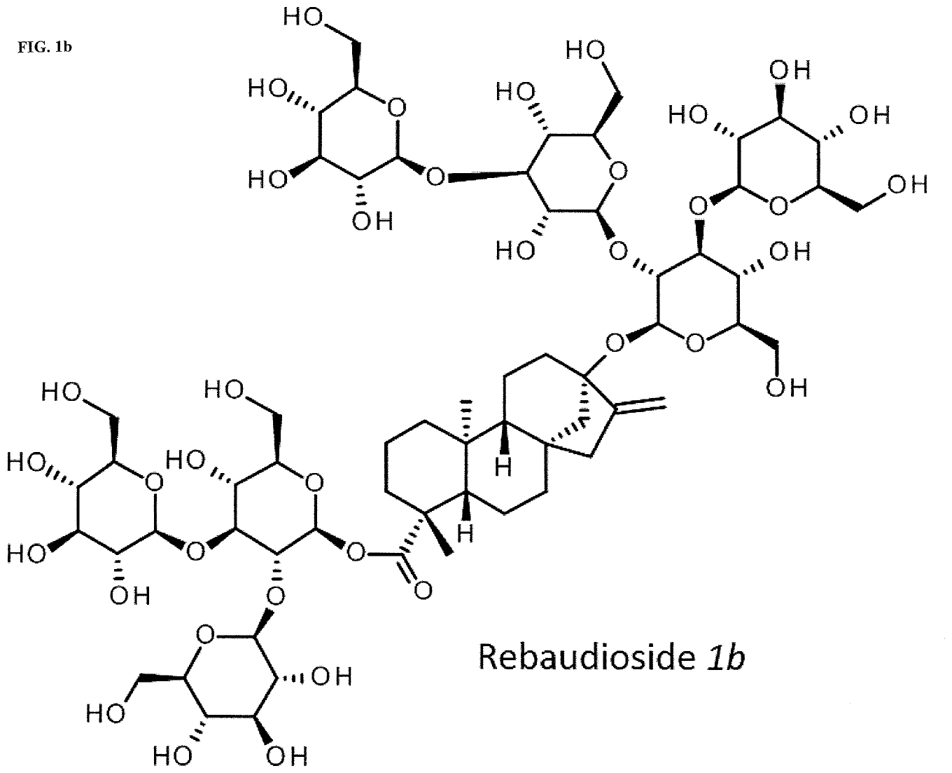

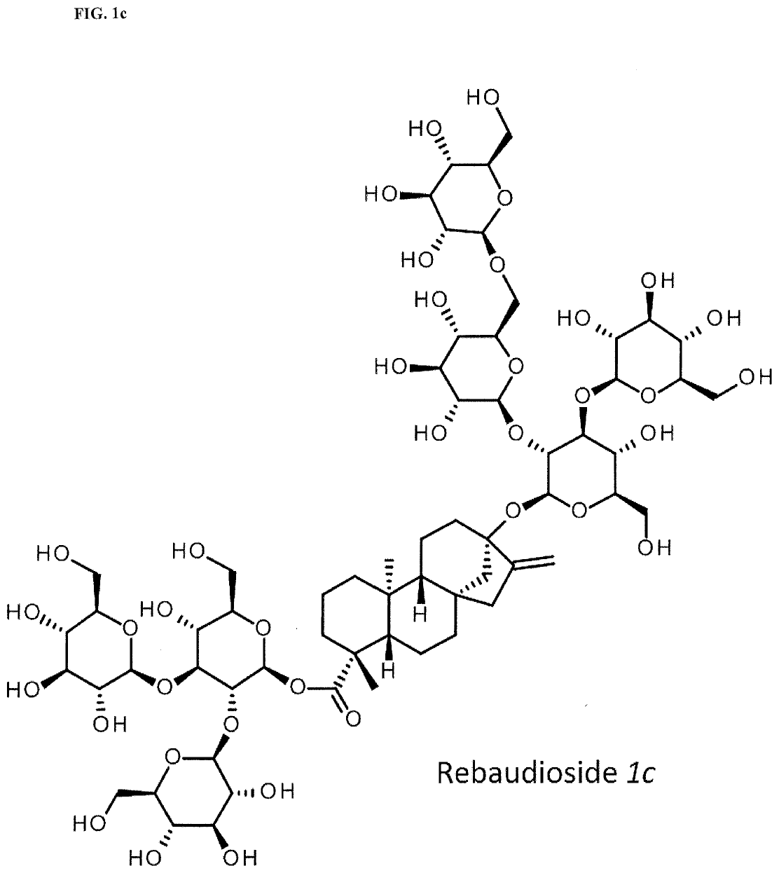

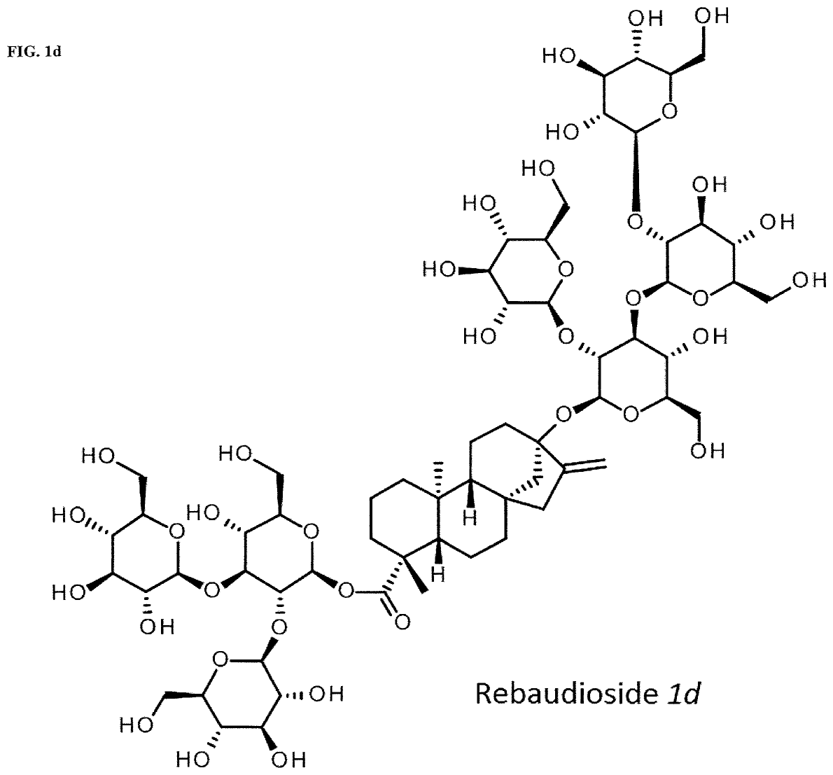

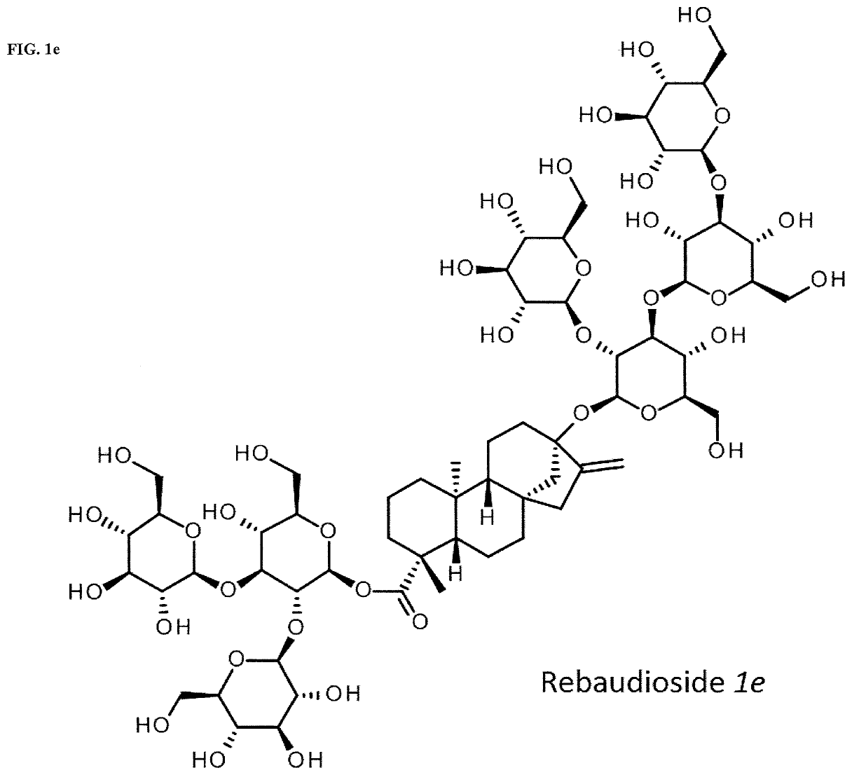

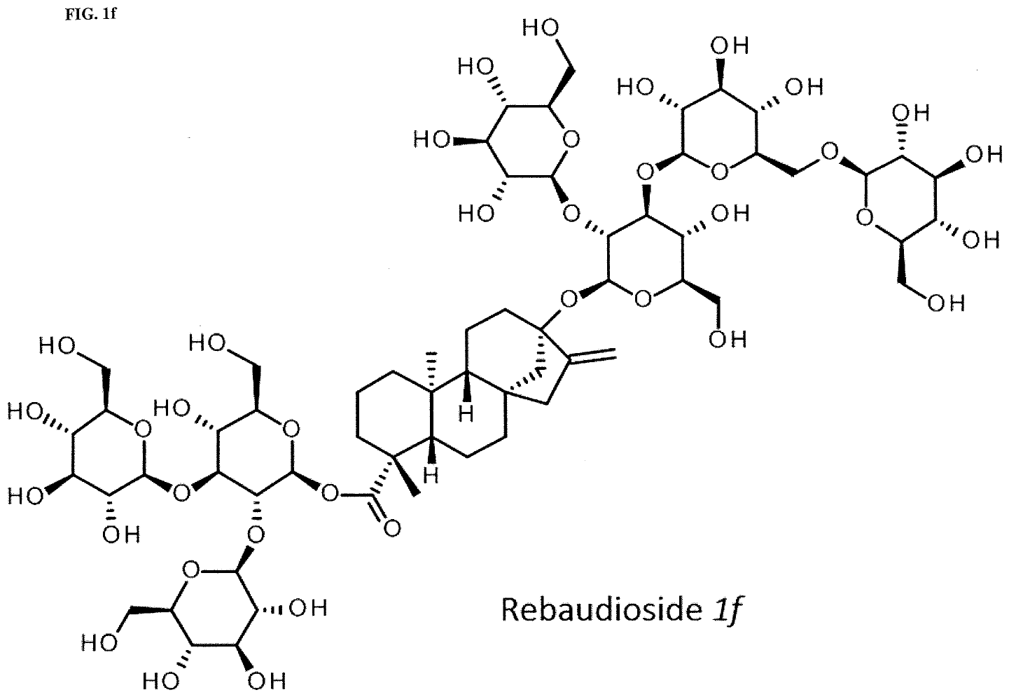

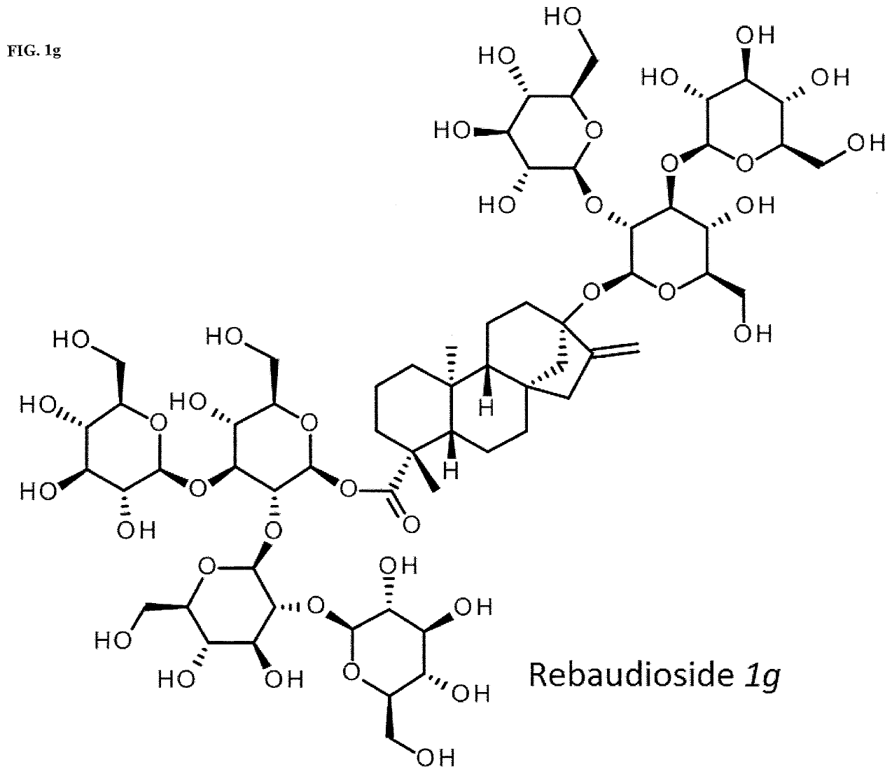

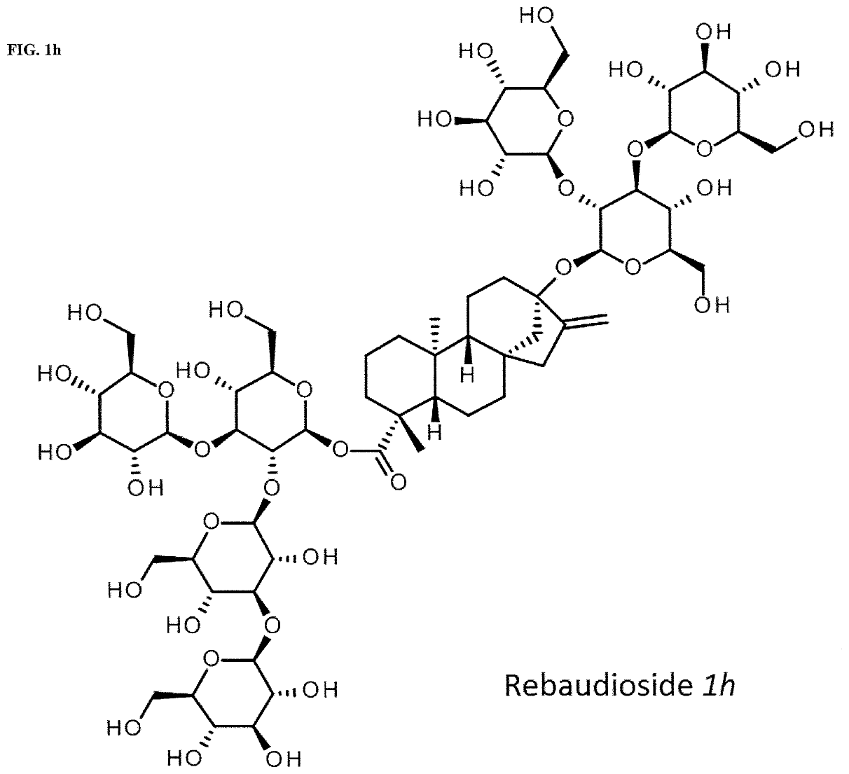

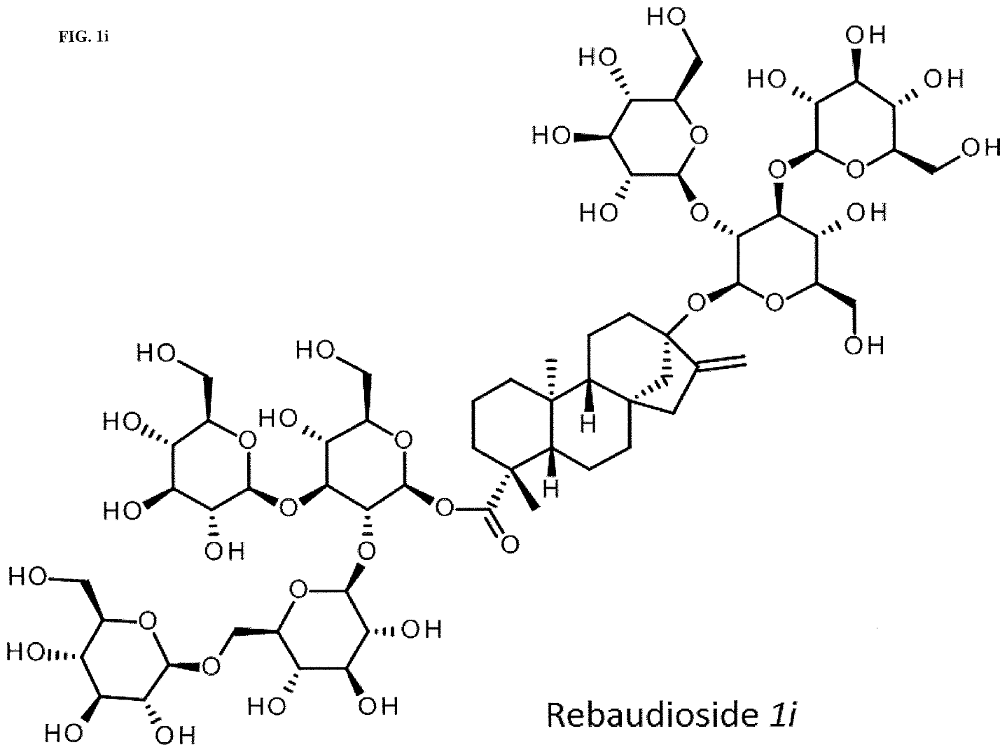

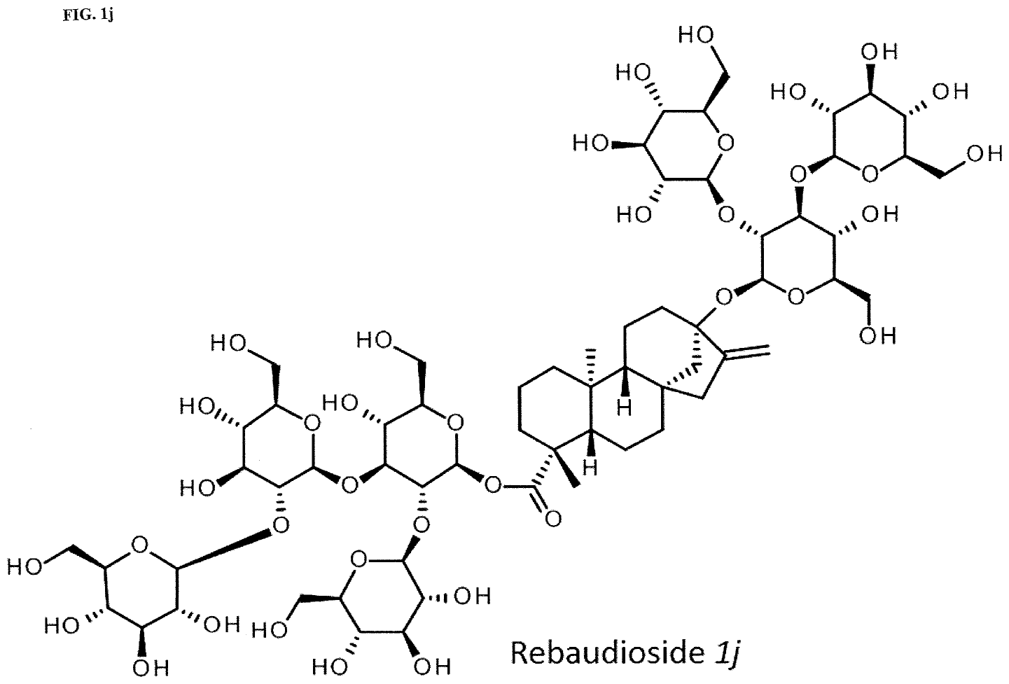

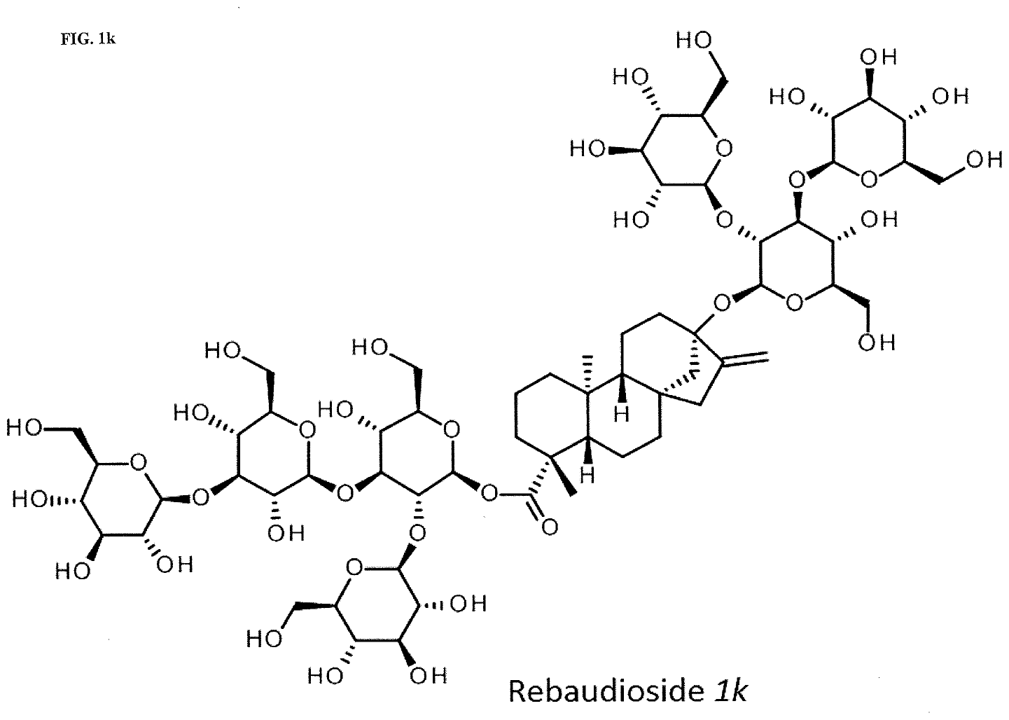

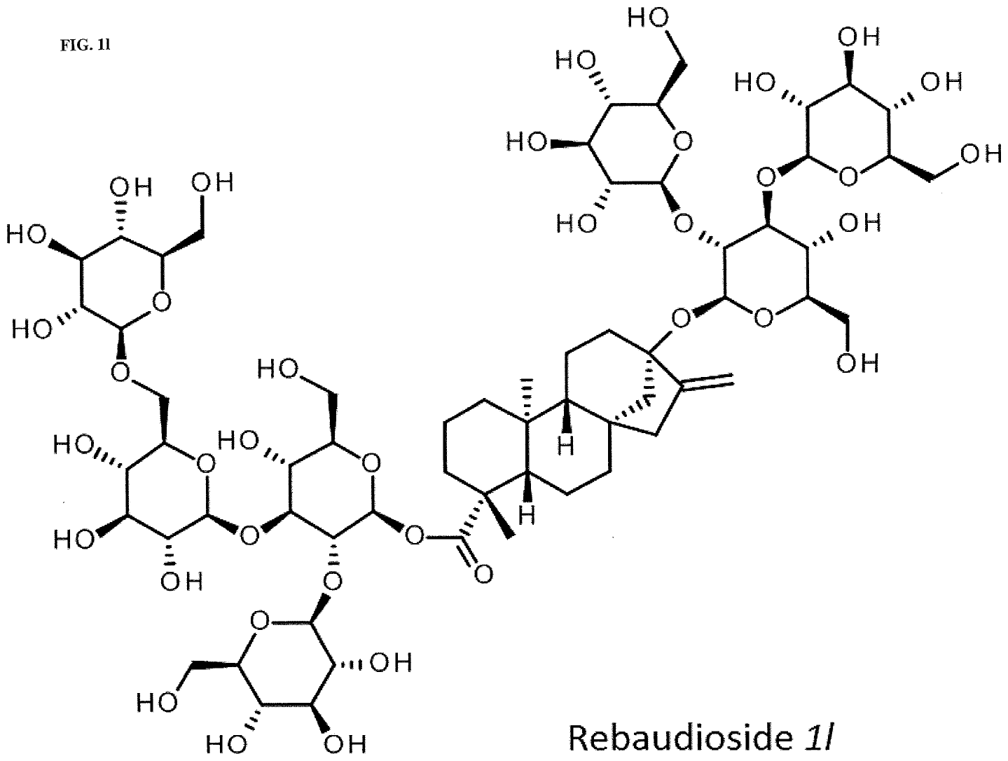

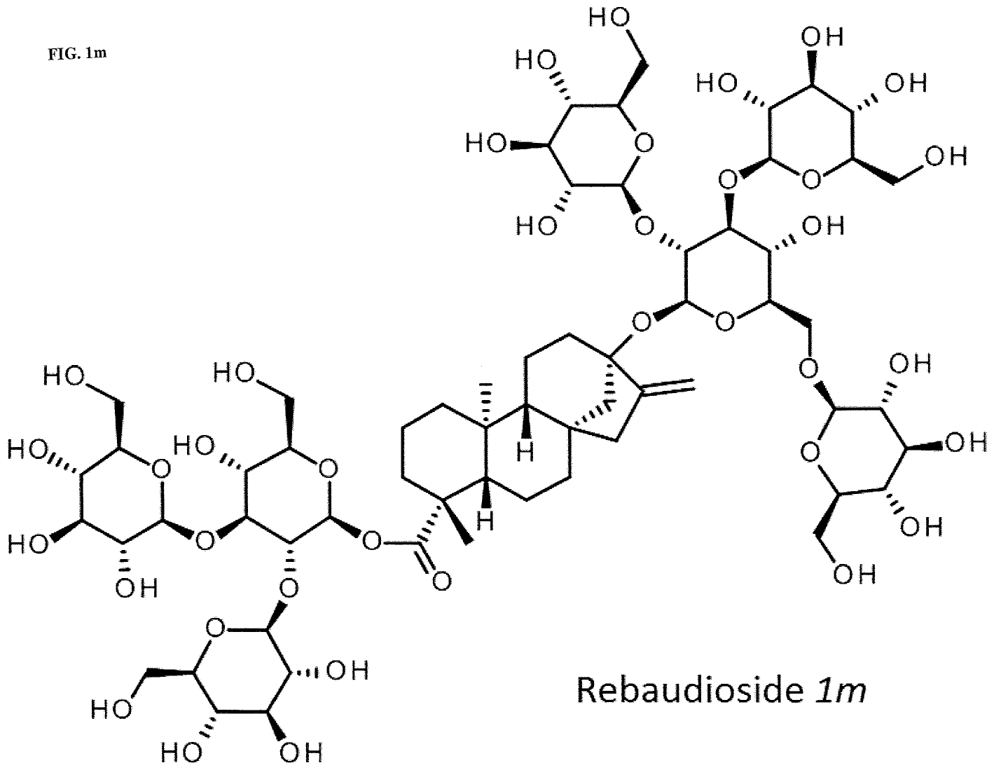

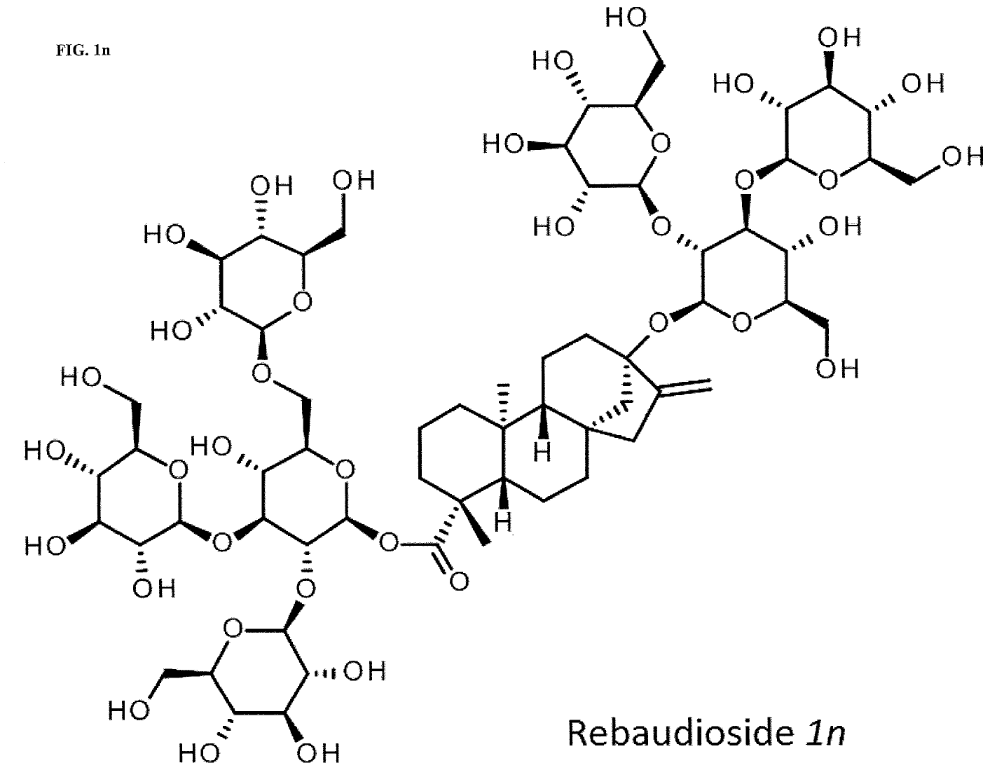

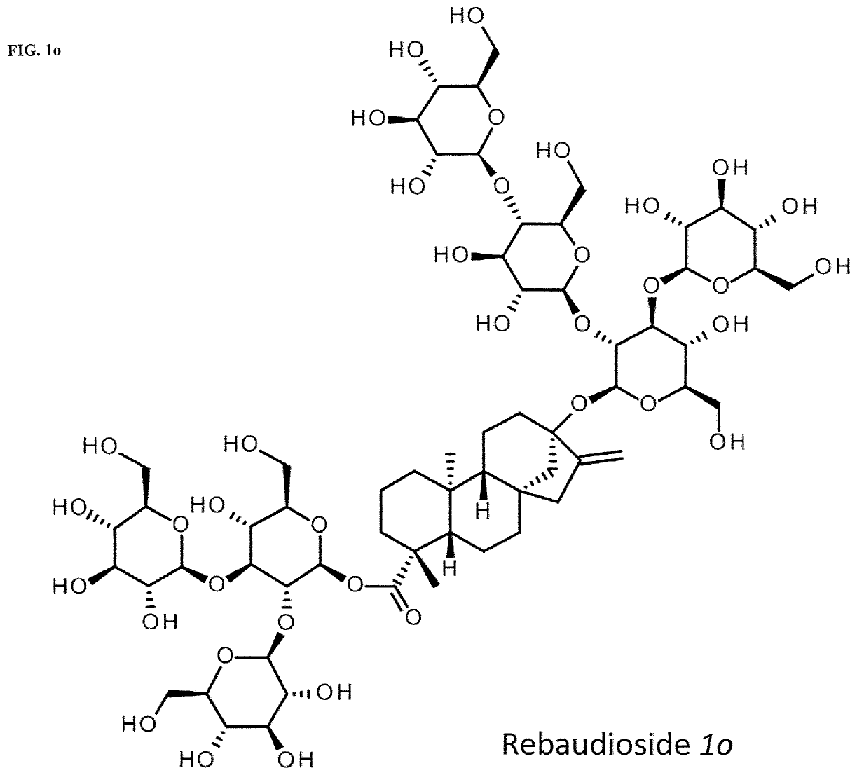

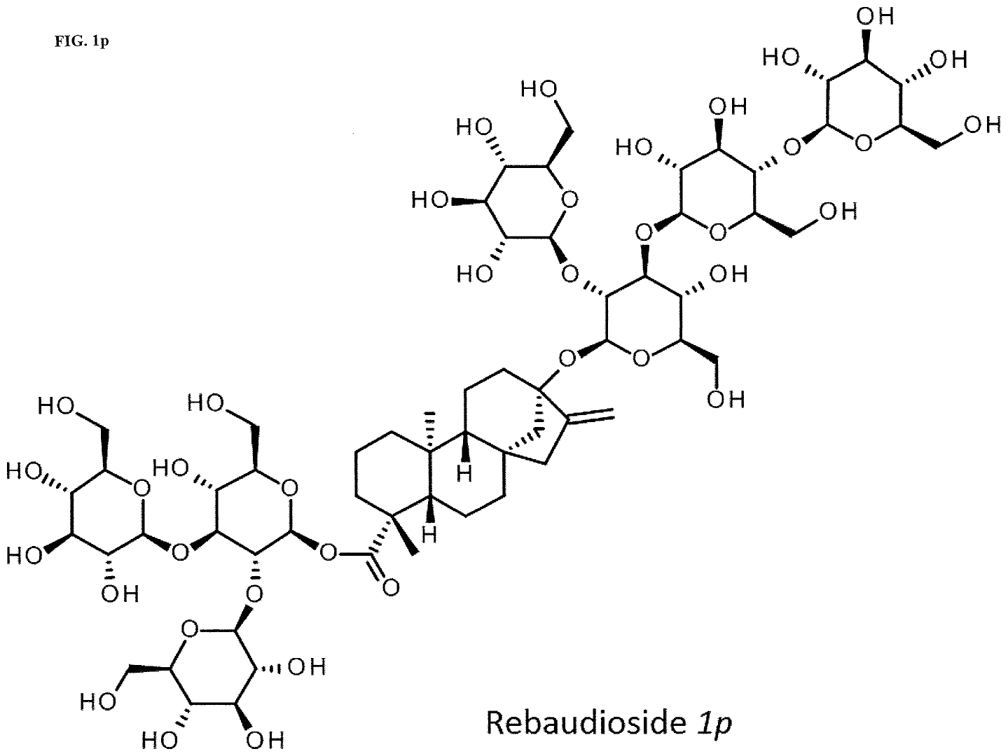

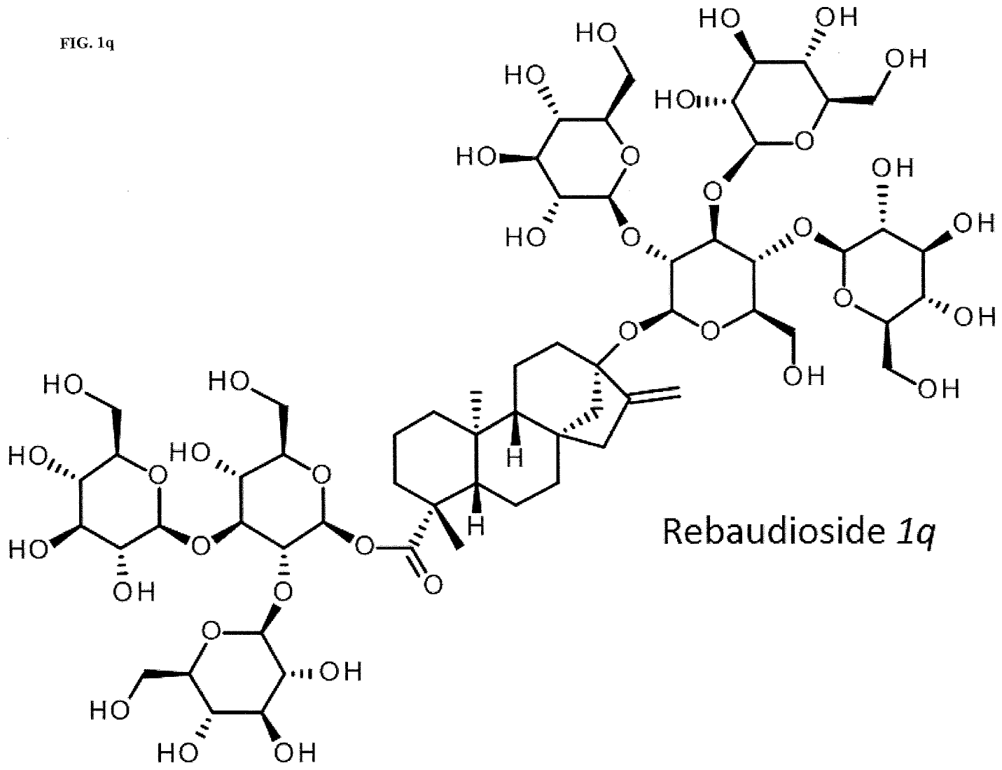

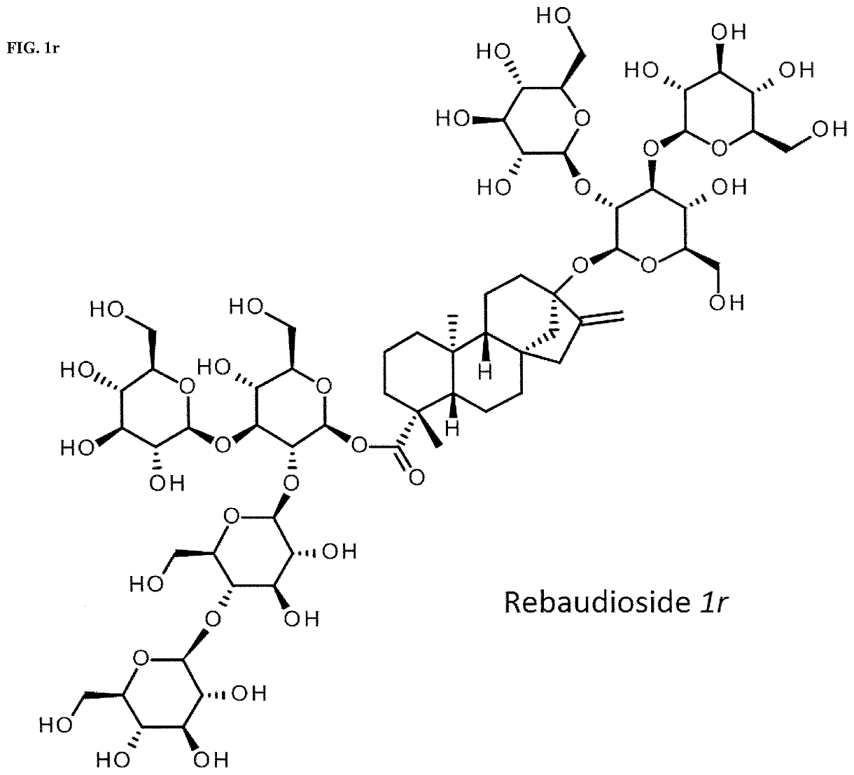

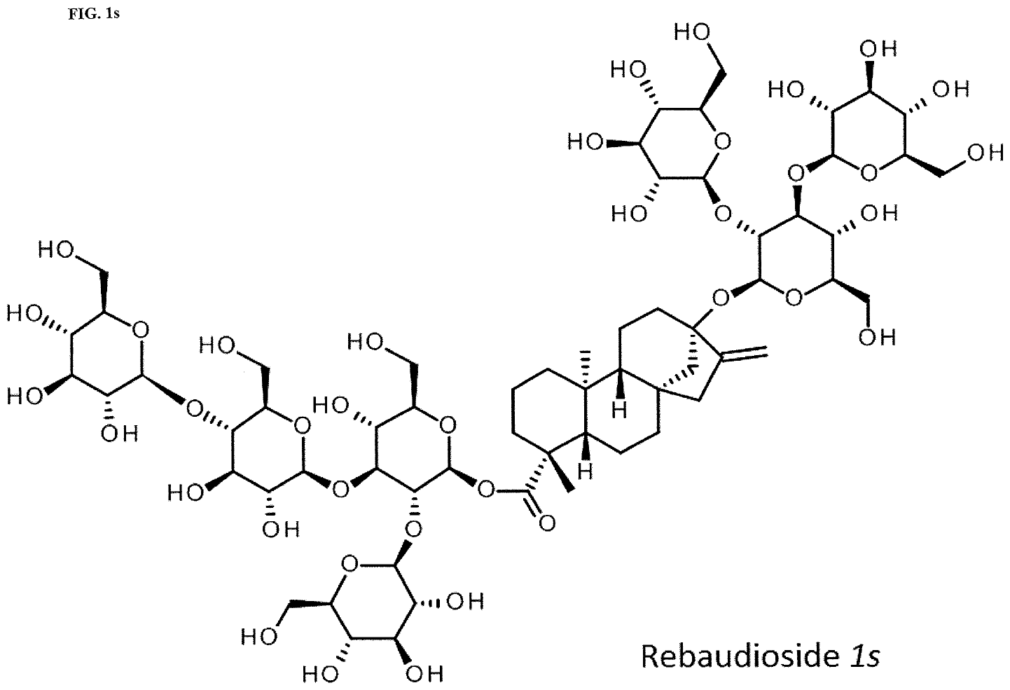

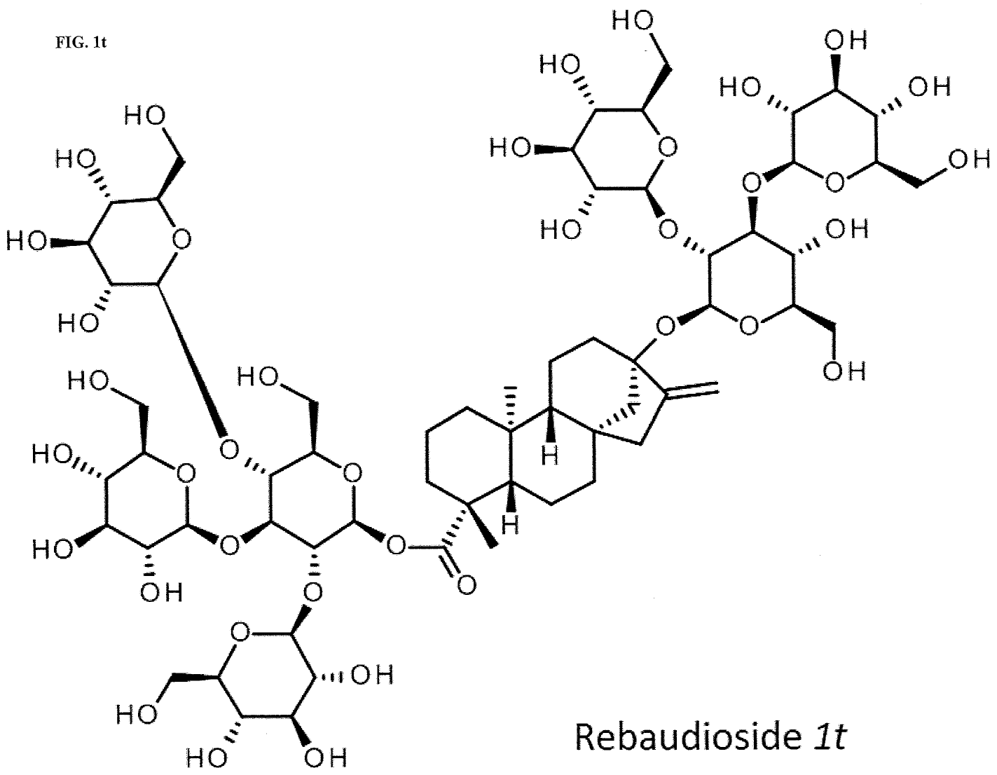

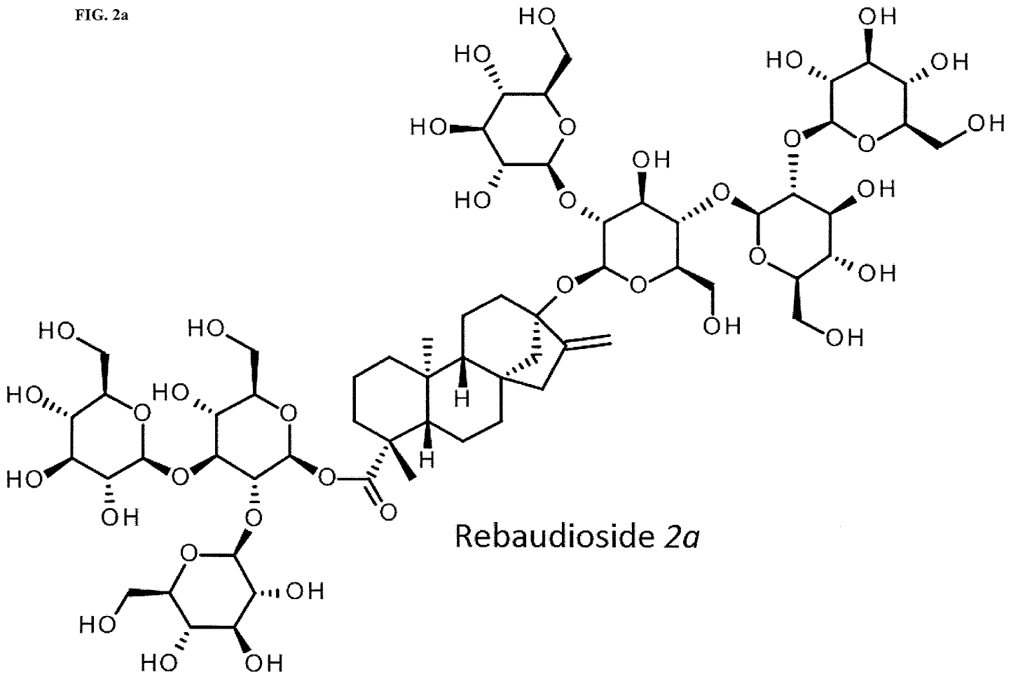

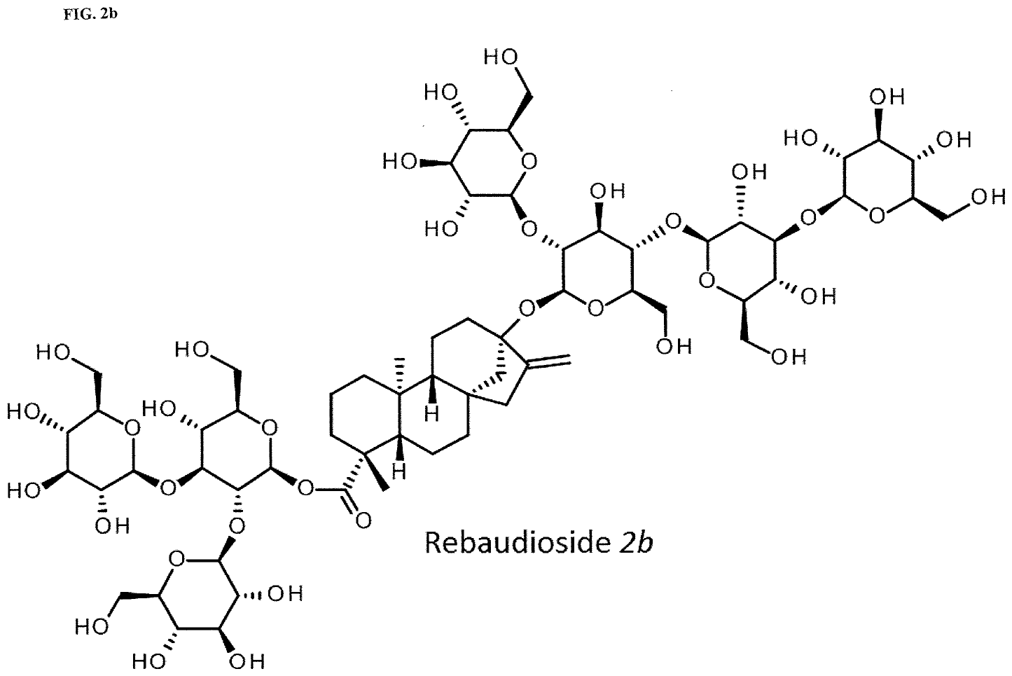

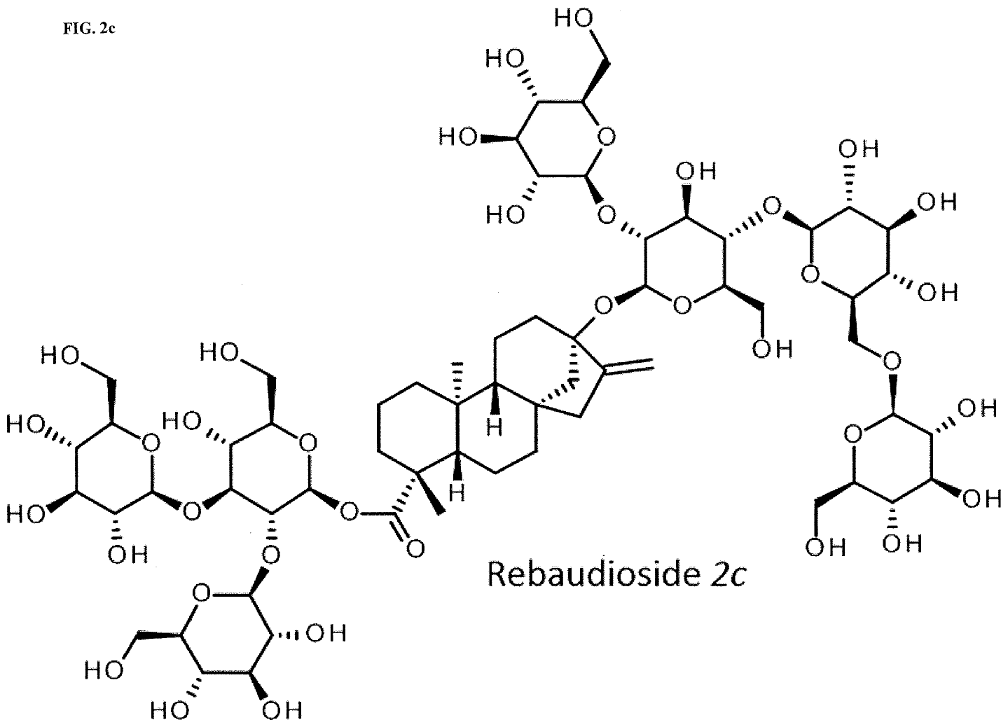

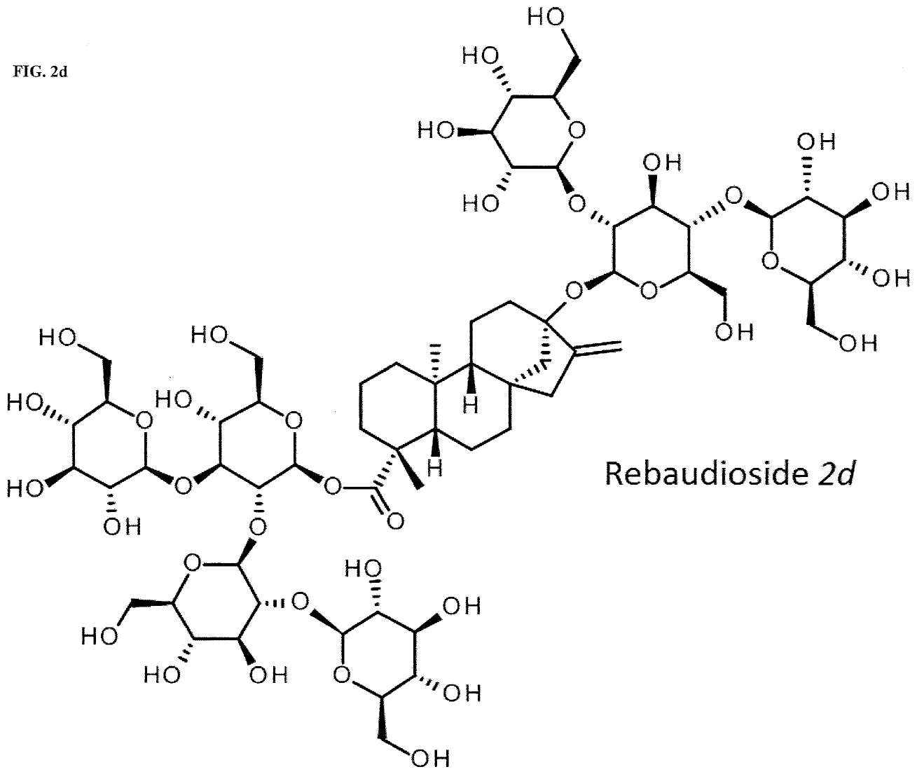

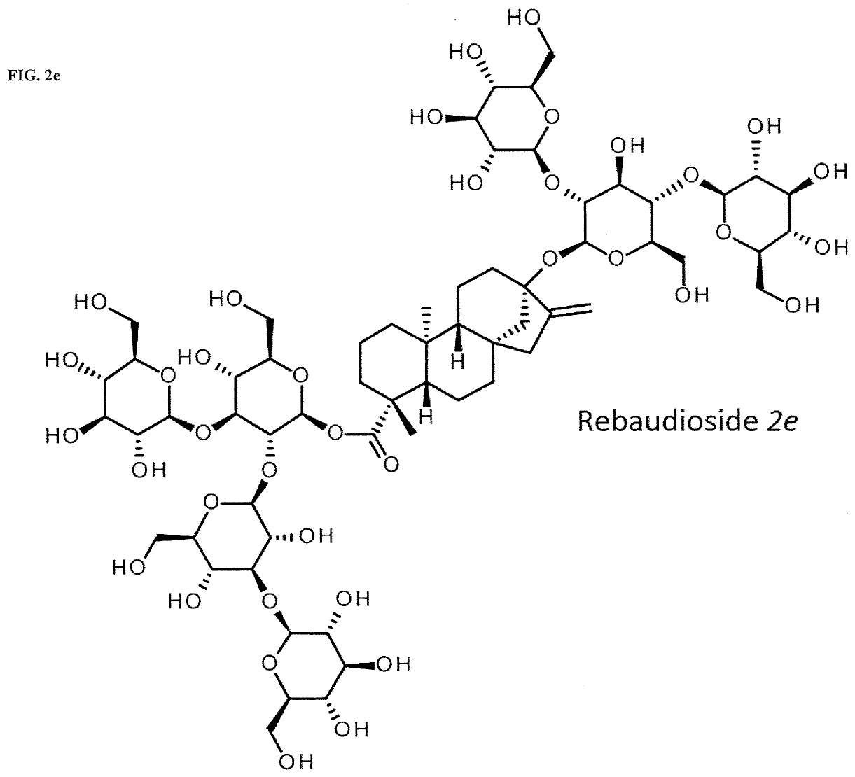

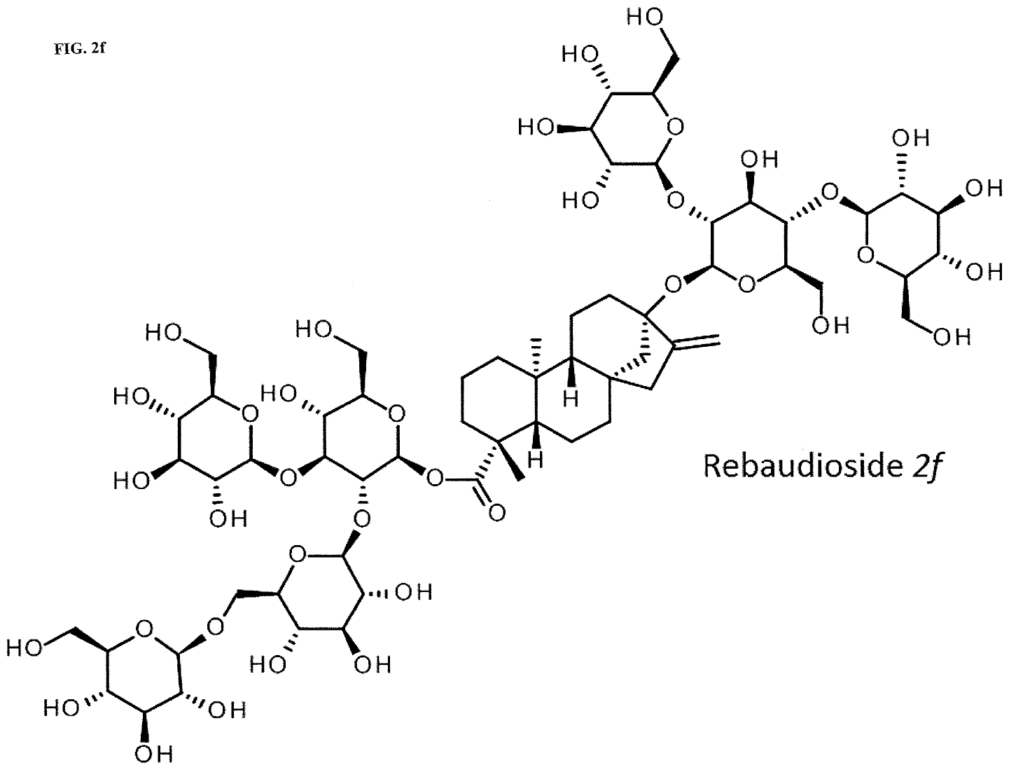

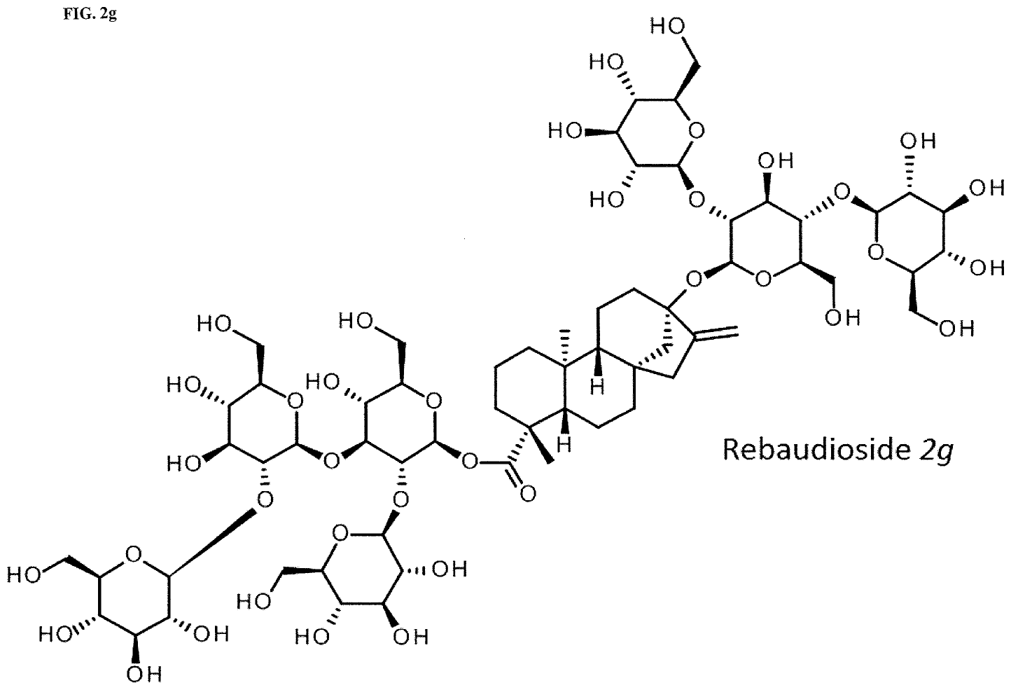

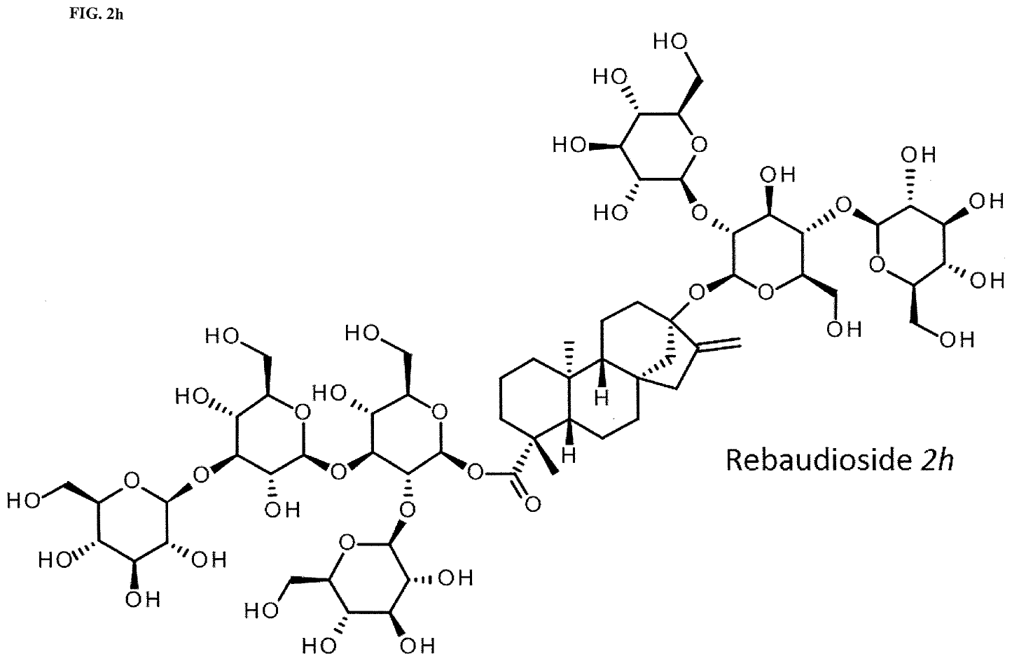

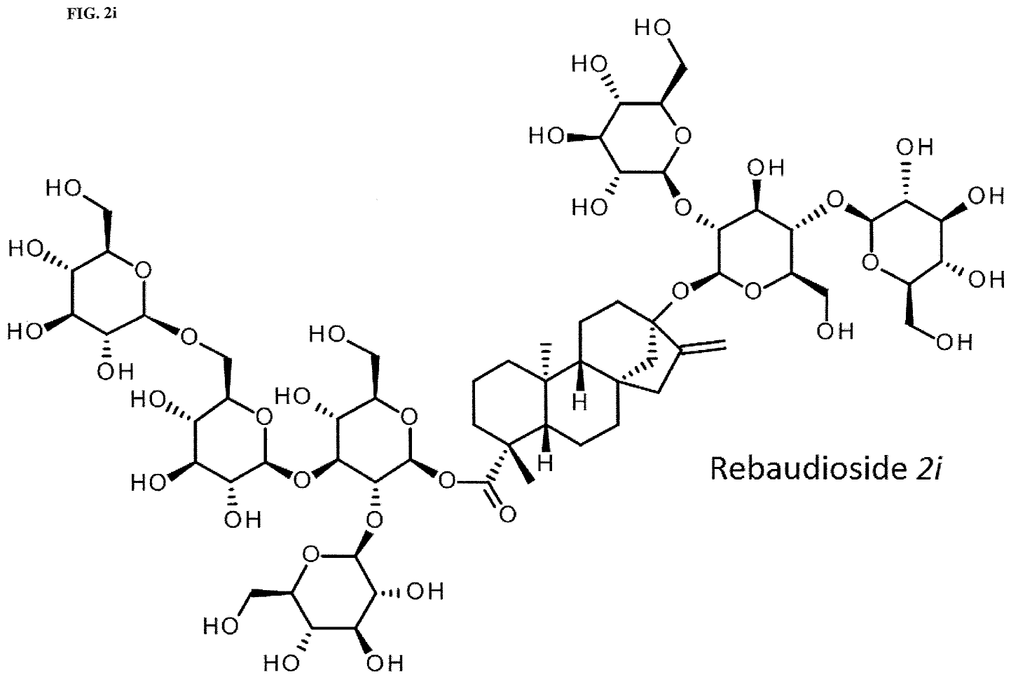

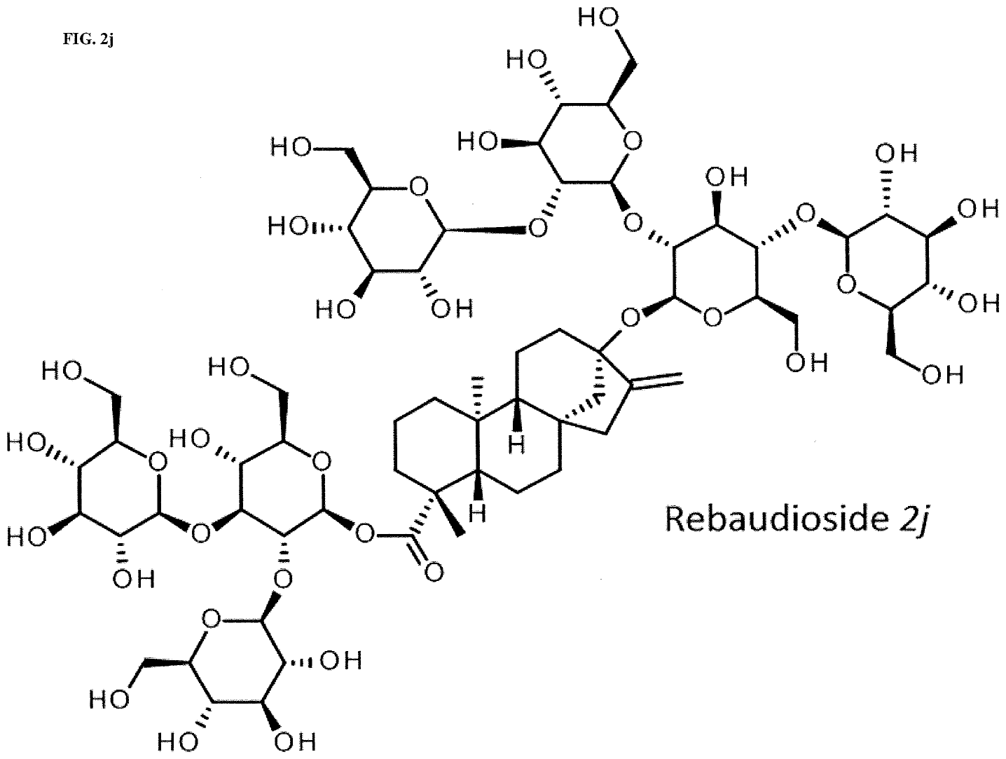

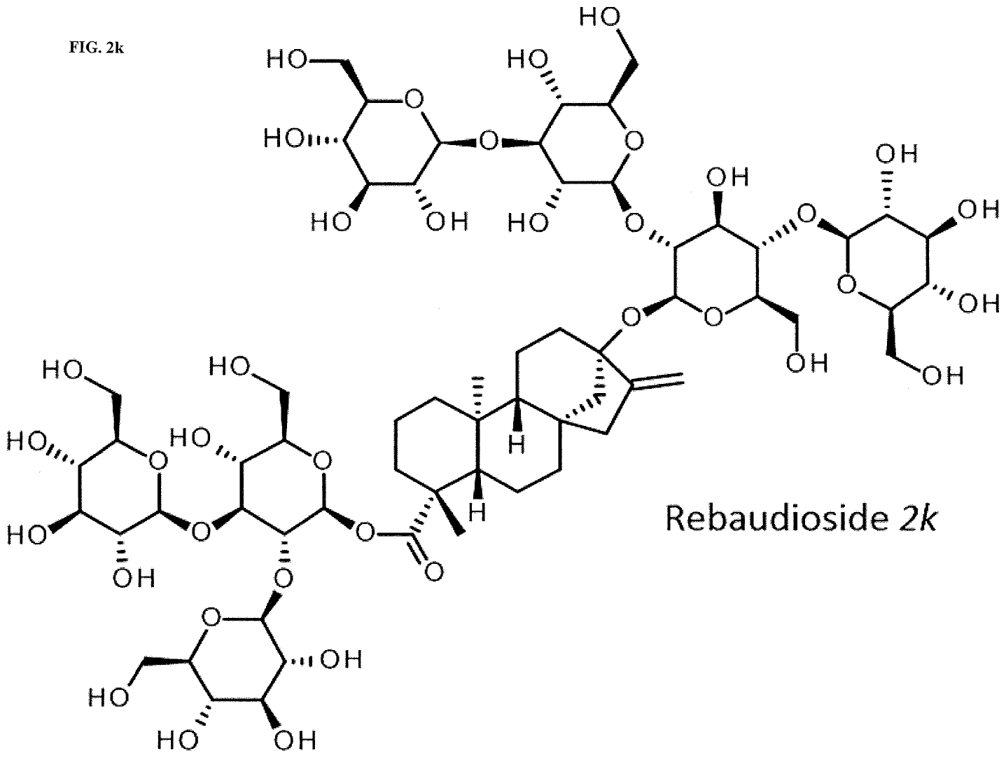

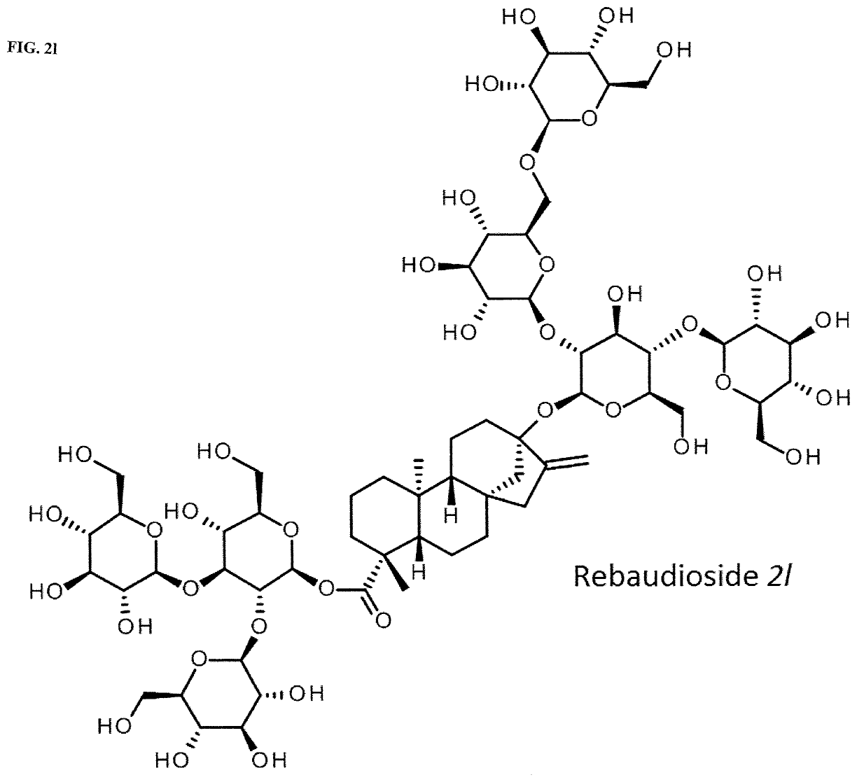

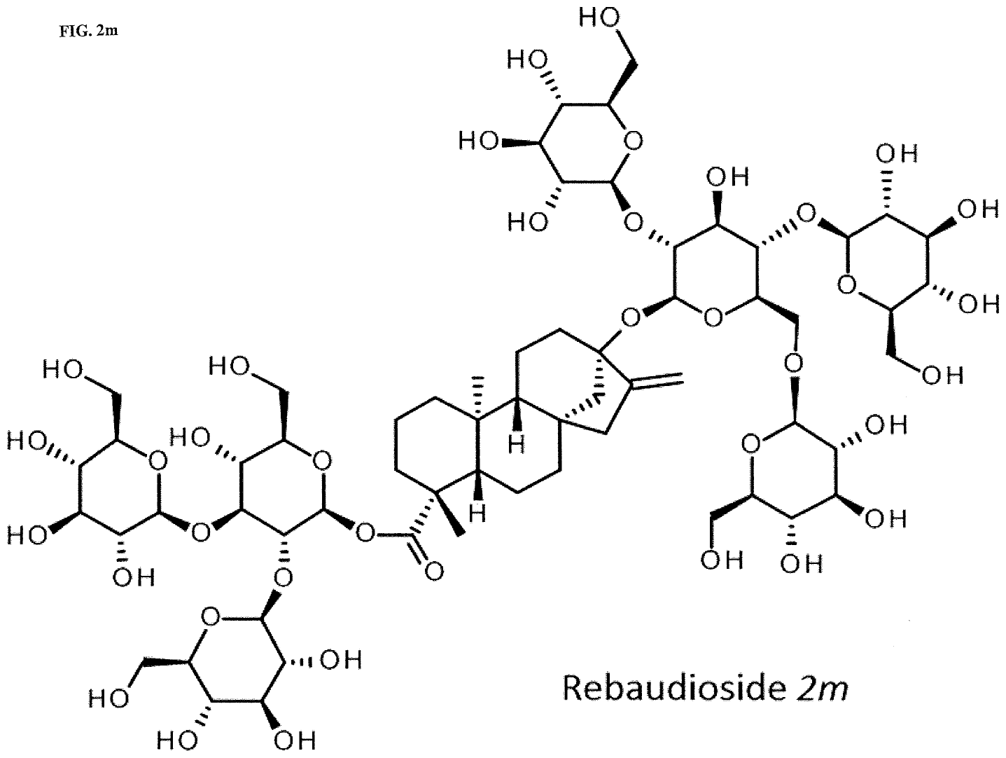

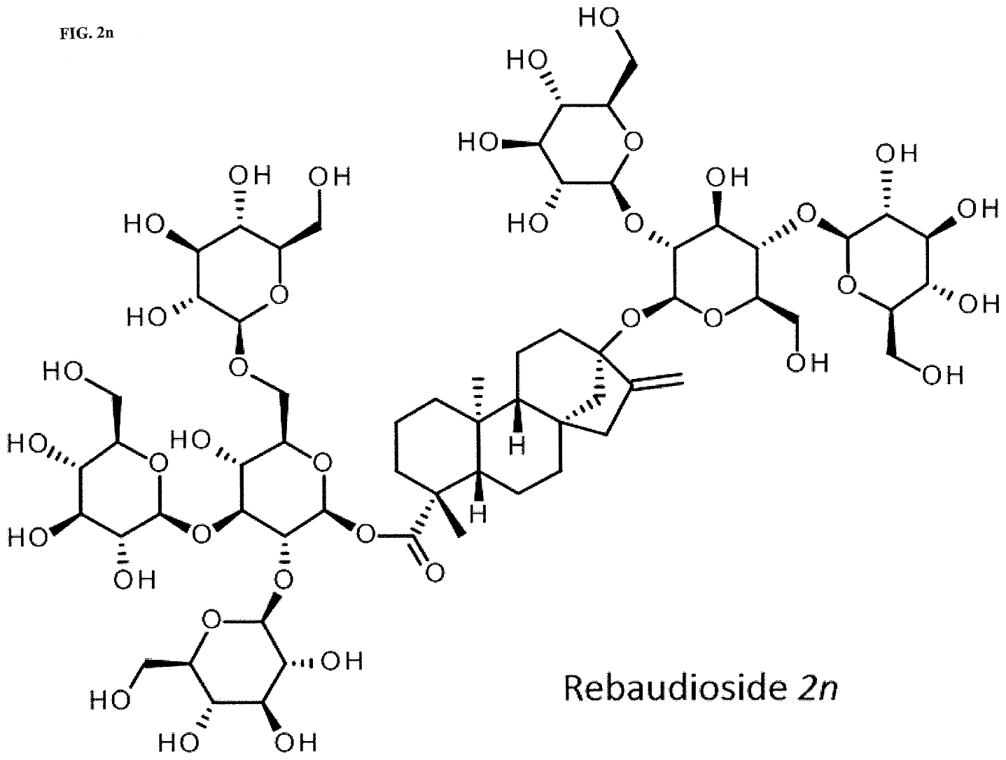

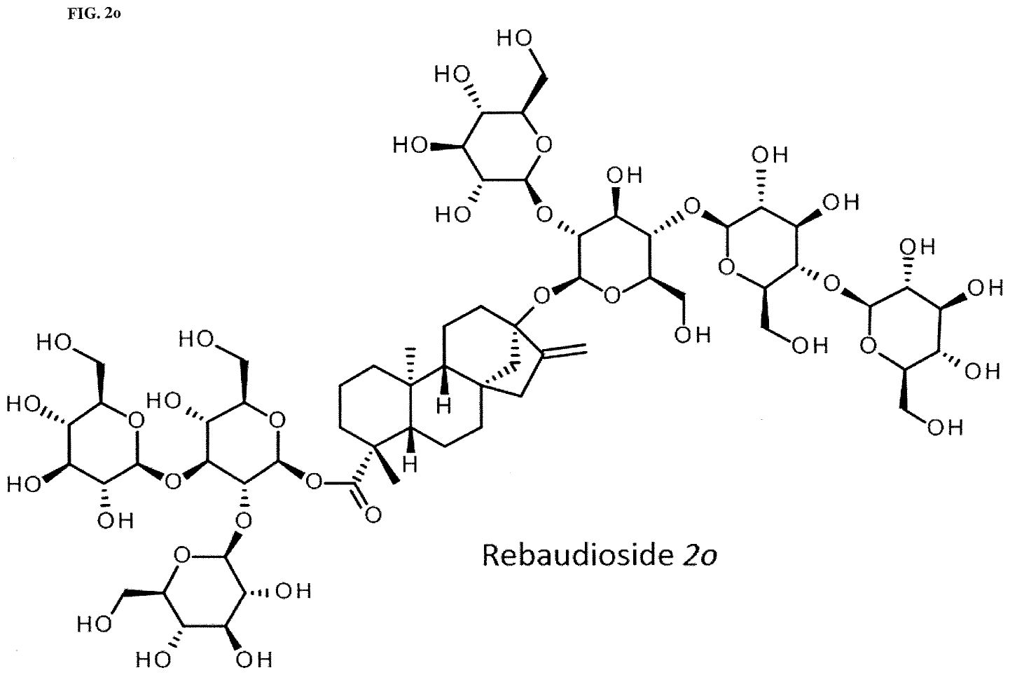

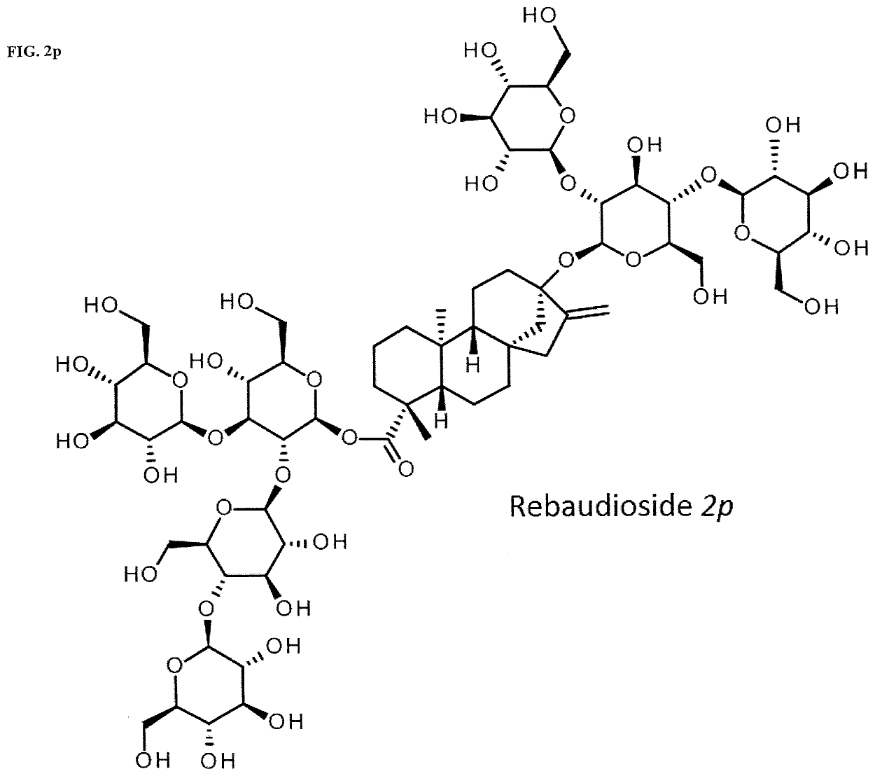

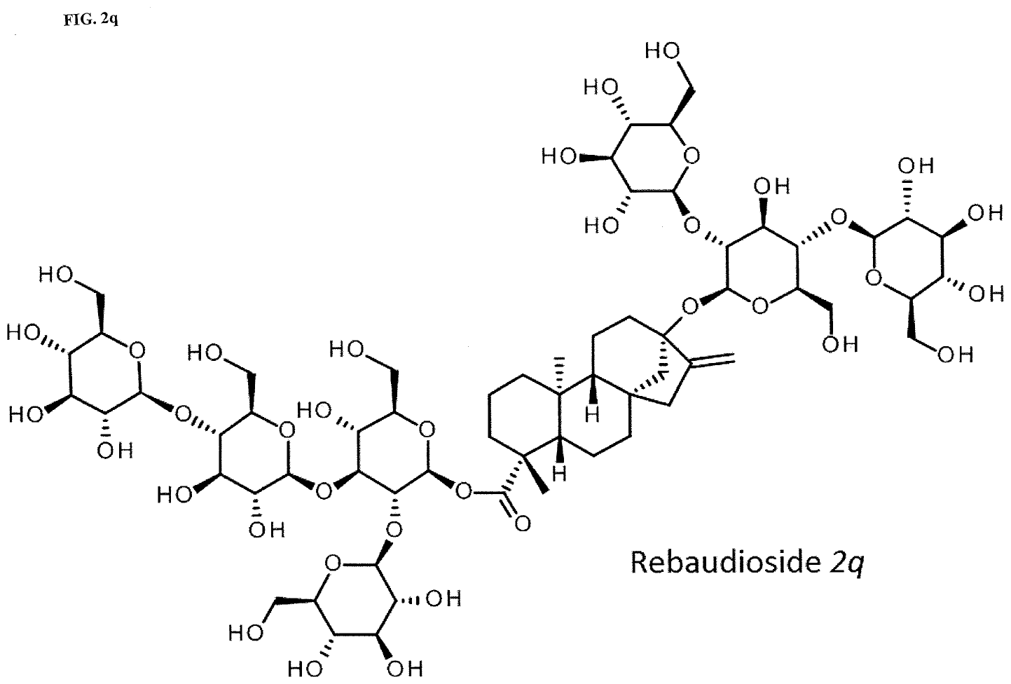

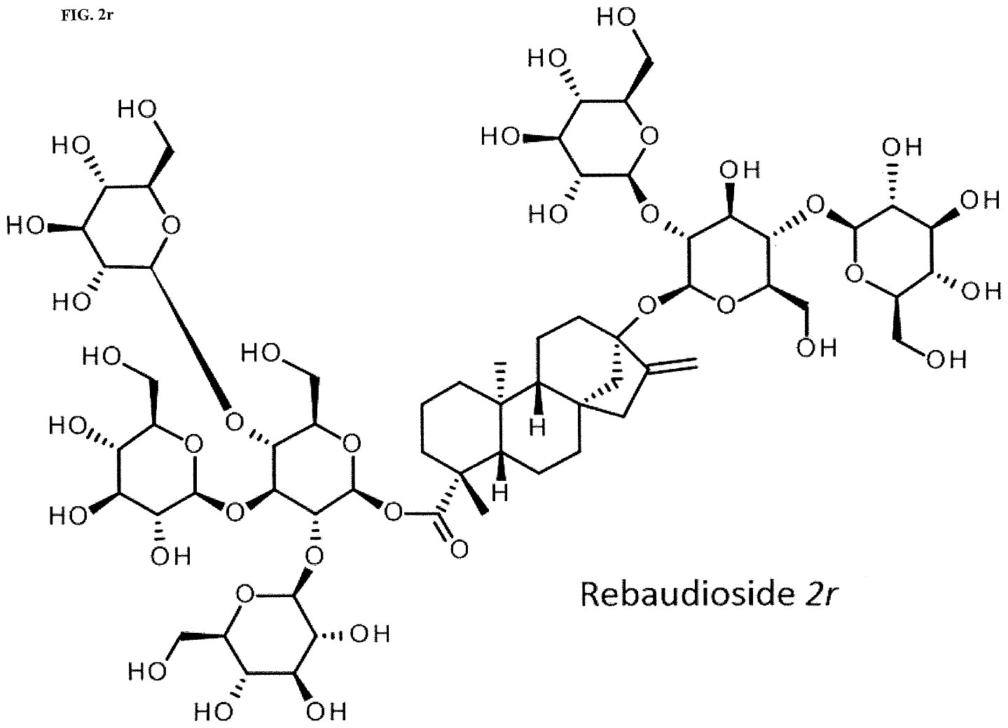

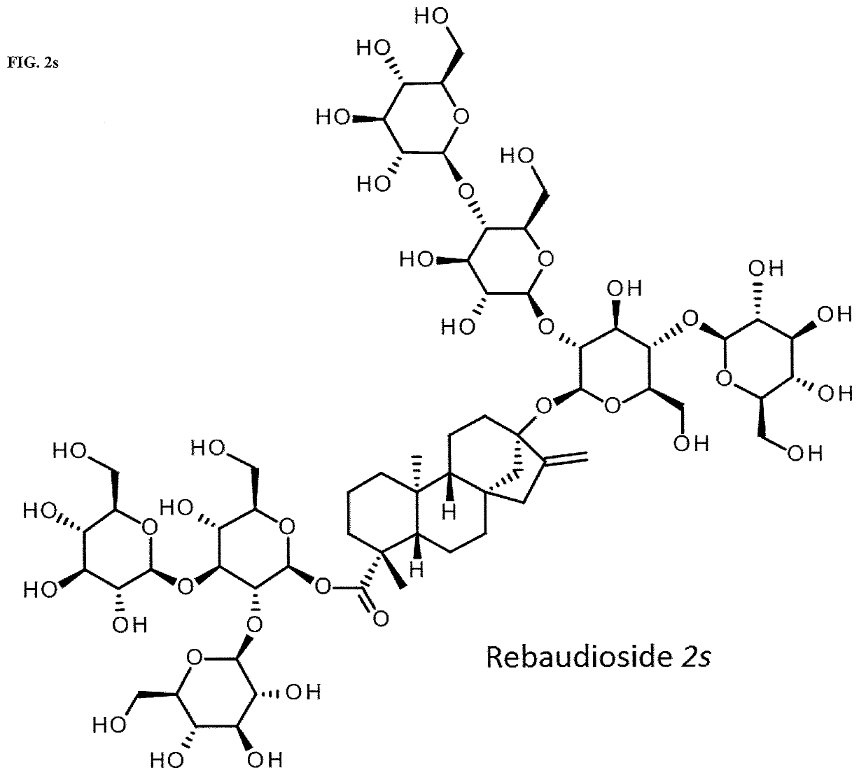

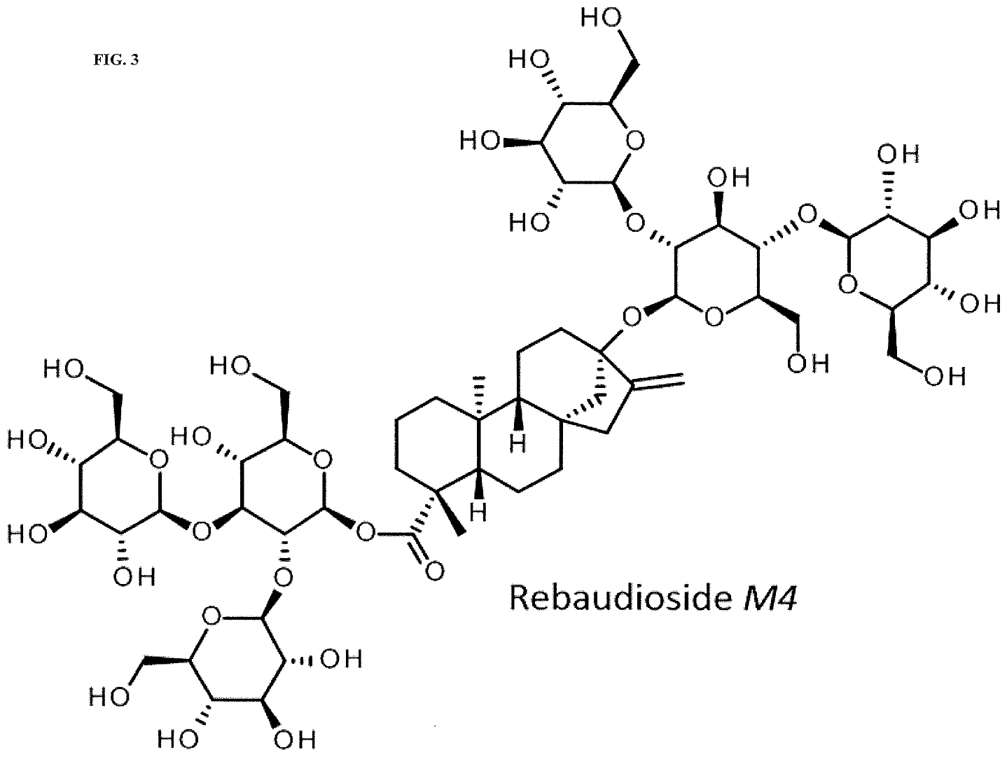

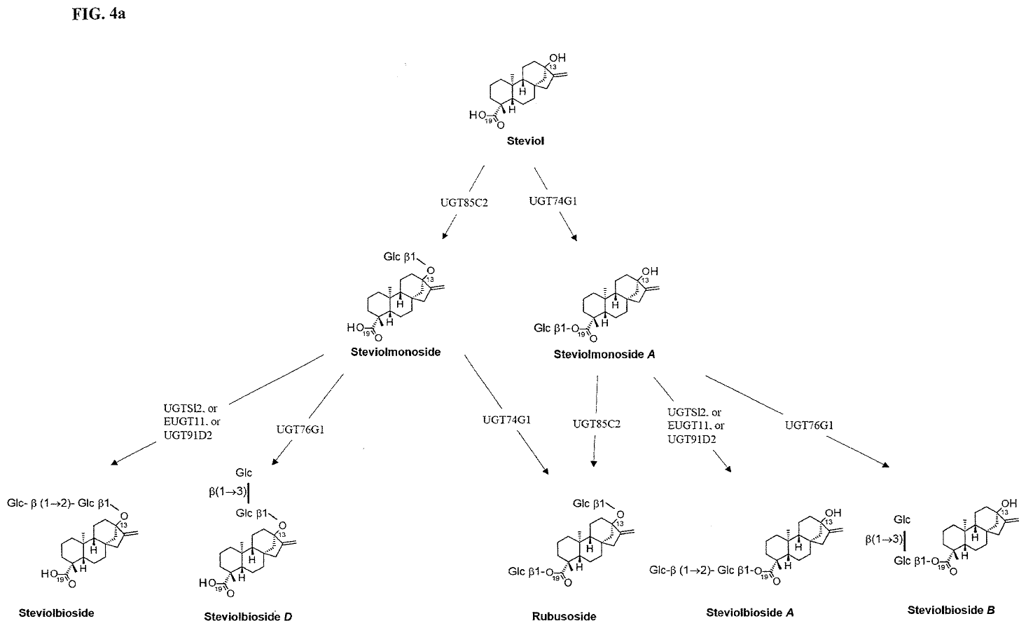

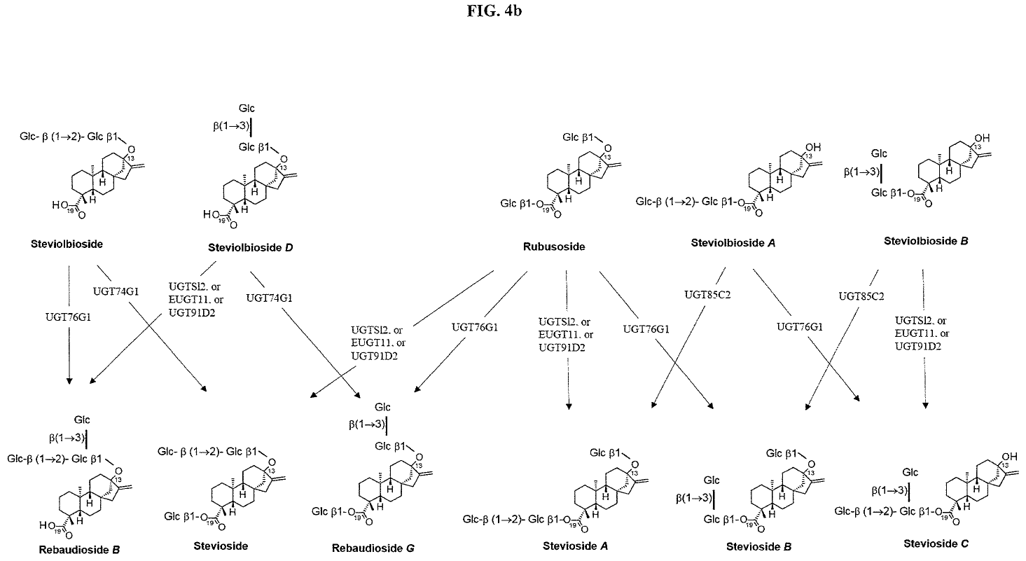

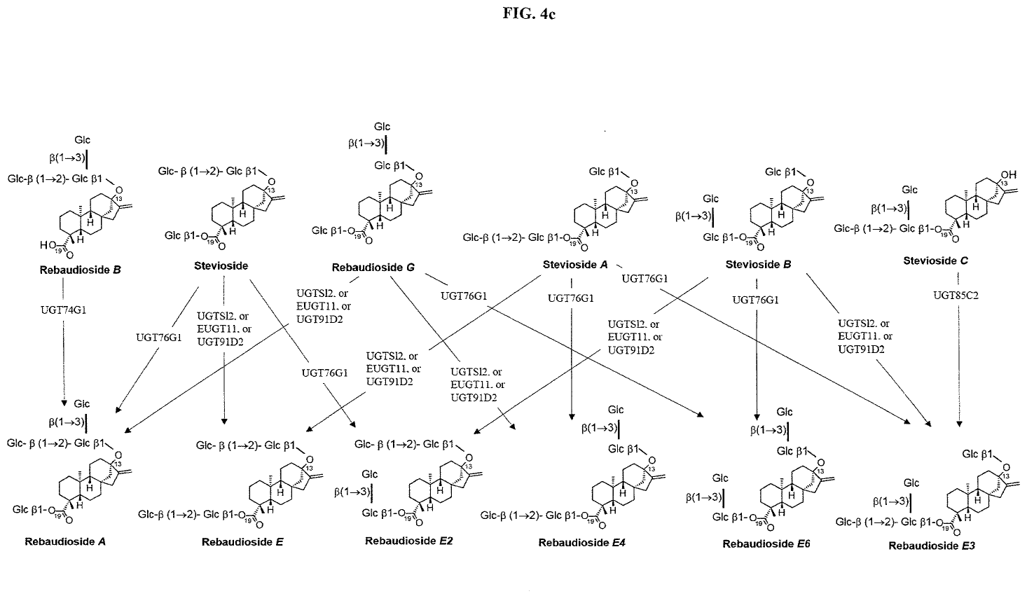

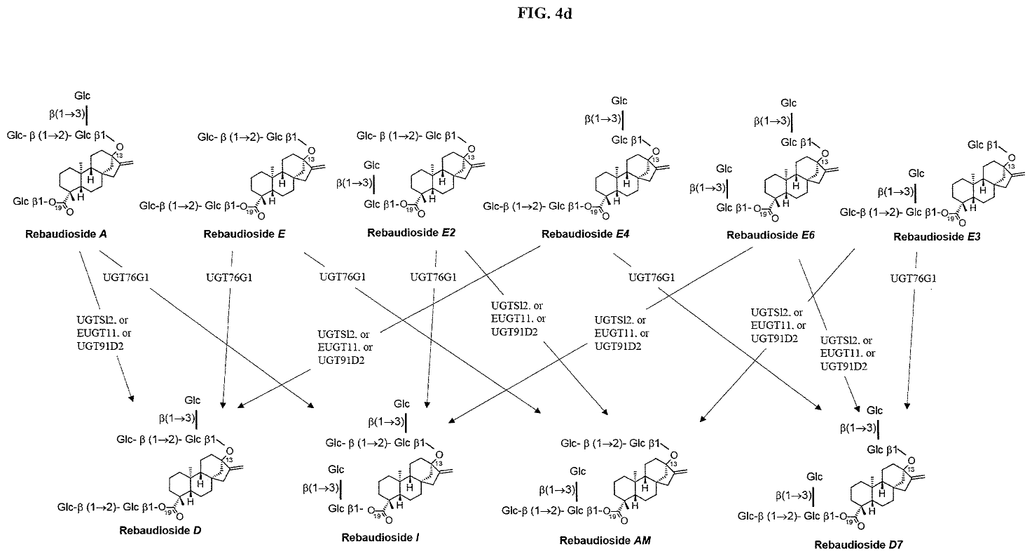

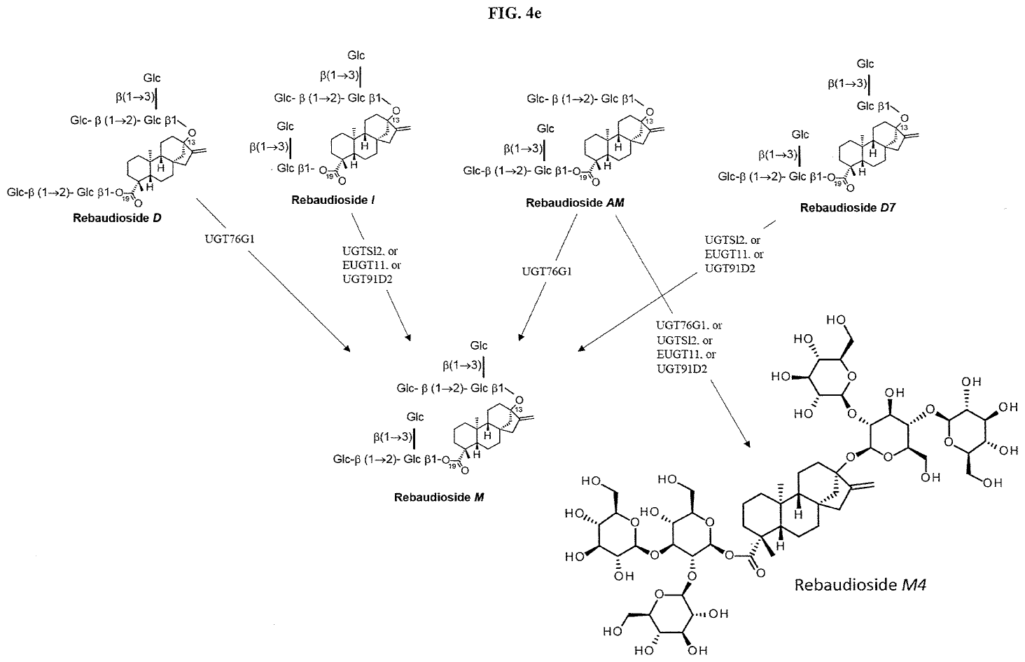

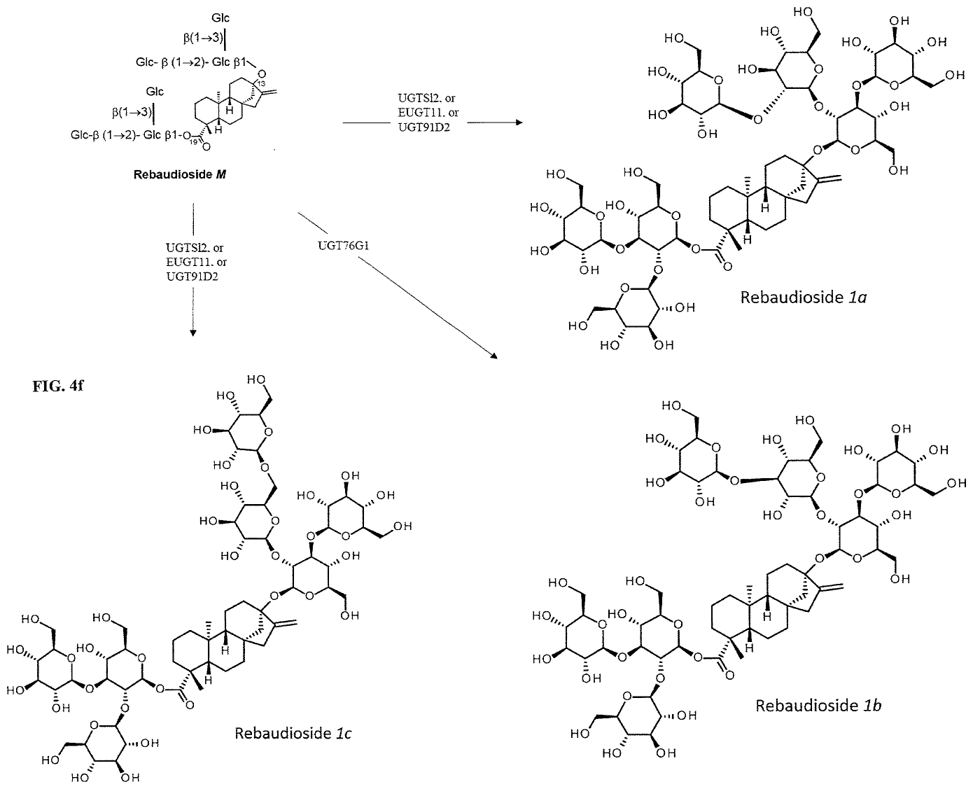

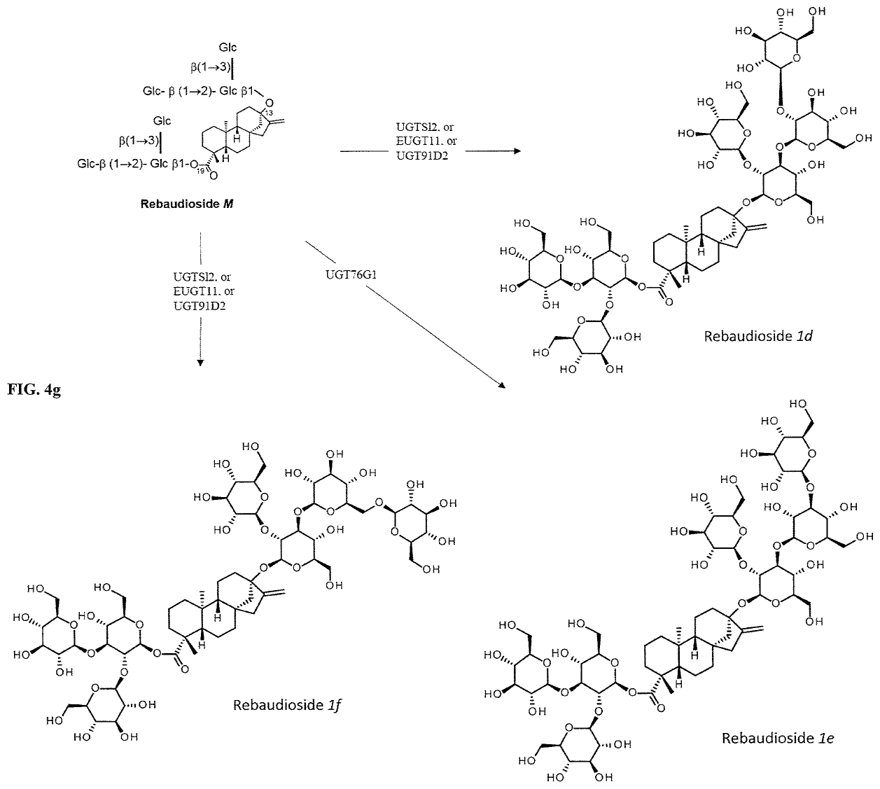

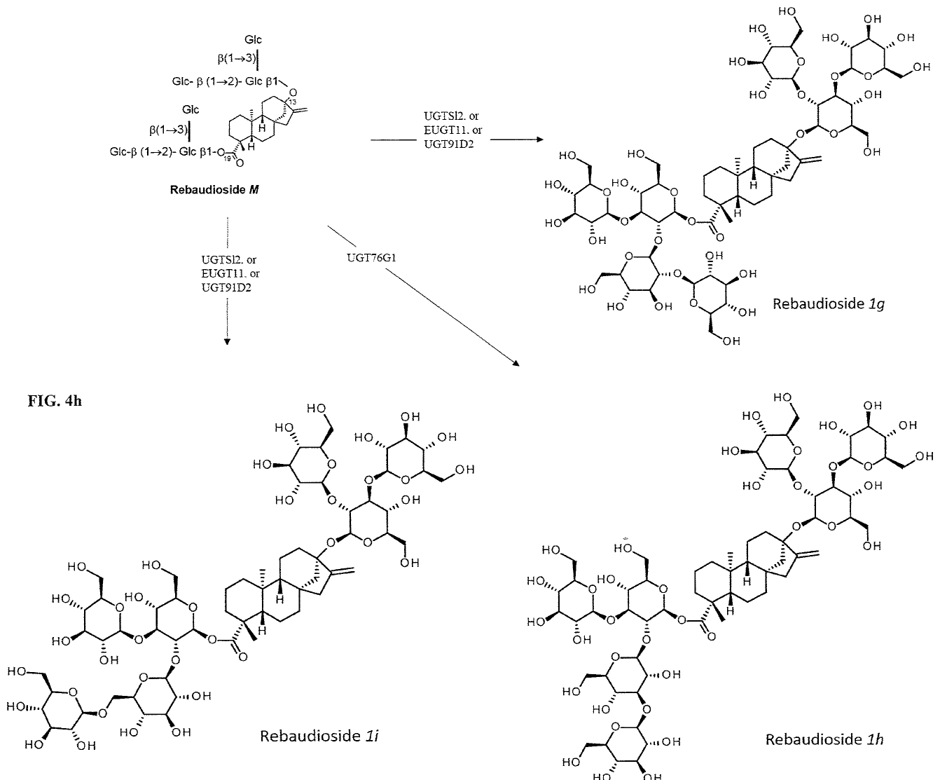

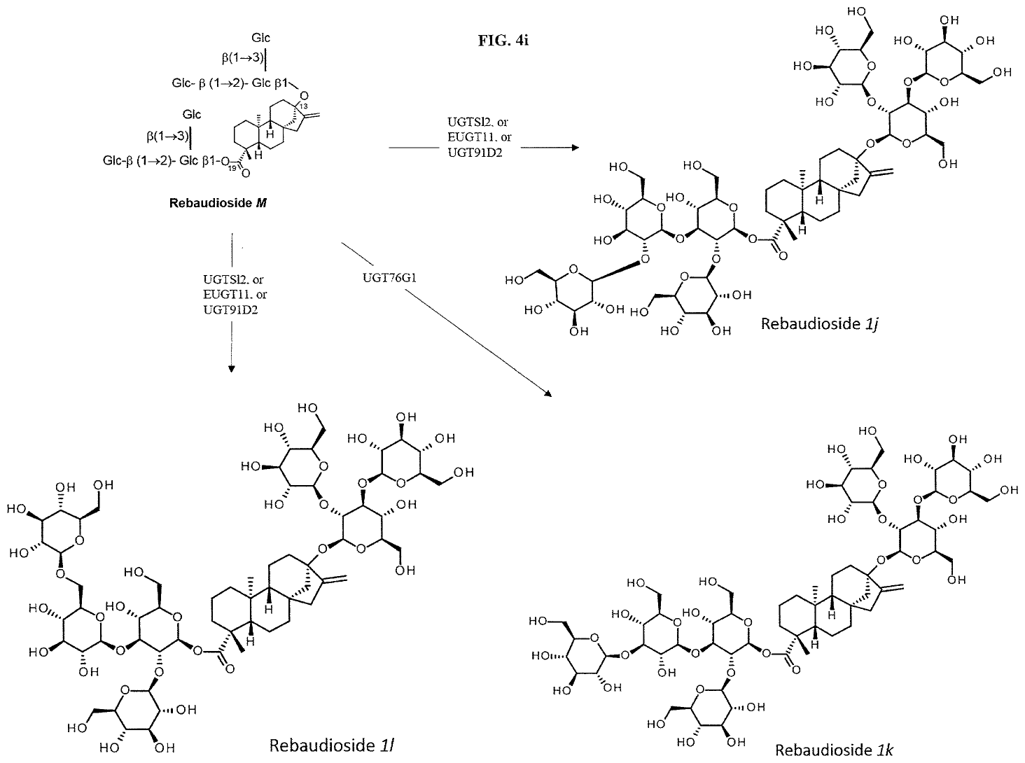

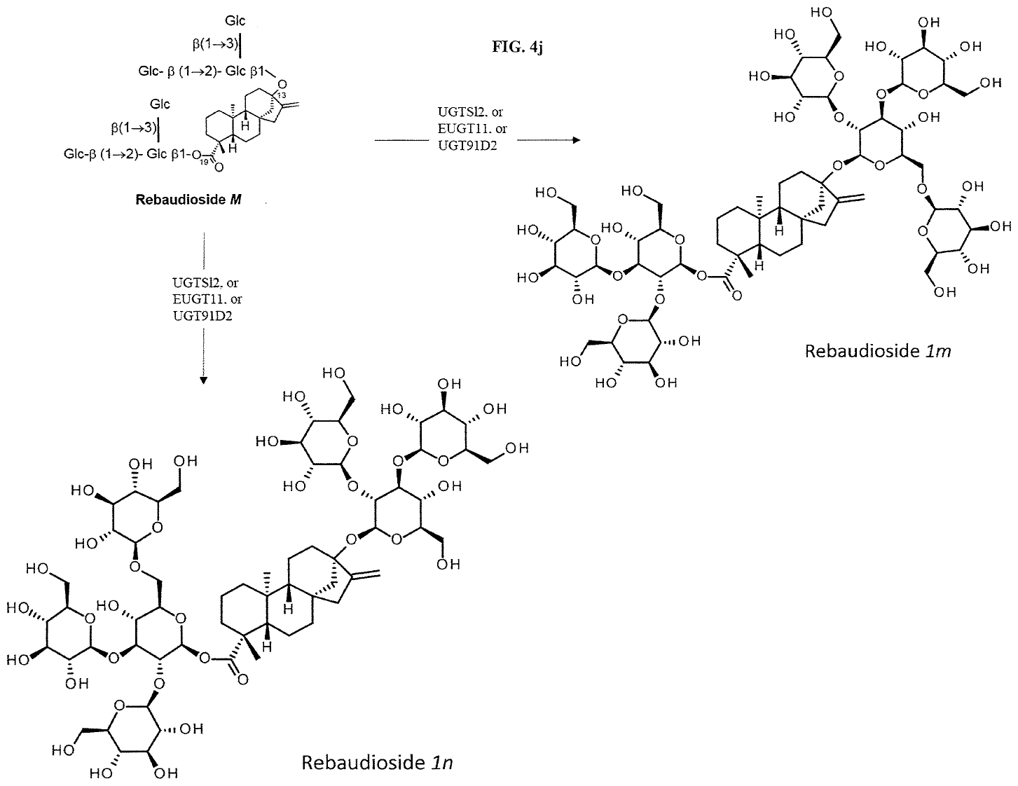

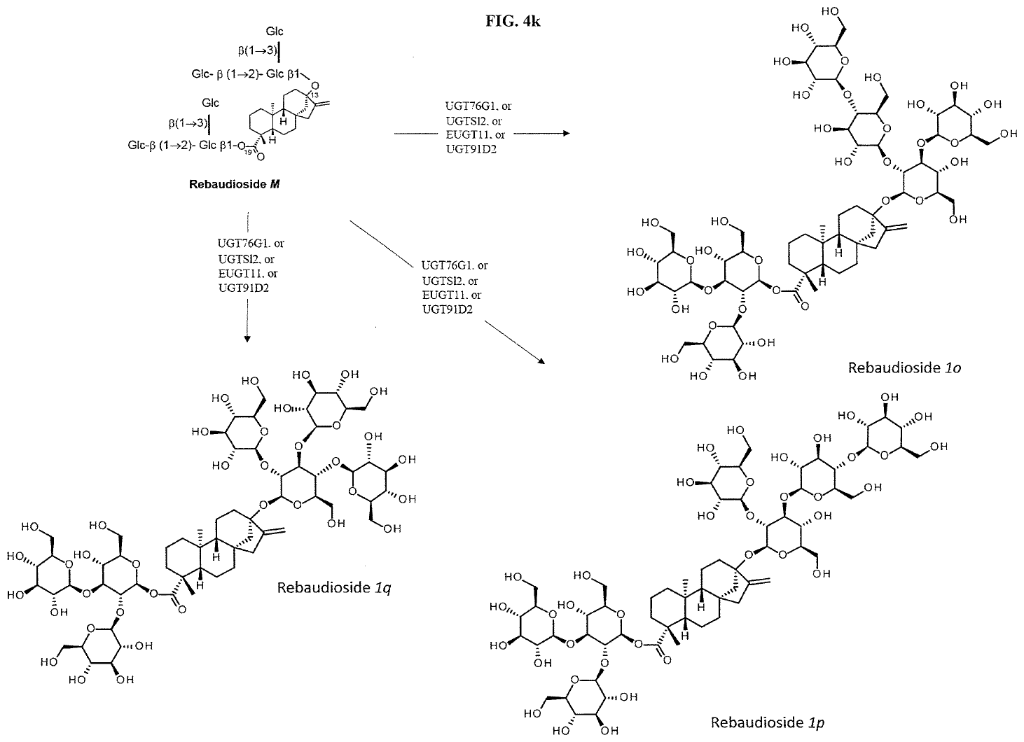

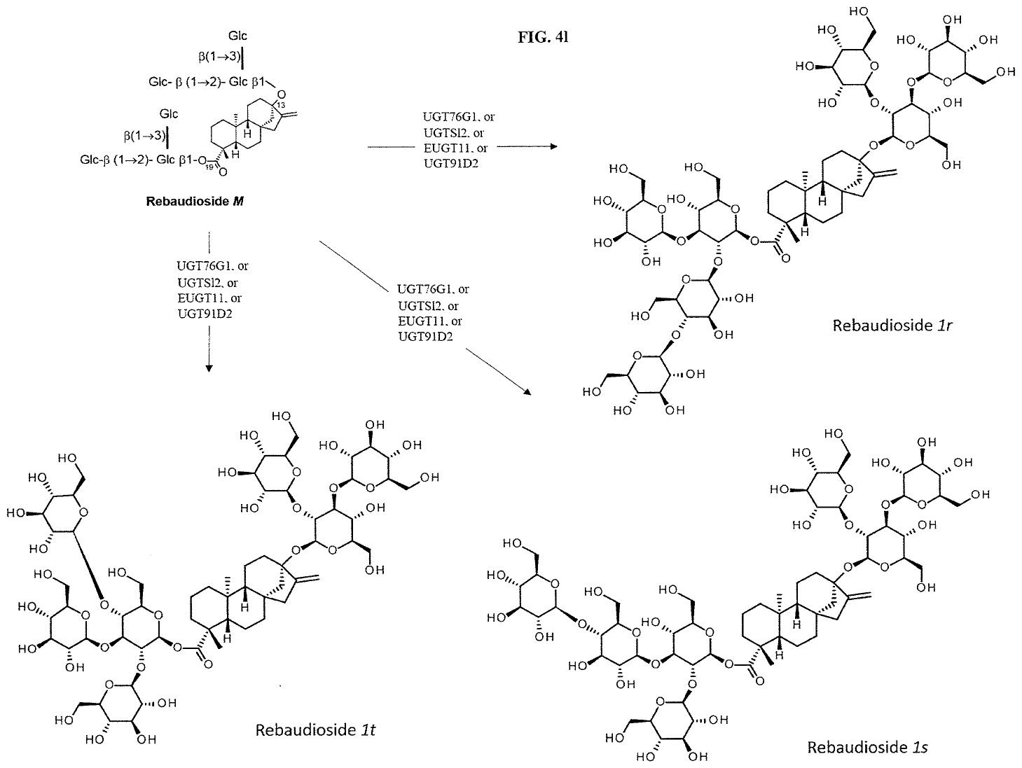

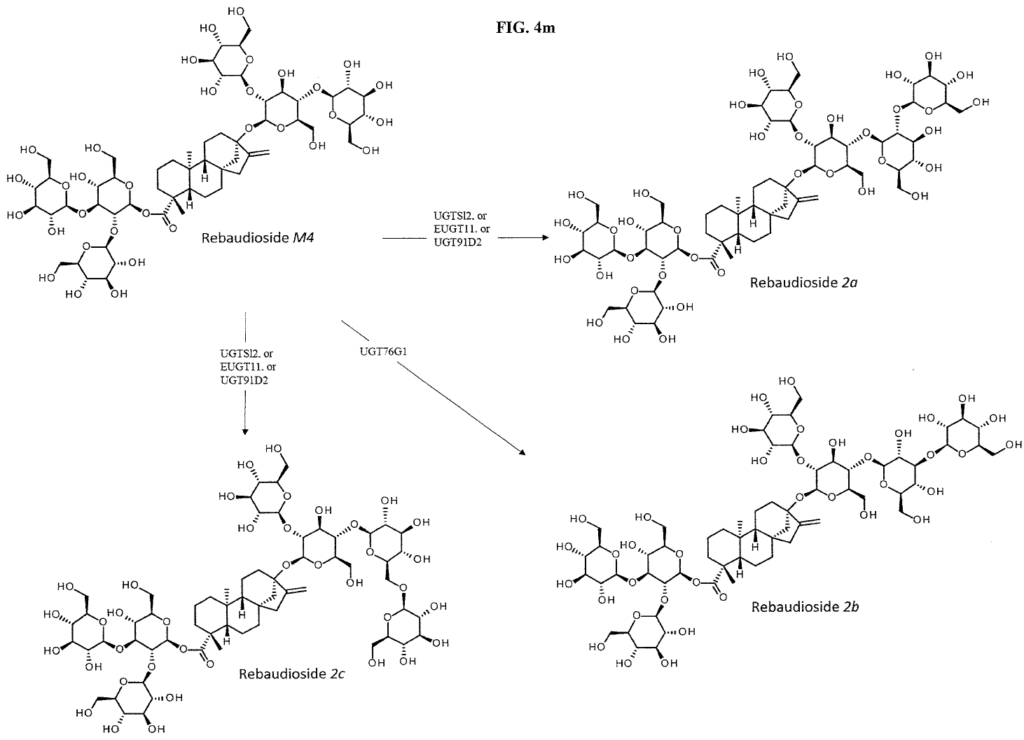

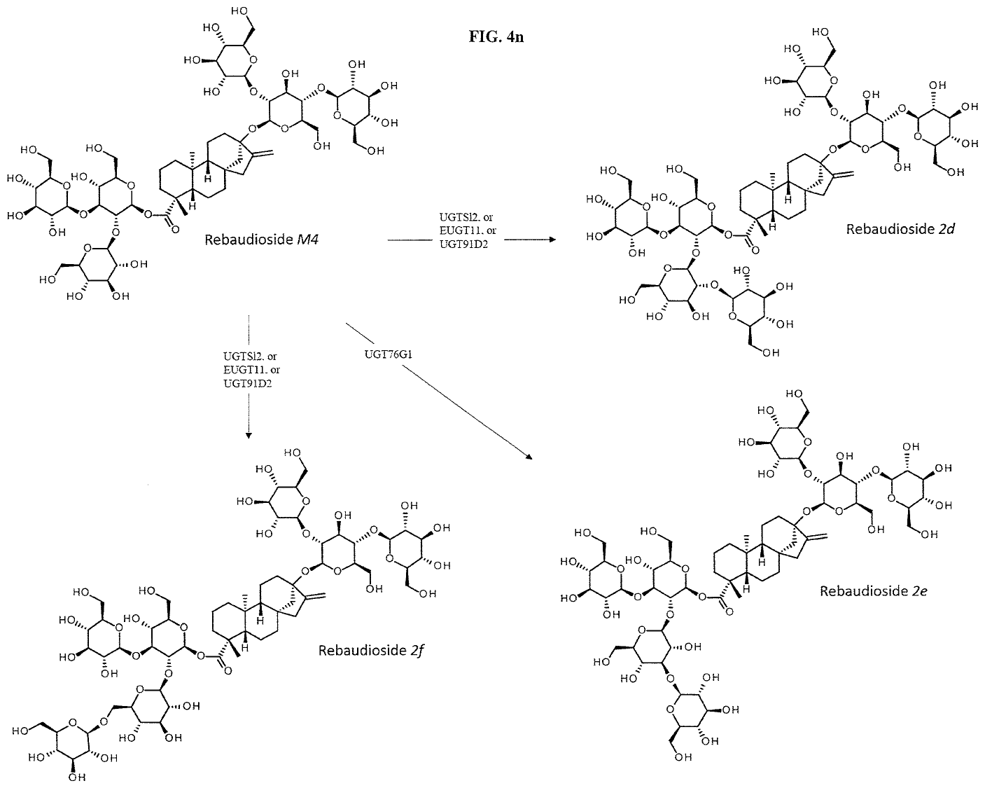

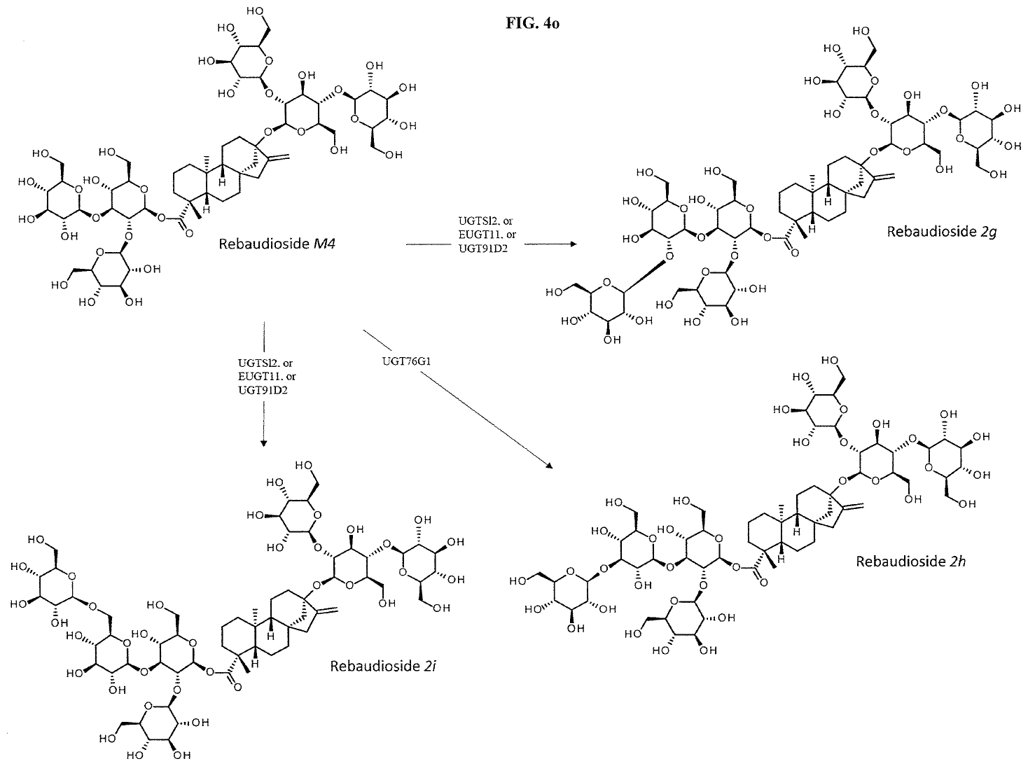

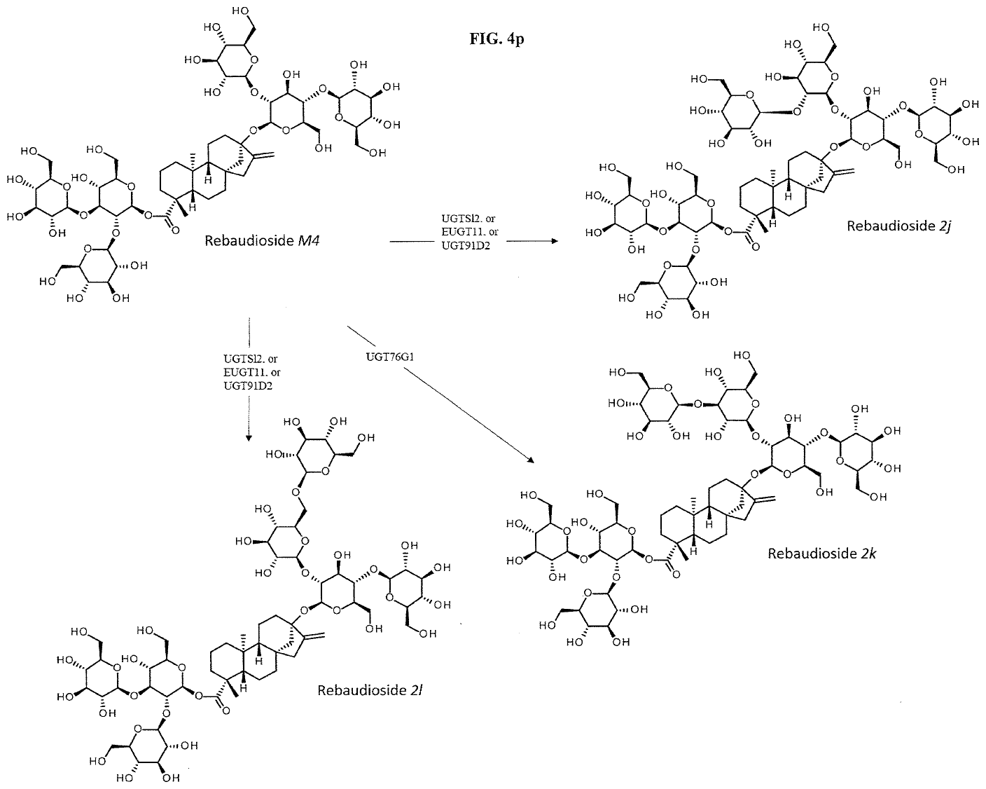

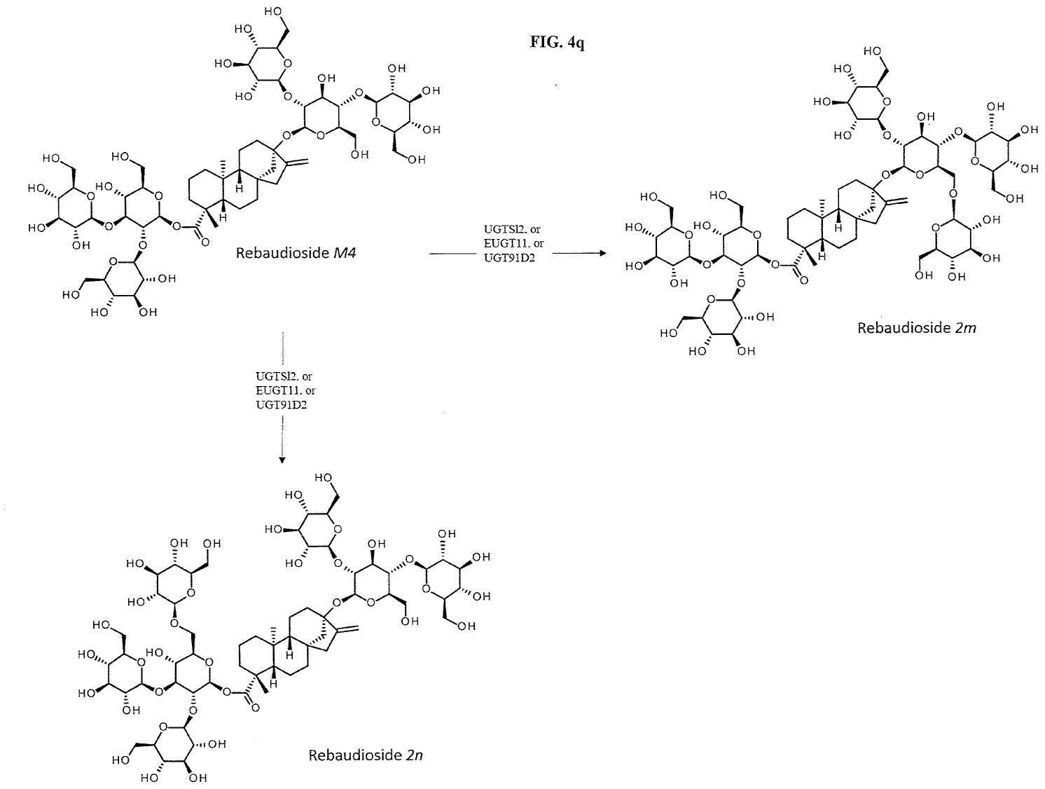

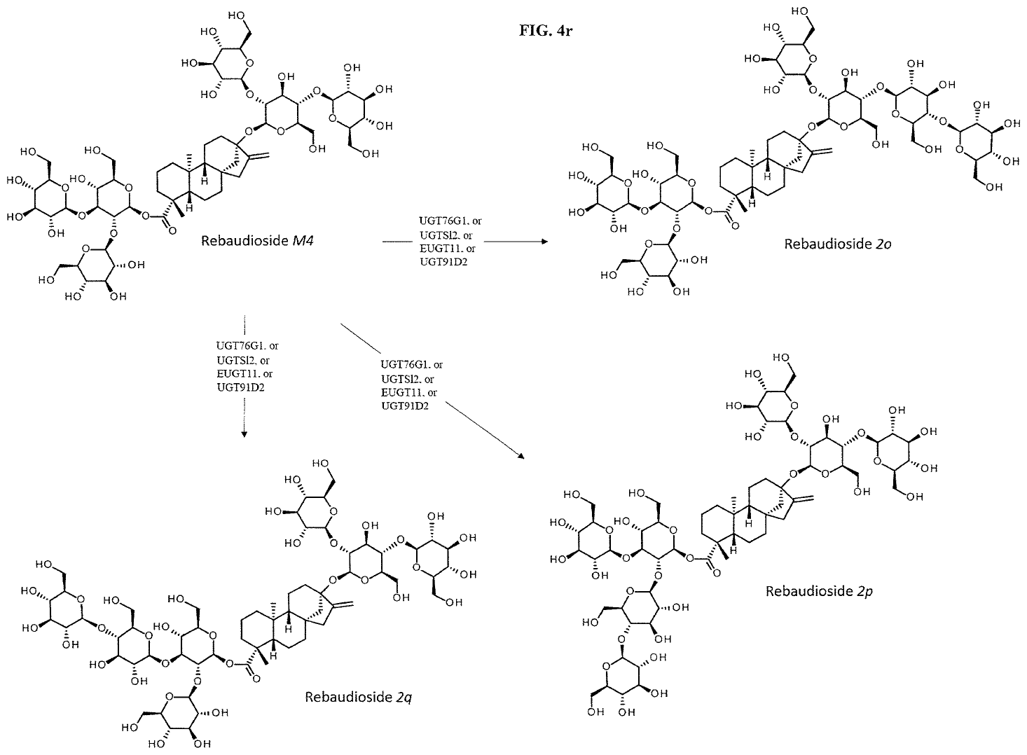

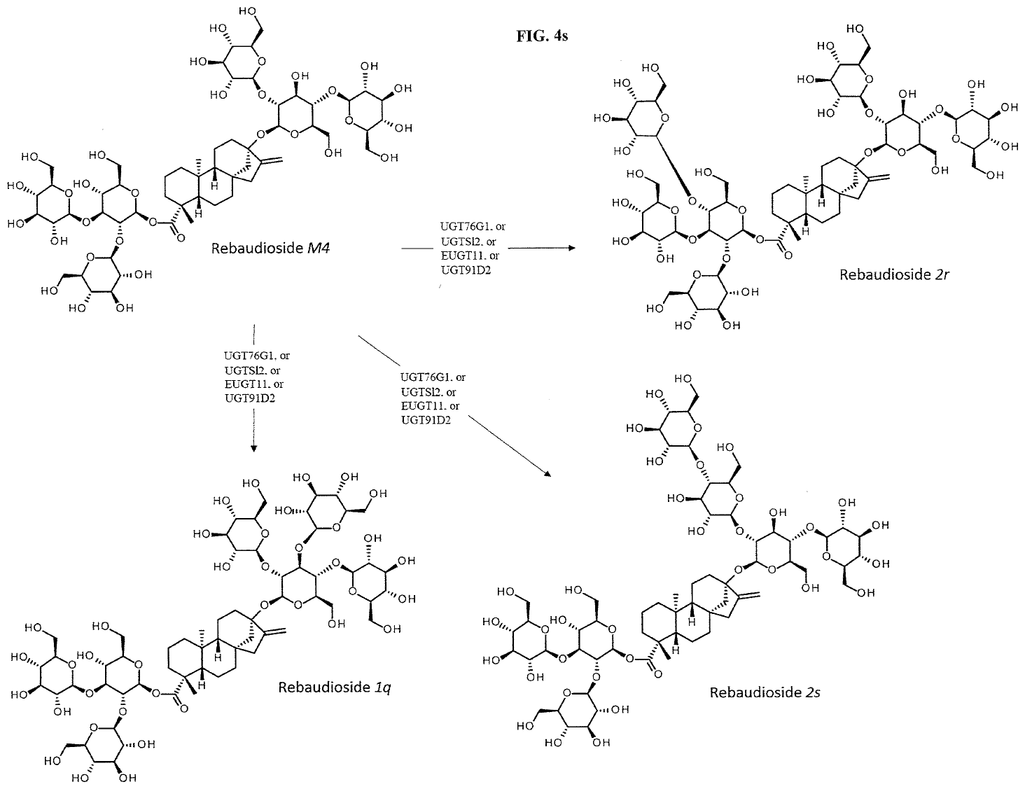

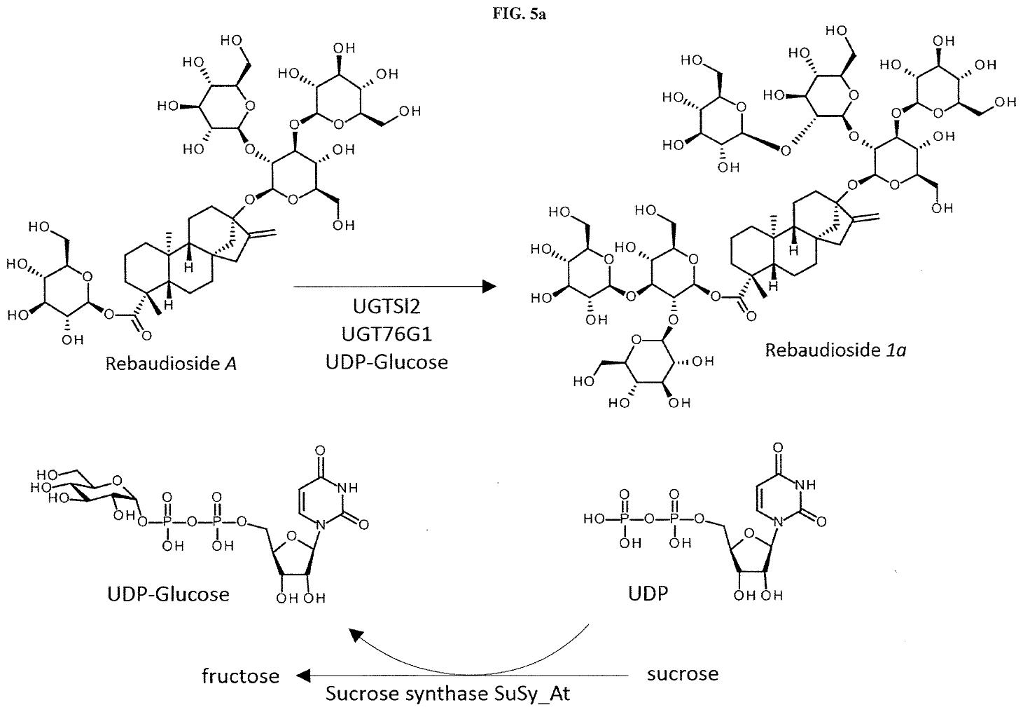

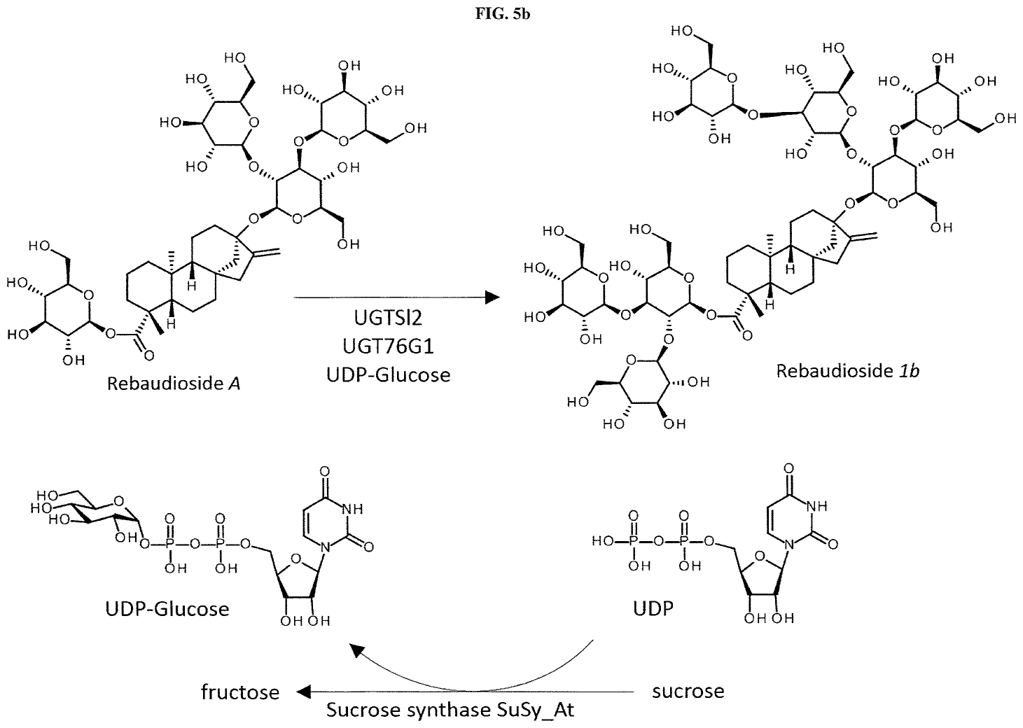

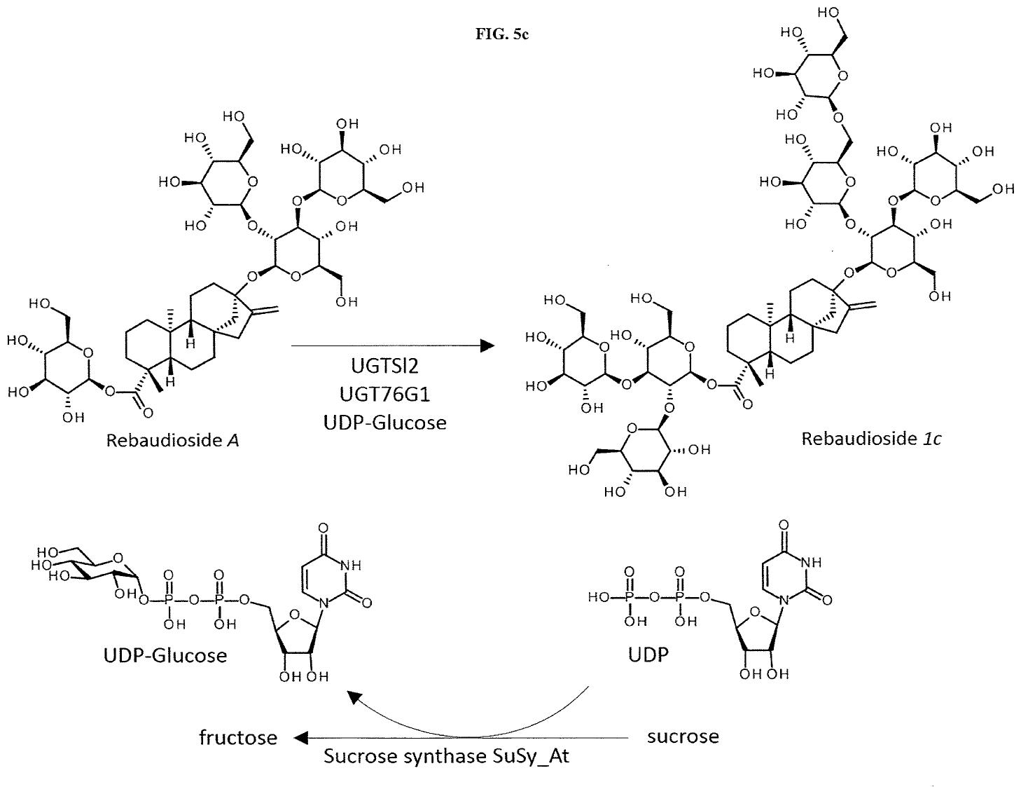

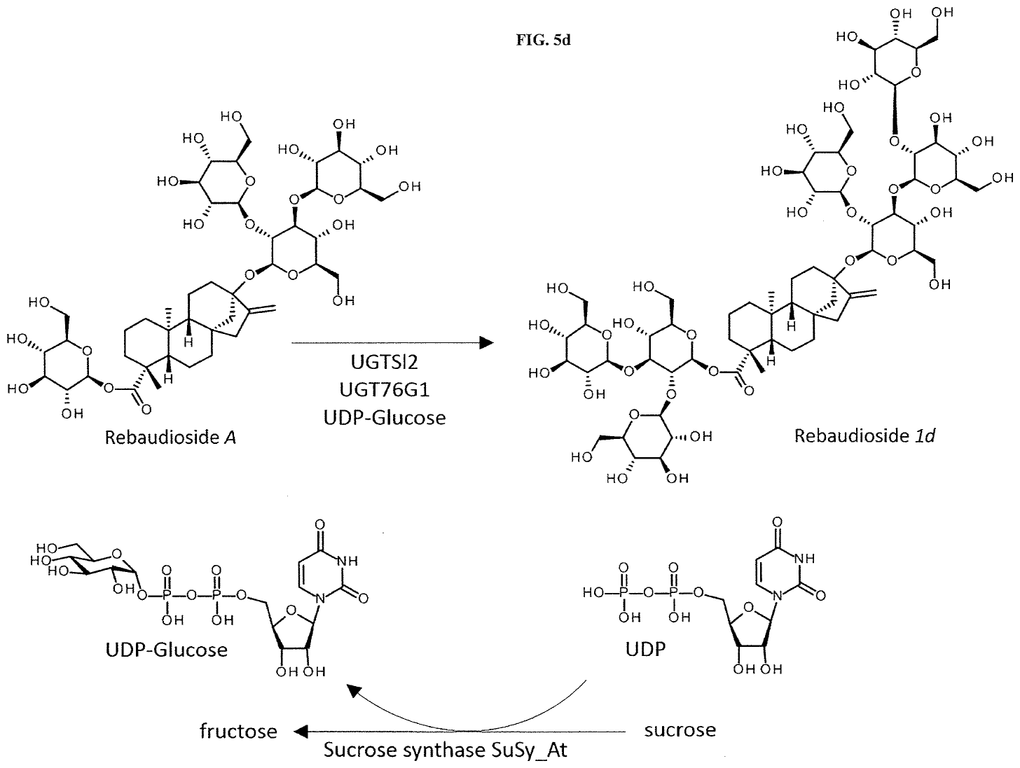

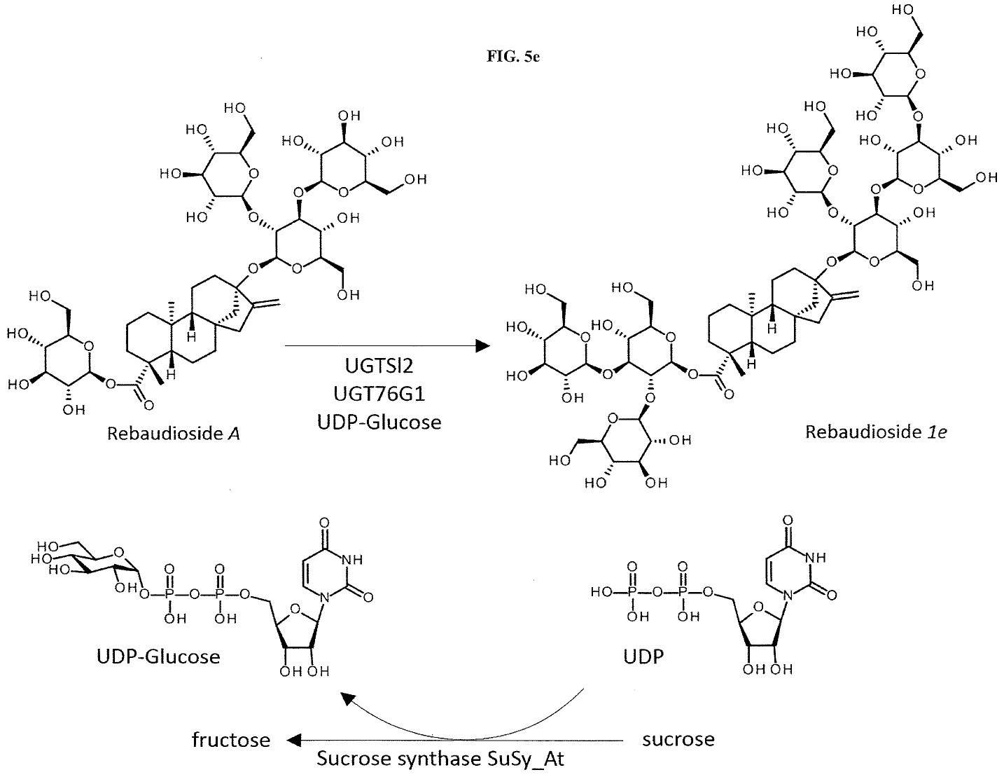

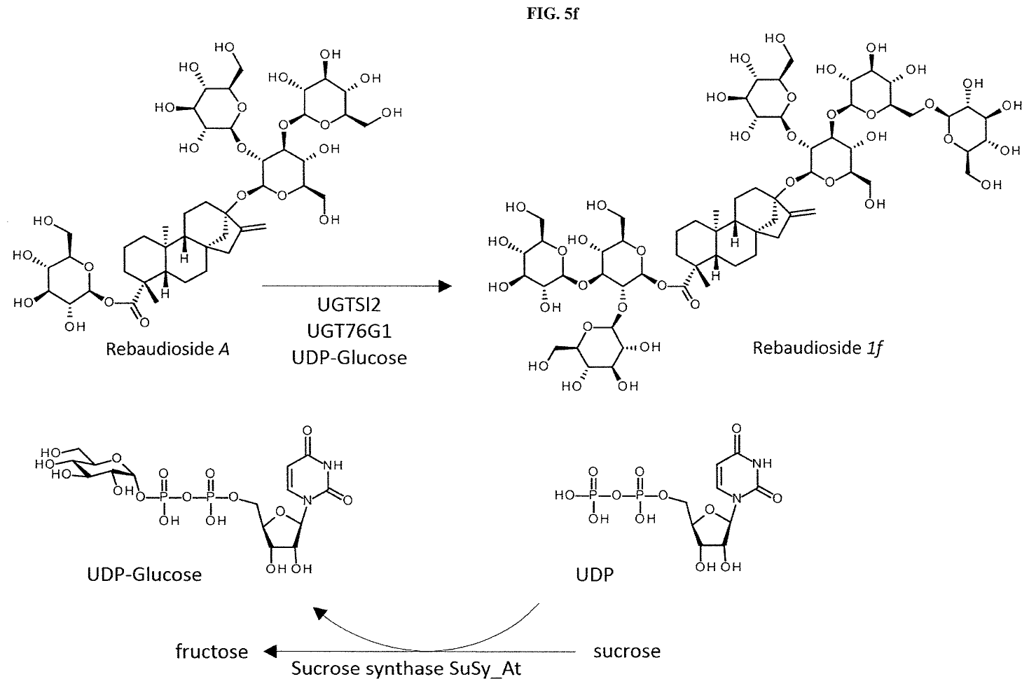

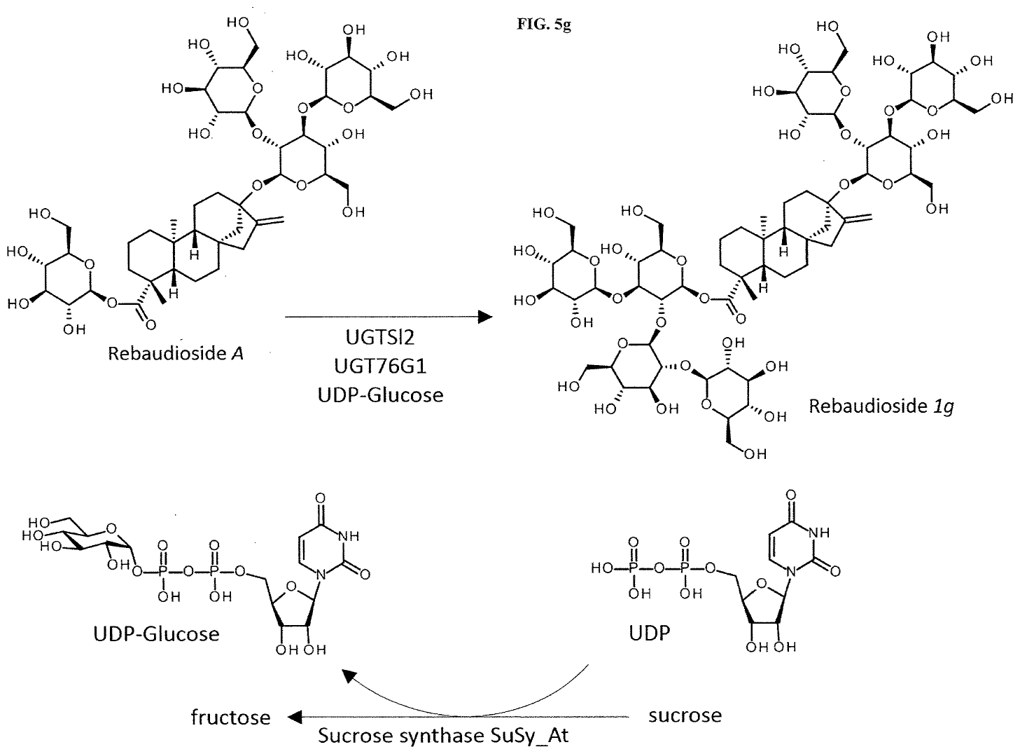

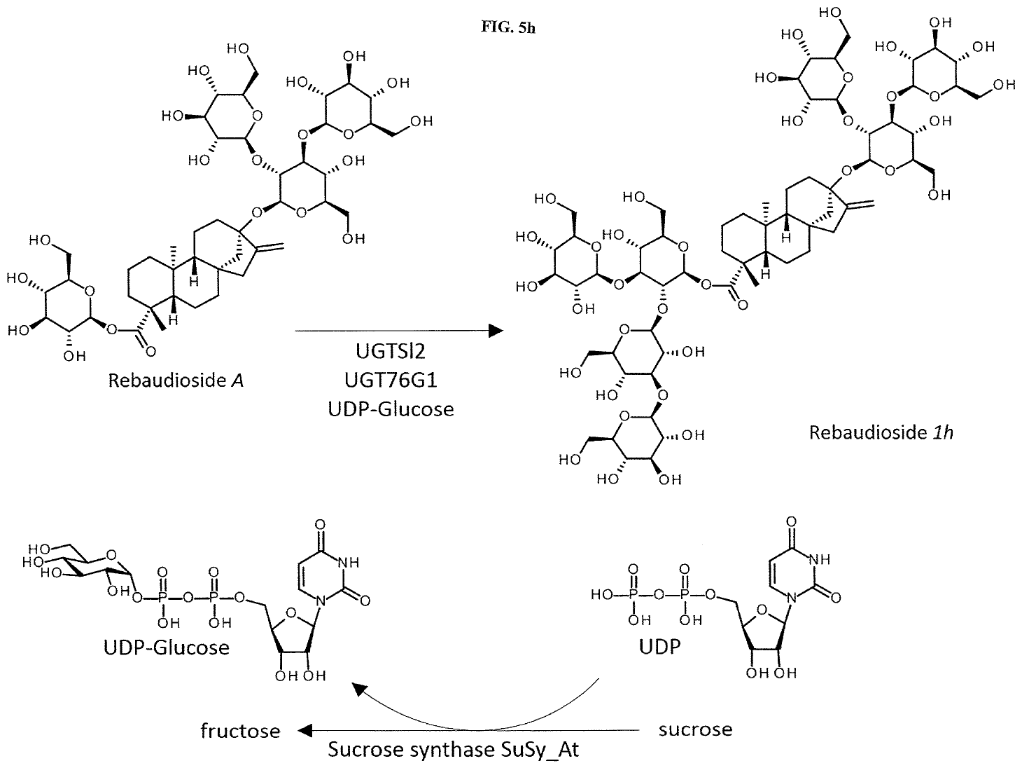

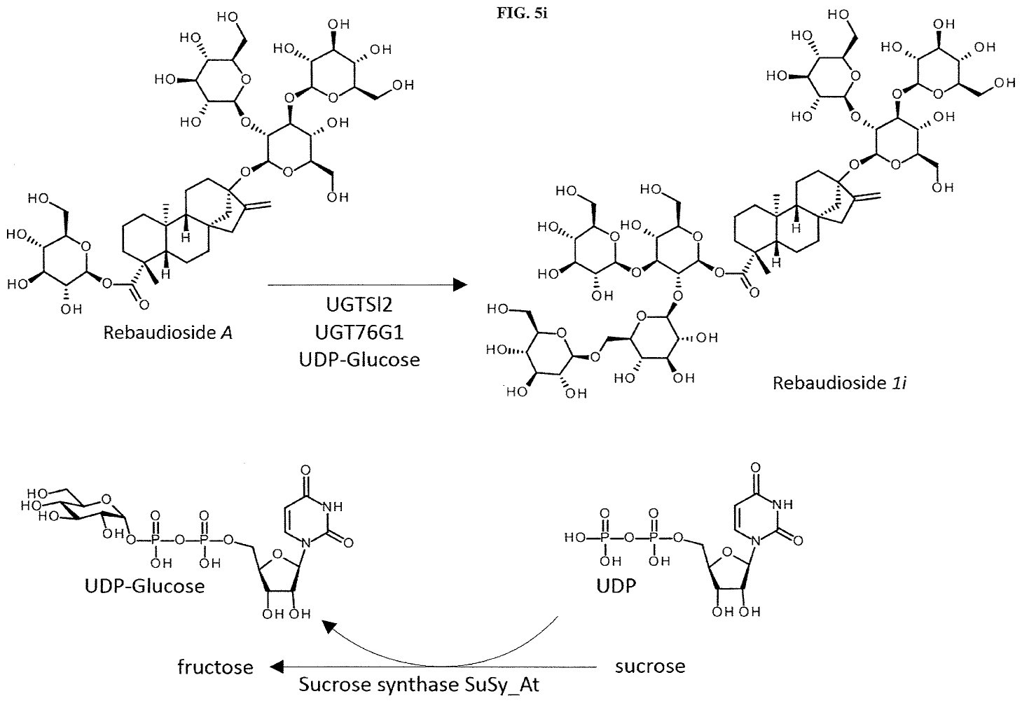

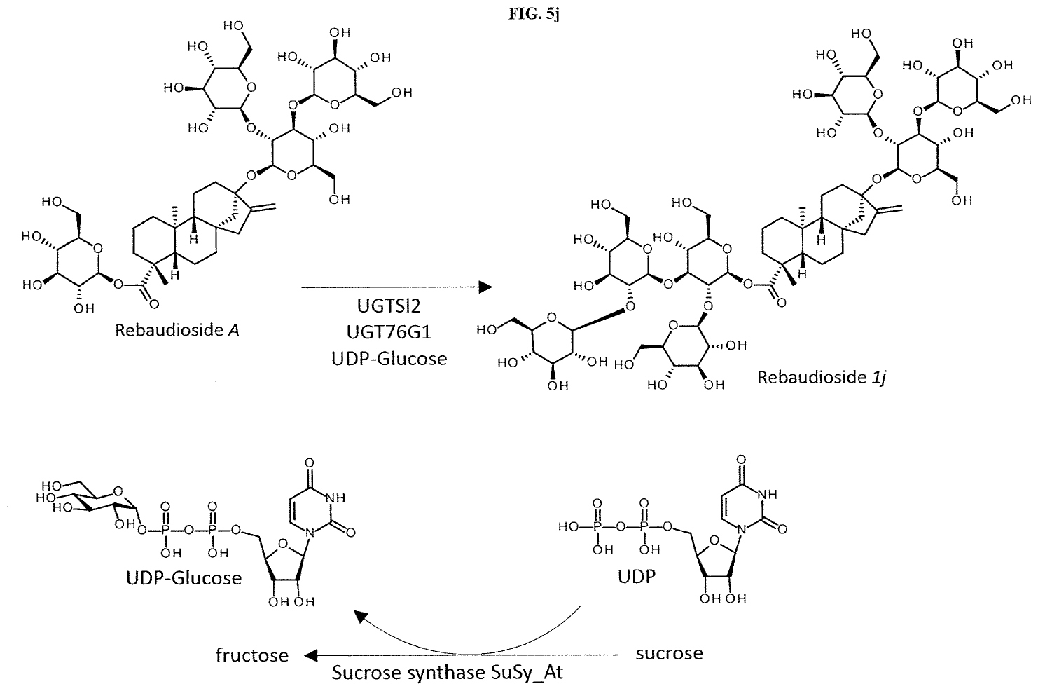

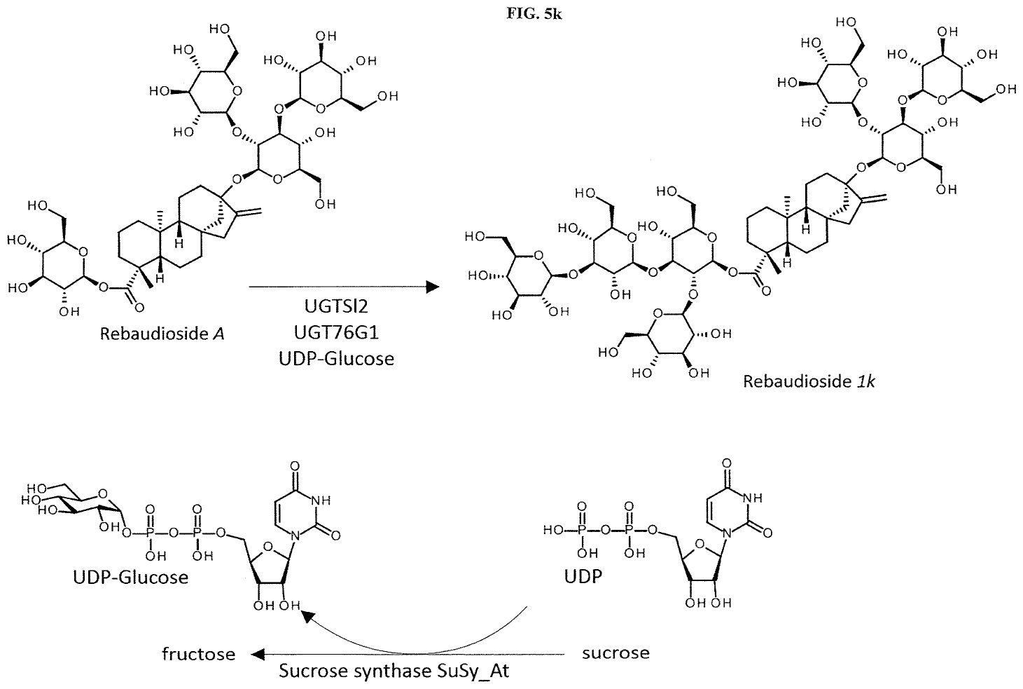

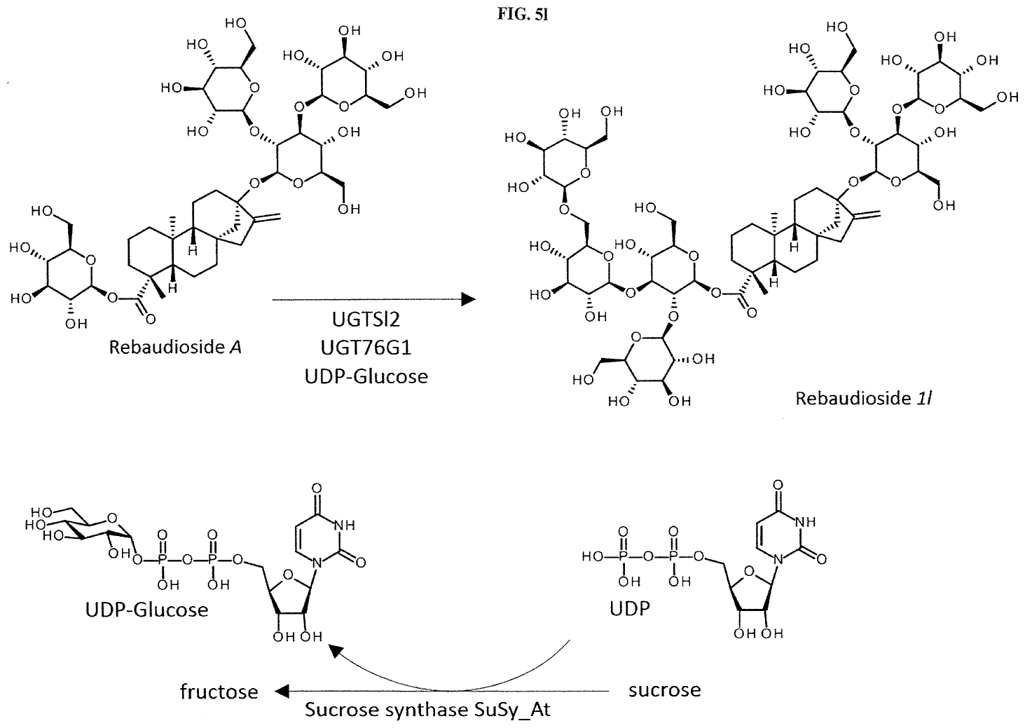

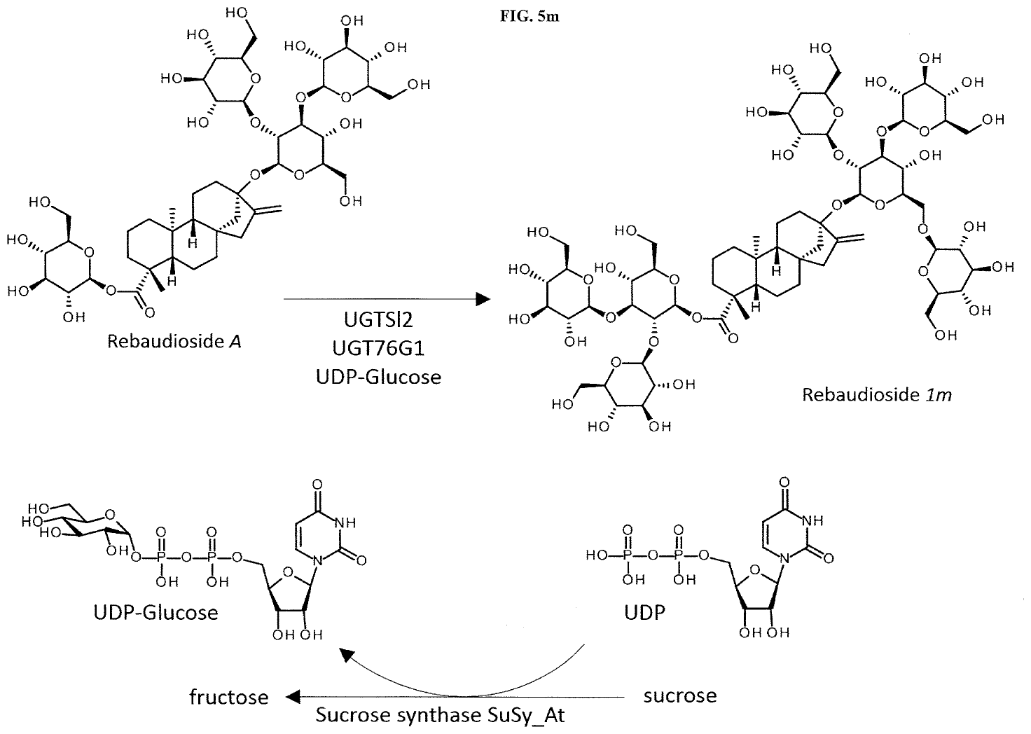

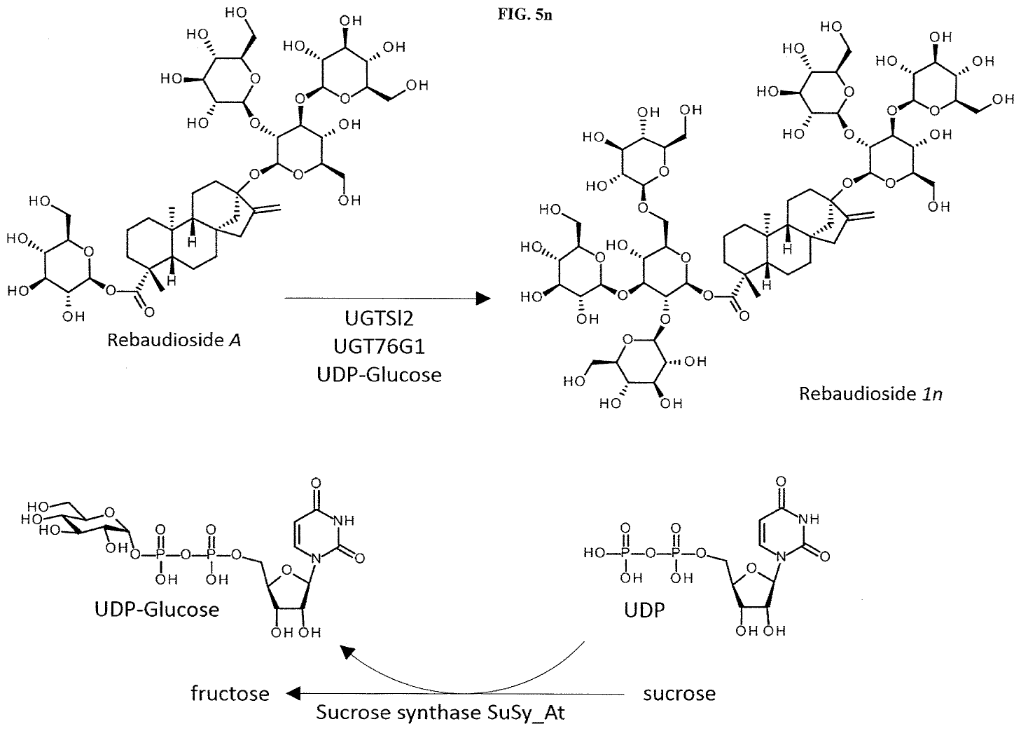

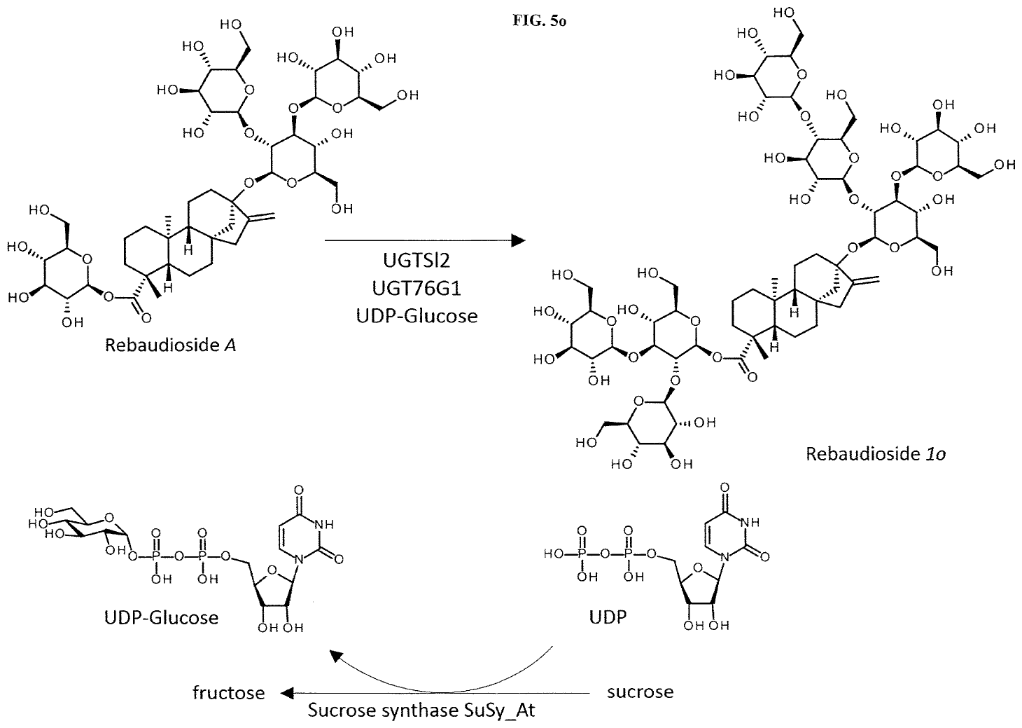

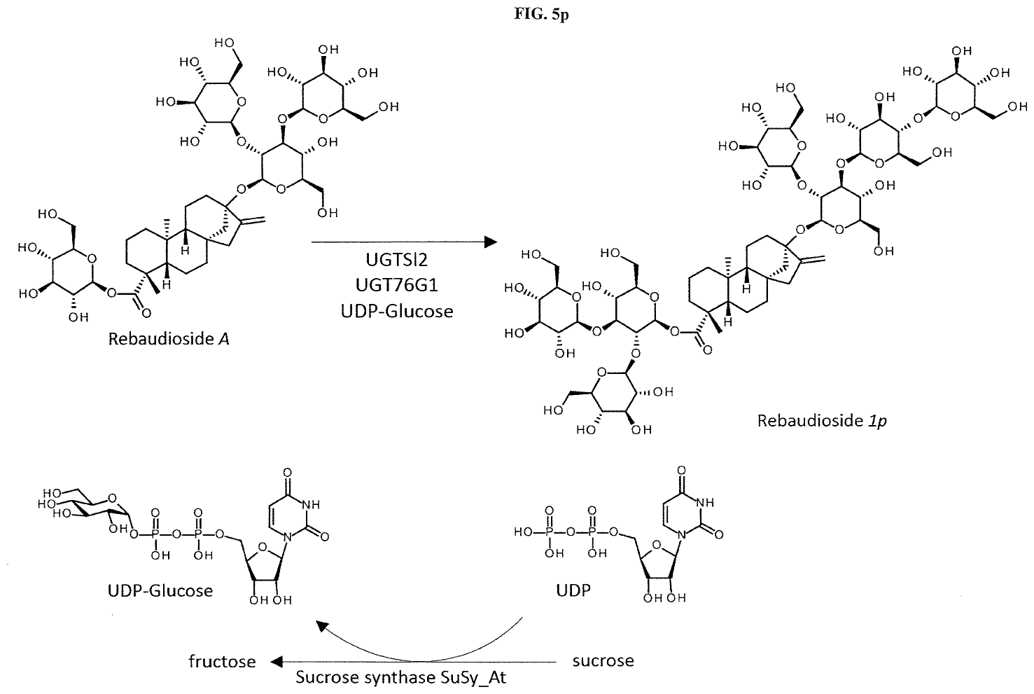

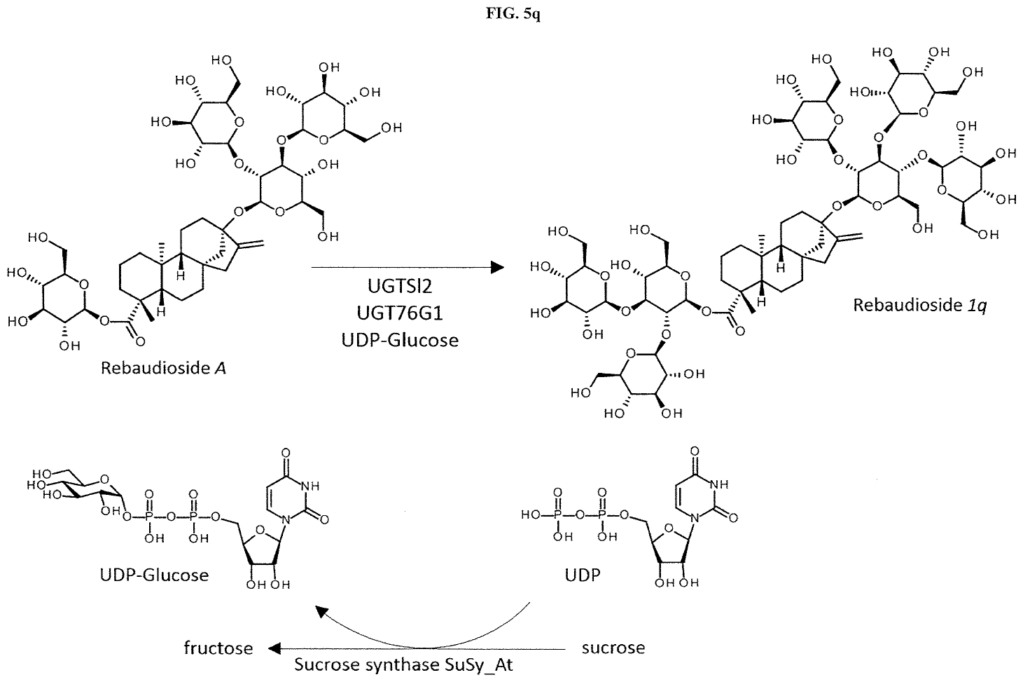

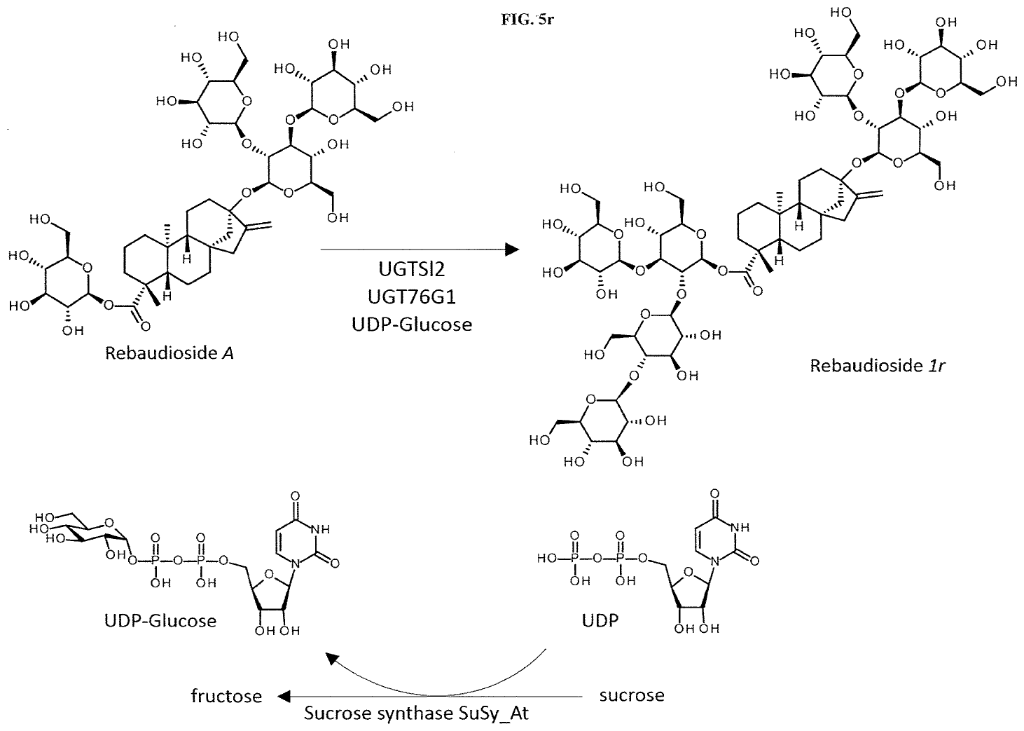

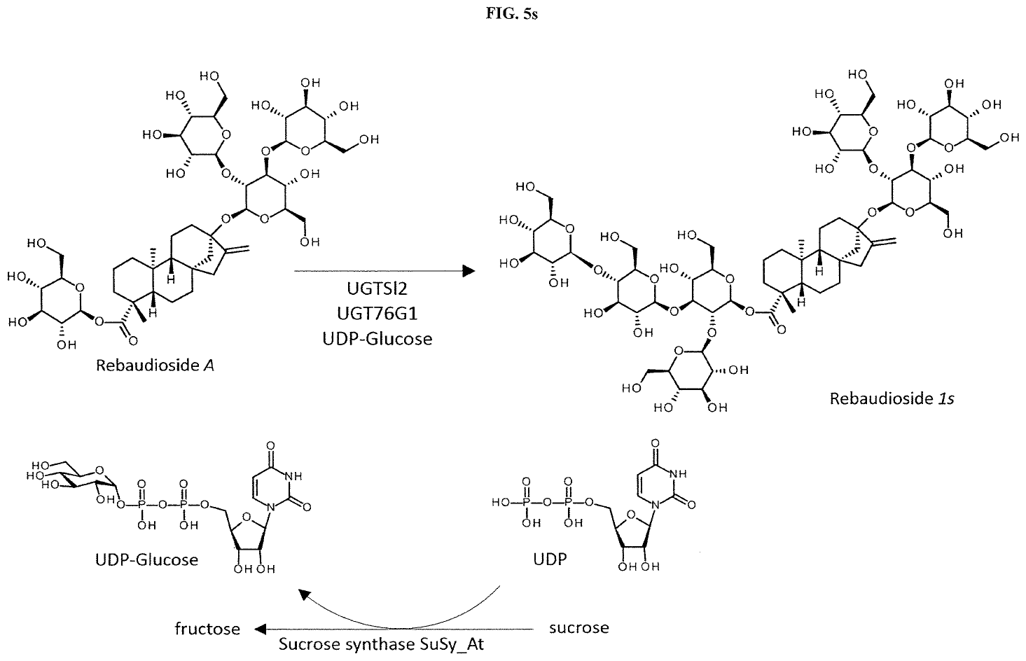

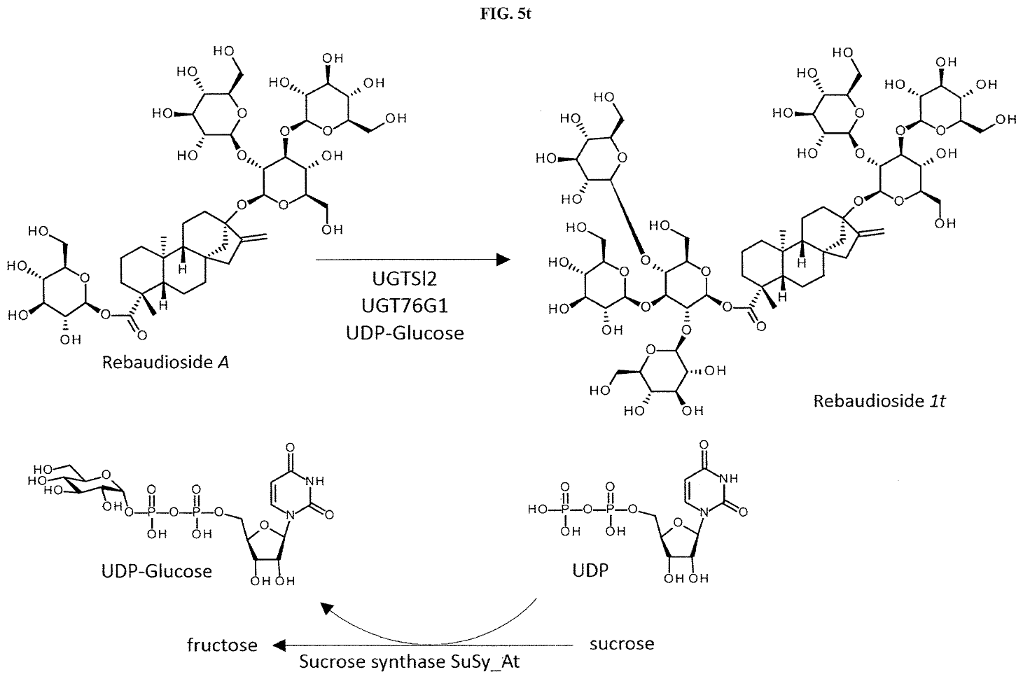

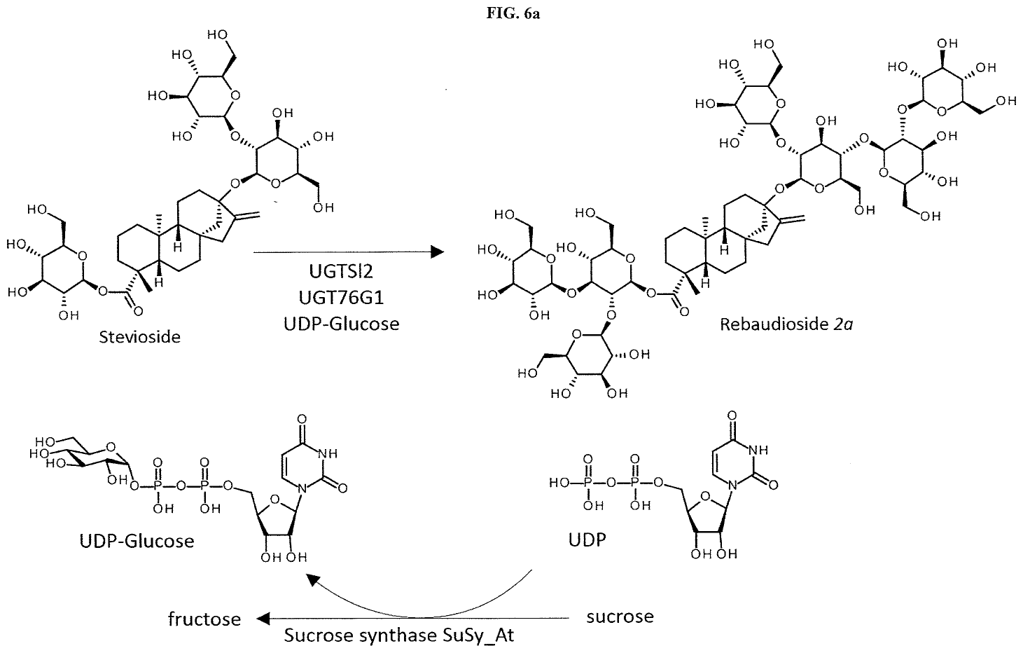

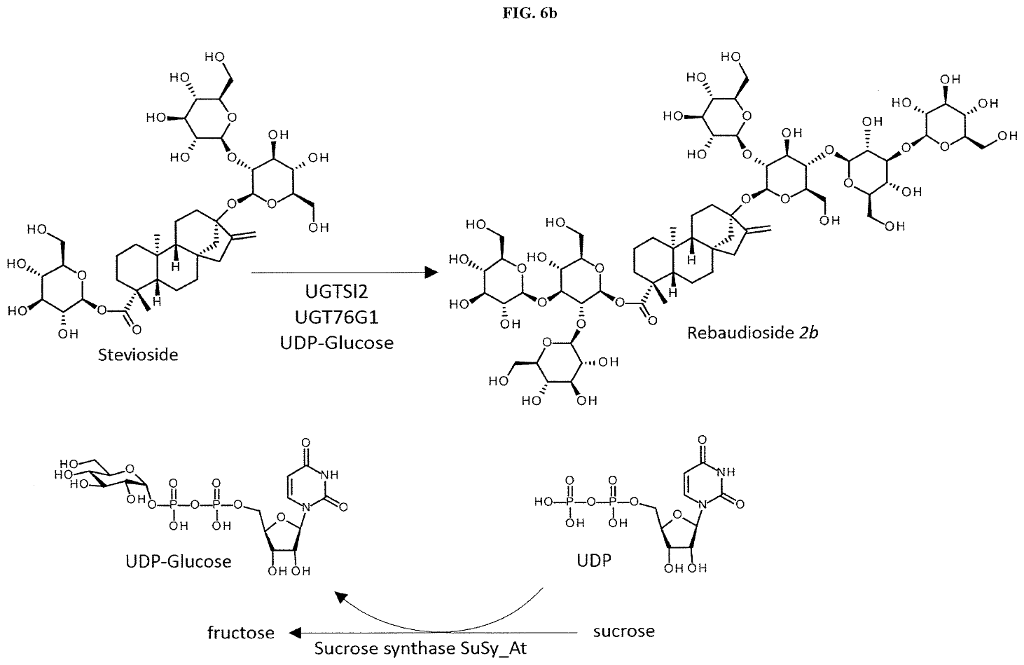

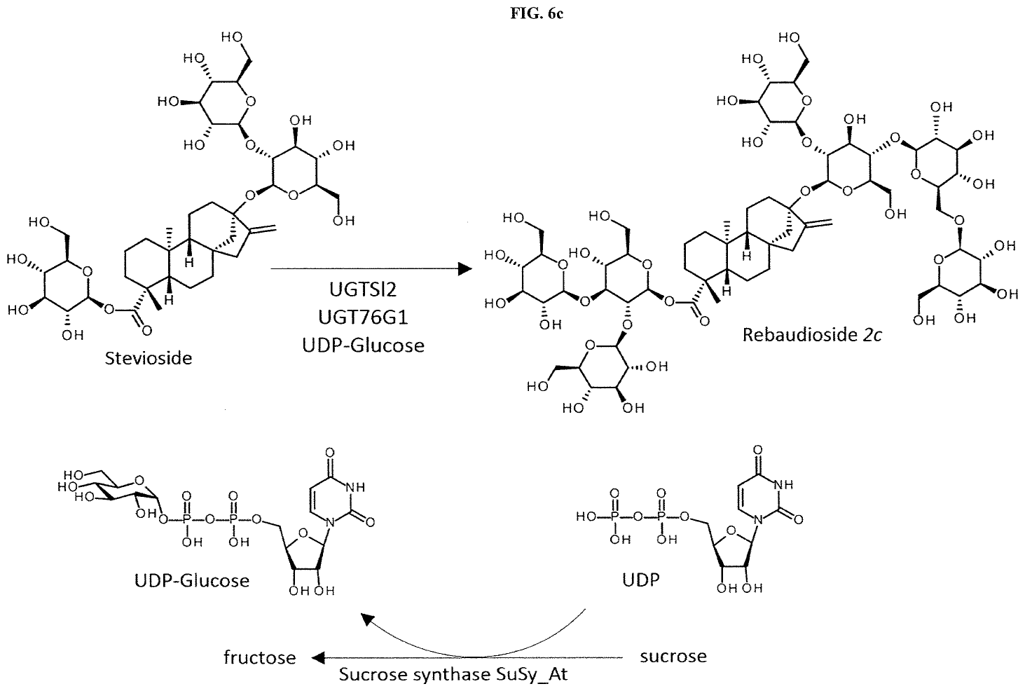

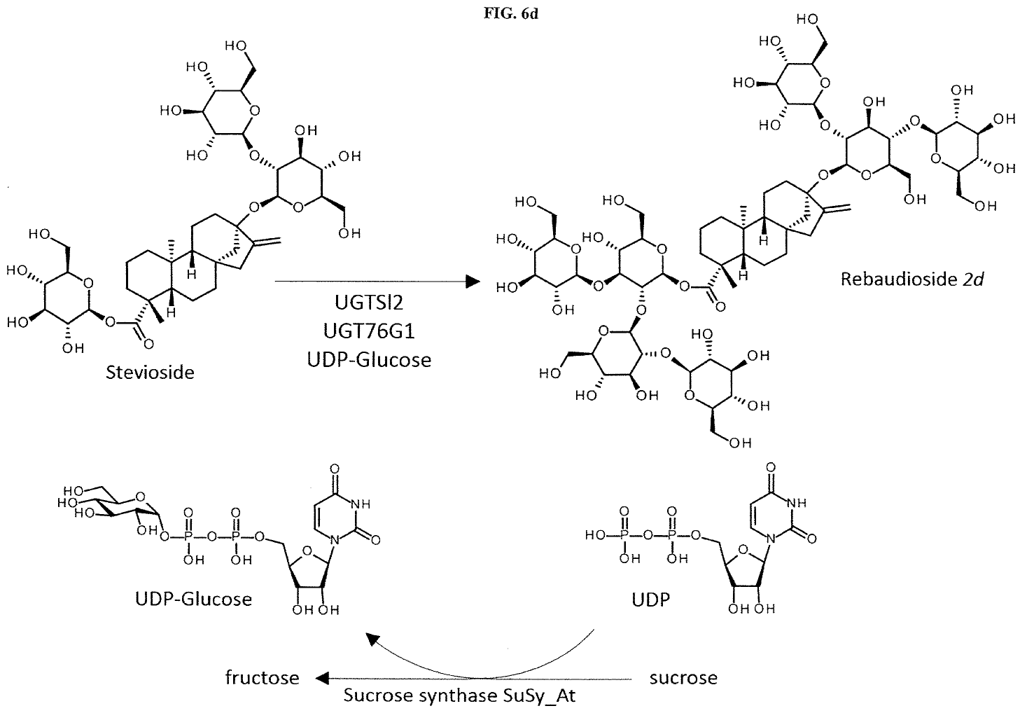

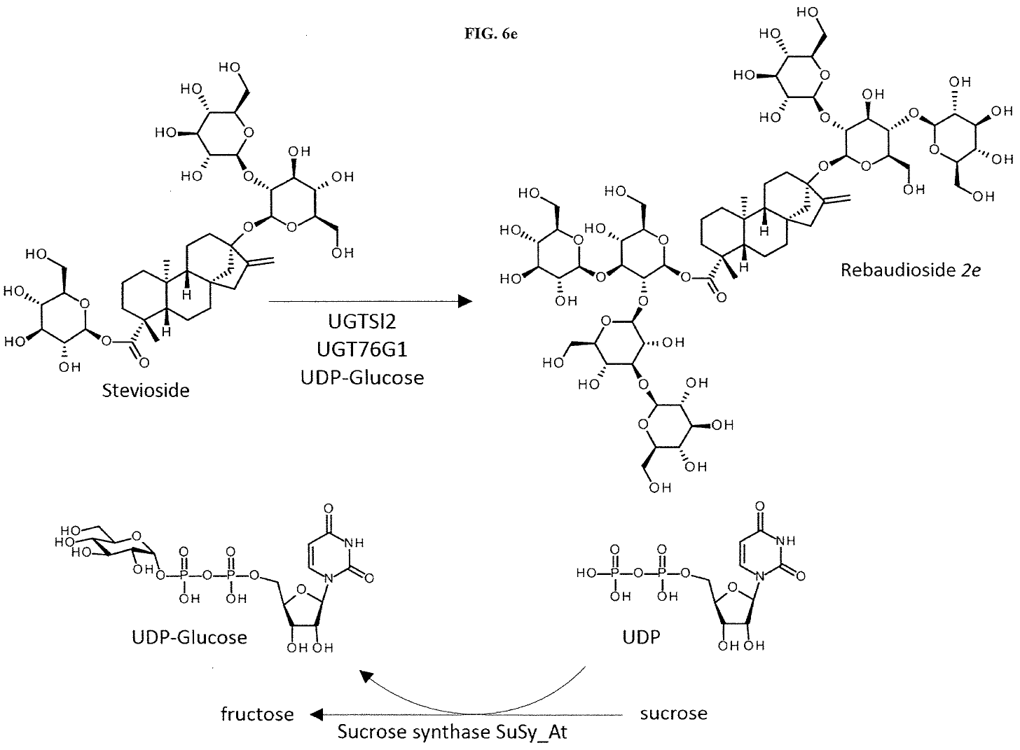

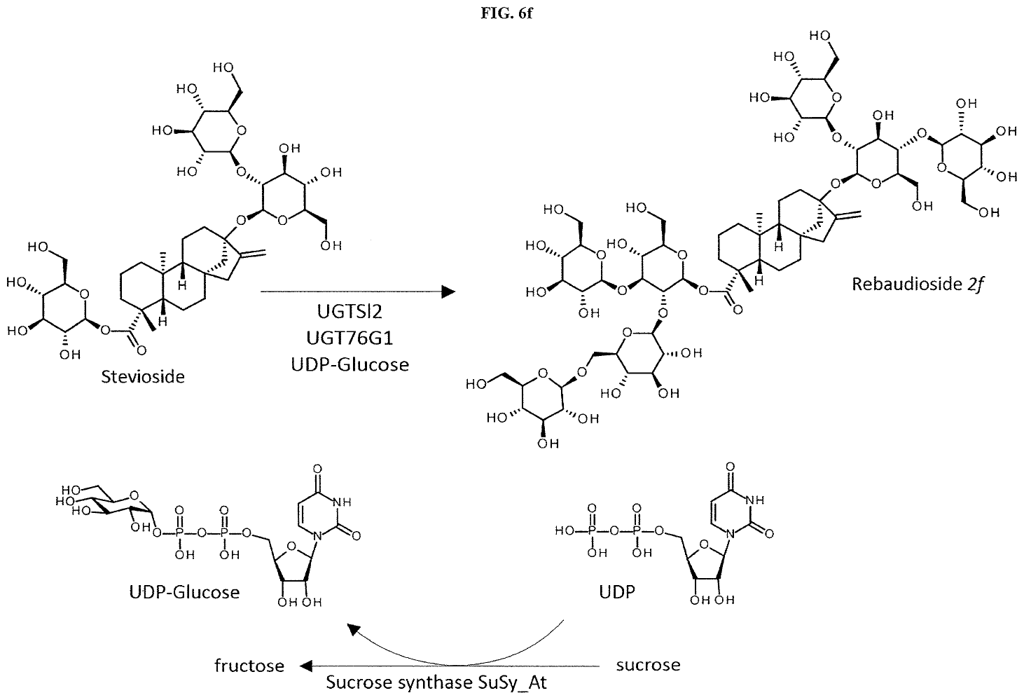

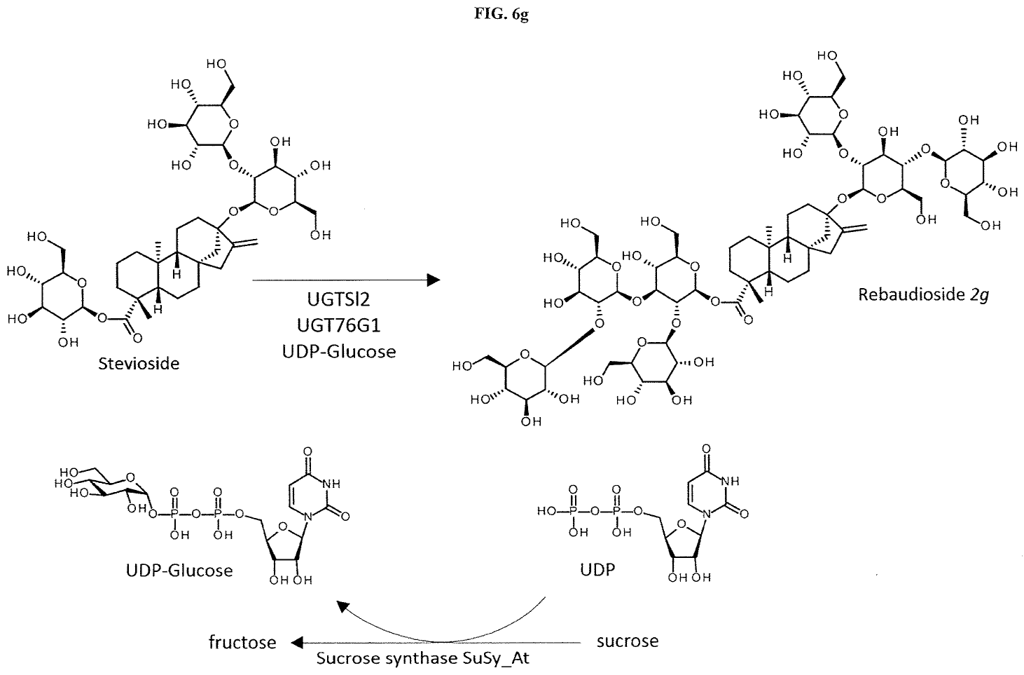

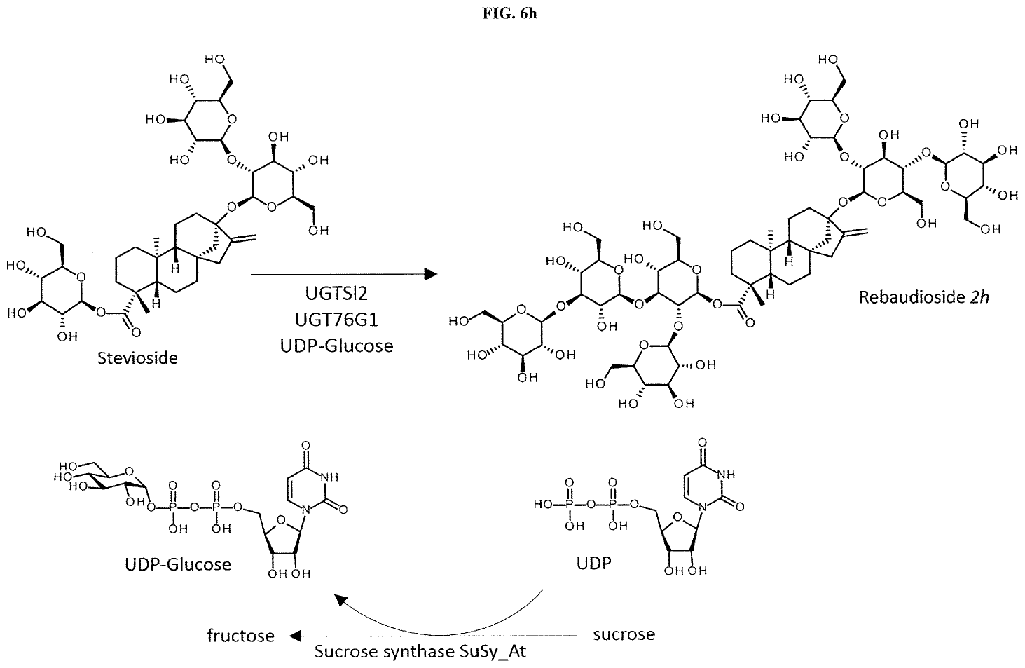

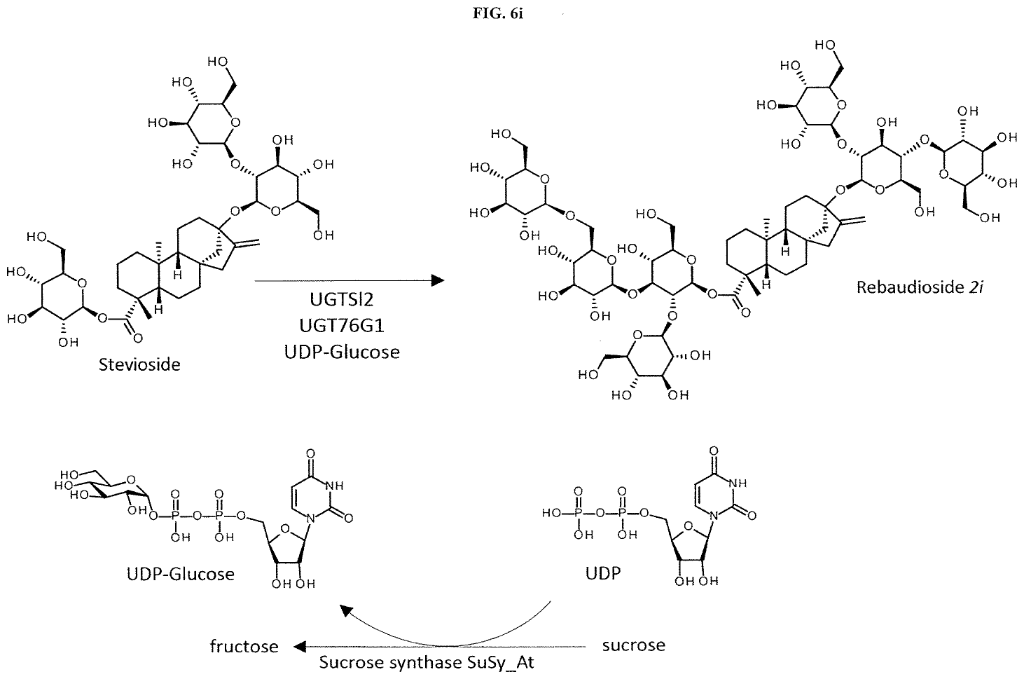

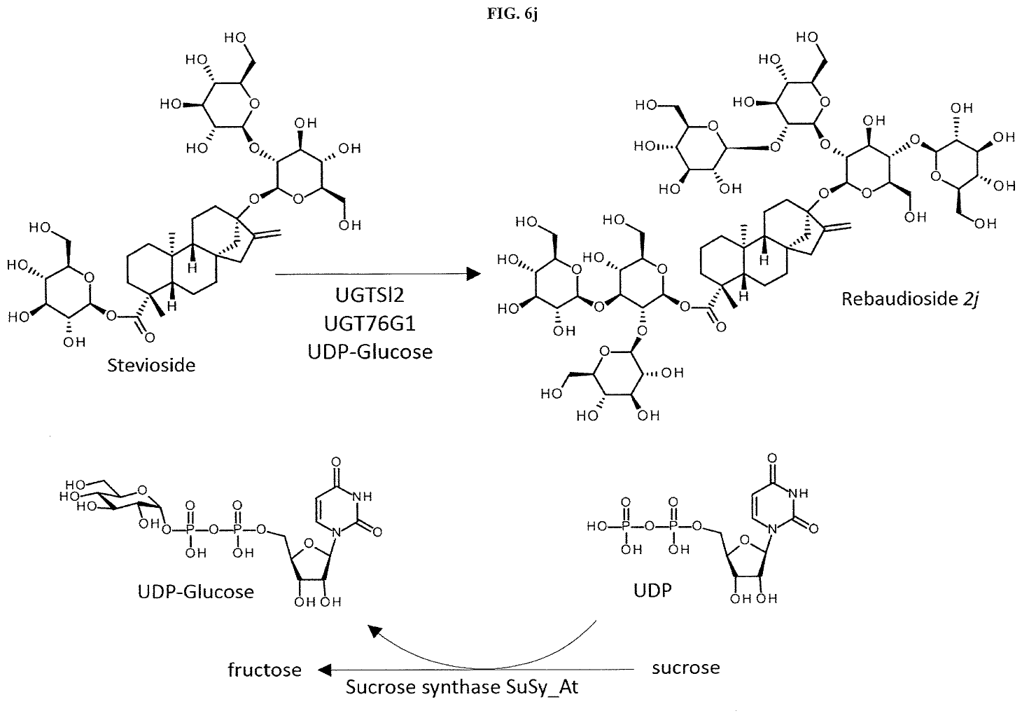

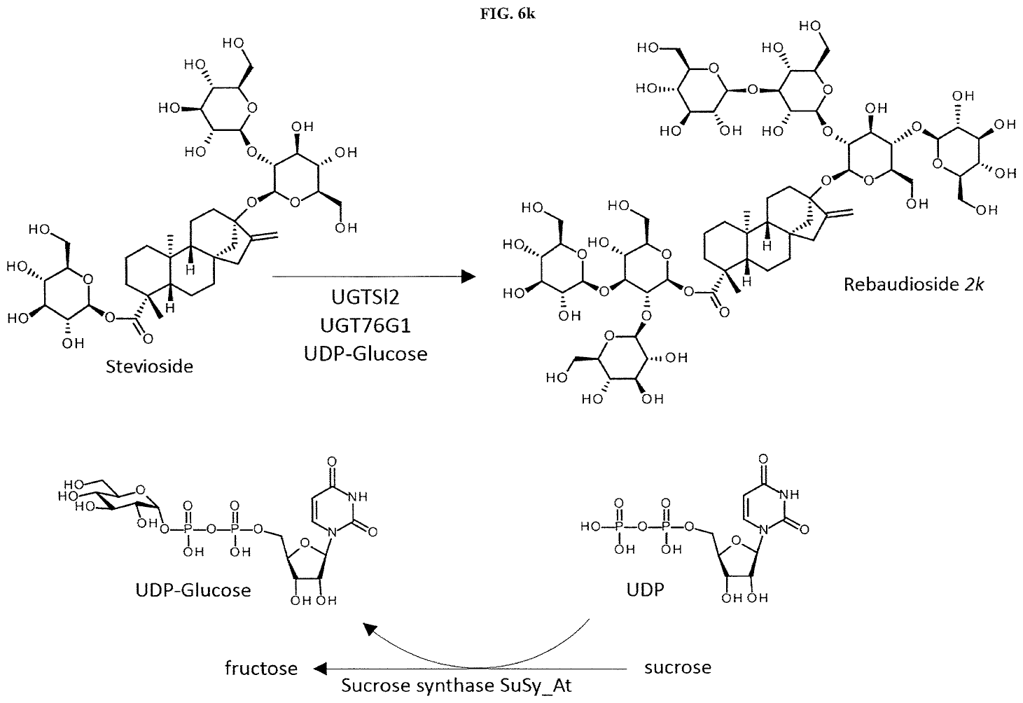

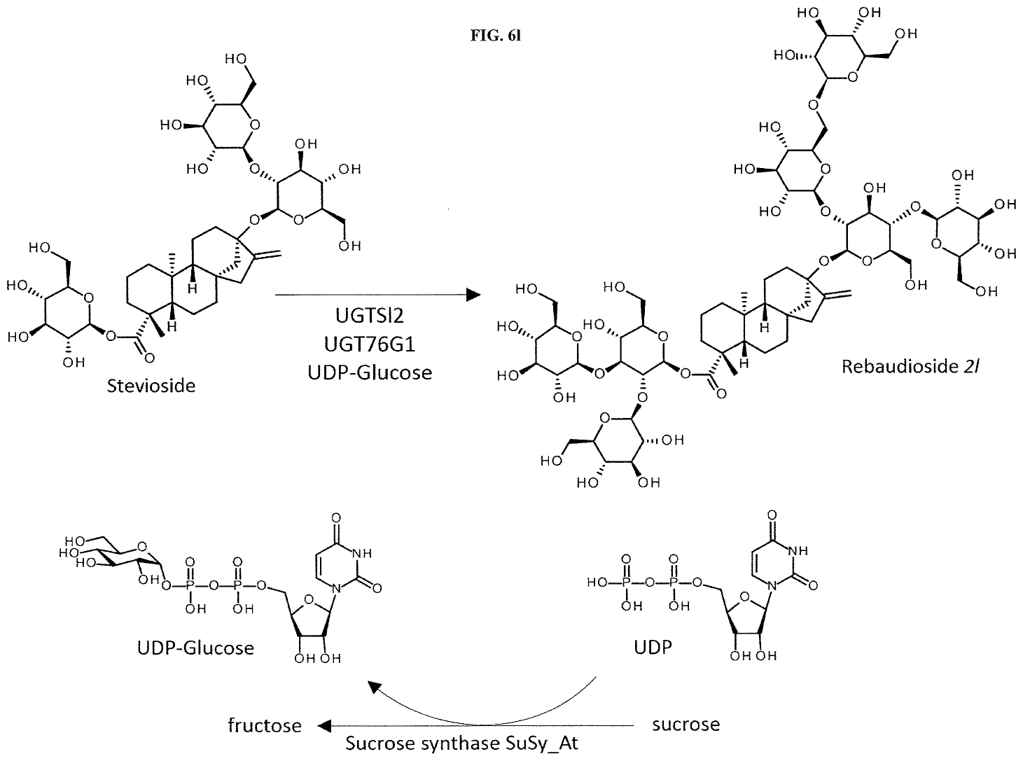

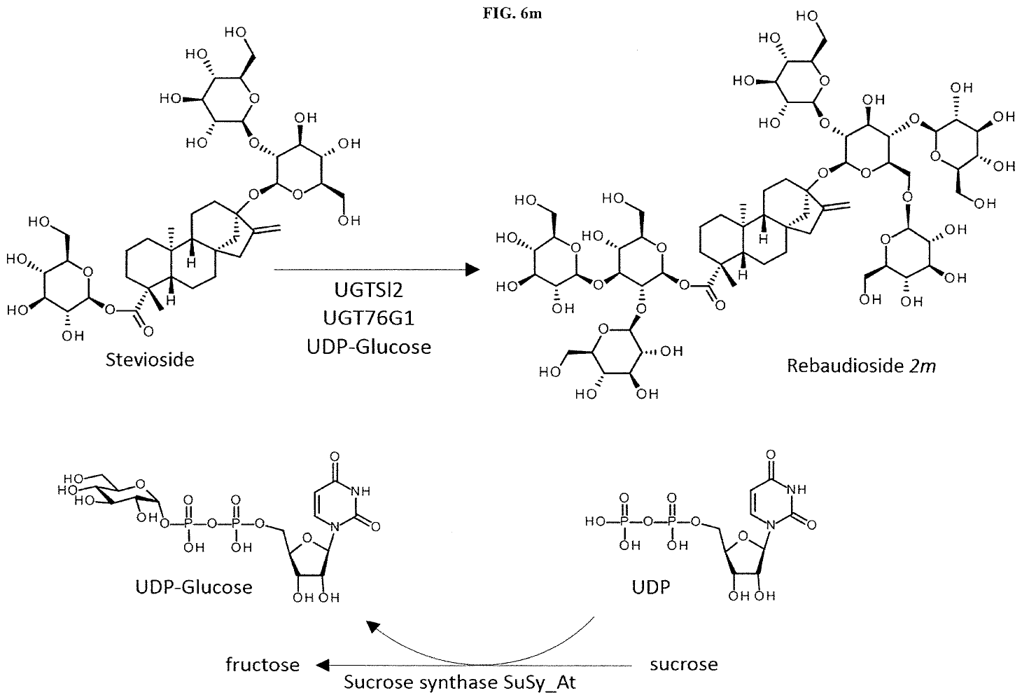

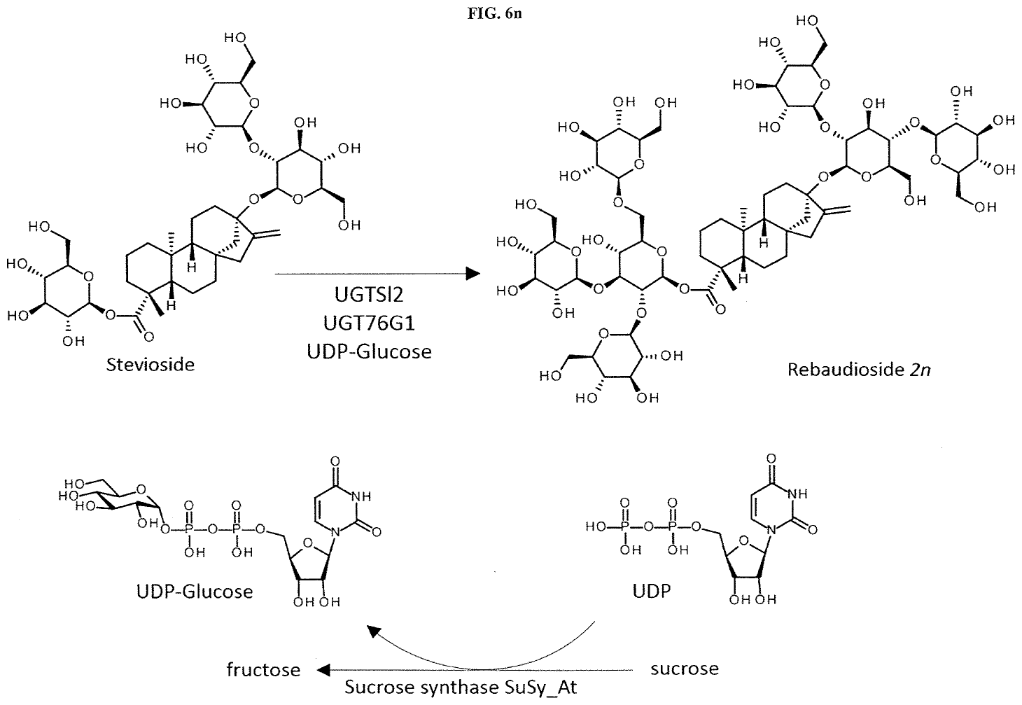

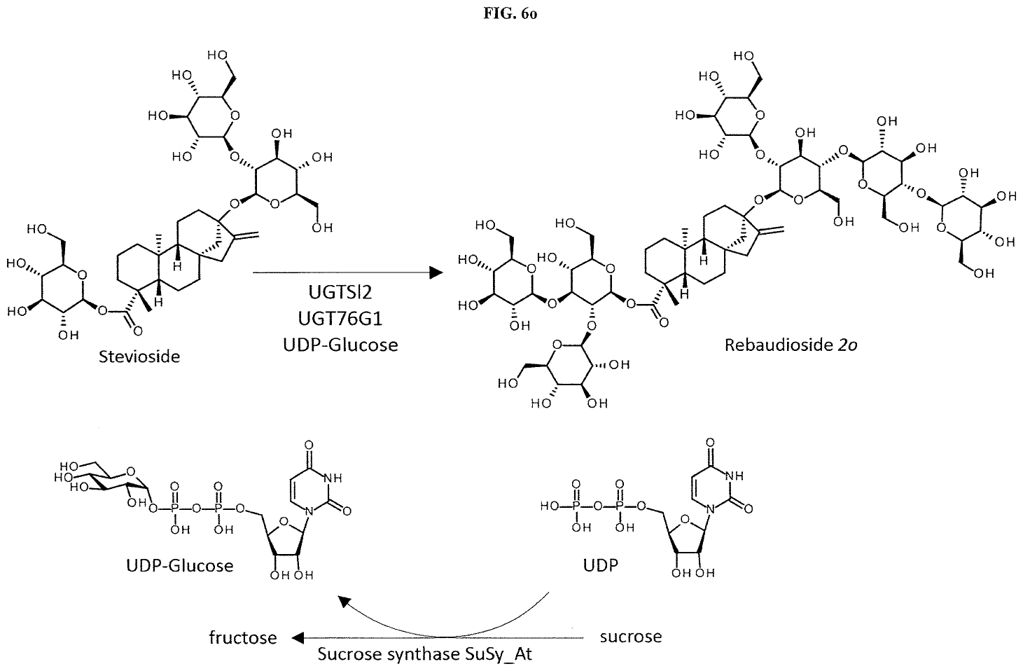

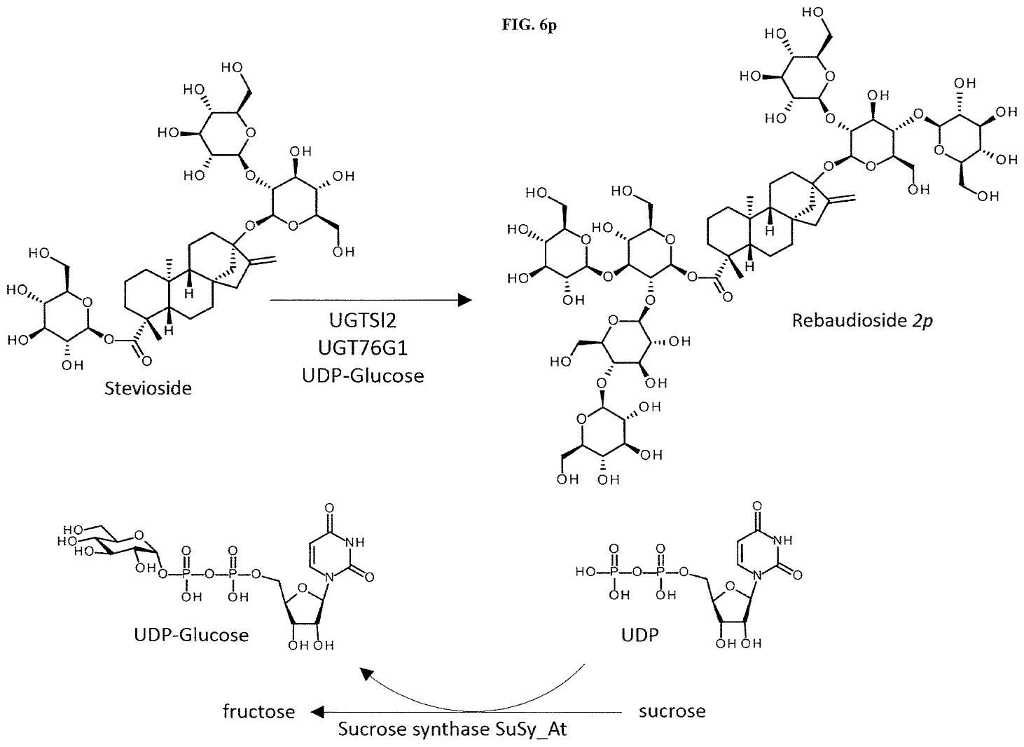

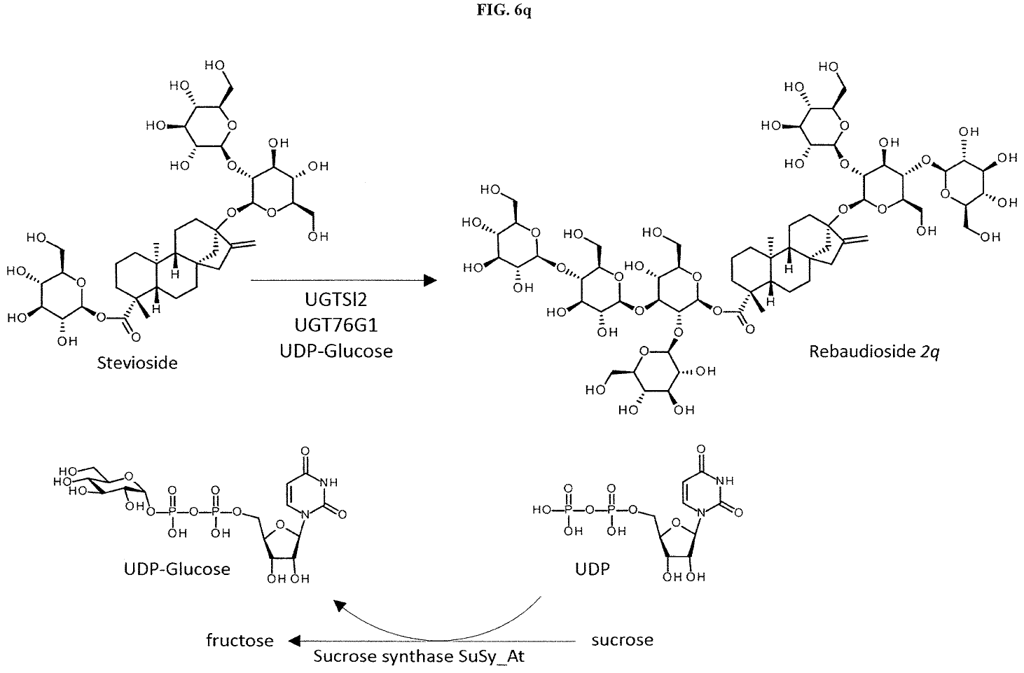

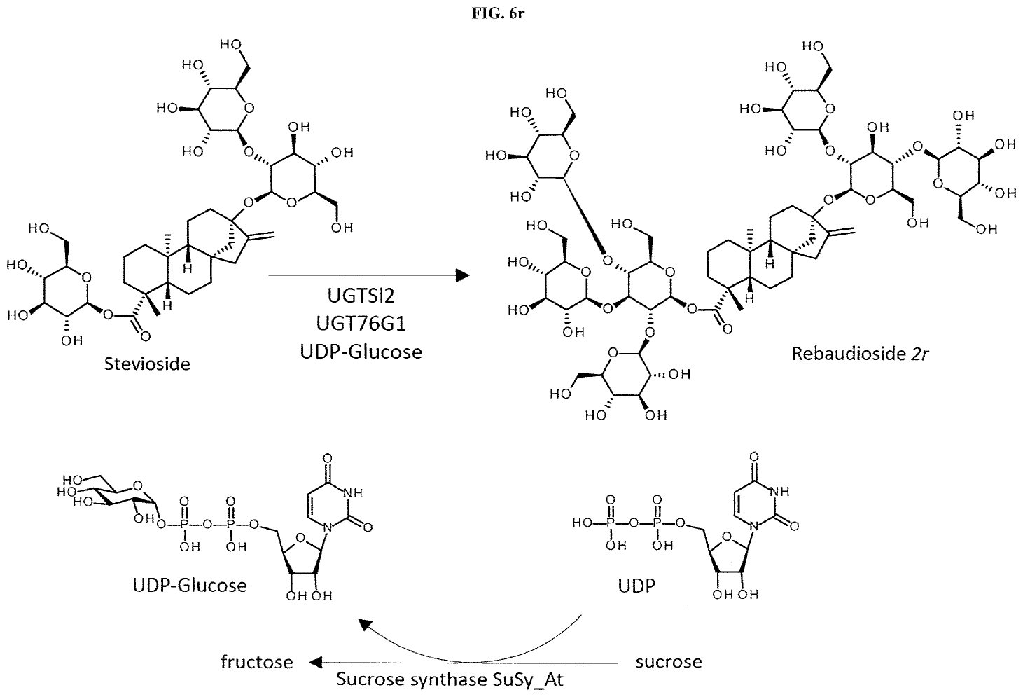

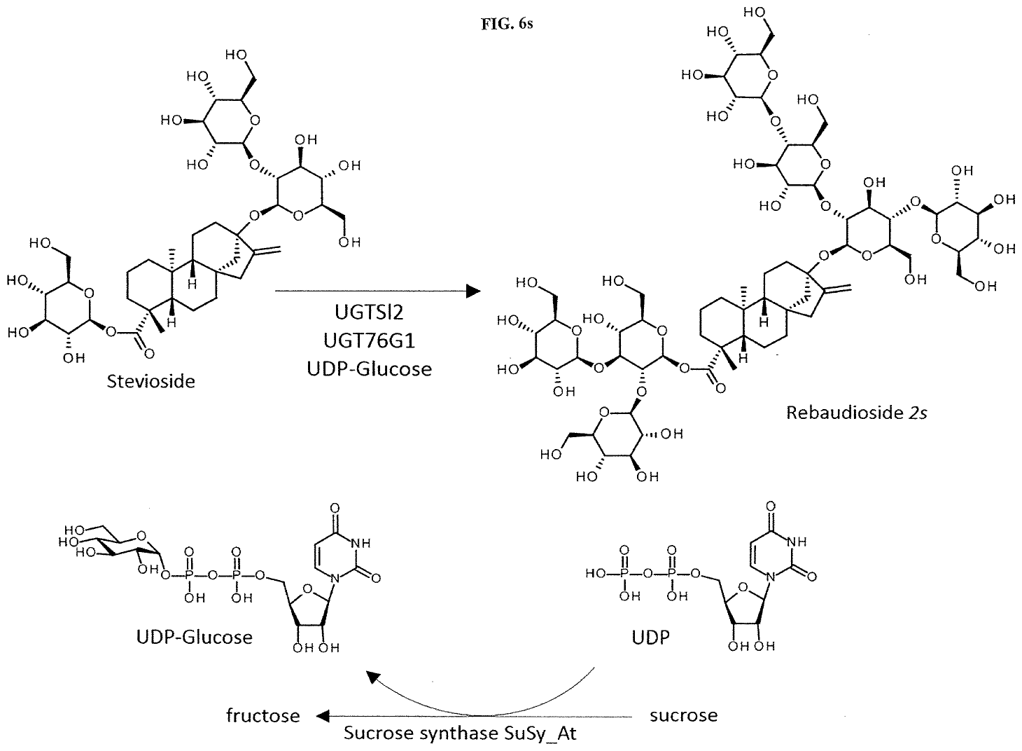

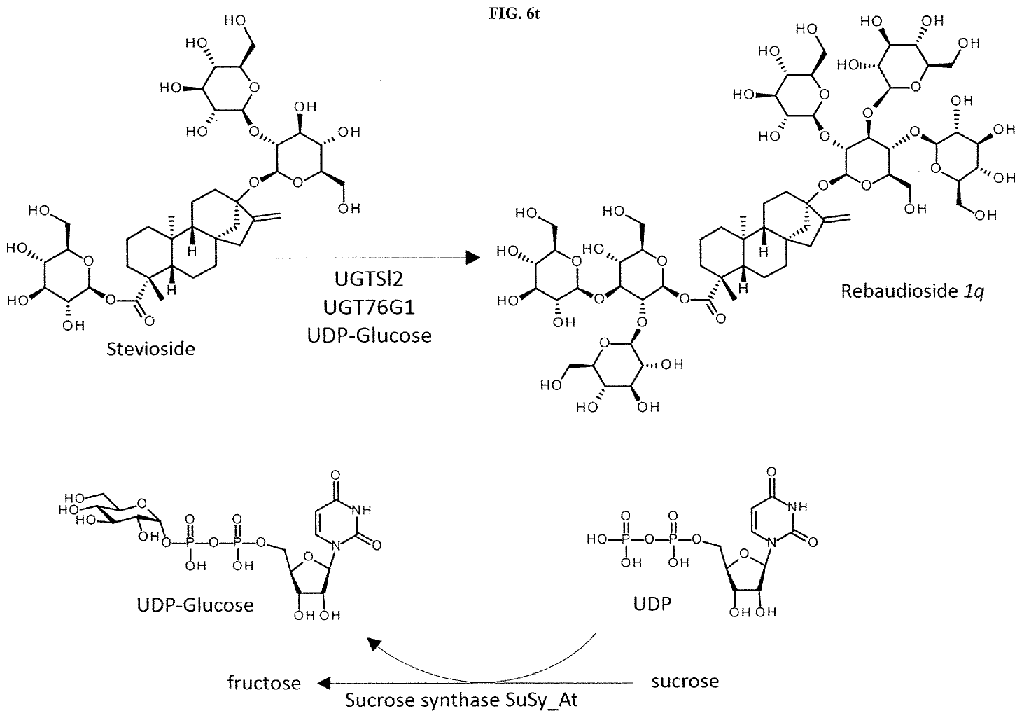

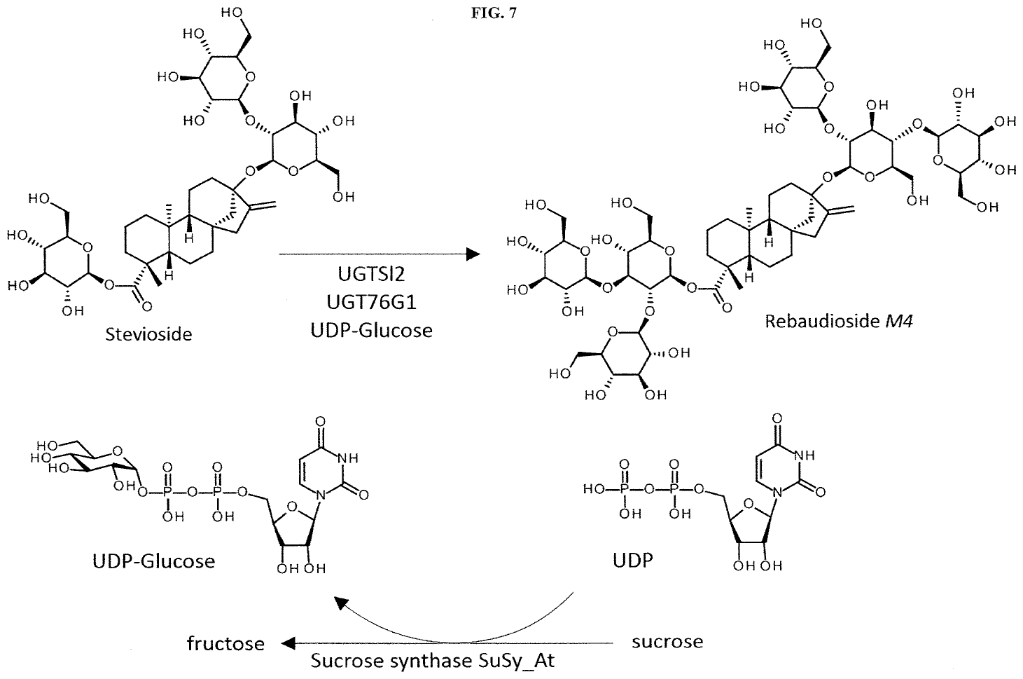

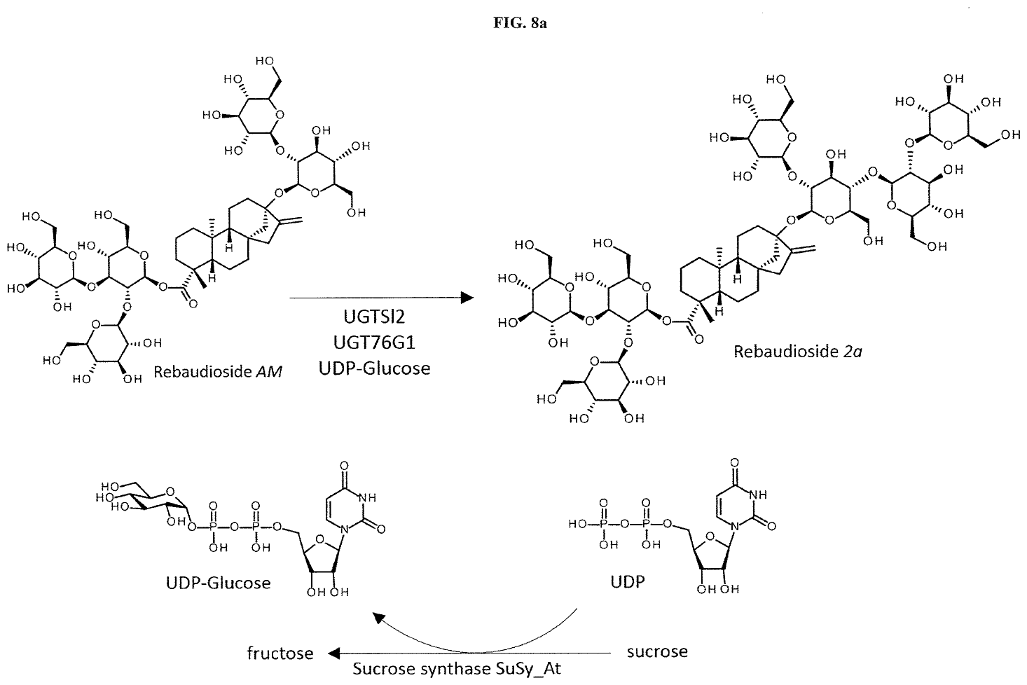

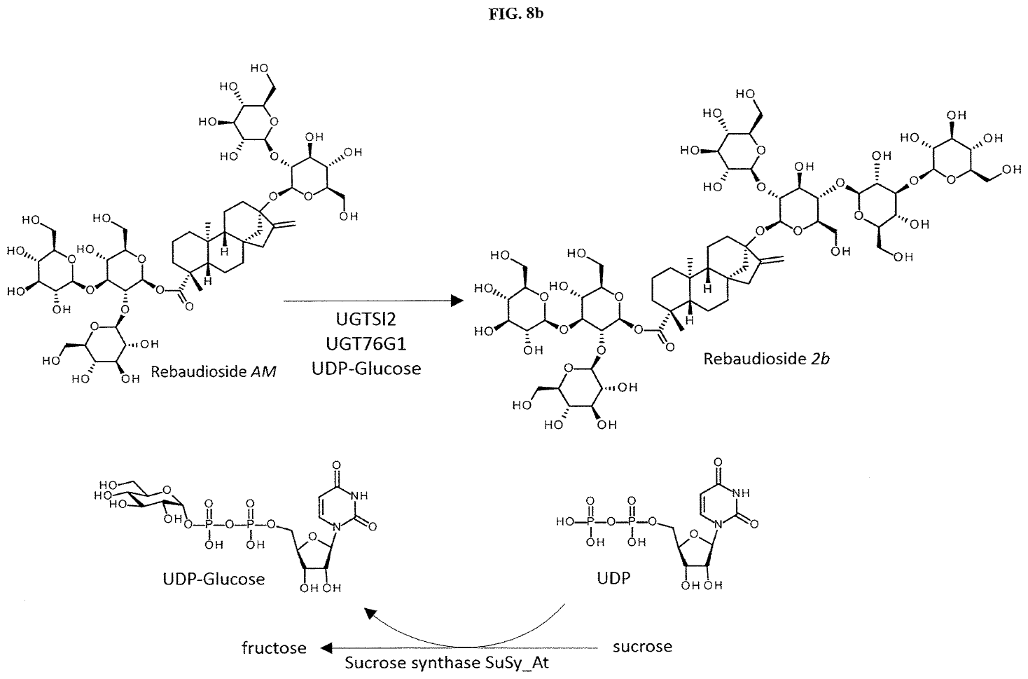

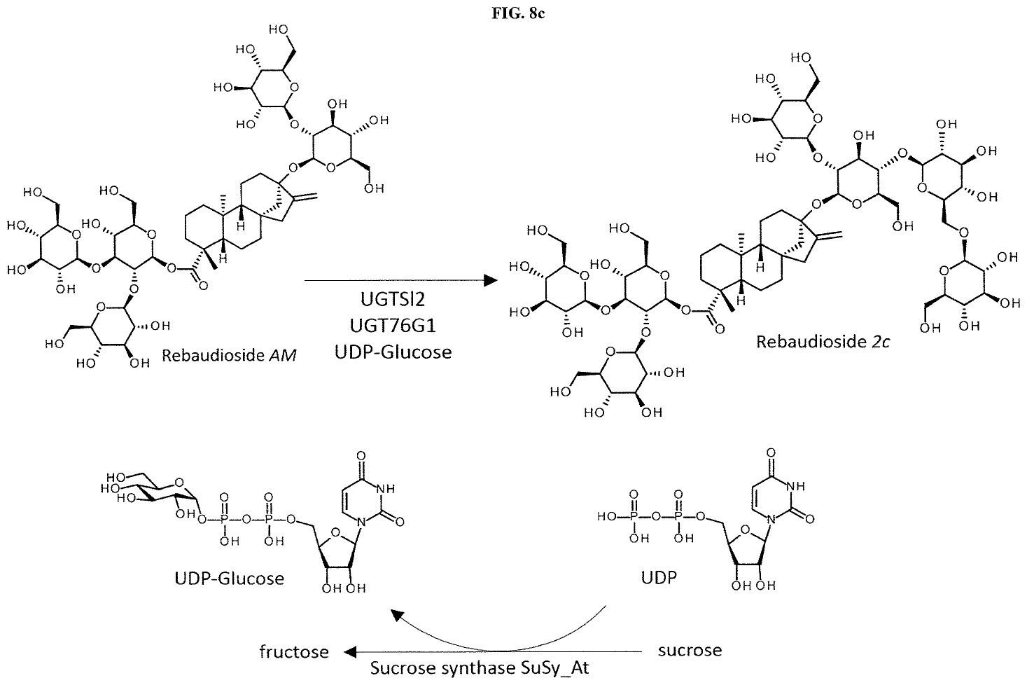

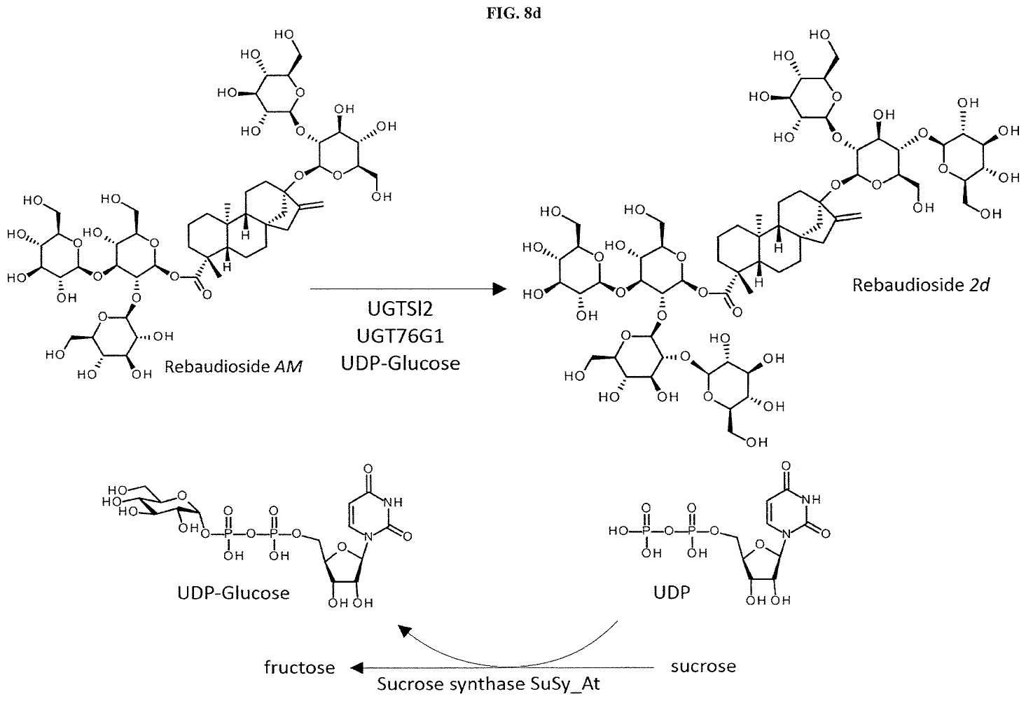

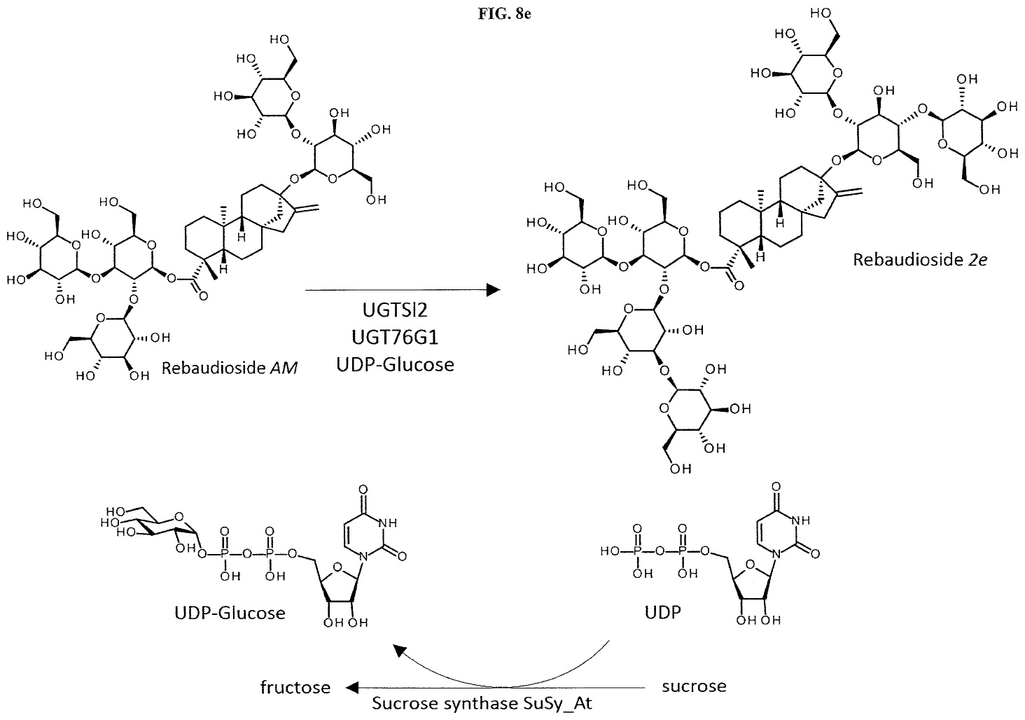

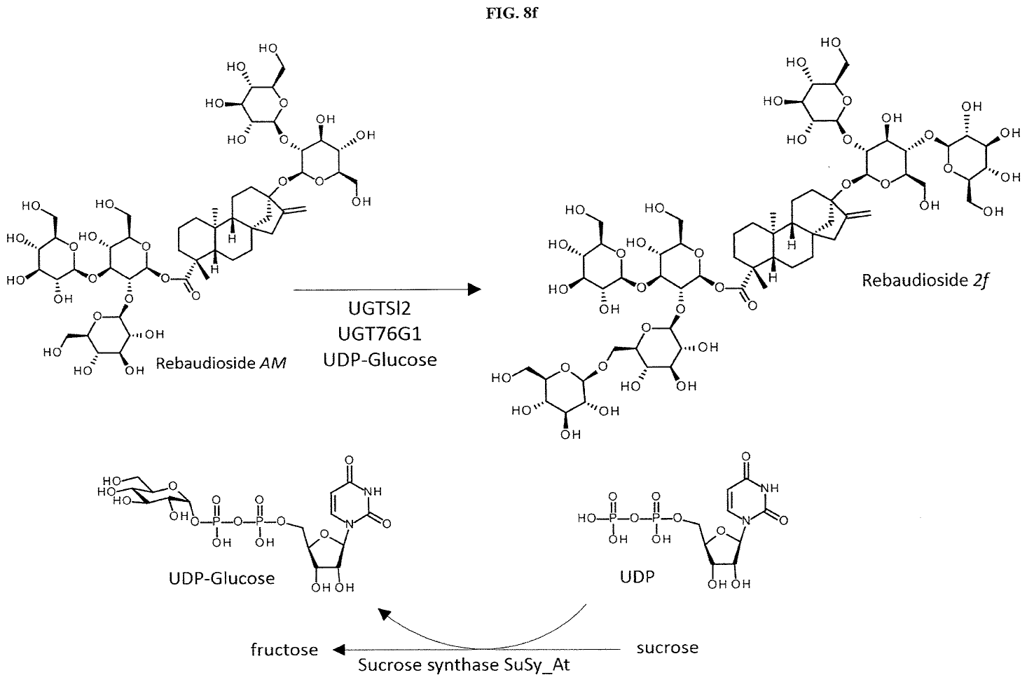

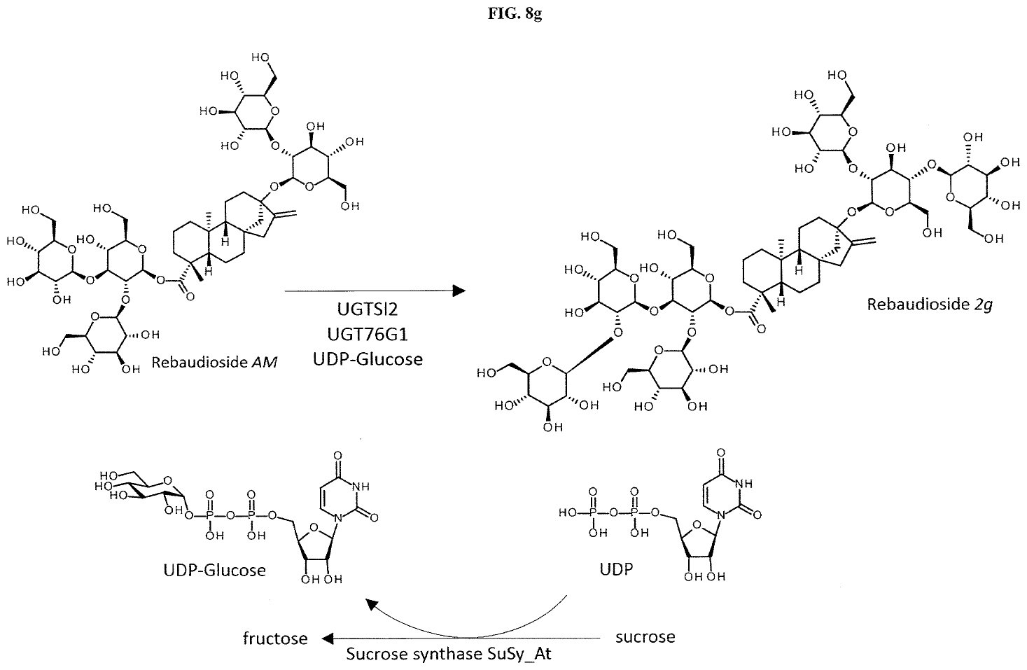

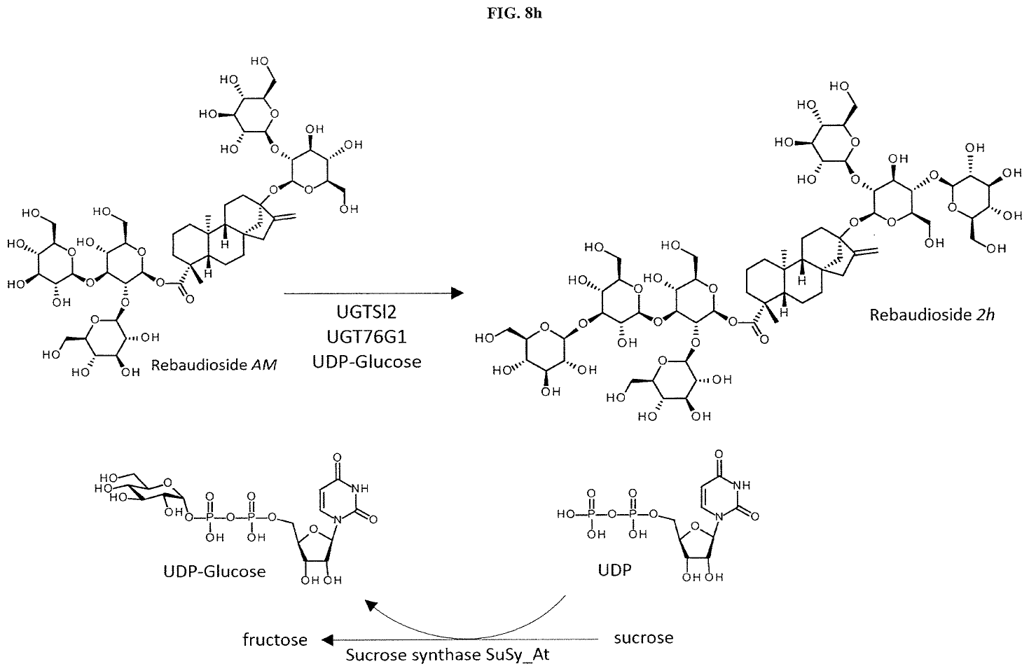

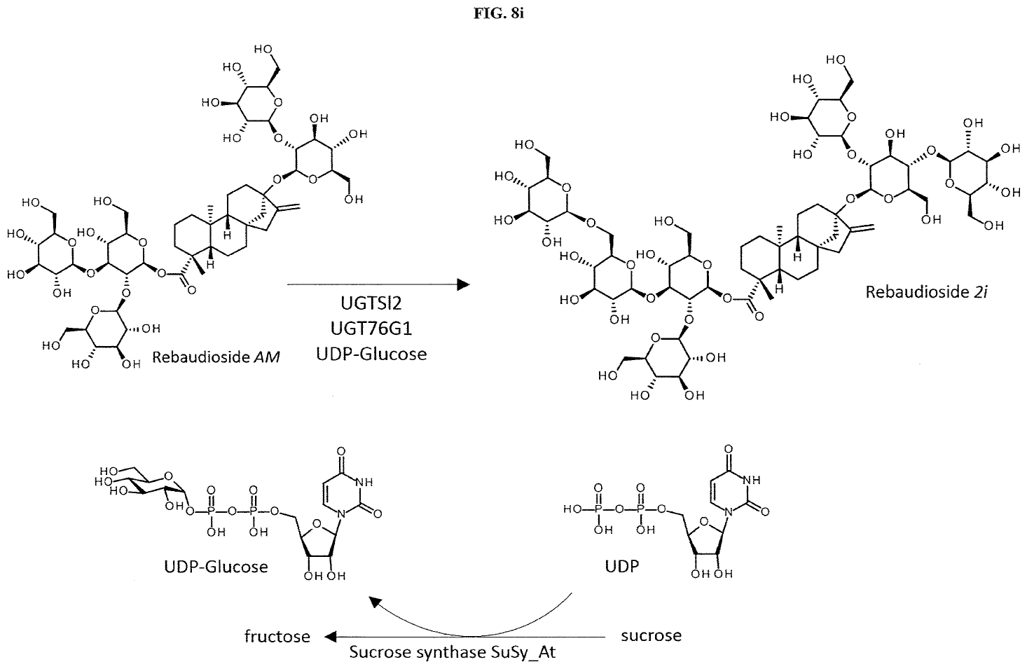

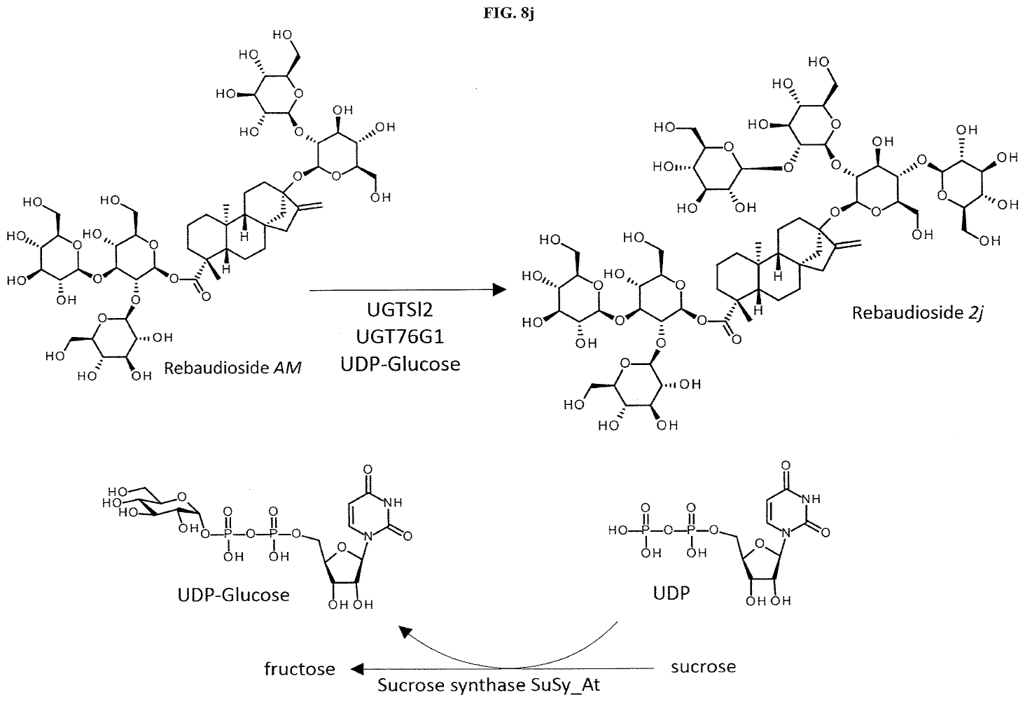

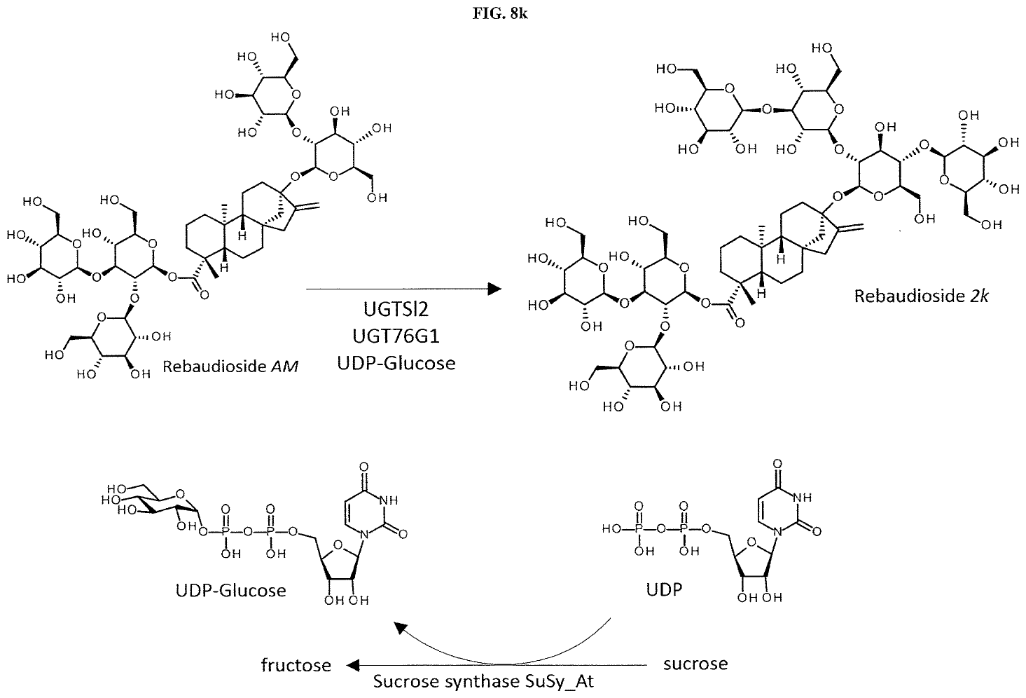

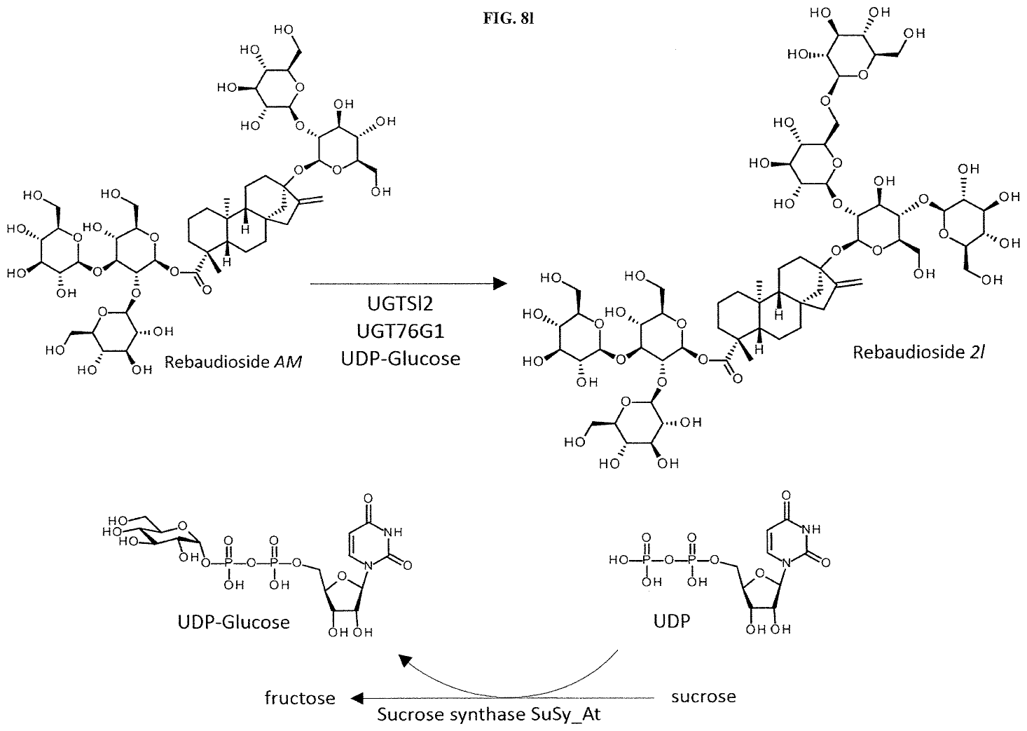

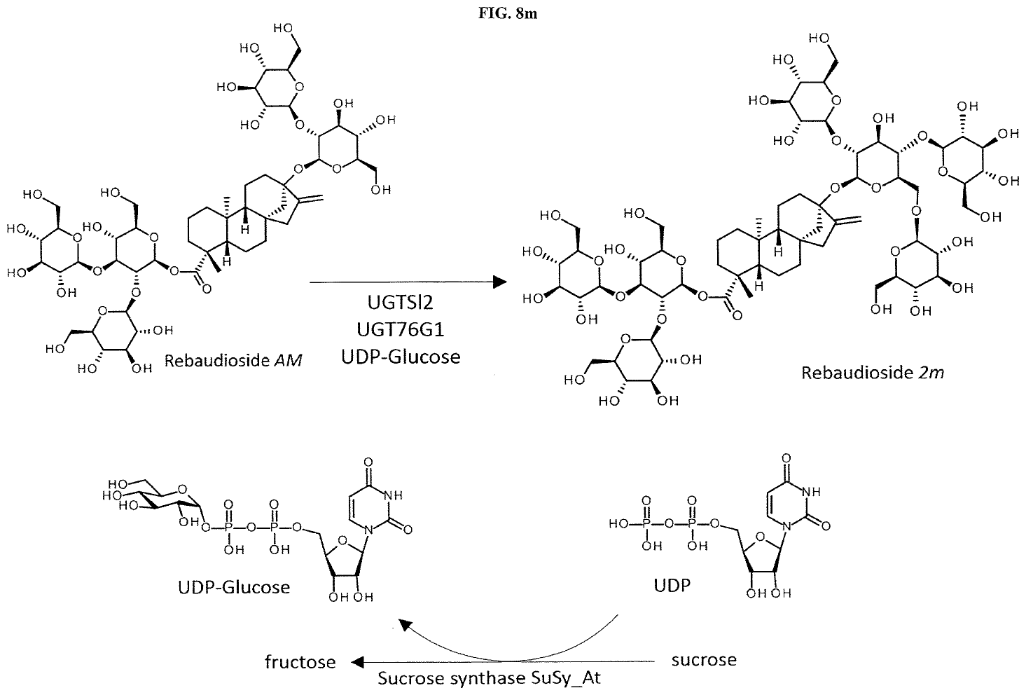

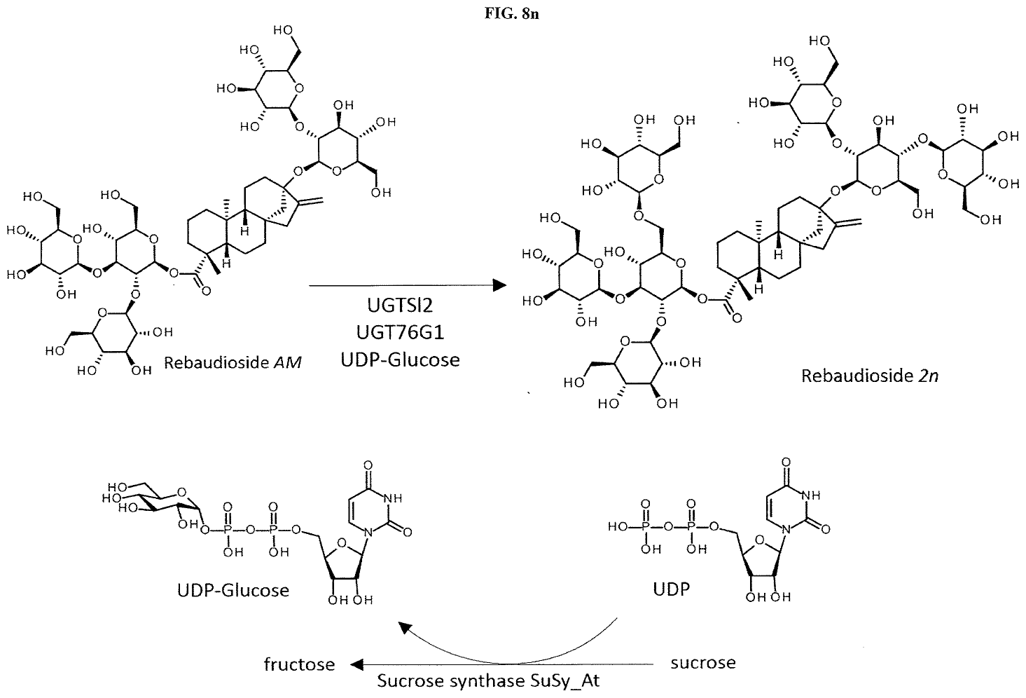

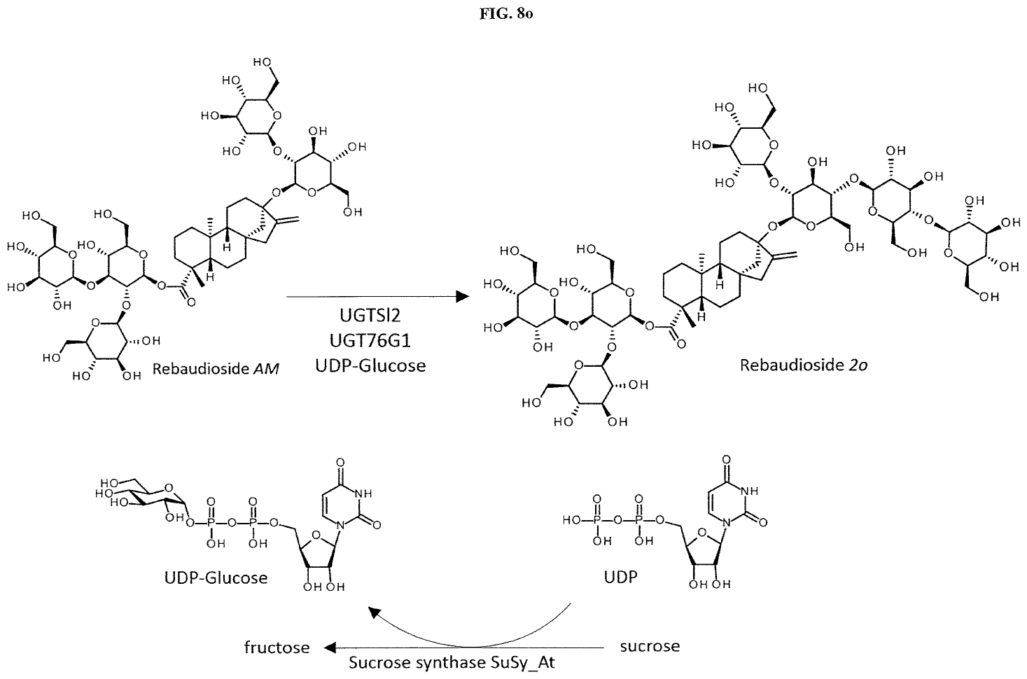

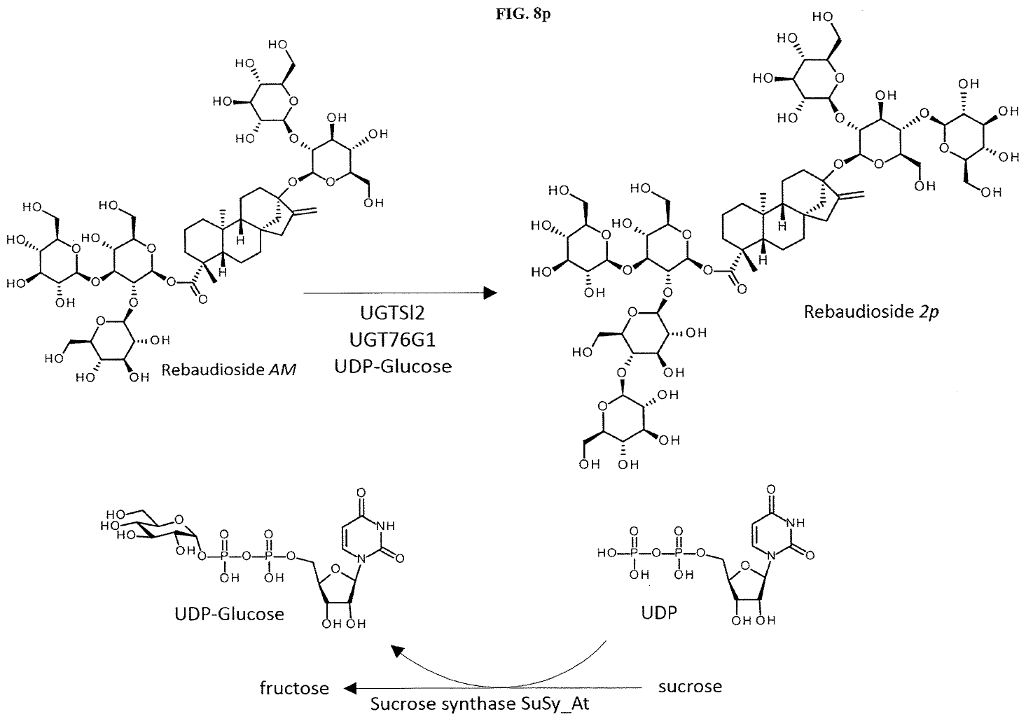

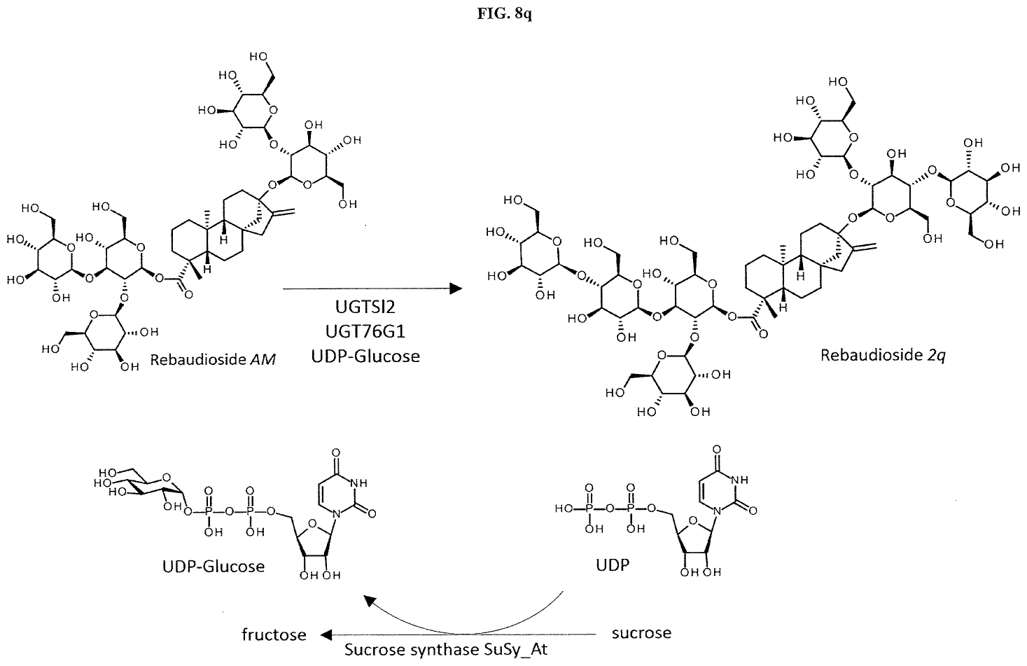

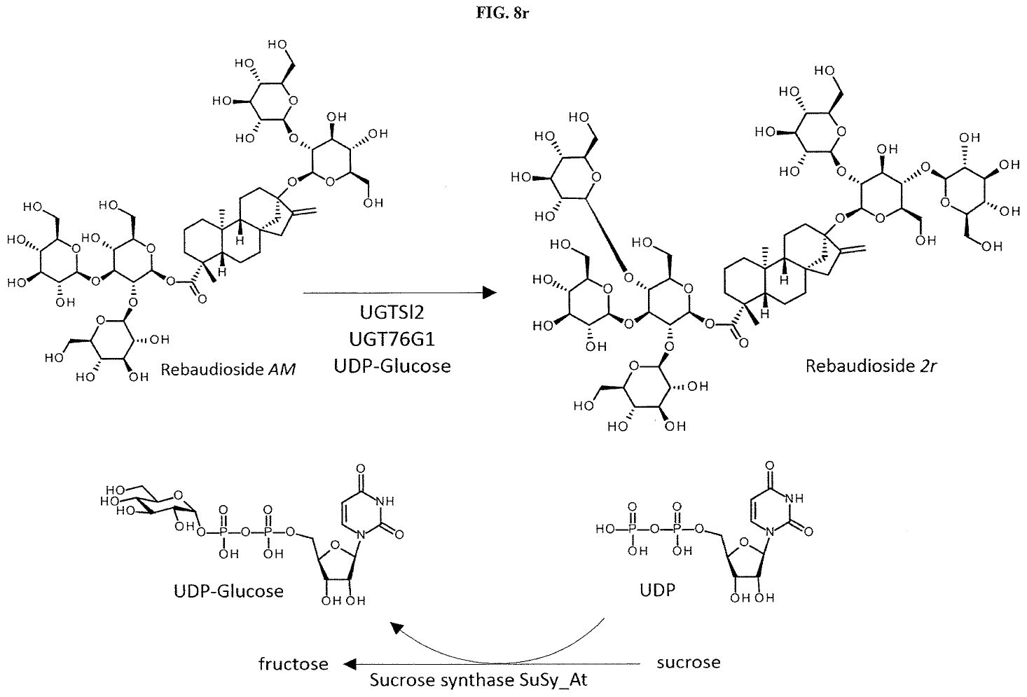

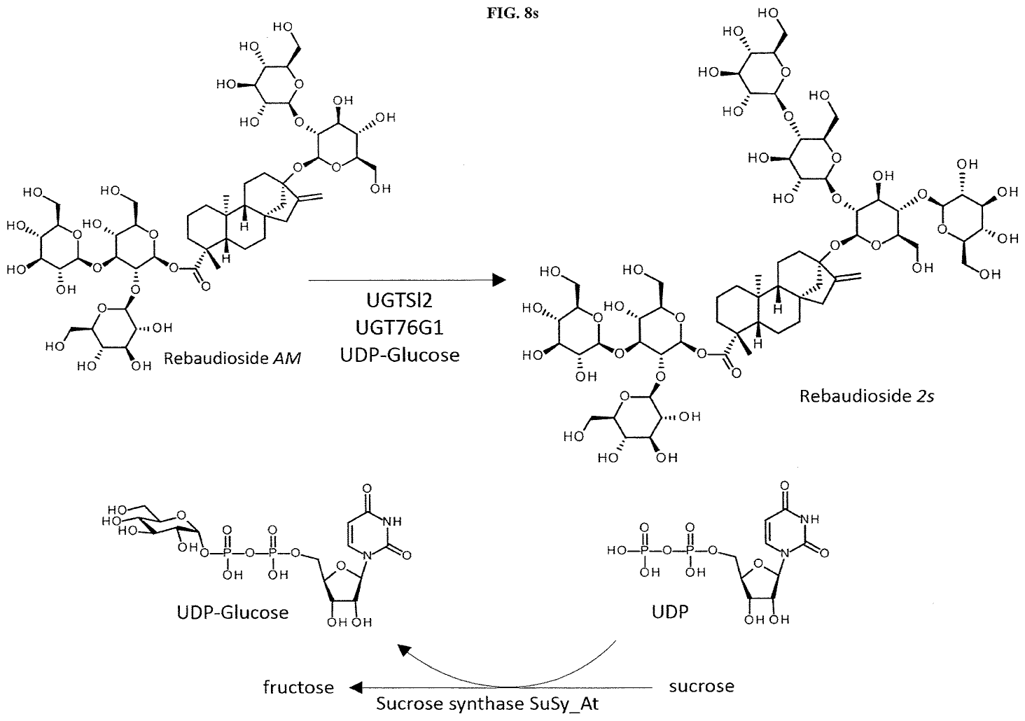

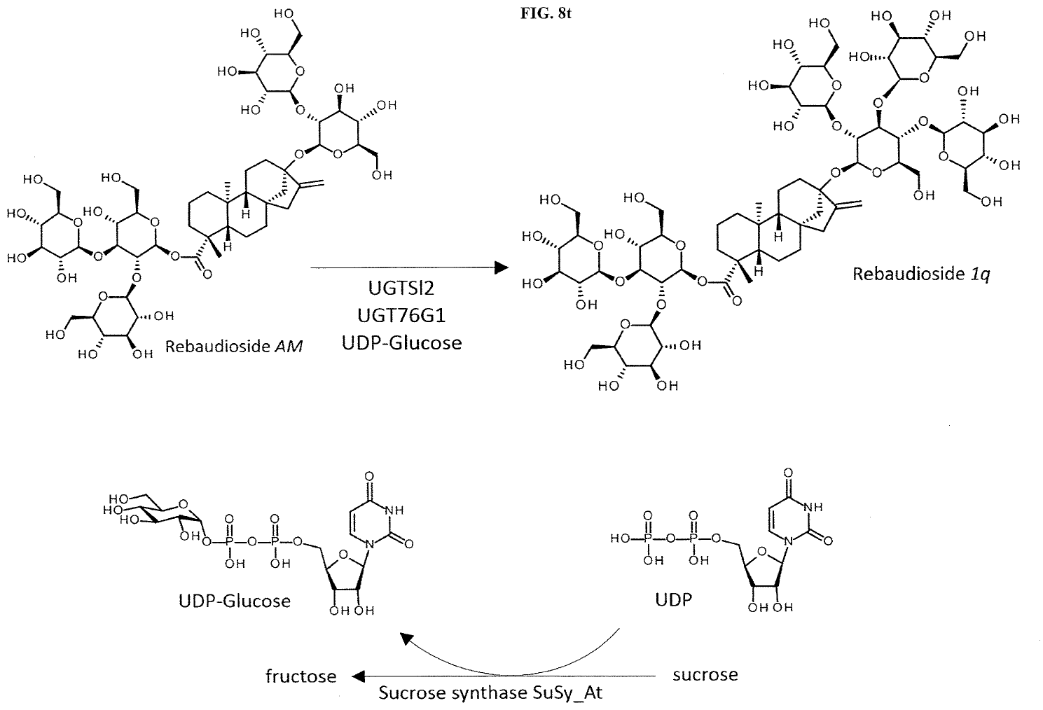

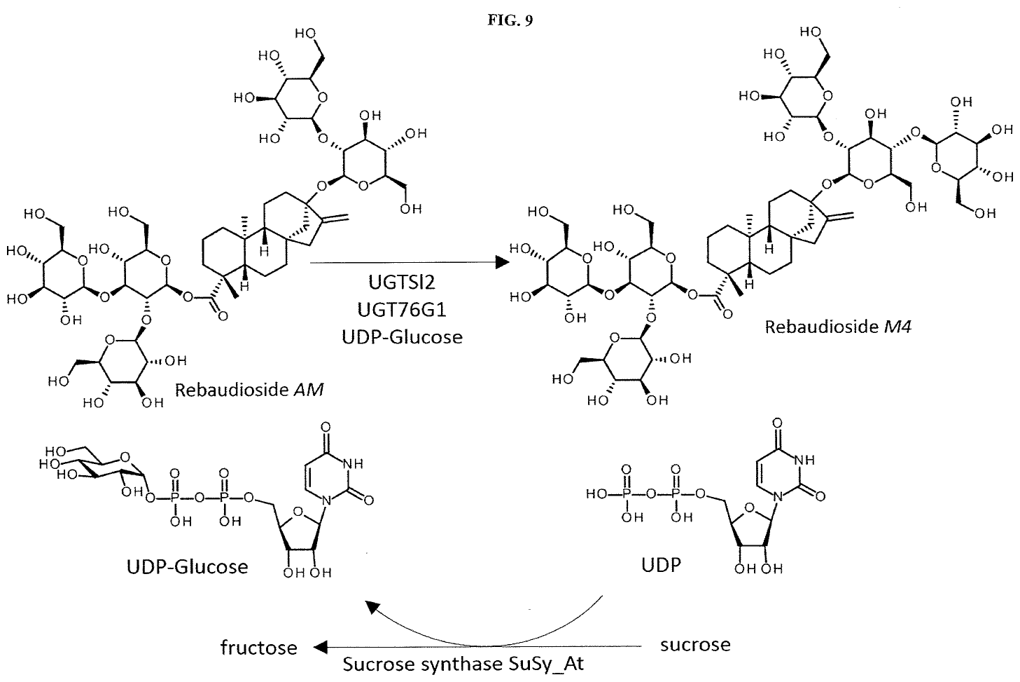

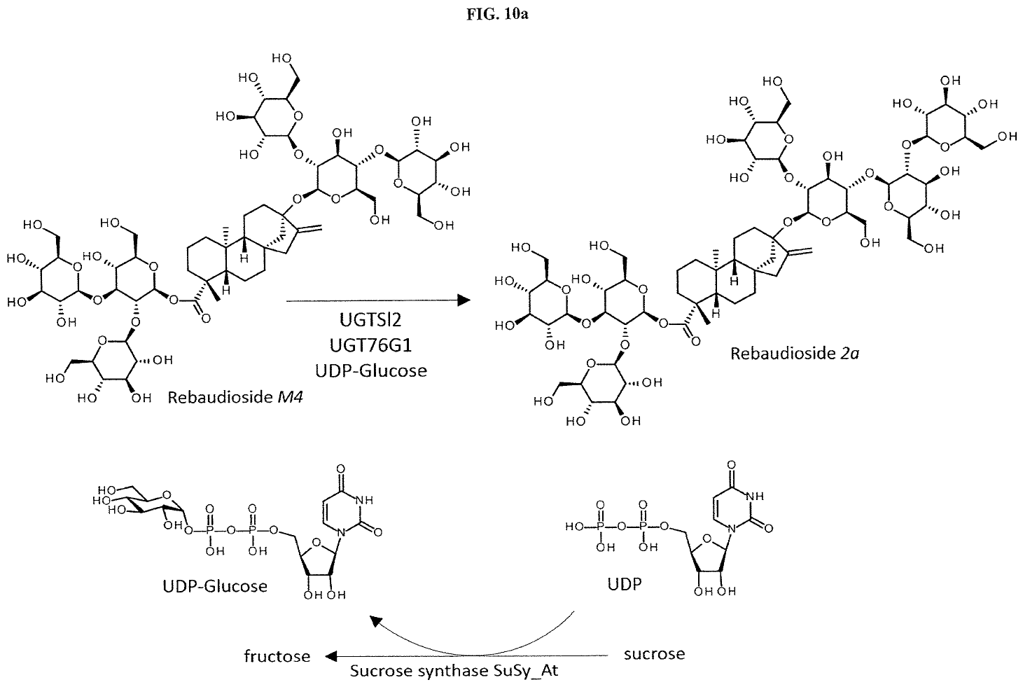

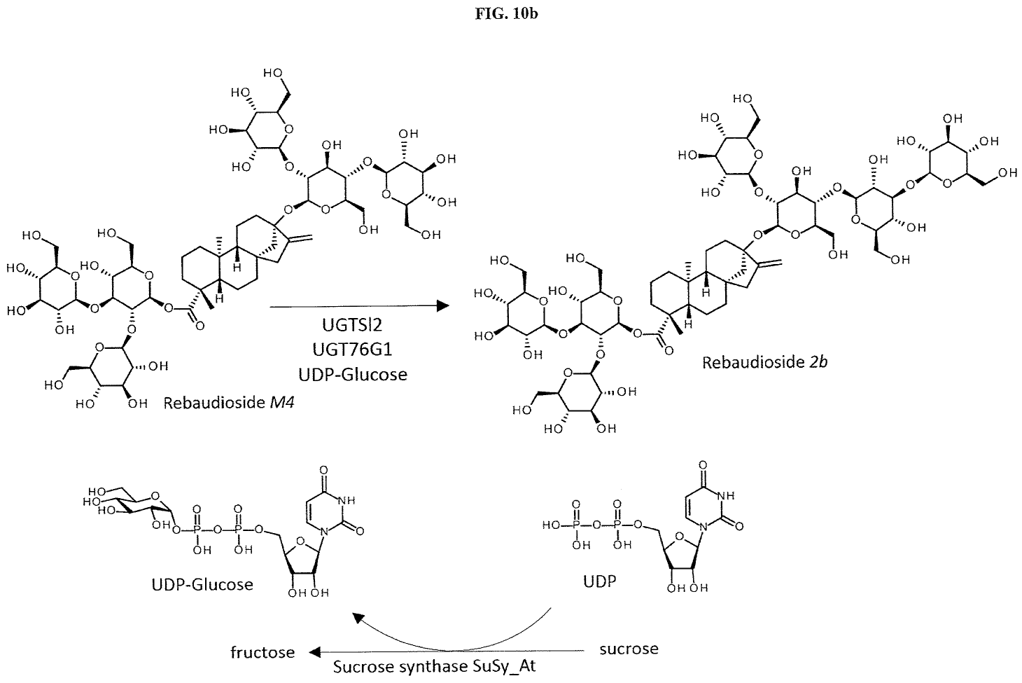

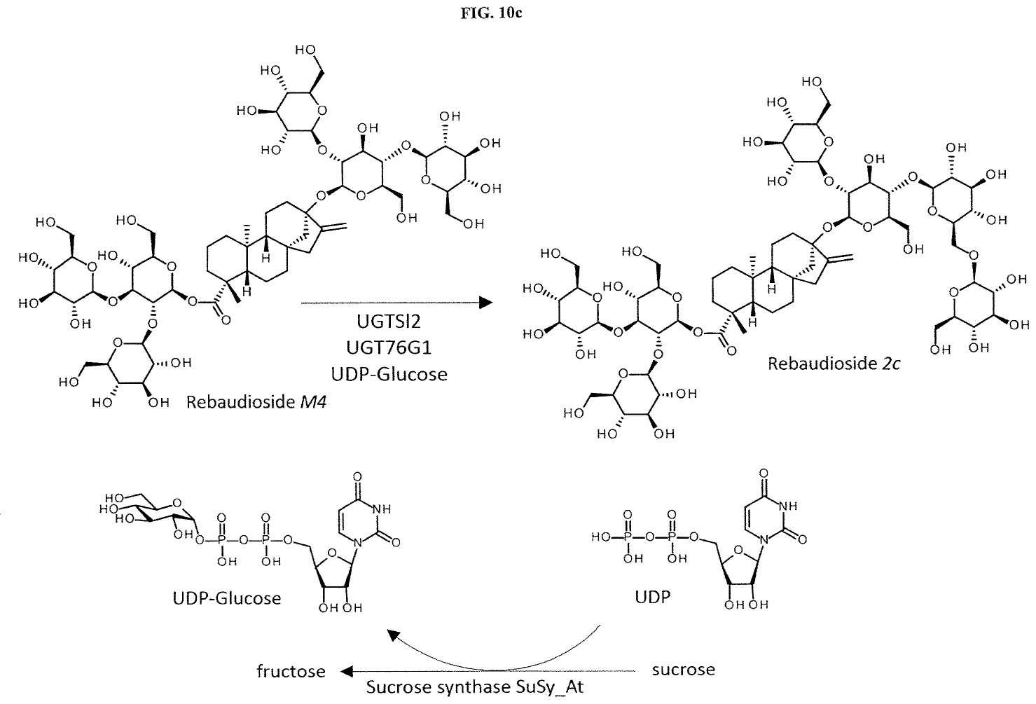

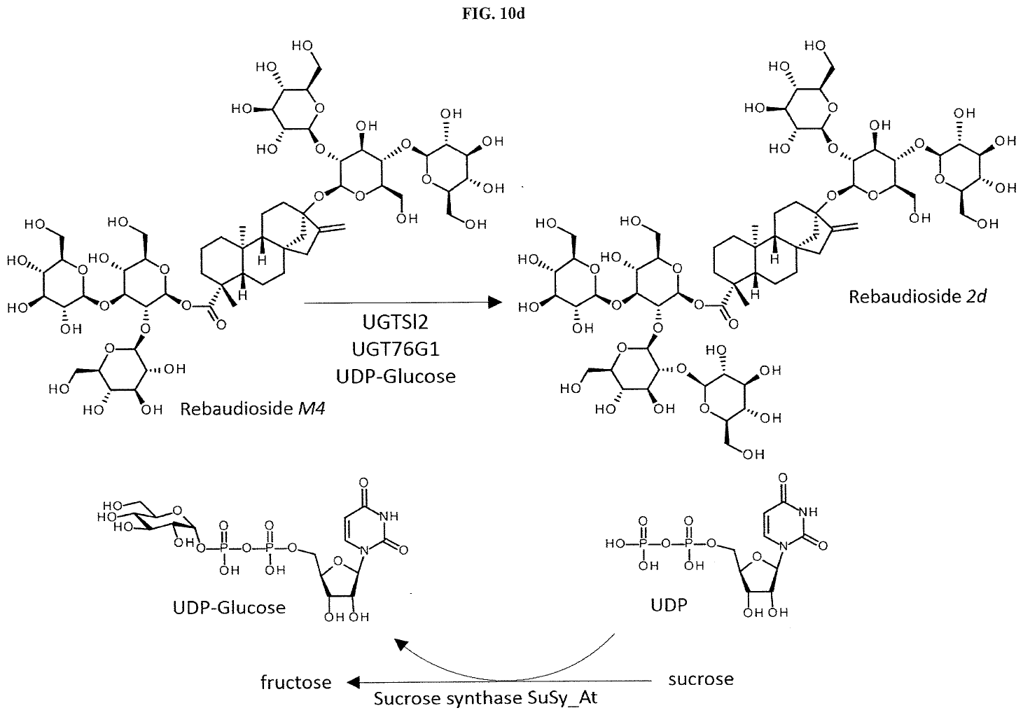

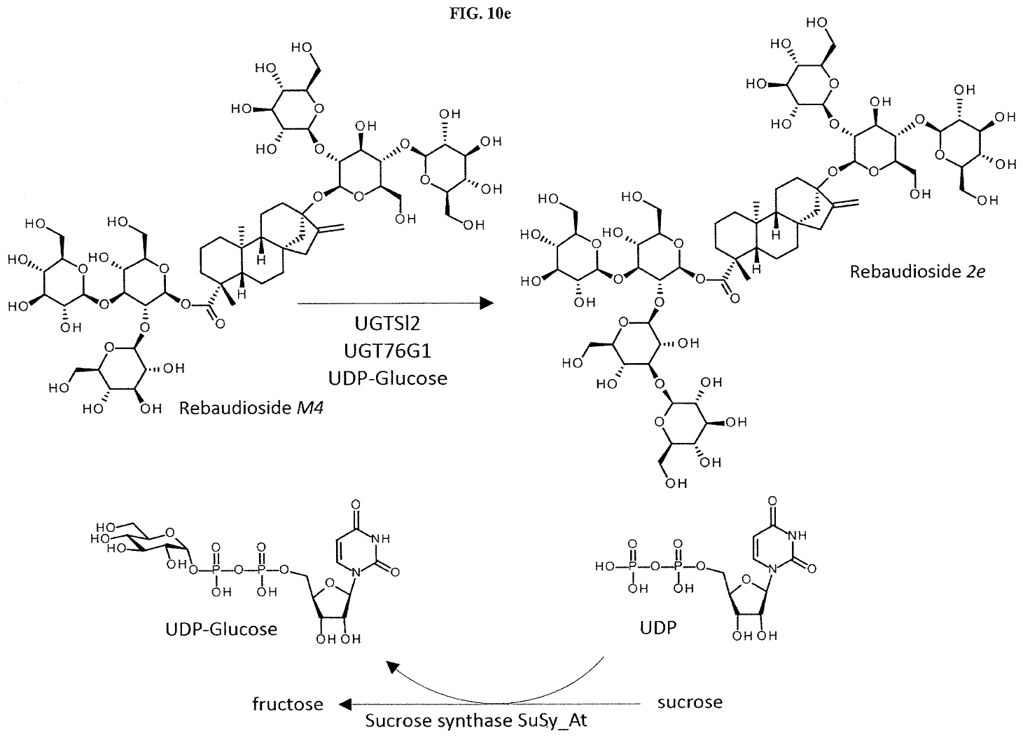

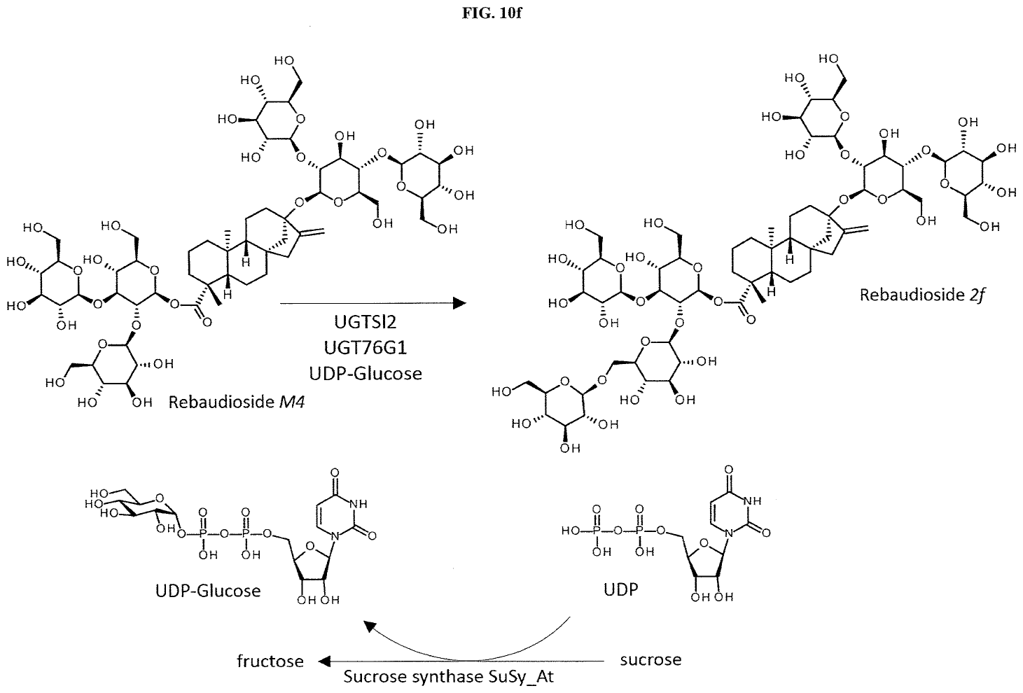

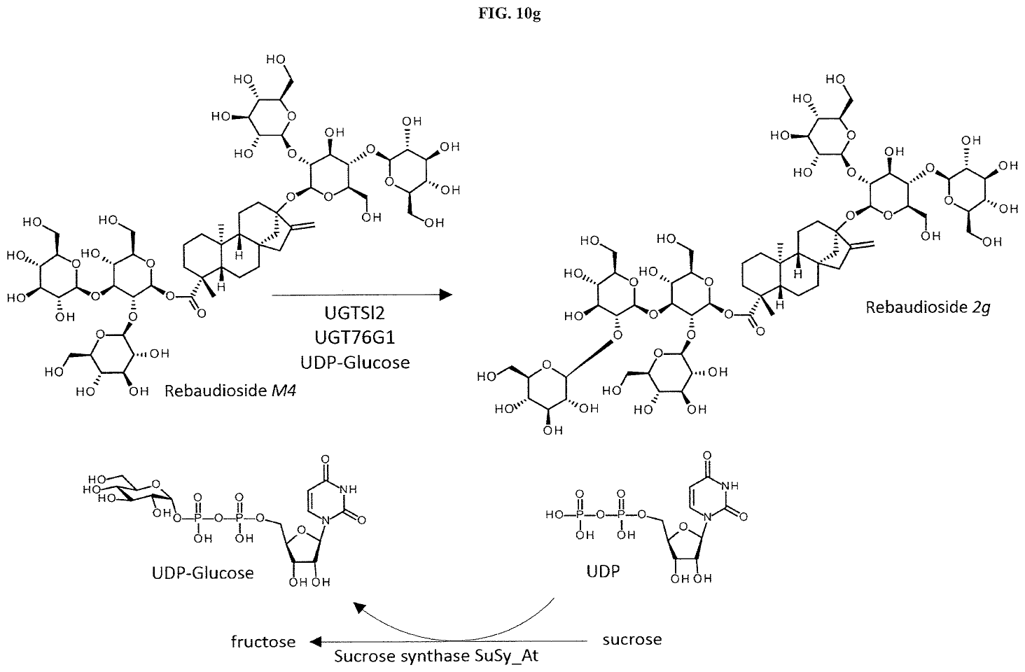

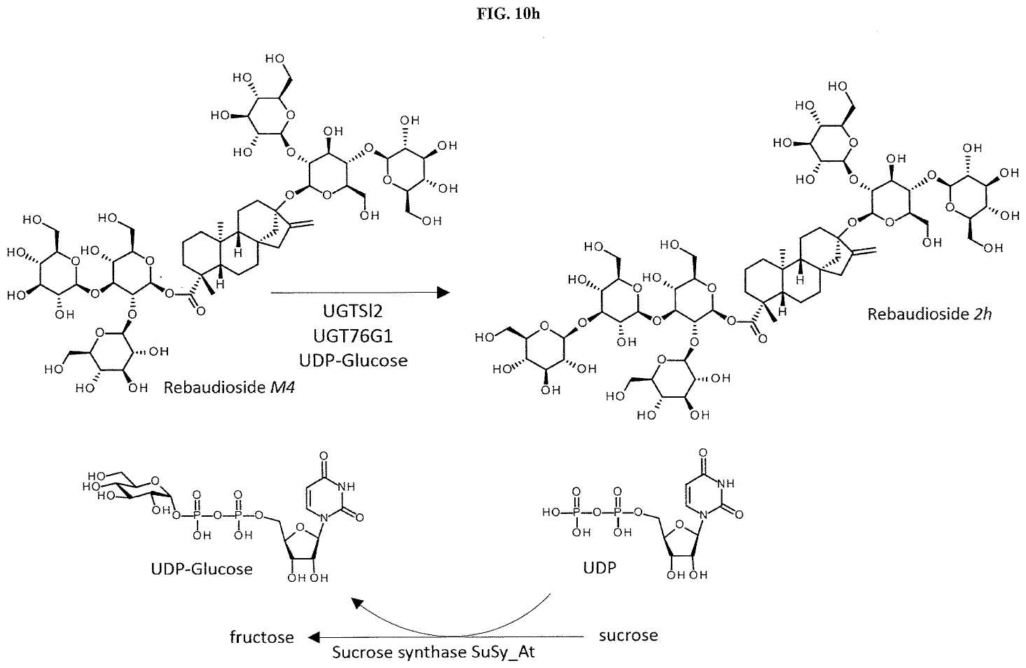

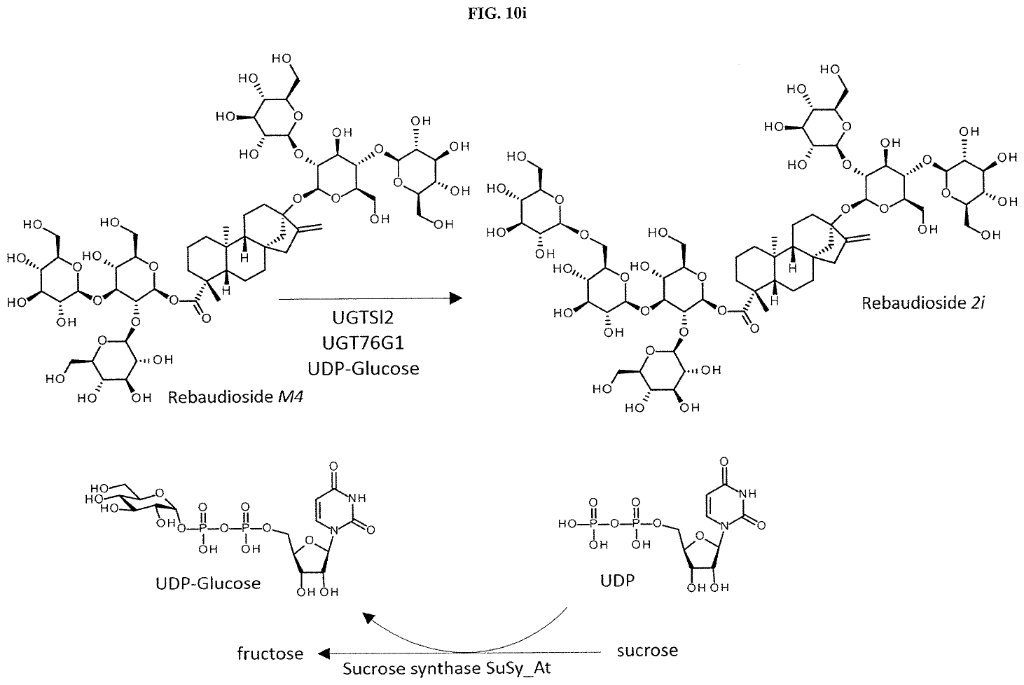

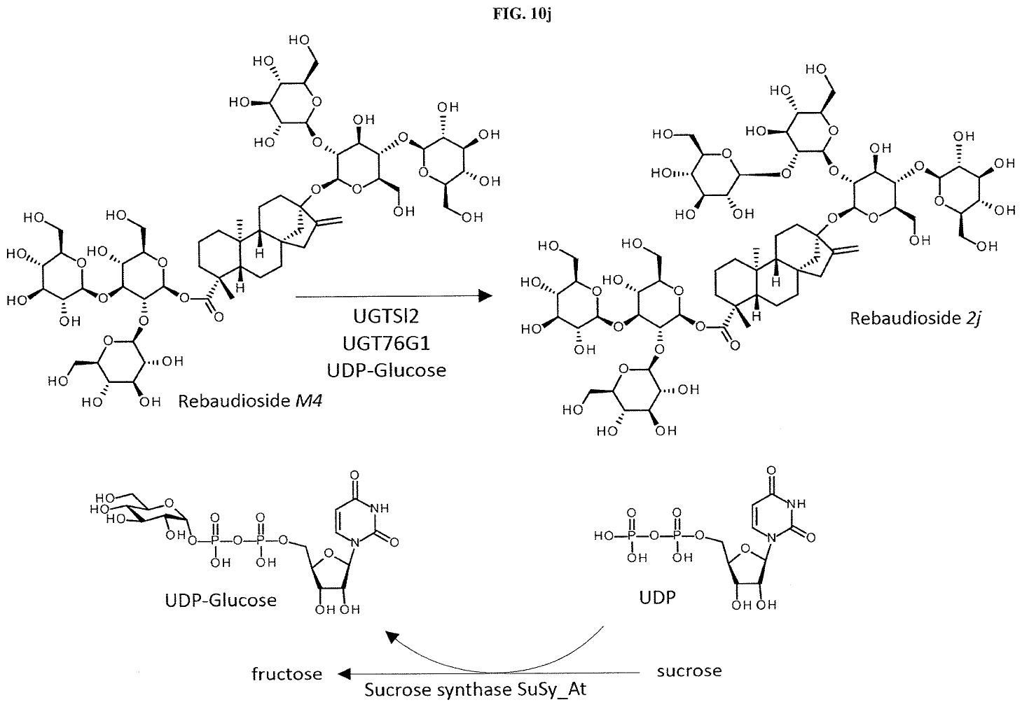

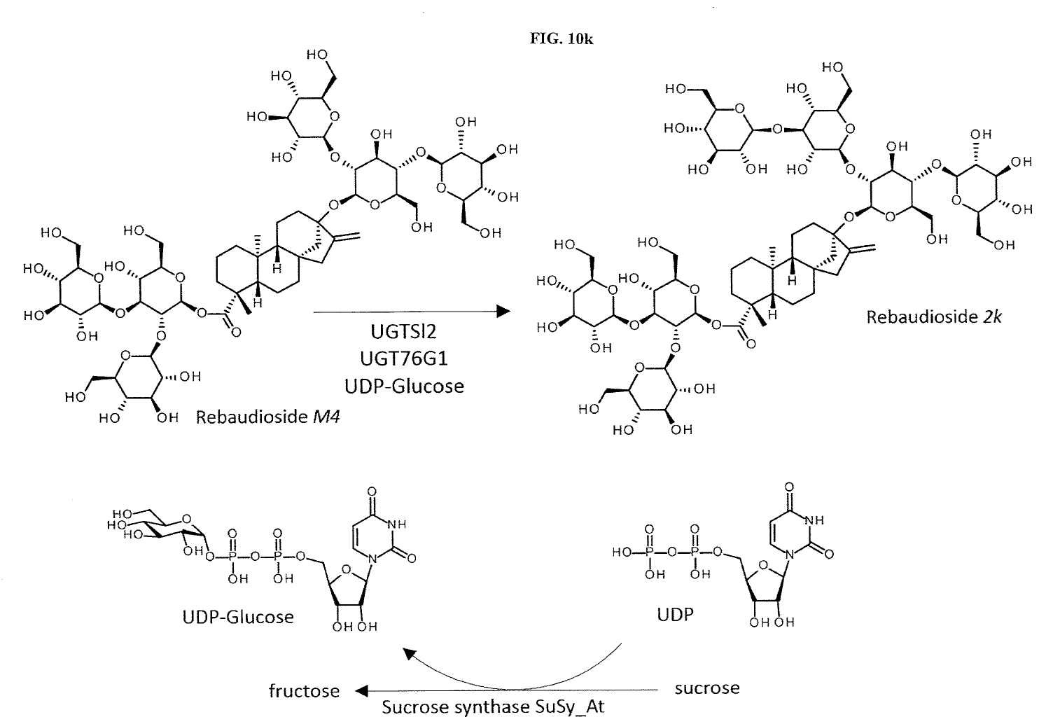

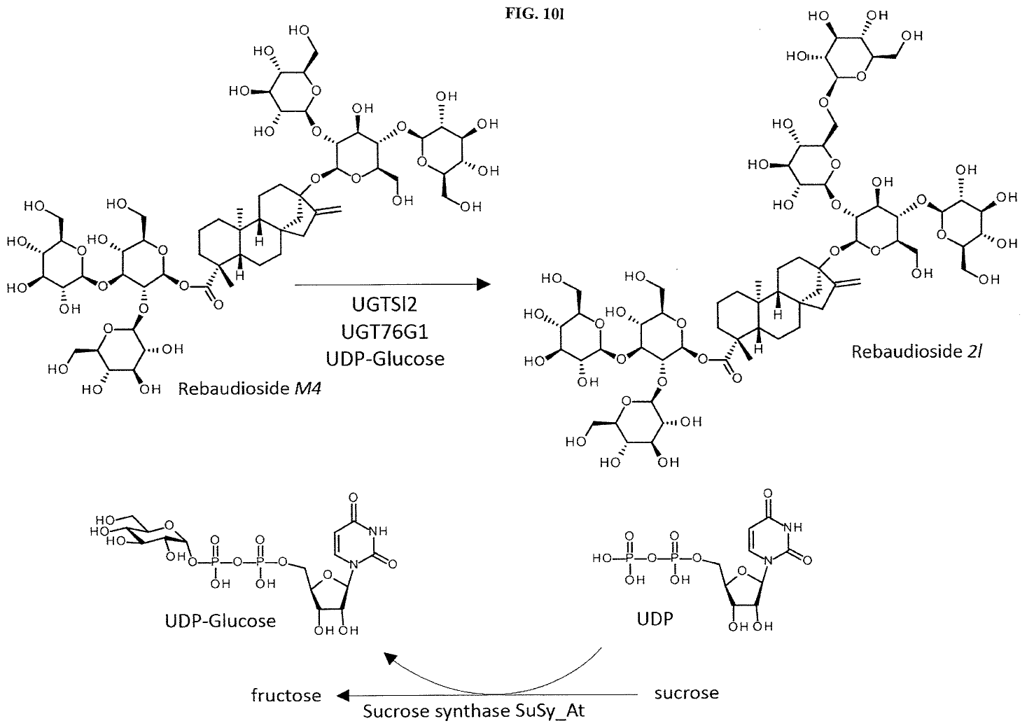

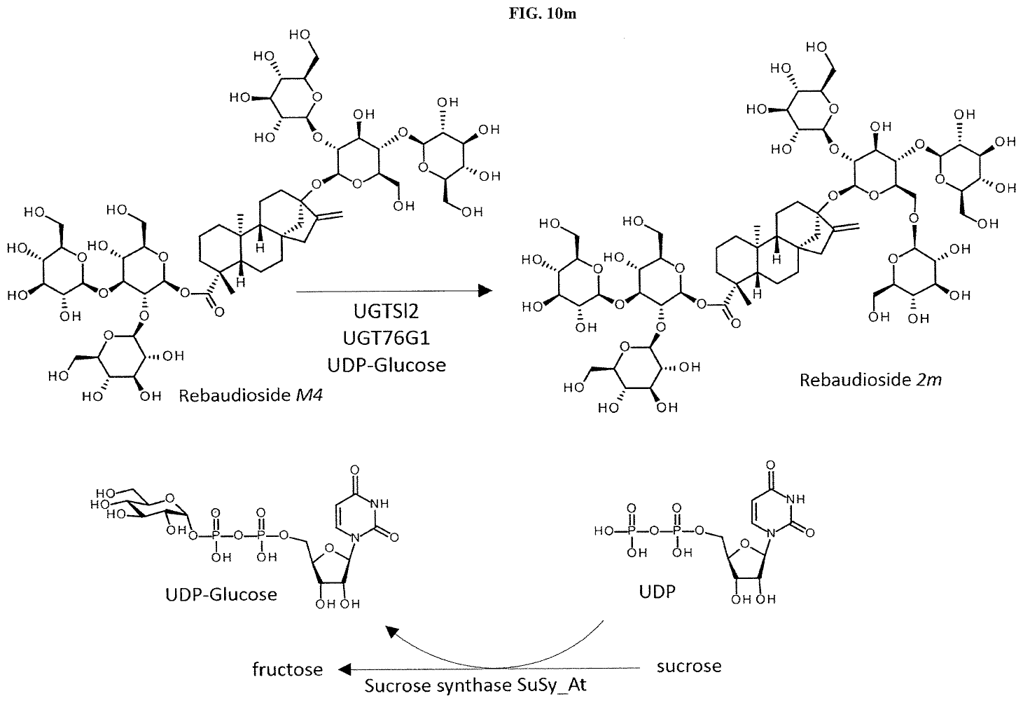

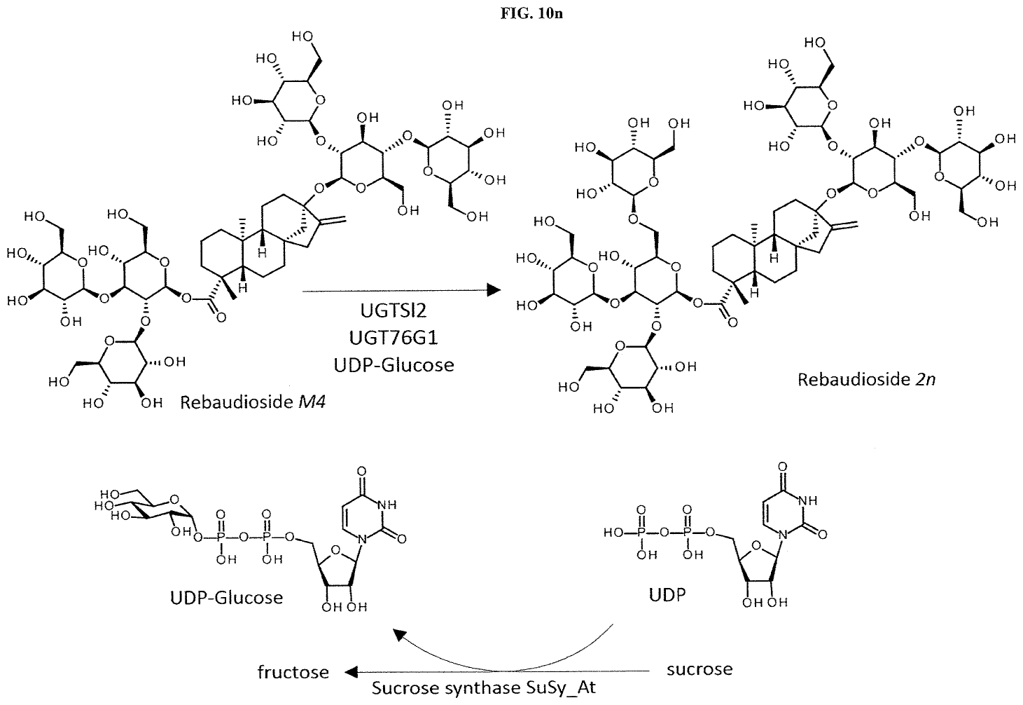

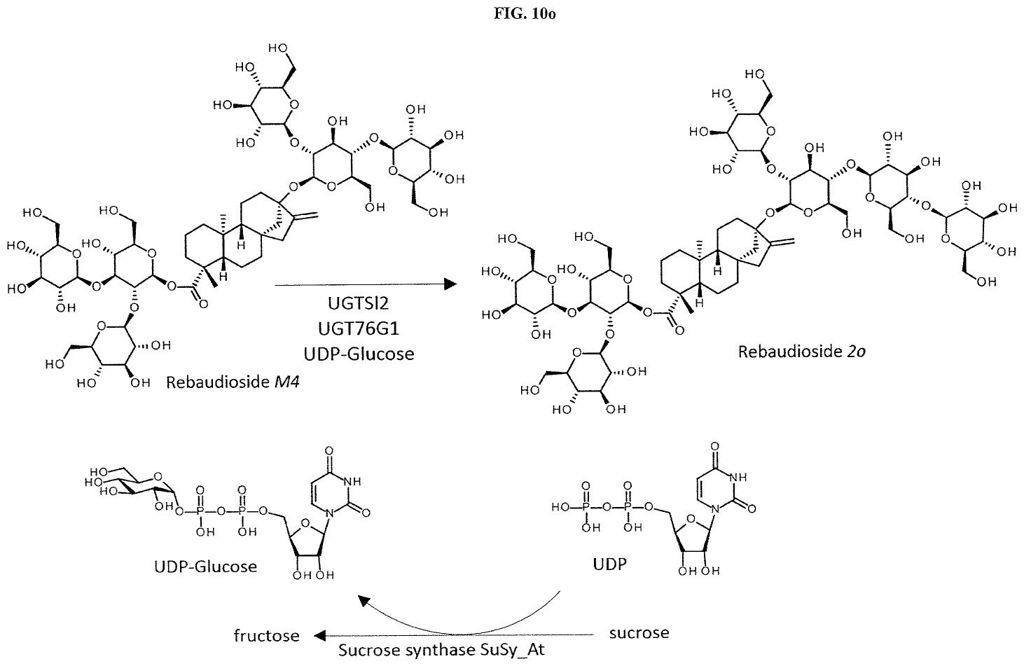

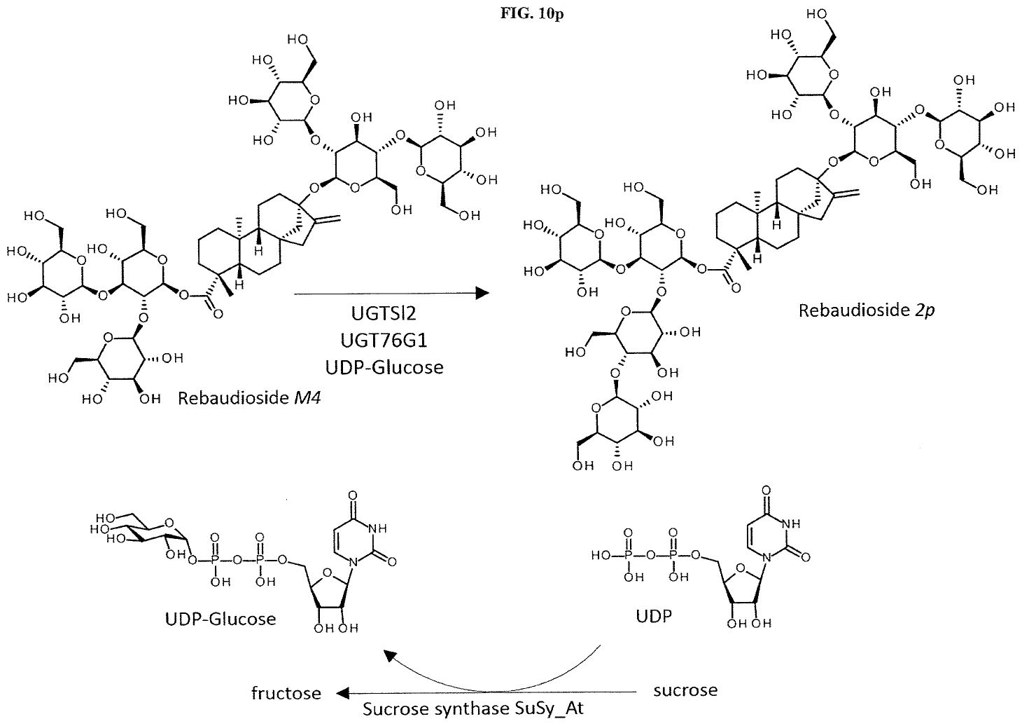

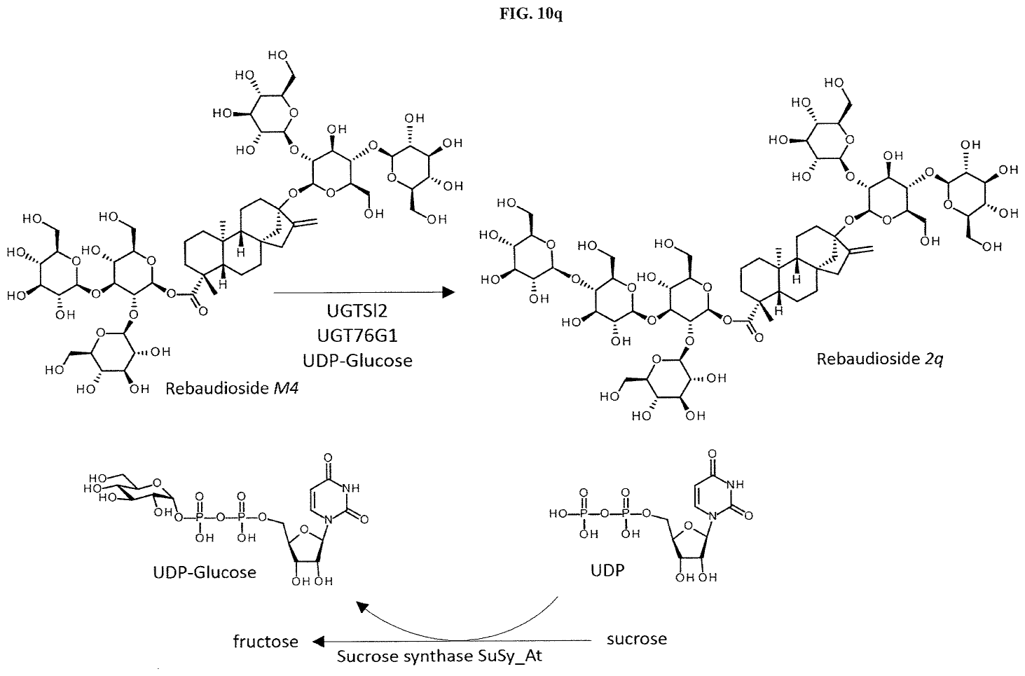

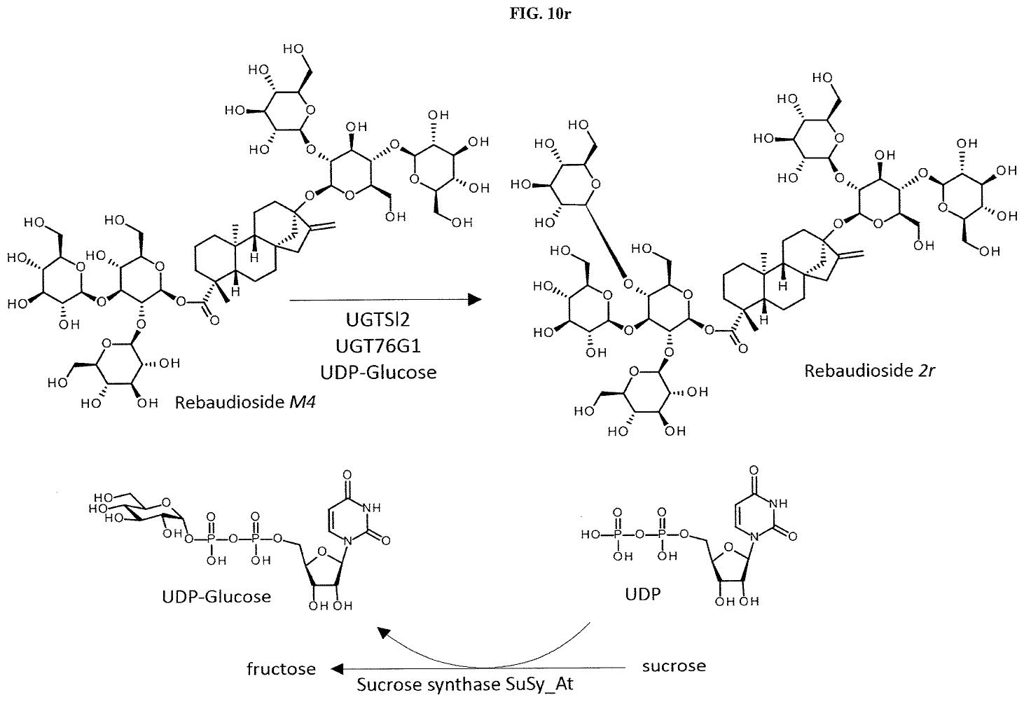

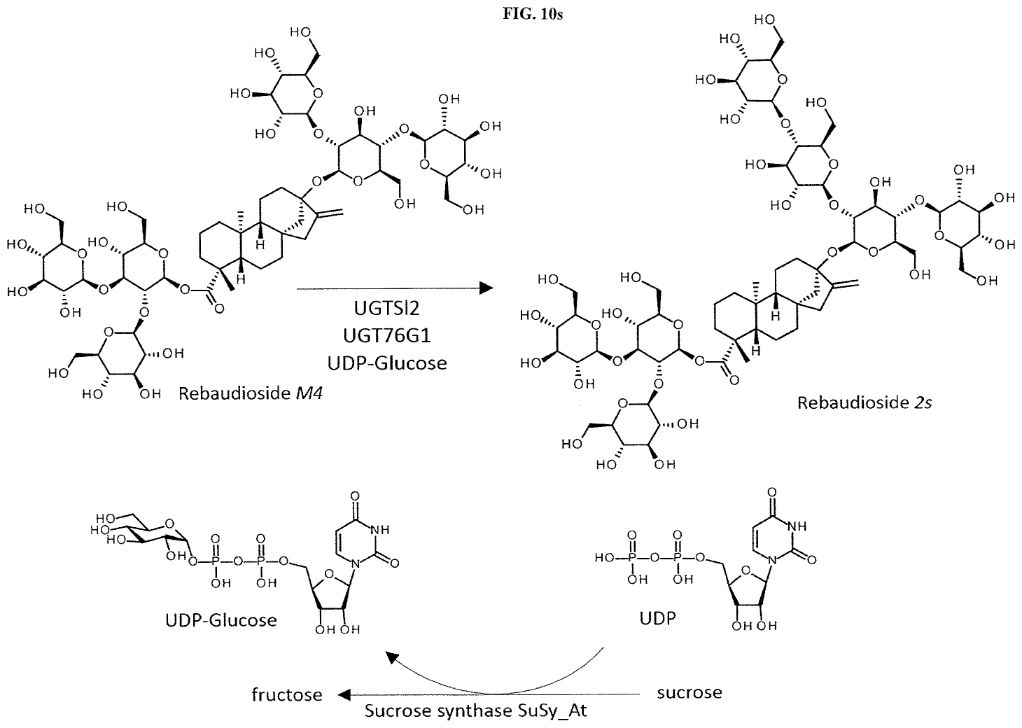

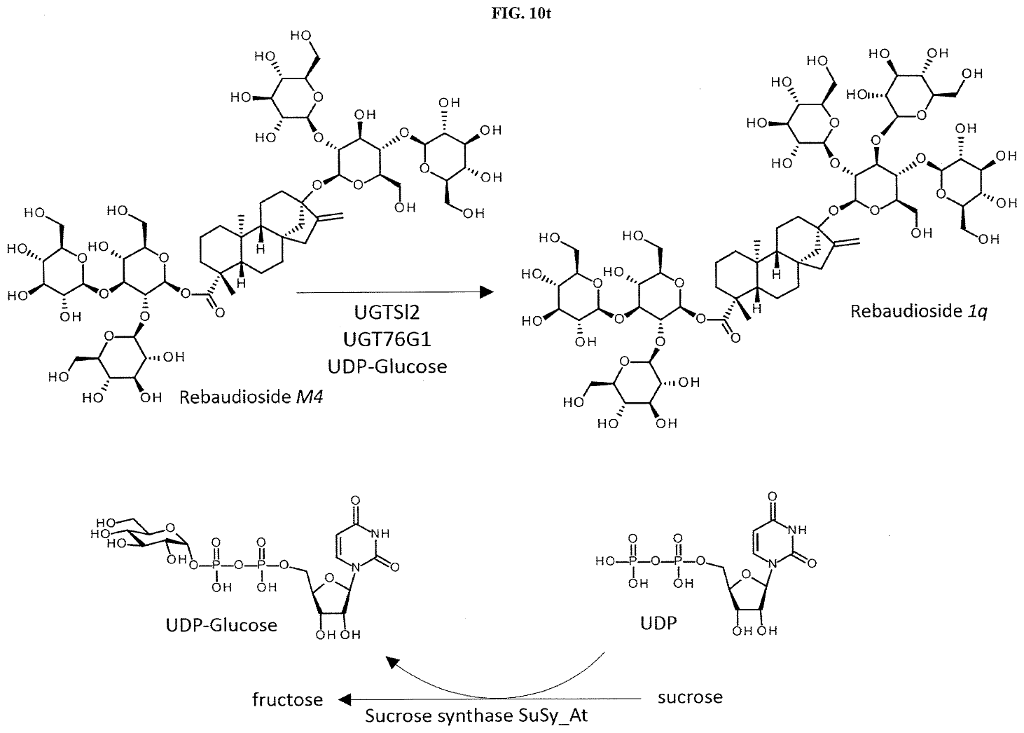

Table 3 shows for each time point the conversion of stevioside into identified rebaudioside species (area percentage). The chromatograms of the starting material stevioside and the reaction mixture at 48 hours are shown in FIG. 1a and FIG. 11b respectively. Those with skill in the art will appreciate that retention times can occasionally vary with changes in solvent and/or equipment.

TABLE 3
Biotransformation of stevioside to reb 2a (rt 6.459),
reb 2m (rt 5.089) and various SvG7

| | % conversion from stevioside | |
|---|---|---|
| | reaction | reaction |
| Peak | time 0 hr | time 48 hr |
| rt 2.651 | 0 | 0.83 |
| rt 2.874 | 0 | 6.79 |
| rt 3.275 | 0 | 0.15 |
| rt 3.570 | 0 | 0.05 |
| rt 3.798 | 0 | 0.18 |
| rt 4.279 | 0 | 0.08 |
| rt 4.477 | 0 | 0.05 |
| rt 4.798 | 0 | 0.04 |
| rt 5.089 | 0 | 0.68 |
| rt 5.350 | 0 | 0.86 |
| rt 5.758 | 0 | 0.28 |
| rt 5.896 | 0 | 1.06 |
| rt 6.261 | 0 | 0.22 |
| rt 6.459 | 0 | 1.37 |
| rt 6.842 | 0 | 0.57 |
| rt 7.952 | 0 | 0.5 |
| rt 8.775 | 0 | 1.07 |
| reb AM | 0 | 65.7 |
| rt 10.346 | 0 | 0.83 |
| rt 11.217 | 0 | 0.54 |
| rt 12.425 | 0 | 0.2 |
| reb M | 0 | 14.97 |
| rt 15.174 | 0 | 0.4 |
| reb A | 12.97 | 0 |
| stevioside | 82.83 | 0 |
| reb B | 0.38 | 1.47 |
| steviolbioside | 3.82 | 1.11 |

### Example 7

### Purification of Rebaudiosides 2a, 2m and Various SvG7

300 mL of the reaction mixture of EXAMPLE 5, (after 48 hrs), was inactivated by adjusting the pH to pH 5.5 with H₃PO₄ and then boiled for 10 minutes and filtered. The filtrate was loaded into a column containing 500 mL YWD03 (Cangzhou Yuanwei, China) resin pre-equilibrated with water. The resin was washed with 2.5 L water and the water effluent from this step was discarded.

The steviol glycosides were eluted from the YWD03 resin column by elution with 2.5 L 70% v/v ethanol/water. The effluent from this step was collected and dried under vacuum at 60° C. to yield 20g of dried solid product. This sample was dissolved in water and subjected to further fractionation and separation by HPLC, using the conditions listed in Table 4 below.

HPLC fractions that corresponded to individual compounds from multiple runs were combined according to retention time. The fractions were freeze-dried.

TABLE 4
Conditions for HPLC

| | |
|---|---|
| Column | Agilent Prodigy 3u ODS(3) 100A, |
| | 4.6 mm × 250 mm, 3 micron |
| Temperature | 40° C. |
| Mobile Phase | Isocratic—Water 77% Acetonitrile 23% |
| Flow rate | 0.5 mL/min |
| Injection | 10 μL |
| Stop time | 45 mins |
| Autosampler | Ambient |
| temperature | |
| Detection | UV at 210 nm |

The purity of obtained fractions was evaluated by analytical HPLC method described in EXAMPLE 6. The chromatogram of purified rebaudioside 2a is shown in FIG. 11c. The chromatogram of purified rebaudioside 2m is shown in FIG. 11d.

### Example 8

### Structure Elucidation of Rebaudioside 2a

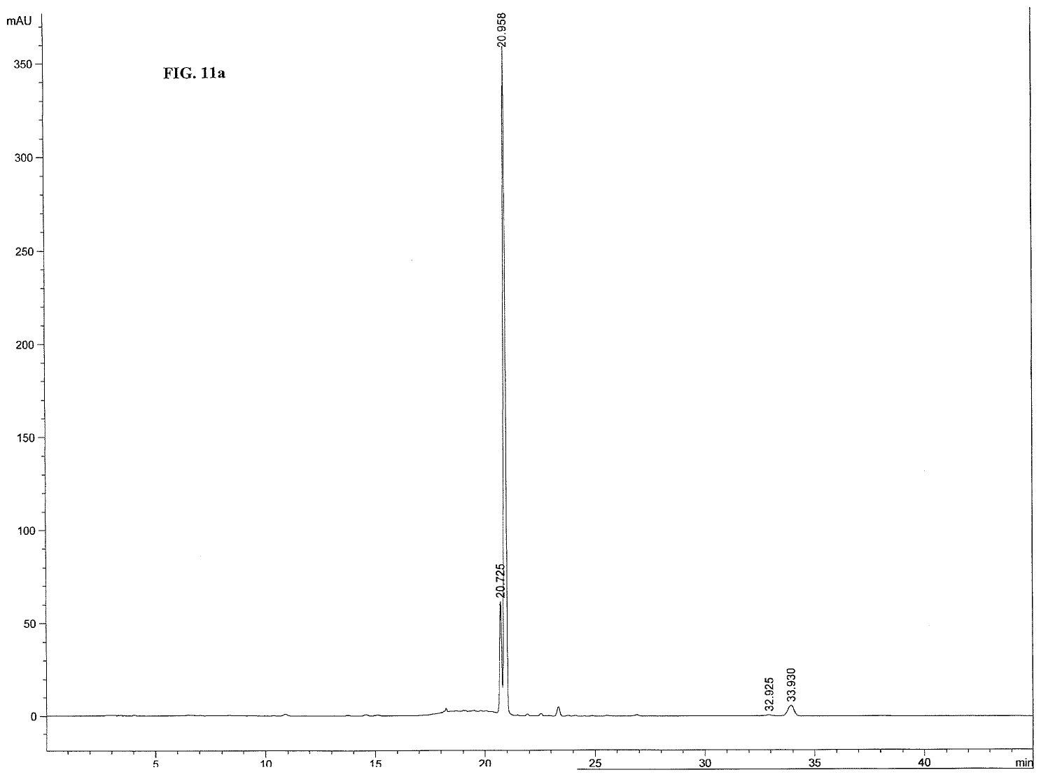

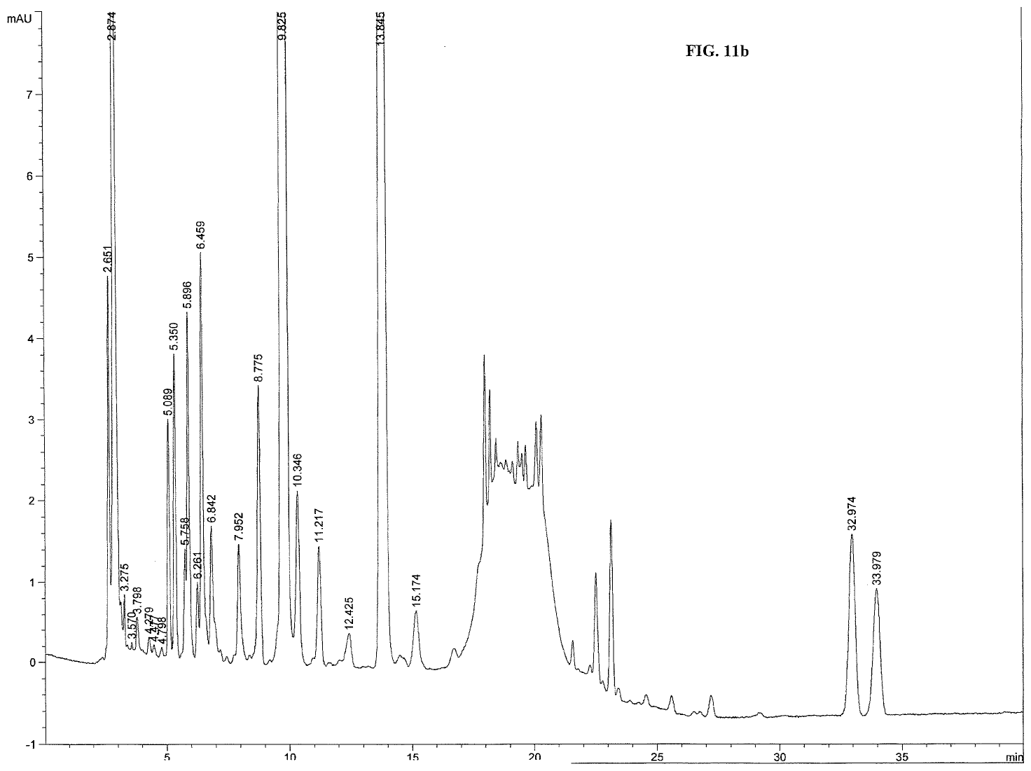

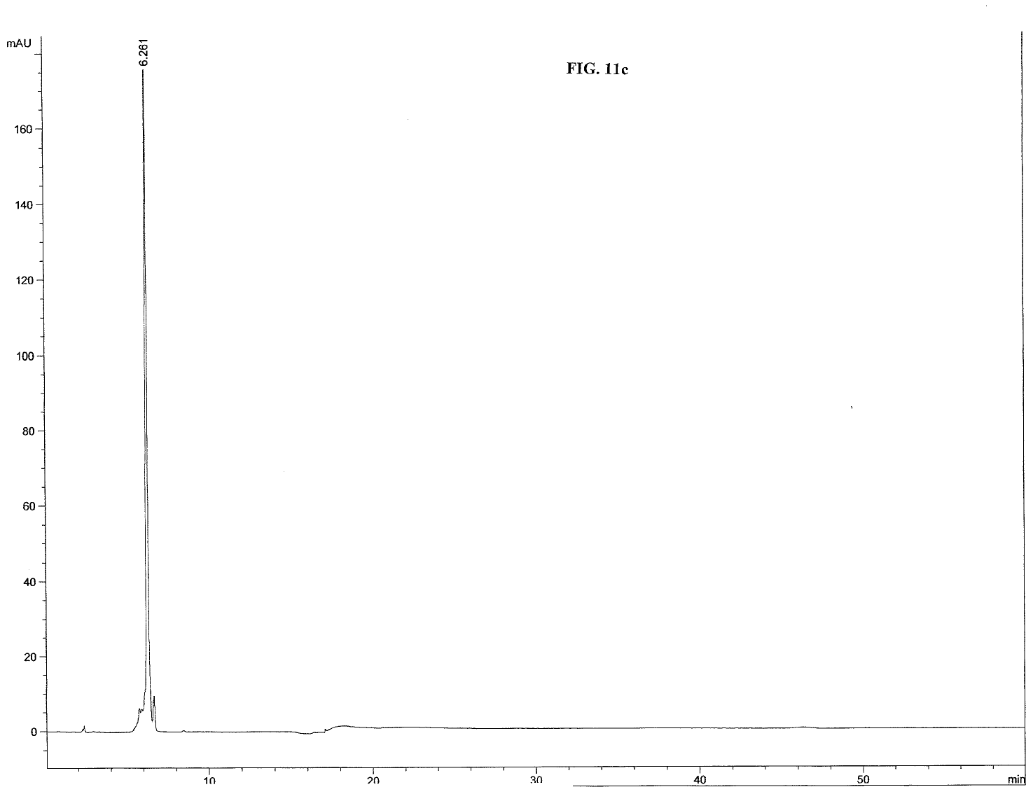

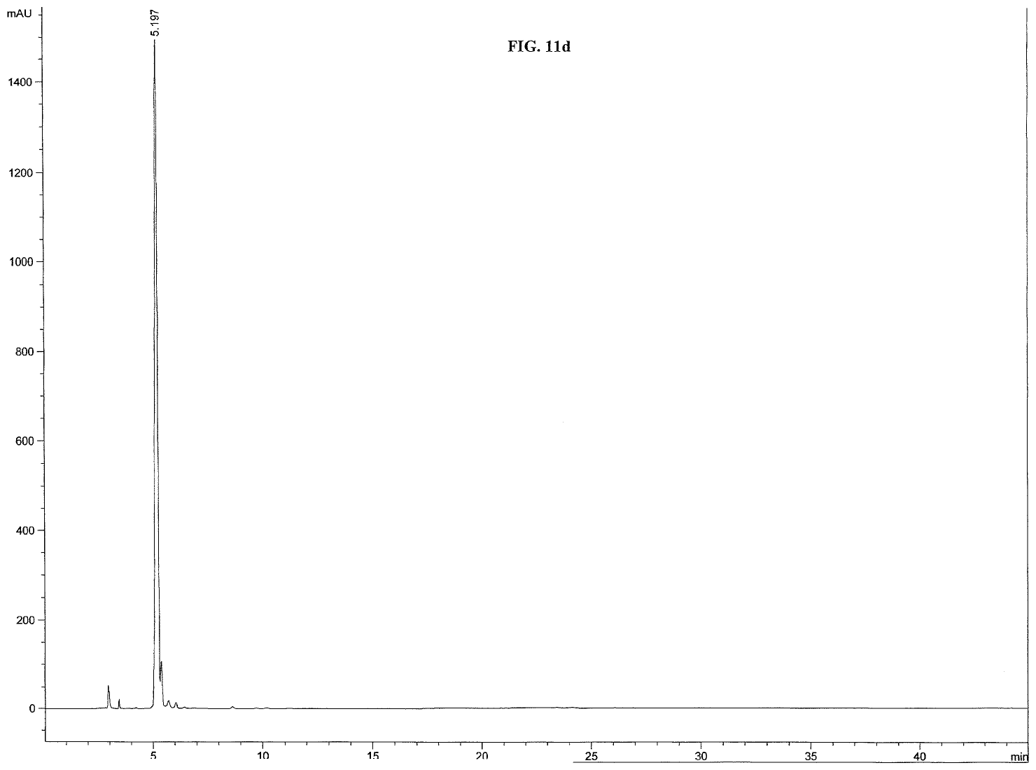

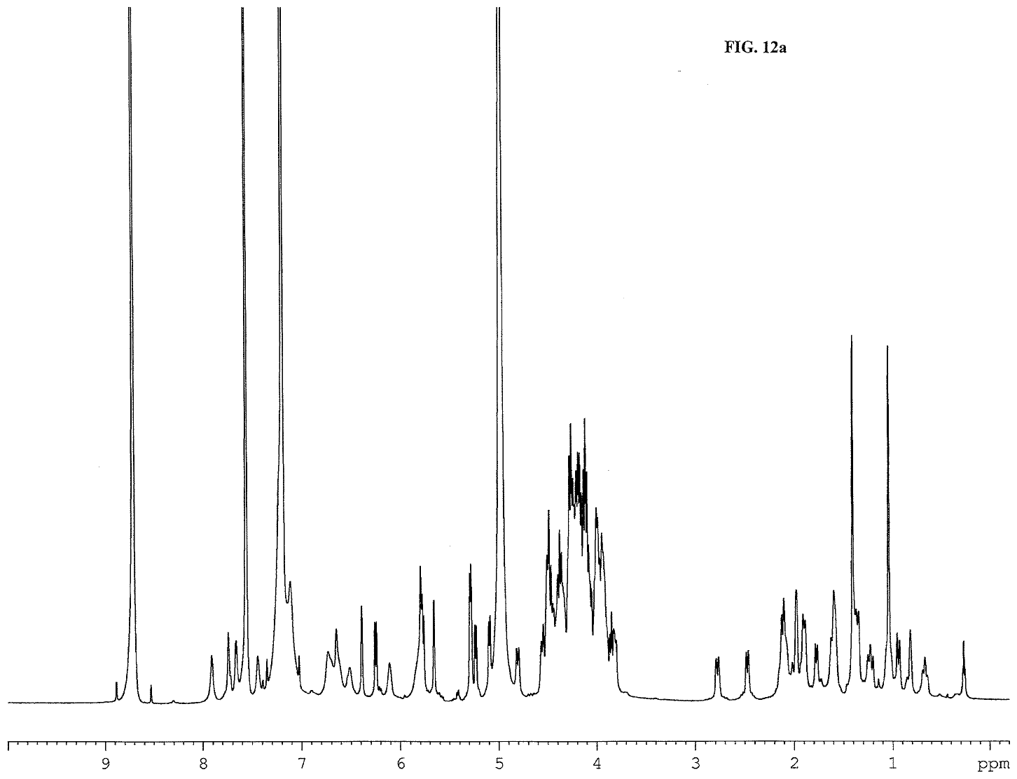

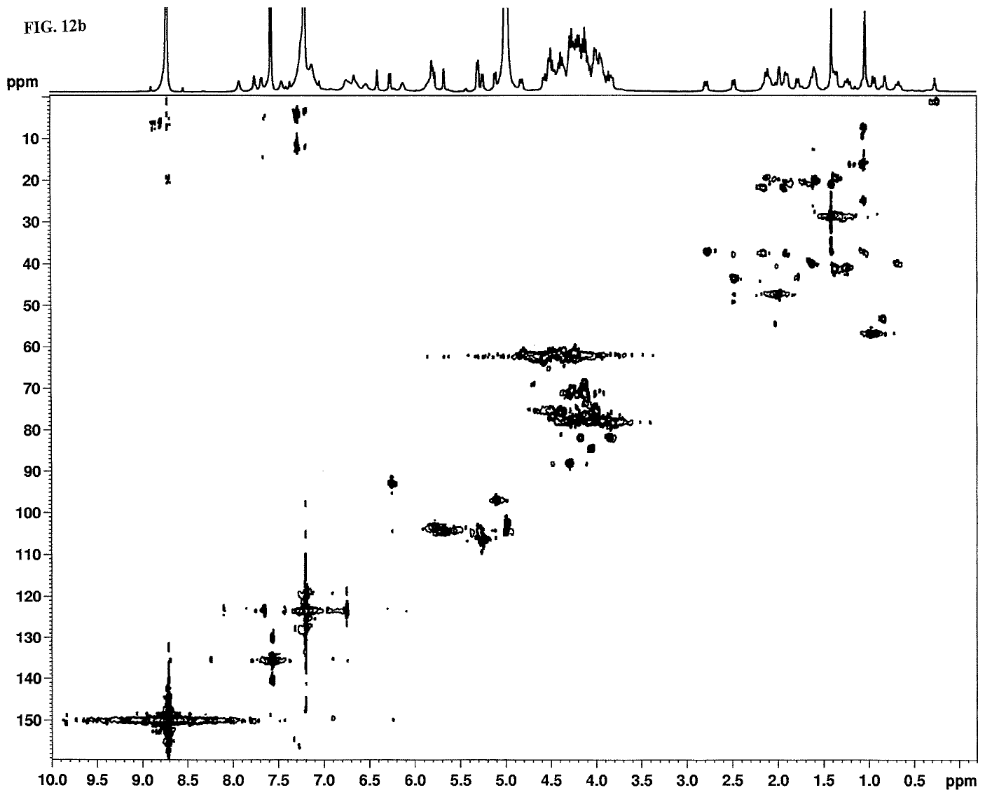

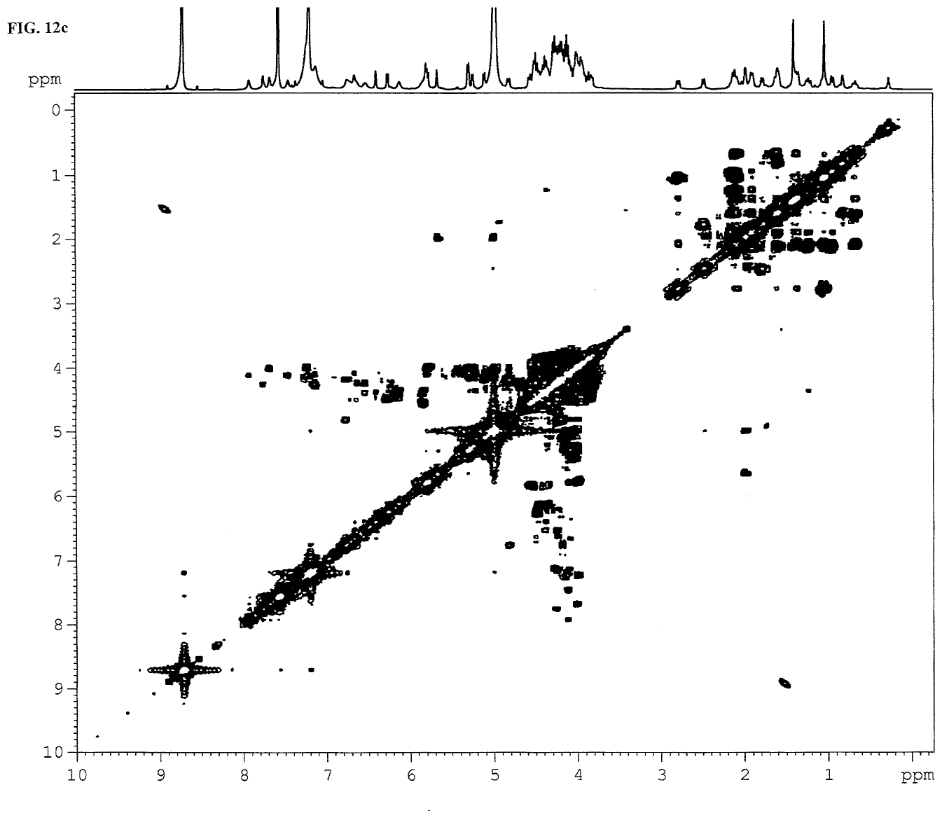

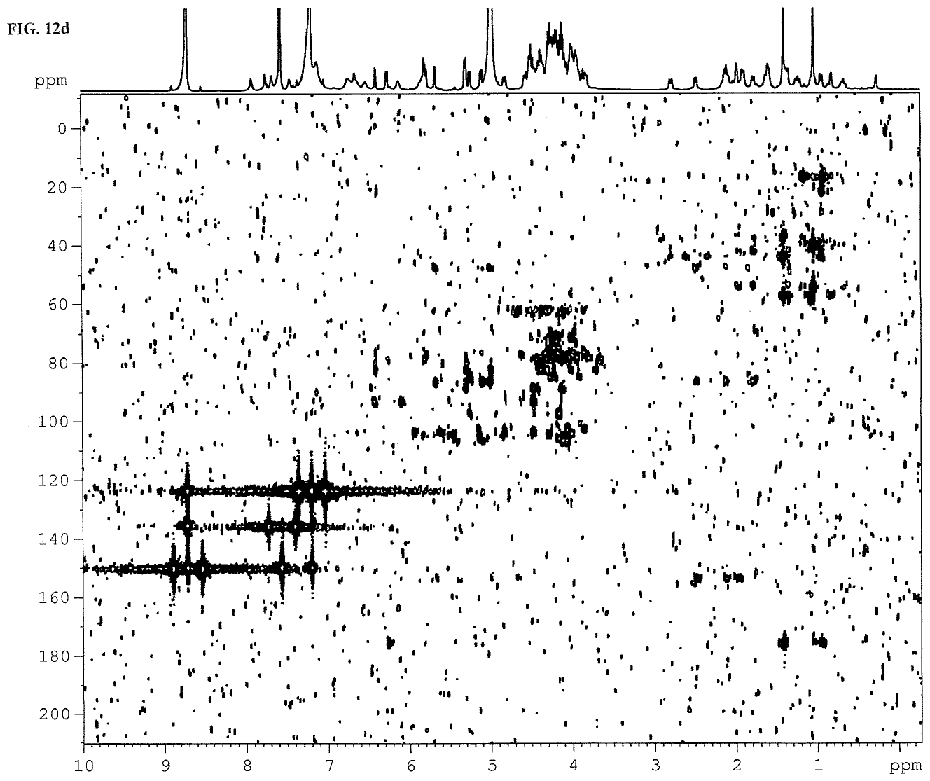

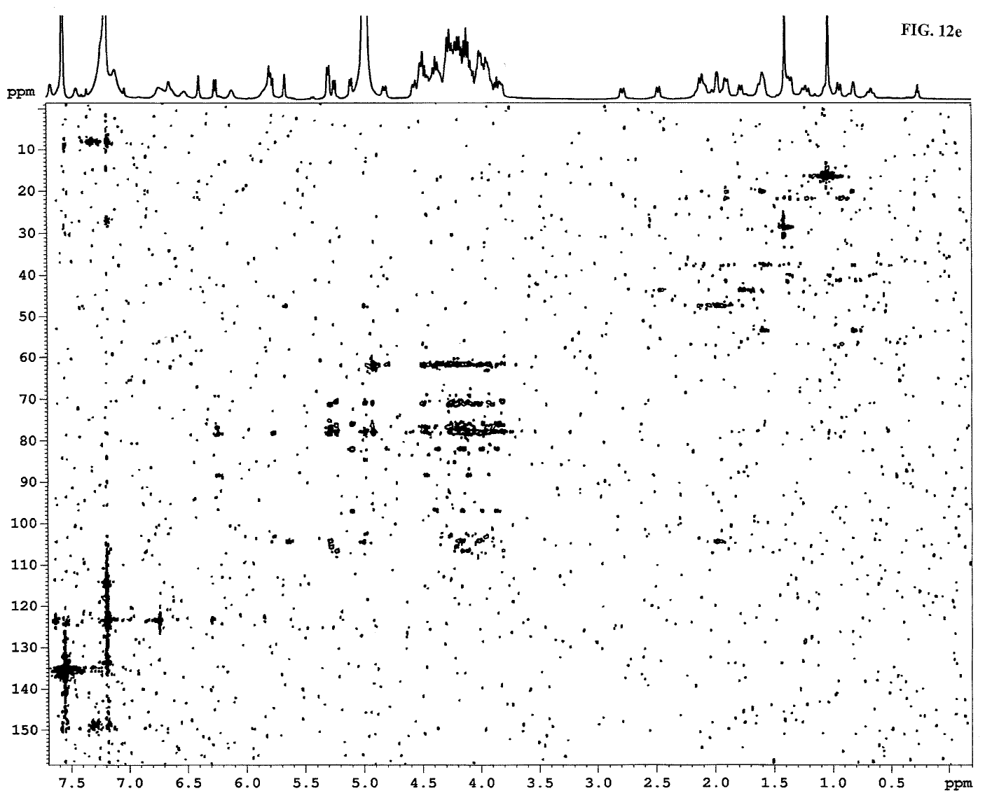

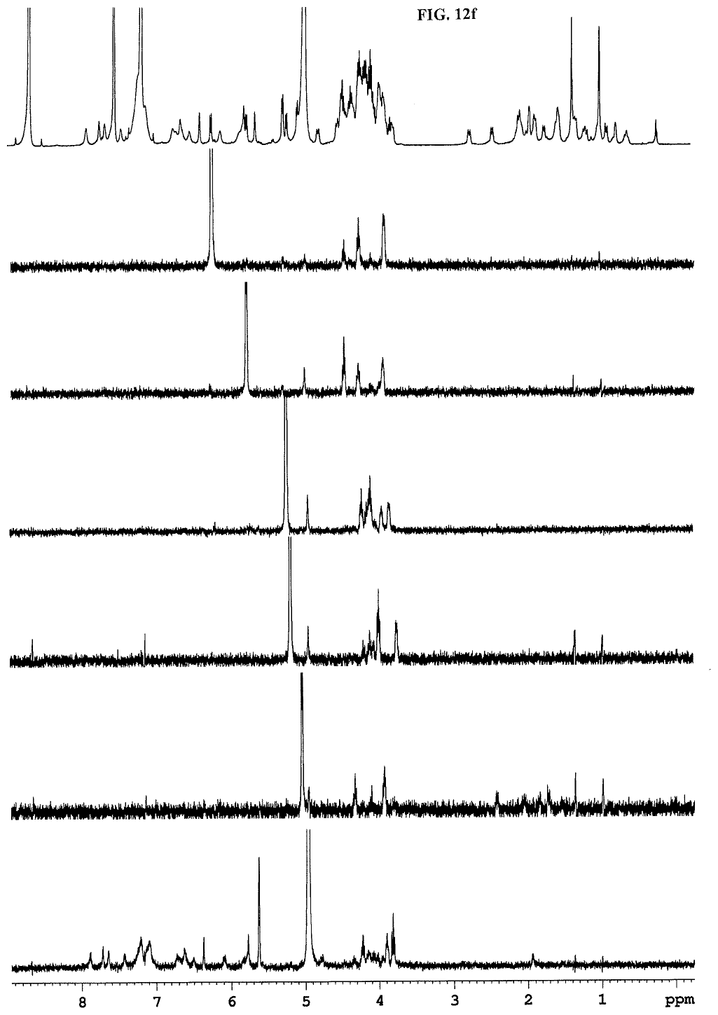

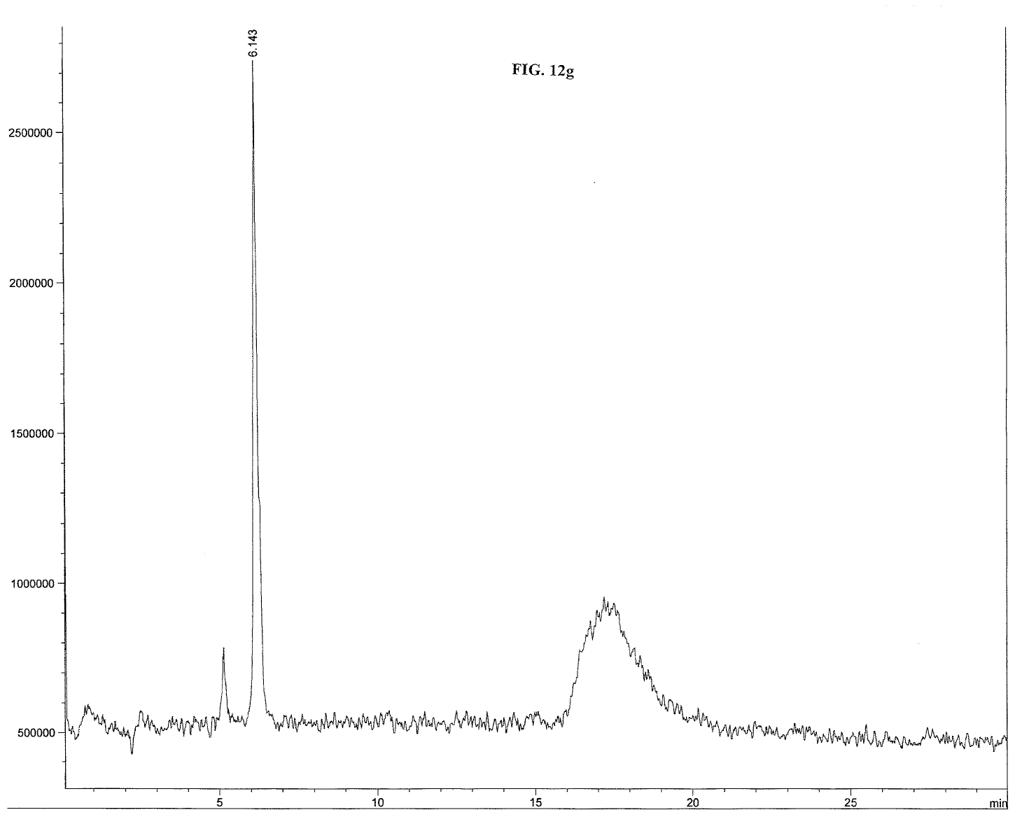

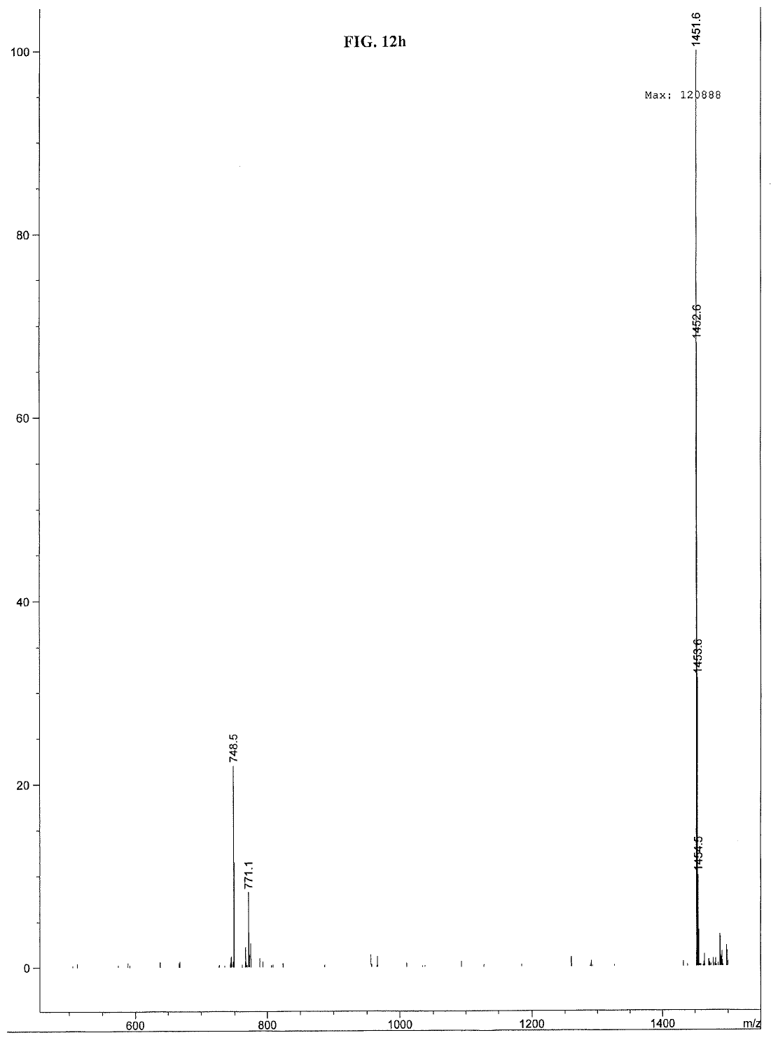

NMR experiments were performed on a Bruker 500 MHz spectrometer, with the sample dissolved in pyridine-d5. Along with signals from the sample, signals from pyridine-d5 at Sc 123.5, 135.5, 149.9 ppm and δ_{H} 7.19, 7.55, 8.71 ppm were observed. ¹H-NMR spectrum of rebaudioside 2a recorded in pyridine-d5 confirmed the excellent quality of the sample (see FIG. 12a). HSQC (see FIG. 12b) shows the presence of an exo-methylene group in the sugar region with a long-range coupling to C-15, observable in the H,H-COSY (FIG. 12c). Other deep-fielded signals of the quaternary carbons (C-13, C-16 and C-19) are detected by the HMBC (FIG. 12d). Correlation of the signals in the HSQC, HMBC and H,H-COSY reveal the presence of steviol glycoside with the following aglycone structure:

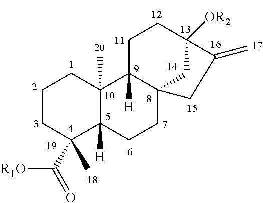

Correlation of HSQC and HMBC shows the presence seven anomeric signals, marked with 1i, 1ii, 1iii, 1iv, 1v, 1vi and 1vii. The coupling constant of the anomeric protons of about 8 Hz, the broad signals of their sugar linkage and the NOE-correlations of the anomeric protons allow the identification of these seven sugars as β-D-glucopyranosides.

Combined data from HSQC and HMBC reveal the sugar-sugar linkages and sugar-aglycone linkages. The assignment of the sugar sequence was confirmed by using the combination of HSQC-TOCSY (FIG. 12e) and NOESY (FIG. 12f).

Altogether, results from NMR experiments above were used to assign the chemical shifts of the protons and carbons of the structure of rebaudioside 2a (see Table 5).

TABLE 5
Chemical shifts of rebaudioside
Aglycone moiety

| Position | δ_{C} [ppm] | | δ_{H} [ppm] | | J [Hz]/(INT) | HMBC (H −> C) |
|---|---|---|---|---|---|---|
| 1 | 40.1 | t | 0.67 | m | | |
| | | | 1.62 | m | | |
| 2 | 19.5 | t | 1.36 | m | | |
| | | | 2.10 | m | | |
| 3 | 37.1 | t | 1.05 | m | | |
| | | | 2.77 | m | | |
| 4 | 44.5 | s | — | | | |
| 5 | 57.1 | d | 0.94 | m | | |
| 6 | 21.8 | t | 1.92 | m | | |
| | | | 2.13 | m | | |
| 7 | 40.9 | t | 1.22 | m | | |
| | | | 1.36 | m | | |
| 8 | 42.1 | s | — | | | |
| 9 | 53.4 | d | 0.82 | m | | |
| 10 | 39.1 | s | — | | | |
| 11 | 20.4 | t | 1.57 | m | | |
| | | | 1.60 | m | | |
| 12 | 37.4 | t | 1.90 | m | | |
| | | | 2.13 | m | | |
| 13 | 86.5 | s | — | | | |
| 14 | 43.4 | t | 1.77 | d | 11.1 | |
| | | | 2.47 | d | 11.1 | |
| 15 | 47.2 | t | 1.96 | d | 16.0 | 7, 8, 9, 14 |
| | | | 1.99 | d | 16.0 | |
| 16 | 153.7 | s | — | | | |
| 17 | 104.4 | t | 4.99 | br s | | 13, 15, 16 |
| | | | 5.67 | br s | | |
| 18 | 28.5 | q | 1.40 | s | (3H) | 3, 4, 5, 19 |
| 19 | 175.7 | s | — | | | |
| 20 | 16.0 | q | 1.04 | s | (3H) | 1, 5, 9, 10 |

| | | | | | |
|---|---|---|---|---|---|
| | | | | HMBC | NOE |
| Position | δ_{C} [ppm] | δ_{H} [ppm] | J [Hz]/(Int) | (H → C) | (H → H) |

Sugar moiety
Sugar I: β-D-Glucopyranoside

| | | | | | | |
|---|---|---|---|---|---|---|
| 1ⁱ | 97.0 | d | 5.09 | d | 7.7 | 13 |
| 2ⁱ | 81.9 | d | 4.16 | m | | |
| 3ⁱ | 75.8 | d | 4.37 | m | | |
| 4ⁱ | 81.9 | d | 3.85 | m | | |
| 5ⁱ | 75.6 | d | 3.97 | m | | |
| 6ⁱ | 61.8 | t | 4.20 | m | | |
| | | | 4.80 | m | | |

Sugar II: β-D-Glucopyranoside

| | | | | | | | |
|---|---|---|---|---|---|---|---|
| 1ⁱⁱ | 105.0 | d | 5.28 | d | 8.0 | 2ⁱ | 2ⁱ |
| 2ⁱⁱ | 76.3 | d | 4.09 | m | | | |
| 3ⁱⁱ | 78.0 | d | 4.29 | m | | | |
| 4ⁱⁱ | 70.8 | d | 4.24 | m | | | |
| 5ⁱⁱ | 78.1 | d | 3.91 | m | | | |
| 6ⁱⁱ | 61.4 | t | 4.39 | m | | | |
| | | | 4.50 | m | | | |

Sugar III: β-D-Glucopyranoside

| | | | | | | | |
|---|---|---|---|---|---|---|---|
| 1ⁱⁱⁱ | 102.4 | d | 4.98 | d | 8.1 | 4ⁱ | 4ⁱ |
| 2ⁱⁱⁱ | 84.8 | d | 4.05 | m | | | |
| 3ⁱⁱⁱ | 77.9 | d | 4.22 | m | | | |
| 4ⁱⁱⁱ | 70.9 | d | 4.12 | m | | | |
| 5ⁱⁱⁱ | 77.6 | d | 3.92 | m | | | |
| 6ⁱⁱⁱ | 62.5 | t | 4.31 | m | | | |
| | | | 4.53 | m | | | |

Sugar IV: β-D-Glucopyranoside

| | | | | | | | |
|---|---|---|---|---|---|---|---|
| 1^{iv} | 106.4 | d | 5.22 | d | 7.8 | 2ⁱⁱⁱ | 2ⁱⁱⁱ |
| 2^{iv} | 76.1 | d | 4.12 | m | | | |
| 3^{iv} | 78.1 | d | 4.06 | m | | | |
| 4^{iv} | 70.4 | d | 4.25 | m | | | |
| 5^{iv} | 78.3 | d | 3.81 | m | | | |
| 6^{iv} | 61.6 | t | 4.24 | m | | | |
| | | | 4.50 | m | | | |

Sugar V: β-D-Glucopyranoside

| | | | | | | |
|---|---|---|---|---|---|---|
| 1^{v} | 93.0 | d | 6.25 | d | 8.1 | 19 |
| 2^{v} | 76.8 | d | 4.47 | m | | |
| 3^{v} | 88.2 | d | 4.28 | m | | |
| 4^{v} | 69.1 | d | 4.12 | m | | |
| 5^{v} | 78.2 | d | 3.92 | m | | |
| 6^{v} | 61.5 | t | 4.16 | m | | |
| | | | 4.33 | m | | |

Sugar VI: β-D-Glucopyranoside

| | | | | | | | |
|---|---|---|---|---|---|---|---|
| 1^{vi} | 103.5 | d | 5.77 | d | 7.8 | 2^{v} | 2^{v} |
| 2^{vi} | 75.4 | d | 4.00 | m | | | |
| 3^{vi} | 77.6 | d | 4.27 | m | | | |
| 4^{vi} | 71.1 | d | 4.27 | m | | | |
| 5^{vi} | 78.0 | d | 3.96 | m | | | |
| 6^{vi} | 62.7 | t | 4.42 | m | | | |
| | | | 4.58 | m | | | |

Sugar VII: β-D-Glucopyranoside

| | | | | | | | |
|---|---|---|---|---|---|---|---|
| 1^{vii} | 104.4 | d | 5.28 | d | 8.0 | 3^{v} | 3^{v} |
| 2^{vii} | 75.0 | d | 4.01 | m | | | |
| 3^{vii} | 77.2 | d | 4.17 | m | | | |
| 4^{vii} | 71.5 | d | 4.10 | m | | | |
| 5^{vii} | 77.9 | d | 4.00 | m | | | |
| 6^{vii} | 61.8 | t | 4.42 | m | | | |
| | | | 4.52 | m | | | |

Correlation of all NMR results indicates rebaudioside 2a with seven β-D-glucoses attached to steviol aglycone, as depicted with the following chemical structure:

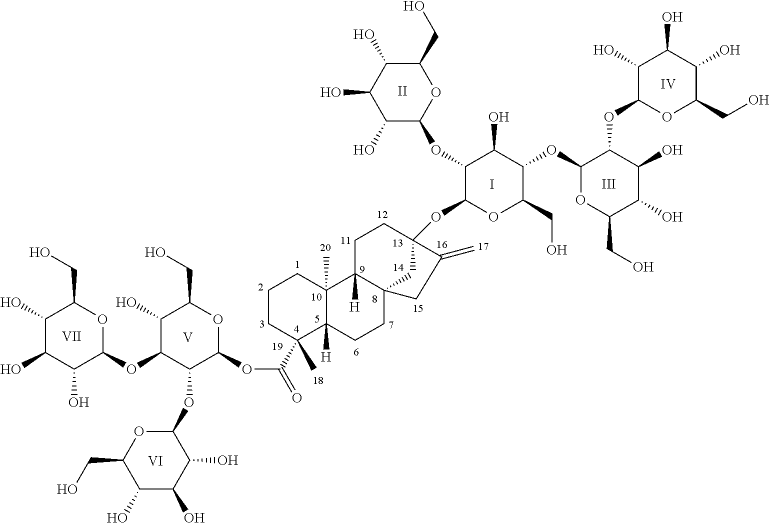

LCMS (FIG. 12g and FIG. 12h) analysis of rebaudioside 2a showed a [M-H]⁻ ion at m/z 1451.6, in good agreement with the expected molecular formula of C₆₂H₁₀₀O₃₈ (calculated for [C₆₂H₉₉O₃₈]⁻ monoisotopic ion: 1451.6). The MS data confirms that rebaudioside 2a has a molecular formula of C₆₂H₁₀₀O₃₈. LCMS analysis was performed in the following conditions listed in Table 6.

TABLE 6
Conditions for LCMS analysis

| Column | Agilent Poroshell 120 SB-C18, | | |
|---|---|---|---|
| | 4.6 mm × 150 mm, 2.7 μm | | |
| Temperature | 40° C. | | |
| Mobile | A: Mobile Phase Premix Solution | | |
| Phase | −25% Acetonitrile: 75% Formic | | |
| | Acid (0.1% in Water) | | |
| | B: Mobile Phase Premix Solution | | |
| | −32% Acetonitrile: 68% Formic | | |
| | Acid (0.1% in Water) | | |
| Gradient | Time (min) | A (%) | B (%) |
| | 0 | 100 | 0 |
| | 12.0 | 100 | 0 |
| | 12.5 | 50 | 50 |
| | 13.0 | 0 | 100 |
| | 60.0 | 0 | 100 |

| | |
|---|---|
| Flow rate | 0.5 mL/min |
| Injection | 2 μL |
| Run time | 45 mins |
| Post time | 5 mins |
| Autosampler | Ambient |
| temperature | |
| Detection | MSD at Negative Scan mode |
| MSD Setting | Mode: ES-API, Negative Polarity |
| | Drying gas flow: 13.0 L/min |
| | Nebulizer Pressure: 30 psig |
| | Drying gas temperature: 270° C. |
| | Fragmentor: 50V |
| | Scan ranges: 500 to 1500 of mass |
| Sample | 1 mg/ml (30% ACN in water) |
| Preparation | |

### Example 9

### Structure Elucidation of Rebaudioside 2m

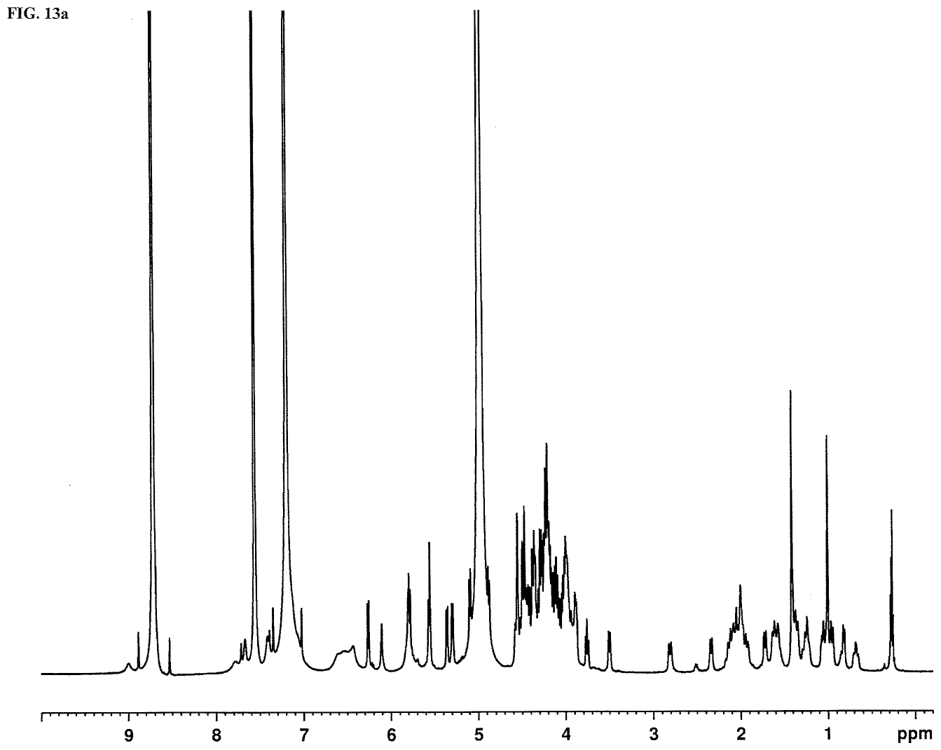

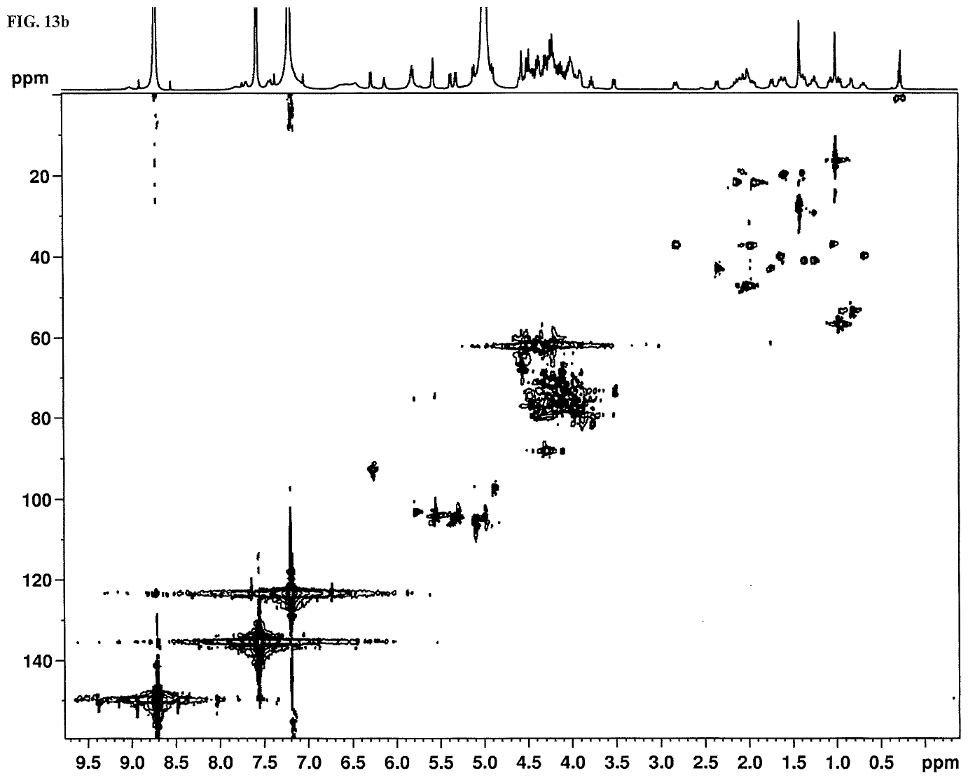

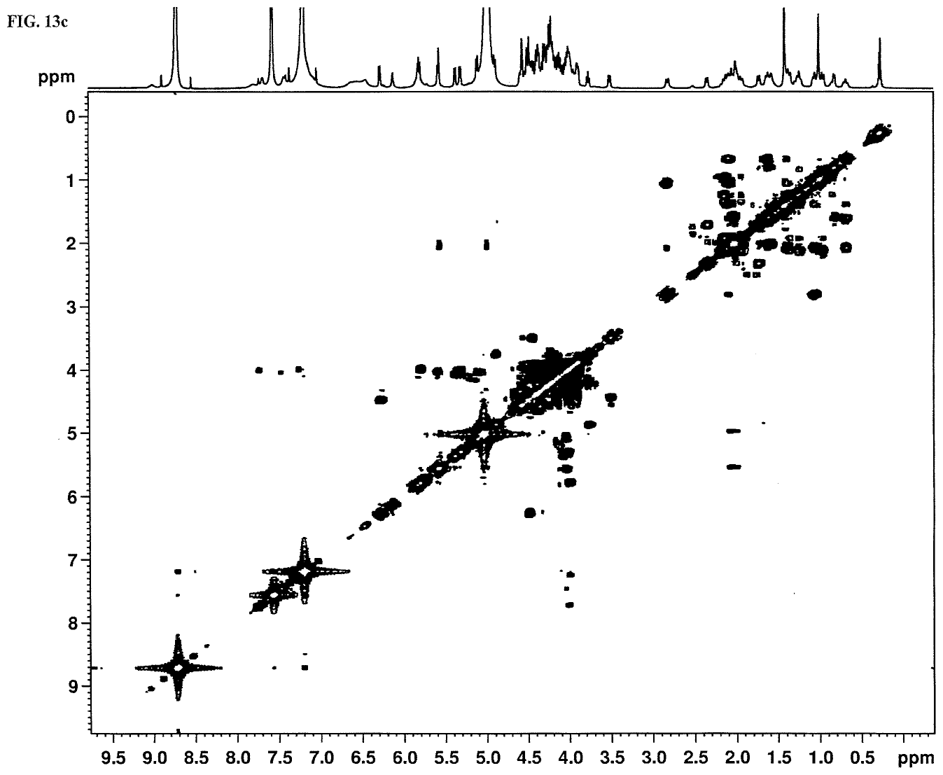

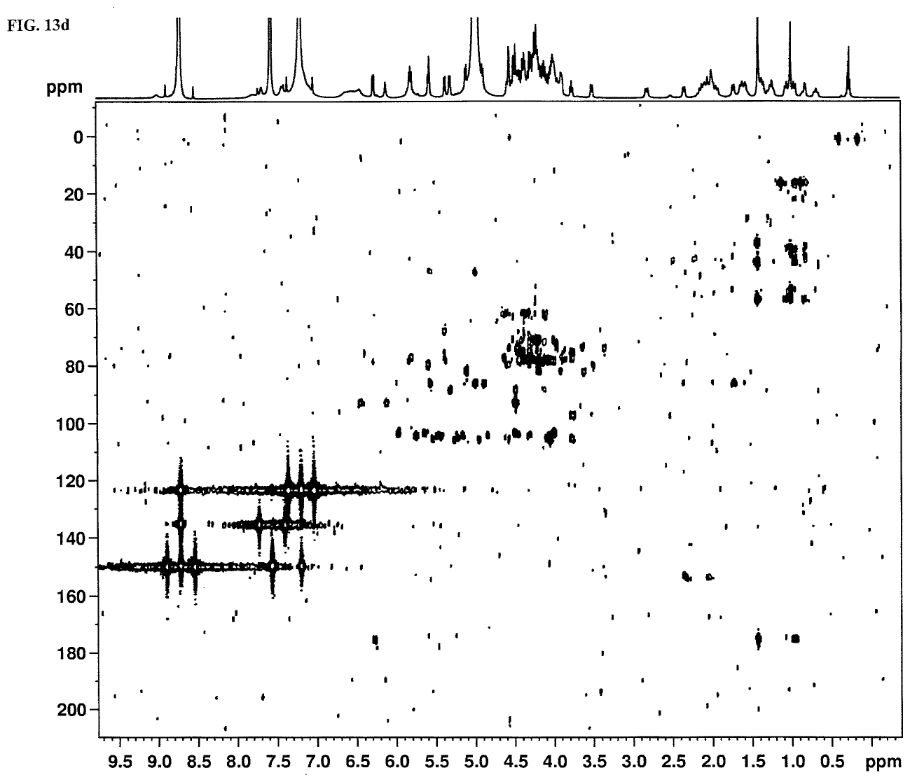

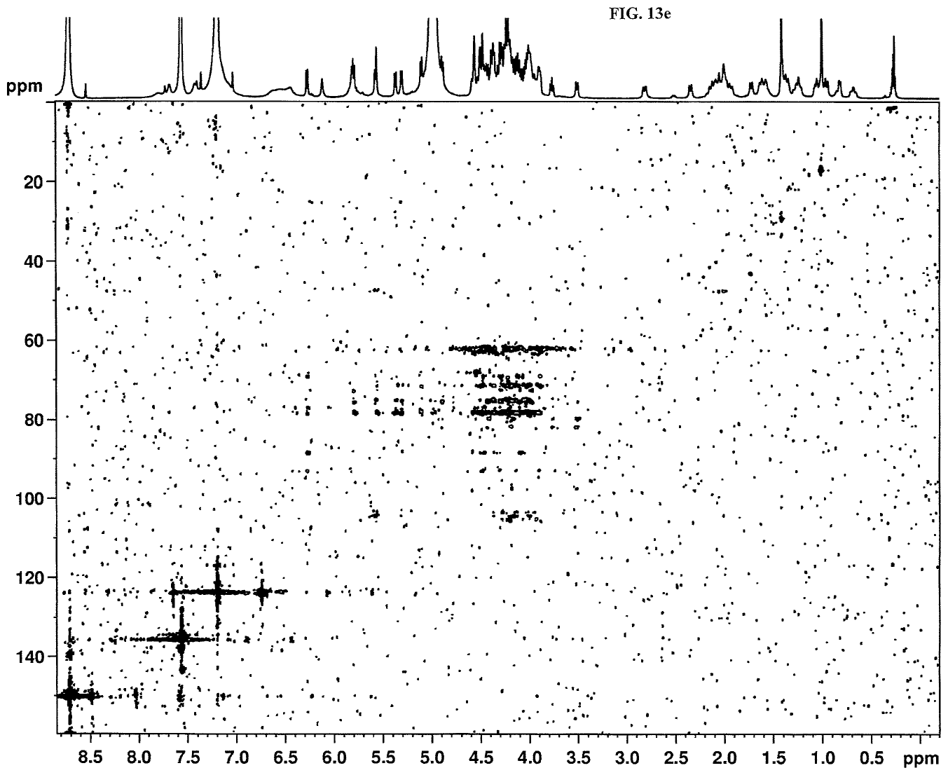

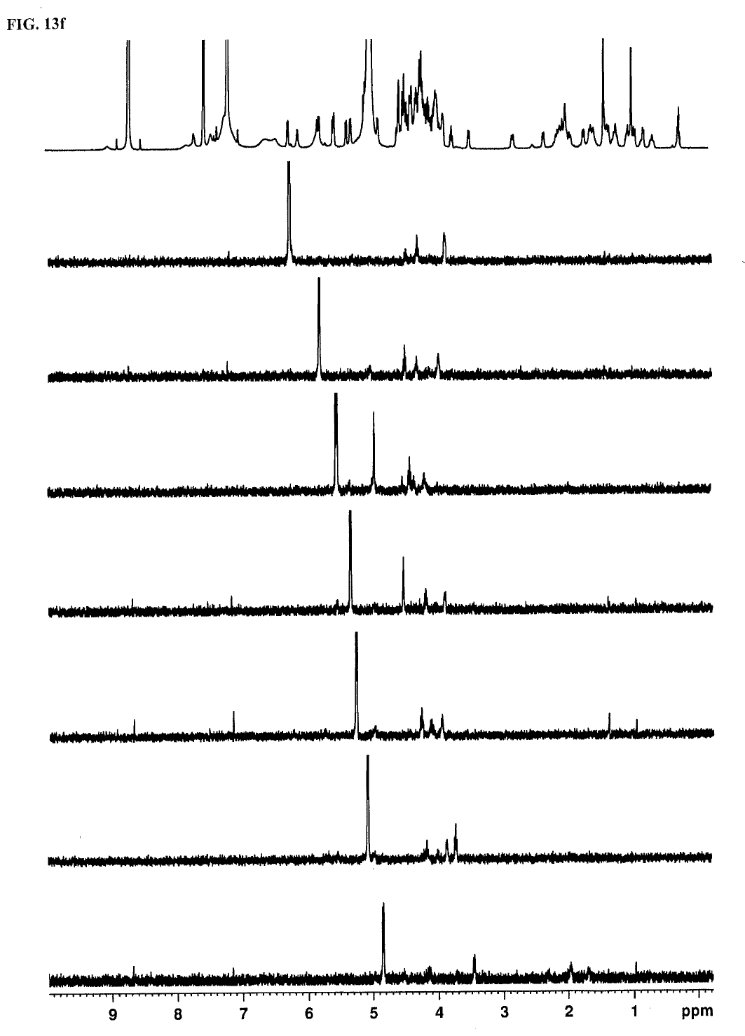

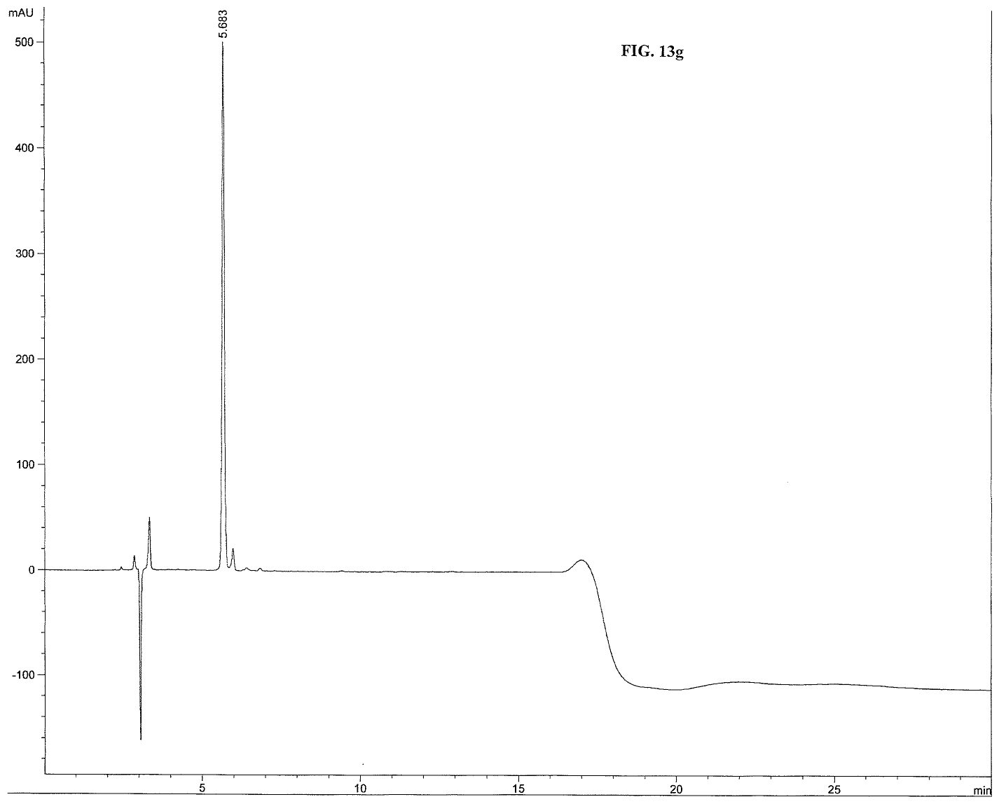

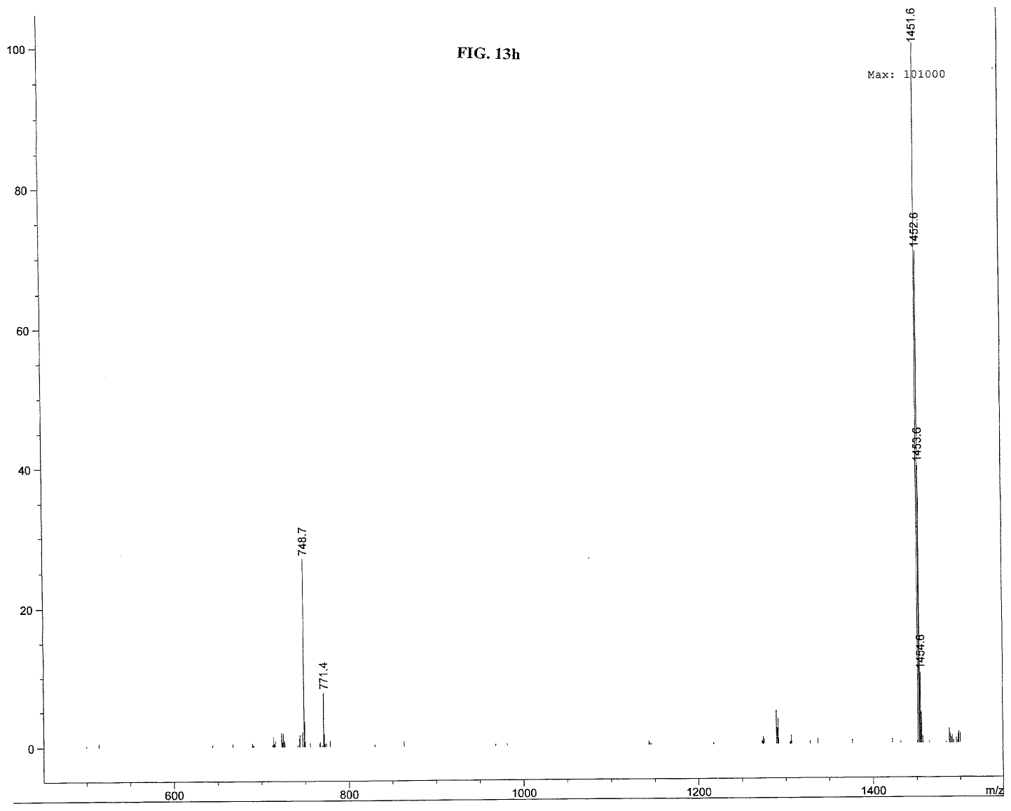

NMR experiments were performed on a Bruker 500 MHz spectrometer, with the sample dissolved in pyridine-d5. Along with signals from the sample, signals from pyridine-d5 at Sc 123.5, 135.5, 149.9 ppm and δ_{H} 7.19, 7.55, 8.71 ppm were observed. ¹H-NMR spectrum of rebaudioside 2m recorded in pyridine-d5 confirmed the excellent quality of the sample (see FIG. 13a). HSQC (see FIG. 13b) shows the presence of an exo-methylene group in the sugar region with a long-range coupling to C-15, observable in the H,H-COSY (FIG. 13c). Other deep-fielded signals of the quaternary carbons (C-13, C-16 and C-19) are detected by the HMBC (FIG. 13d). Correlation of the signals in the HSQC, HMBC and H,H-COSY reveal the presence of steviol glycoside with the following aglycone structure:

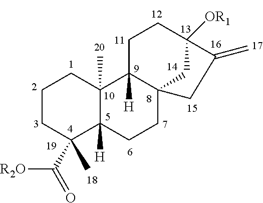

Correlation of HSQC and HMBC shows the presence seven anomeric signals, marked with 1i, 1ii, 1iii, 1iv, 1v, 1vi and 1vii. The coupling constant of the anomeric protons of about 8 Hz, the broad signals of their sugar linkage and the NOE-correlations of the anomeric protons allow the identification of these seven sugars as β-D-glucopyranosides.

Combined data from HSQC and HMBC reveal the sugar-sugar linkages and sugar-aglycone linkages. The assignment of the sugar sequence was confirmed by using the combination of HSQC-TOCSY (FIG. 13e) and NOESY (FIG. 13f).

Altogether, results from NMR experiments above were used to assign the chemical shifts of the protons and carbons of the structure of rebaudioside 2m (see Table 7).

TABLE 7
Chemical shifts of rebaudioside 2m
Aglycone moiety

| Position | δ_{C} [ppm] | | δ_{H} [ppm] | | J [Hz]/(INT) | HMBC (H −> C) |
|---|---|---|---|---|---|---|
| 1 | 40.0 | t | 0.68 | m | | |
| | | | 1.63 | m | | |
| 2 | 19.3 | t | 1.38 | m | | |
| | | | 2.06 | m | | |
| 3 | 37.1 | t | 1.04 | m | | |
| | | | 2.81 | m | | |
| 4 | 44.0 | s | — | | | |
| 5 | 56.9 | d | 0.96 | m | | |
| 6 | 21.8 | t | 1.93 | m | | |
| | | | 2.13 | m | | |
| 7 | 41.1 | t | 1.25 | m | | |
| | | | 1.36 | m | | |
| 8 | 41.6 | s | — | | | |
| 9 | 53.6 | d | 0.82 | m | | |
| 10 | 40.4 | s | — | | | |
| 11 | 19.8 | t | 1.58 | m | | |
| | | | 1.61 | m | | |
| 12 | 37.5 | t | 1.96 | m | | |
| | | | 1.99 | m | | |
| 13 | 86.2 | s | — | | | |
| 14 | 43.1 | t | 1.73 | d | 10.9 | |
| | | | 2.34 | d | 10.9 | |
| 15 | 47.5 | t | 1.96 | d | 15.9 | 7, 8, 9, 14 |
| | | | 2.02 | d | 15.9 | |
| 16 | 153.4 | s | — | | | |
| 17 | 104.3 | t | 4.98 | br s | | 13, 15, 16 |
| | | | 5.55 | br s | | |
| 18 | 28.6 | q | 1.42 | s | (3H) | 3, 4, 5, 19 |
| 19 | 175.3 | s | — | | | |
| 20 | 16.3 | q | 1.01 | s | (3H) | 1,5, 9, 10 |

| | | | | | |
|---|---|---|---|---|---|
| | | | | HMBC | NOE |
| Position | δ_{C} [ppm] | δ_{H} [ppm] | J [Hz]/(Int) | (H → C) | (H → H) |

Sugar moiety
Sugar I: β-D-Glucopyranoside

| | | | | | | | |
|---|---|---|---|---|---|---|---|
| 1ⁱ | 97.3 | d | 4.88 | d | 7.8 | 13 | 3ⁱ, 5ⁱ |
| 2ⁱ | 81.9 | d | 3.75 | m | | | |
| 3ⁱ | 75.3 | d | 4.18 | m | | | |
| 4ⁱ | 79.6 | d | 4.40 | m | | | |
| 5ⁱ | 74.0 | d | 3.50 | m | | | |
| 6ⁱ | 67.9 | t | 4.55 | m | (2H) | | 5ⁱ, 1^{iv} |

Sugar II: β-D-Glucopyranoside

| | | | | | | | |
|---|---|---|---|---|---|---|---|
| 1ⁱⁱ | 105.7 | d | 5.10 | d | 8.0 | 2ⁱ | 2ⁱ, 3ⁱⁱ, 5ⁱⁱ |
| 2ⁱⁱ | 74.6 | d | 4.02 | m | | | |
| 3ⁱⁱ | 77.9 | d | 4.18 | m | | | |
| 4ⁱⁱ | 71.4 | d | 4.20 | m | | | |
| 5ⁱⁱ | 78.1 | d | 3.87 | m | | | |
| 6ⁱⁱ | 61.8 | t | 4.19 | m | | | |
| | | | 4.46 | m | | | |

Sugar III: β-D-Glucopyranoside

| | | | | | | | |
|---|---|---|---|---|---|---|---|
| 1ⁱⁱⁱ | 104.3 | d | 5.57 | d | 8.1 | 4ⁱ | 4ⁱ, 3ⁱⁱⁱ. 5ⁱⁱⁱ |
| 2ⁱⁱⁱ | 75.2 | d | 4.03 | m | | | |
| 3ⁱⁱⁱ | 77.1 | d | 4.35 | m | | | |
| 4ⁱⁱⁱ | 71.4 | d | 4.23 | m | | | |
| 5ⁱⁱⁱ | 76.8 | d | 4.19 | m | | | |
| 6ⁱⁱⁱ | 63.3 | t | 4.34 | m | | | |
| | | | 4.56 | m | | | |

Sugar IV: β-D-Glucopyranoside

| | | | | | | | |
|---|---|---|---|---|---|---|---|
| 1^{iv} | 104.9 | d | 5.37 | d | 8.0 | 6ⁱ | 6ⁱ, 3^{iv}, 5^{iv} |
| 2^{iv} | 75.0 | d | 4.06 | m | | | |
| 3^{iv} | 78.3 | d | 4.29 | m | | | |
| 4^{iv} | 71.1 | d | 4.27 | m | | | |
| 5^{iv} | 78.0 | d | 3.99 | m | | | |
| 6^{iv} | 62.1 | t | 4.22 | m | | | |
| | | | 4.36 | m | | | |

Sugar V: β-D-Glucopyranoside

| | | | | | | | |
|---|---|---|---|---|---|---|---|
| 1^{v} | 92.8 | d | 6.27 | d | 8.1 | 19 | 3^{v}, 5^{v} |
| 2^{v} | 77.0 | d | 4.47 | m | | | |
| 3^{v} | 88.2 | d | 4.28 | m | | | |
| 4^{v} | 69.0 | d | 4.10 | m | | | |
| 5^{v} | 78.1 | d | 3.88 | m | | | |
| 6^{v} | 61.7 | t | 4.22 | m | | | |
| | | | 4.36 | m | | | |

Sugar VI: β-D-Glucopyranoside

| | | | | | | | |
|---|---|---|---|---|---|---|---|
| 1^{vi} | 103.2 | d | 5.78 | d | 7.8 | 2^{v} | 2^{v}, 3^{vi}, 5^{vi} |
| 2^{vi} | 75.6 | d | 3.99 | m | | | |
| 3^{vi} | 78.2 | d | 4.25 | m | | | |
| 4^{vi} | 72.7 | d | 4.10 | m | | | |
| 5^{vi} | 78.0 | d | 3.93 | m | | | |
| 6^{vi} | 62.6 | t | 4.29 | m | | | |
| | | | 4.40 | m | | | |

Sugar VII: β-D-Glucopyranoside

| | | | | | | | |
|---|---|---|---|---|---|---|---|
| 1^{vii} | 104.4 | d | 5.30 | d | 8.0 | 3^{v} | 3^{v}, 3^{vii}, 5^{vii} |
| 2^{vii} | 75.3 | d | 4.00 | m | | | |
| 3^{vii} | 78.1 | d | 4.12 | m | | | |
| 4^{vii} | 71.2 | d | 4.12 | m | | | |
| 5^{vii} | 78.1 | d | 3.97 | m | | | |
| 6^{vii} | 62.0 | t | 4.26 | m | | | |
| | | | 4.46 | m | | | |

Correlation of all NMR results indicates rebaudioside 2m with seven β-D-glucoses attached to steviol aglycone, as depicted with the following chemical structure:

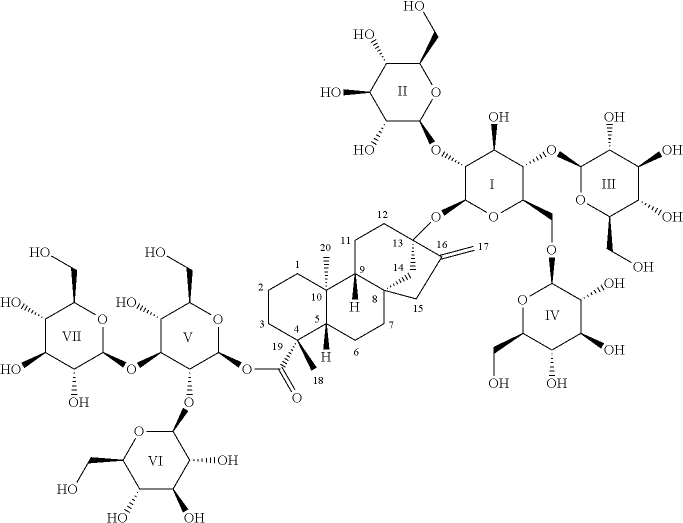

LCMS (FIG. 13g and FIG. 13h) analysis of rebaudioside 2m showed a [M-H]⁻ ion at m/z 1451.6, in good agreement with the expected molecular formula of C₆₂H₁₀₀O₃₈ (calculated for [C₆₂H₉₉O₃₈]⁻ monoisotopic ion: 1451.6). The MS data confirms that rebaudioside 2m has a molecular formula of C₆₂H₁₀₀O₃₈. LCMS analysis was performed in the conditions listed in Table 6.

Although the invention and its advantages have been described in detail, it should be understood that various changes, substitutions and alterations can be made herein without departing from the spirit and scope of the invention as defined by the appended claims. Moreover, the scope of the application is not intended to be limited to the particular embodiments of the invention described in the specification. As one of ordinary skill in the art will readily appreciate from the disclosure of the invention, the compositions, processes, methods, and steps, presently existing or later to be developed that perform substantially the same function or achieve substantially the same result as the corresponding embodiments described herein may be utilized according to the invention.

1. Steviol glycosides I-XL with the following formulae: 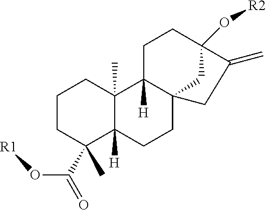
wherein R1 and R2 sugar chains are defined in the following table;

| No. | R1 | R2 |
|---|---|---|
| I | 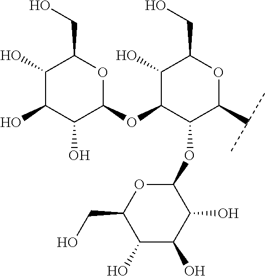 | 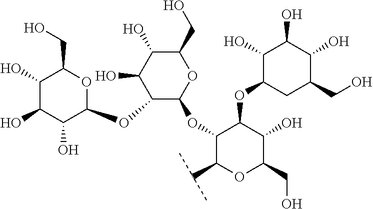 |
| II | 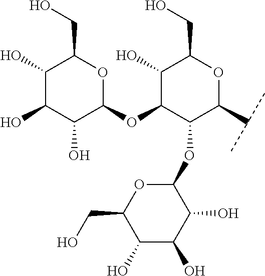 | 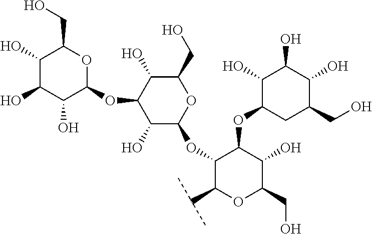 |
| III | 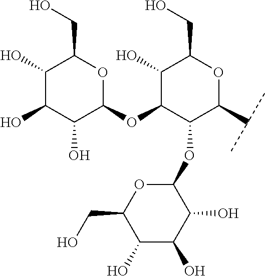 | 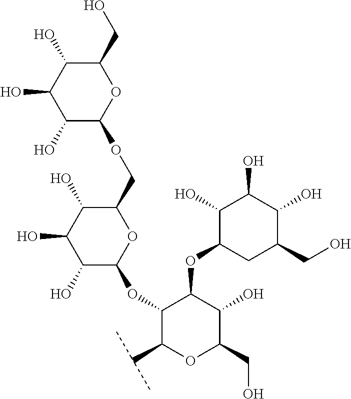 |
| IV | 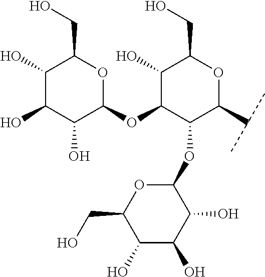 | 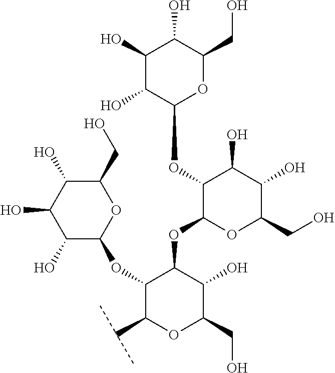 |
| V | 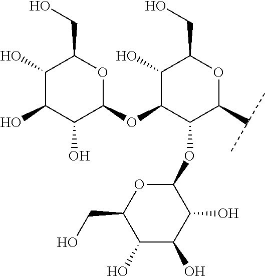 | 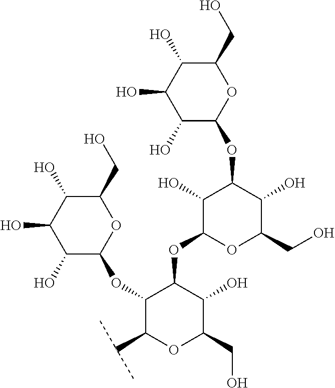 |
| VI | 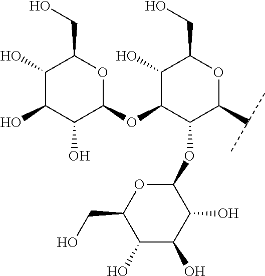 | 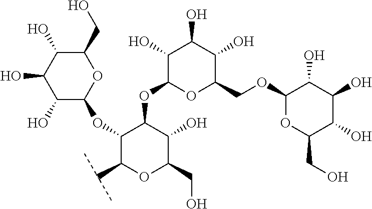 |
| VII | 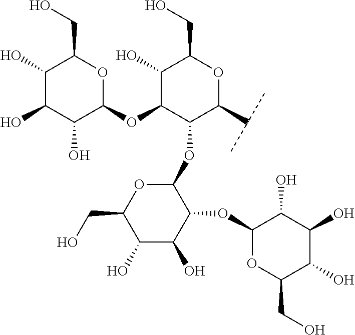 | 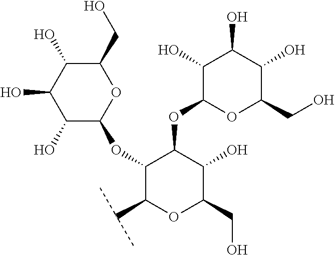 |
| VIII | 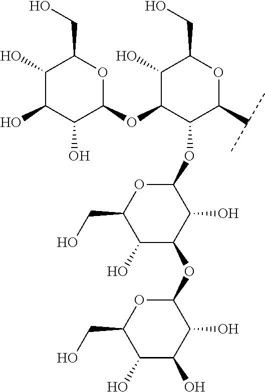 | 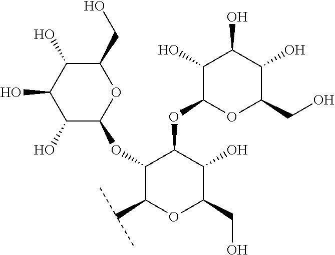 |
| IX | 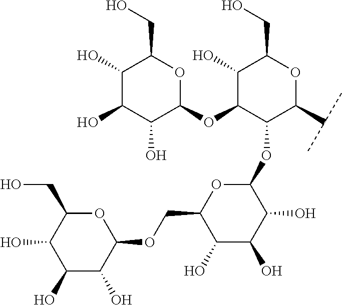 | 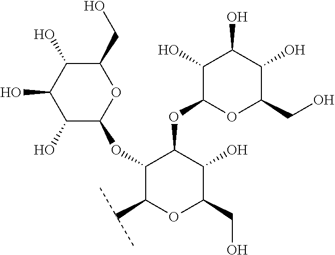 |
| X | 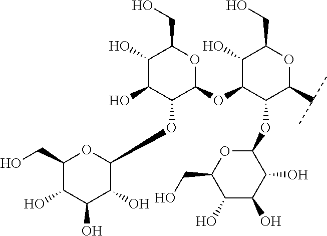 | 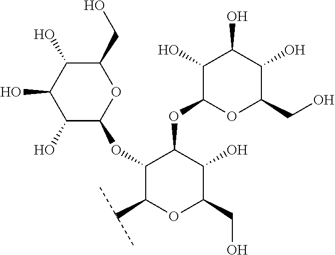 |
| XI | 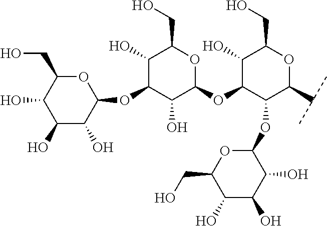 | 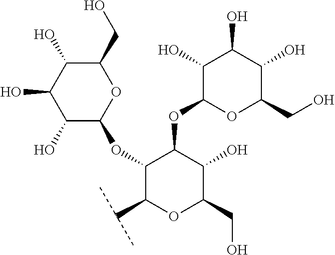 |
| XII | 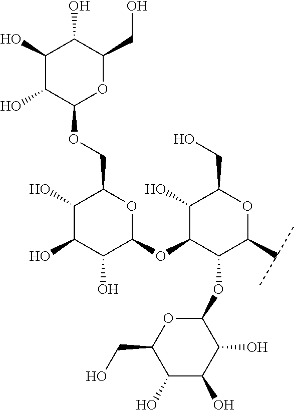 | 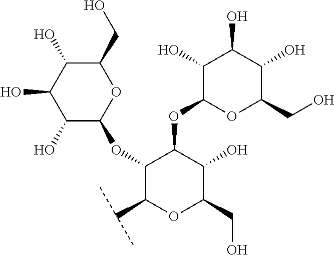 |
| XIII | 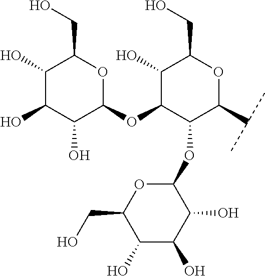 | 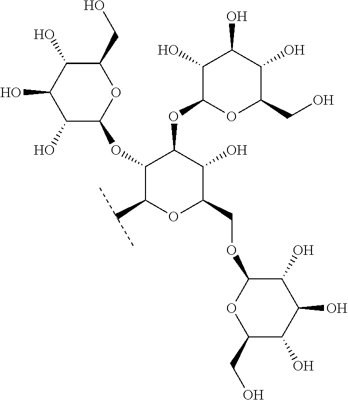 |
| XIV | 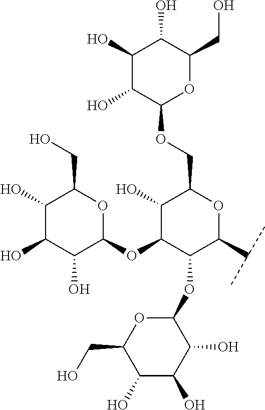 | 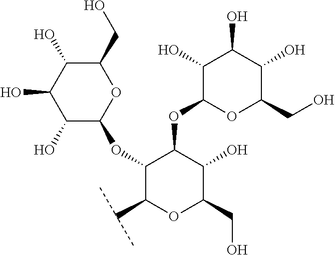 |
| XV | 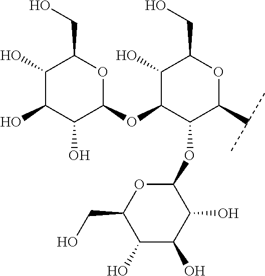 | 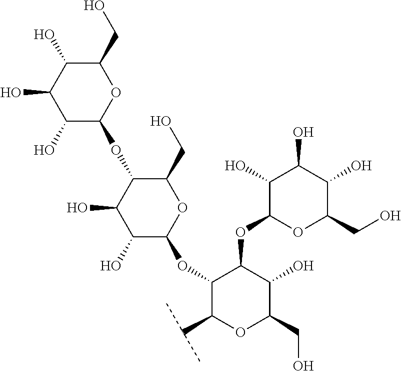 |
| XVI | 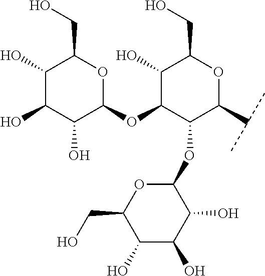 | 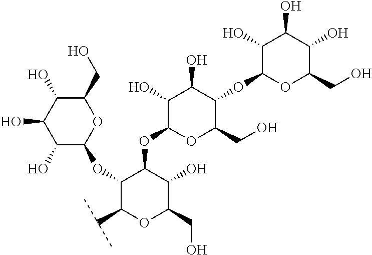 |
| XVII | 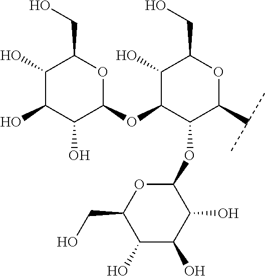 | 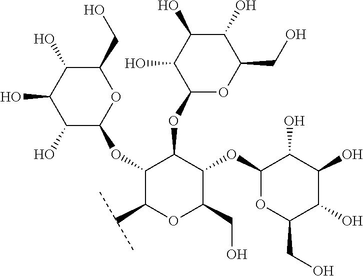 |
| XVIII | 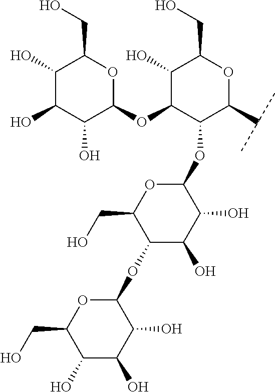 | 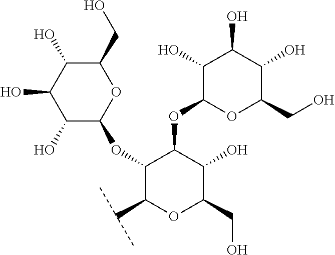 |
| XIX | 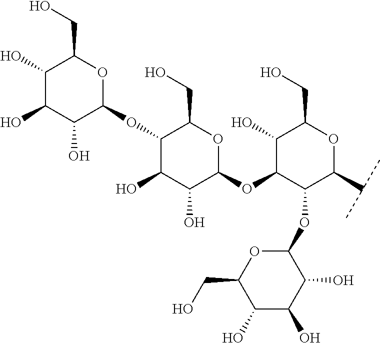 | 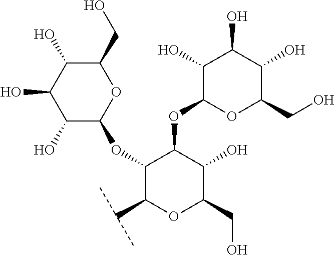 |
| XX | 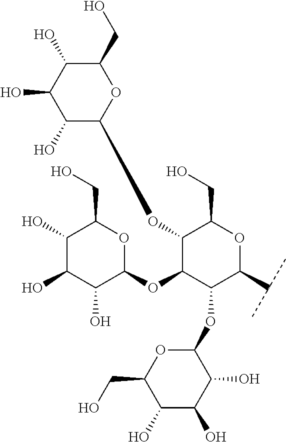 | 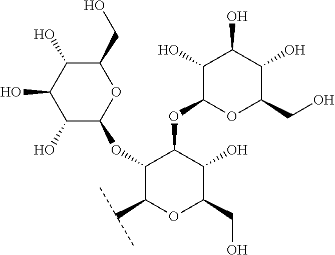 |
| XXI | 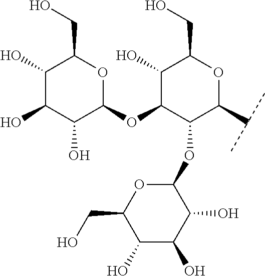 | 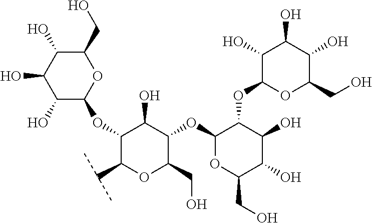 |
| XXII | 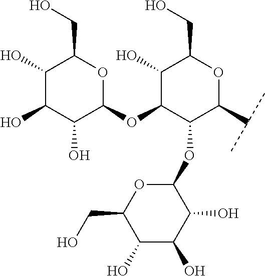 | 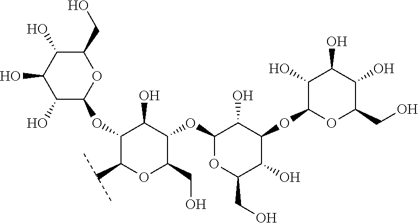 |
| XXIII | 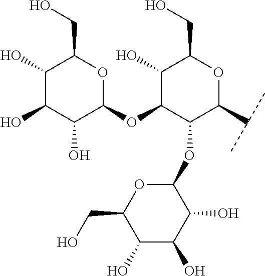 | 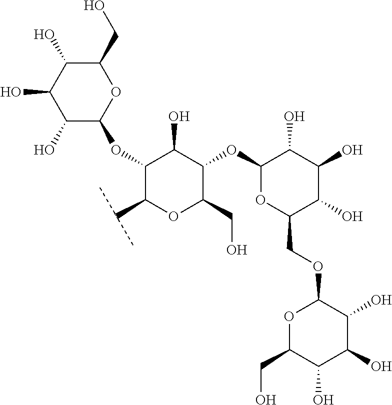 |
| XXIV | 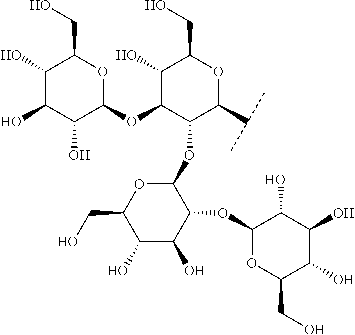 | 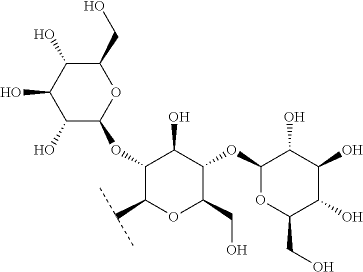 |
| XXV | 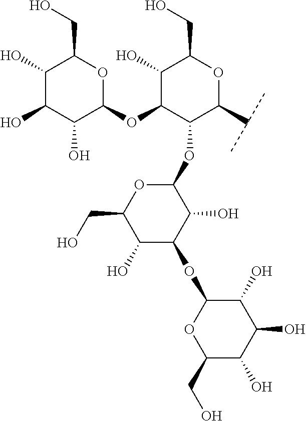 | 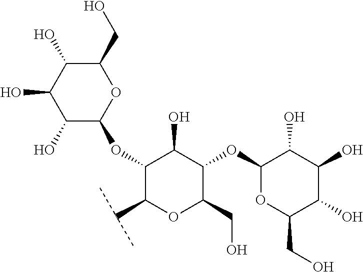 |
| XXVI | 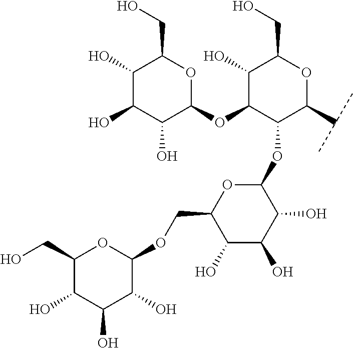 | 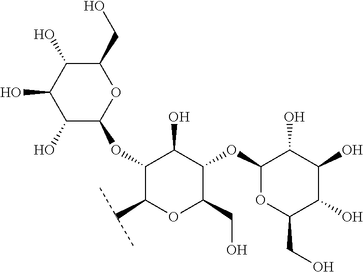 |
| XXVII | 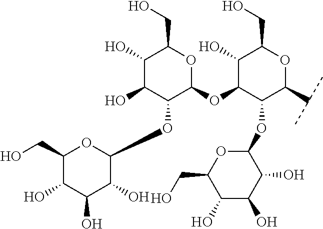 | 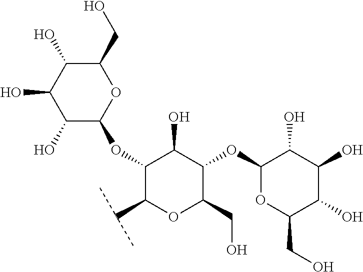 |
| XXVIII | 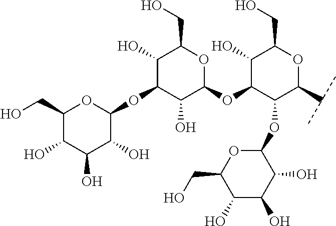 | 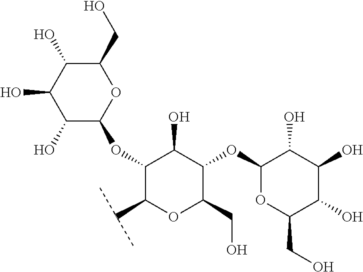 |
| XXIX | 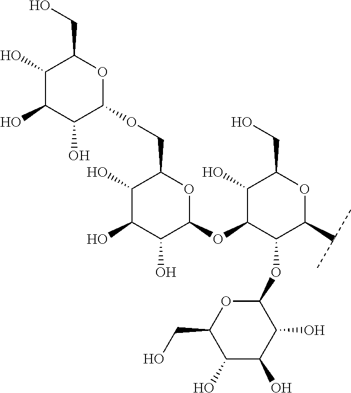 | 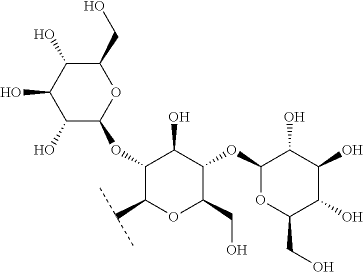 |
| XXX | 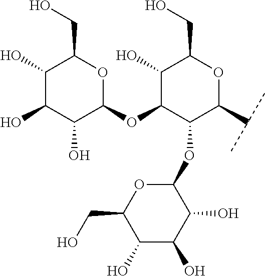 | 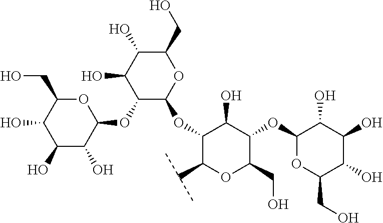 |
| XXXI | 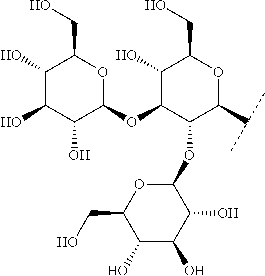 | 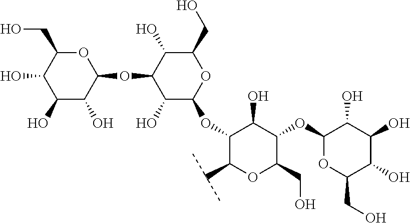 |
| XXXII | 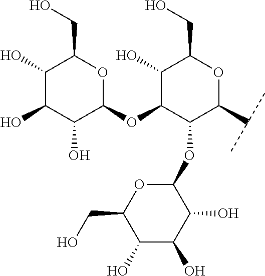 | 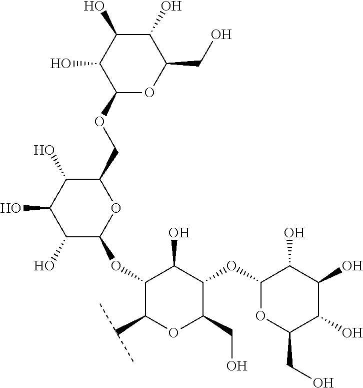 |
| XXXIII | 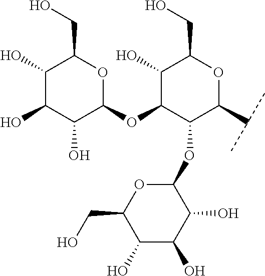 | 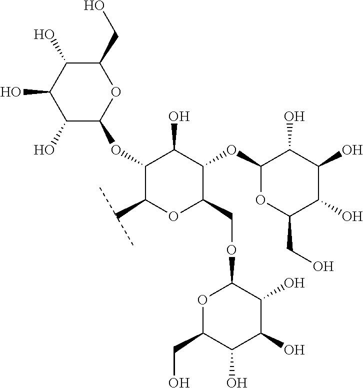 |
| XXXIV | 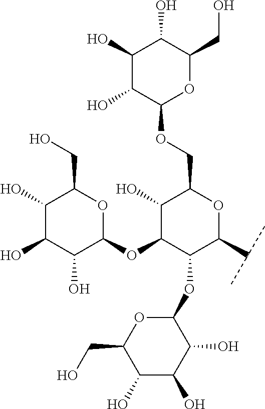 | 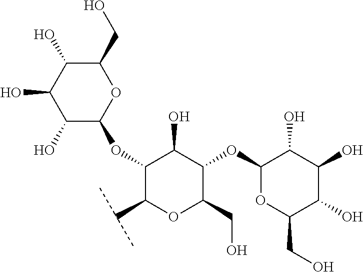 |
| XXXV | 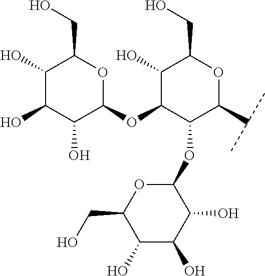 | 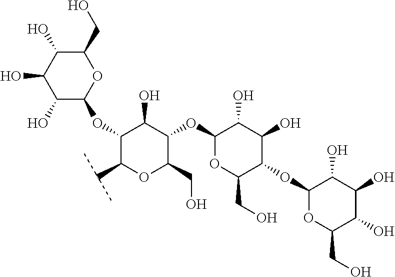 |
| XXXVI | 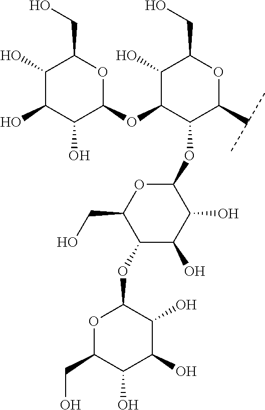 | 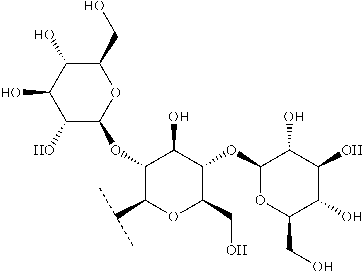 |
| XXXVII | 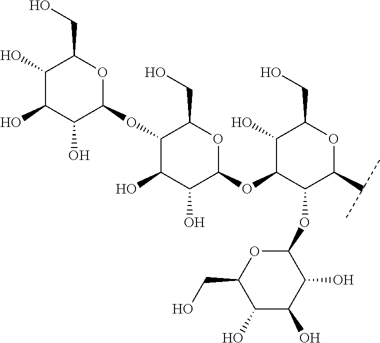 | 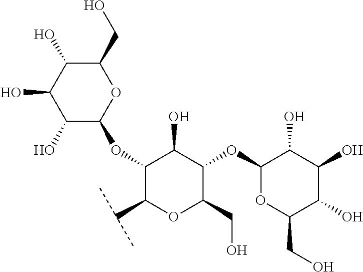 |
| XXXVIII | 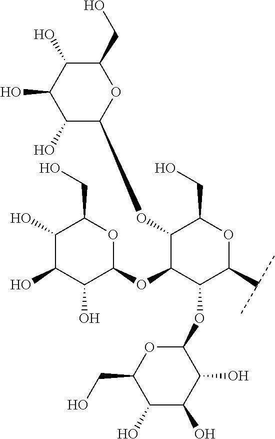 | 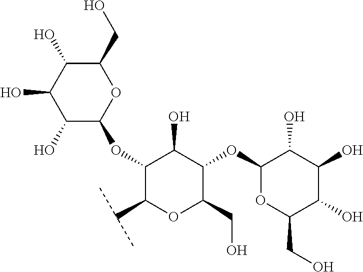 |
| XXXIX | 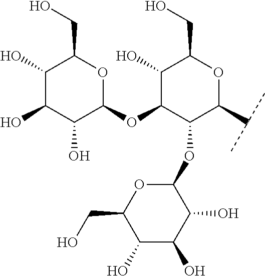 | 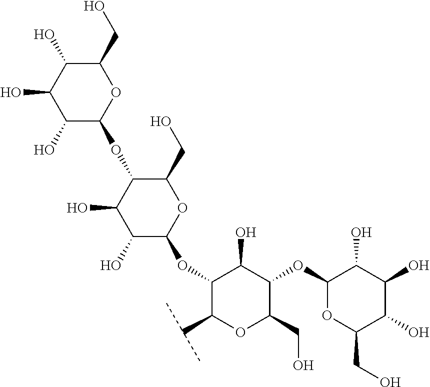 |
| XL | 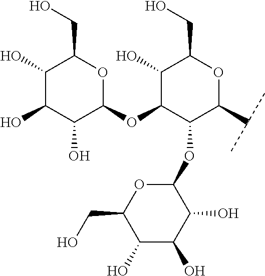 | 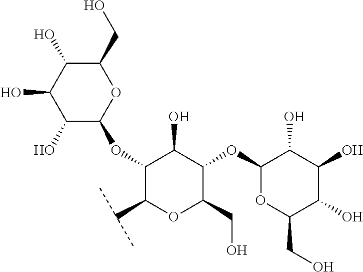 |

2. A method for producing at least one steviol glycoside of claim 1, comprising
a. providing a starting composition comprising an organic compound with at least one carbon atom;
b. providing an enzyme preparation or microorganism containing at least one enzyme selected from steviol biosynthesis enzymes, NDP-glucosyltransferases and optionally NDP-glucose recycling enzymes;
c. contacting the enzyme preparation or microorganism with a medium containing the starting composition to produce a medium comprising at least one steviol glycoside of claim 1.

3. A method for producing at least one steviol glycoside of claim 1, comprising the steps of:
a. providing a starting composition comprising an organic compound with at least one carbon atom;
b. providing a biocatalyst comprising at least one enzyme selected from steviol biosynthesis enzymes, NDP-glucosyltransferases and optionally NDP-glucose recycling enzymes;
c. contacting the biocatalyst with a medium containing the starting composition to produce a medium comprising at least one steviol glycoside of claim 1.

4. The method of claim 2 further comprising the step of:
d. separating at least one steviol glycoside from the medium to provide a highly purified composition of at least one steviol glycoside.

5. The method of claim 2, wherein the starting composition is selected from the group consisting of steviol, steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, other steviol glycosides, polyols, carbohydrates, and combinations thereof.

6. The method of claim 2, wherein the microorganism is selected from the group consisting of E. coli, Saccharomyces sp., Aspergillus sp., Pichia sp., Bacillus sp., and Yarrowia sp.

7. The method of claim 3, wherein the biocatalyst is an enzyme, or a cell comprising one or more enzyme, capable of converting the starting composition to at least one steviol glycoside.

8. The method of claim 2, wherein the enzyme is selected from the group consisting of a mevalonate (MVA) pathway enzyme, a 2-C-methyl-D-erythritol-4-phosphate pathway (MEP/DOXP) enzyme, geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase, UGT74G1, UGT85C2, UGT91D2, EUGT11, UGTS12, UGT76G1, UGlyT91C1 or mutant variant thereof having >85% amino-acid sequence identity, >86% amino-acid sequence identity, >87% amino-acid sequence identity, >88% amino-acid sequence identity, >89% amino-acid sequence identity, >90% amino-acid sequence identity, >91% amino-acid sequence identity, >92% amino-acid sequence identity, >93% amino-acid sequence identity, >94% amino-acid sequence identity, >95% amino-acid sequence identity, >96% amino-acid sequence identity, >97% amino-acid sequence identity, >98% amino-acid sequence identity, >99% amino-acid sequence identity.

9. The method of claim 4, wherein the content of at least one steviol glycoside in the highly purified composition of at least one steviol glycoside is greater than about 95% by weight on a dry basis.

10. A consumable product comprising at least one steviol glycoside of claim 1, wherein the product is selected from the group consisting of a food, a beverage, a pharmaceutical composition, a tobacco product, a nutraceutical composition, an oral hygiene composition, and a cosmetic composition.

11. The consumable product of claim 10, wherein the product is selected from the group consisting of beverages; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco and medicinal products.

12. The consumable product of claim 10, further comprising at least one additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, flavonoids, alcohols, polymers and combinations thereof.

13. The consumable product of claim 10, further comprising at least one functional ingredient selected from the group consisting of saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

14. The consumable product of claim 10, further comprising a compound selected from the group consisting of steviolmonoside, steviolmonoside A, steviolbioside, steviolbioside A, steviolbioside B, steviolbioside C, steviolbioside D, steviolbioside E, rubusoside, dulcoside A, dulcoside B, dulcoside C, dulcoside D, stevioside, stevioside A, stevioside B, stevioside C, stevioside D, stevioside E, stevioside E2, stevioside F, stevioside G, stevioside H, rebaudioside A, rebaudioside A2, rebaudioside A3, rebaudioside A4, rebaudioside B, rebaudioside B2, rebaudioside C, rebaudioside C2, rebaudioside C3, rebaudioside C4, rebaudioside C5, rebaudioside C6, rebaudioside D, rebaudioside D2, rebaudioside D3, rebaudioside D4, rebaudioside D5, rebaudioside D6, rebaudioside D7, rebaudioside D8, rebaudioside E, rebaudioside E2, rebaudioside E3, rebaudioside E4, rebaudioside E5, rebaudioside E6, rebaudioside E7, rebaudioside F, rebaudioside F2, rebaudioside F3, rebaudioside G, rebaudioside H, rebaudioside H2, rebaudioside H3, rebaudioside H4, rebaudioside H5, rebaudioside H6, rebaudioside I, rebaudioside 12, rebaudioside 13, rebaudioside J, rebaudioside K, rebaudioside K2, rebaudioside KA, rebaudioside L, rebaudioside M, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4, rebaudioside Q, rebaudioside Q2, rebaudioside Q3, rebaudioside R, rebaudioside S, rebaudioside T, rebaudioside T, rebaudioside U, rebaudioside U2, rebaudioside V, rebaudioside V2, rebaudioside V3, rebaudioside W, rebaudioside W2, rebaudioside W3, rebaudioside Y, rebaudioside Z1, rebaudioside Z2, rebaudioside AM, SvG7, NSF-02, Mogroside V, siratose, Luo Han Guo, allulose, D-allose, D-tagatose, erythritol, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, siamenoside, sucralose, potassium acesulfame, aspartame, alitame, saccharin, cyclamate, neotame, dulcin, suosan advantame, gymnemic acid, hodulcin, ziziphin, lactisole, glutamate, aspartic acid, glycine, alanine, threonine, proline, serine, lysine, tryptophan, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols, sugar alcohols, L-sugars, L-sorbose, L-arabinose, trehalose, galactose, rhamnose, various cyclodextrins, cyclic oligosaccharides, various types of maltodextrins, dextran, sucrose, glucose, ribulose, fructose, threose, xylose, lyxose, altrose, mannose, idose, lactose, maltose, invert sugar, isotrehalose, neotrehalose, isomaltulose, erythrose, deoxyribose, gulose, talose, erythrulose, xylulose, cellobiose, amylopectin, glucosamine, mannosamine, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, isomerized liquid sugars such as high fructose corn syrups, coupling sugars, soybean oligosaccharides, D-psicose, D-ribose, L-glucose, L-fucose, D-turanose, D-leucrose.

15. A method for enhancing the sweetness of a beverage or food product, comprising a sweetener comprising:
a. providing a beverage or food product comprising a sweetener; and
b. adding a sweetness enhancer comprising at least one steviol glycoside of claim 1, wherein at least one steviol glycoside of claim 1 is present in a concentration at or below the sweetness recognition threshold.

16. A method for modifying the flavor of a beverage or food product, comprising
a. providing a beverage or food product, and
b. adding a composition comprising at least one steviol glycoside of claim 1.

17. A method for suppressing foaming of a beverage or food product, comprising
a. providing a beverage or a food product, and
b. adding a foam suppressor comprising at least one steviol glycoside of claim 1.


SEQUENCE LISTING (3 sequences)

SEQ ID NO: 1 - protein, 808 aa, Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLDFLRLHSHQGKNLMLSEKIQNLNTLQHTLRKAEEYLAELKSETLYEEFEAKFEEIGLERGWGDNAERVLDMIRLLLDLLEAPDPSTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENDEHLCVLKDKKKPILFTMARLDRVKNLSGLVEWYGKNTRLRELVNLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADLLADFFTKCKEDPSHWDEISKGGLQRIEEKYTWQIYSQRLLTLTGVYGFWKHVSNLDRLEHRRYLEMFYALKYRPLAQAVPLAQDD

SEQ ID NO: 2 - protein, 442 aa, Solanum lycopersicum
MATNLRVLMFPWLAYGHISPFLNIAKQLADRGFLIYLCSTRINLESIIKKIPEKYADSIHLIELQLPELPELPPHYHTTNGLPPHLNPTLHKALKMSKPNFSRILQNLKPDLLIYDVLQPWAEHVANEQGIPAGKLLVSCAAVFSYFFSFRKNPGVEFPFPAIHLPEVEKVKIREILAKEPEEGGRLDEGNKQMMLMCTSRTIEAKYIDYCTELCNWKVVPVGPPFQDLITNDADNKELIDWLGTKPENSTVFVSFGSEYFLSKEDMEEIAFALEASNVNFIWVVRFPKGEERNLEDALPEGFLERIGERGRVLDKFAPQPRILNHPSTGGFISHCGWNSVMESIDFGVPIIAMPIHNDQPINAKLMVELGVAVEIVRDDDGKIHRGEIAEALKSVVTGETGEILRAKVREISKNLKSIRDEEMDAVAEELIQLCRNSNKSK

SEQ ID NO: 3 - protein, 458 aa, Stevia rebaudiana
MENKTETTVRRRRRIILFPVPFQGHINPILQLANVLYSKGFAITILHTNFNKPKTSNYPHFTFRFILDNDPQDERISNLPTHGPLAGMRIPIINEHGADELRRELELLMLASEEDEEVSCLITDALWYFAQDVADSLNLRRLVLMTSSLFNFHAHVSLPQFDELGYLDPDDKTRLEEQASGFPMLKVKDIKSAYSNWQIGKEILGKMIKQTKASSGVIWNSFKELEESELETVIREIPAPSFLIPLPKHLTASSSSLLDHDRTVFEWLDQQAPSSVLYVSFGSTSEVDEKDFLEIARGLVDSGQSFLWVVRPGFVKGSTWVEPLPDGFLGERGKIVKWVPQQEVLAHPAIGAFWTHSGWNSTLESVCEGVPMIFSSFGGDQPLNARYMSDVLRVGVYLENGWERGEVVNAIRRVMVDEEGEYIRQNARVLKQKADVSLMKGGSSYESLESLVSYISSL